(19) United States
(12) Patent Application Publication
(10) Pub. No.: US 2026/0024670 A1
(43) Pub. Date: Jan. 22, 2026

(54) METHOD OF IDENTIFYING A CAUSAL RELATIONSHIP

(71) Applicant: City University of Hong Kong, Hong Kong (CN)

(72) Inventors: Kei Hang Katie CHAN, Hong Kong (CN); Qian HE, Hong Kong (CN); Jundong LIU, Hong Kong (CN)

(21) Appl. No.: 19/270,740

(22) Filed: Jul. 16, 2025

Related U.S. Application Data
(60) Provisional application No. 63/671,855, filed on Jul. 16, 2024.

(51) Int. Cl. G16H 70/40; G16B 5/00; G16B 25/10; G16B 40/20

(52) U.S. Cl. CPC G16H 70/40; G16B 5/00; G16B 25/10; G16B 40/20

(57) ABSTRACT

A method of identifying a causal relationship between a drug and an unrelated disease, the method comprising: obtaining genetic instruments for therapeutic targets of the drug; identifying genes (or alleles, or variants thereof) associated with the genetic instruments; obtaining effect sizes of genetic instruments for targets associated with the unrelated disease; identifying genes (or alleles, or variants thereof) associated with the genetic instruments; harmonising the genes obtained previously and the genes identified previously; identifying overlapping genes in the harmonised genes obtained previously; and performing two-sample Mendelian randomization using the overlapping genes identified in step e), wherein the results obtained from the two-sample Mendelian randomization identify the causal relationship, if present.

### CROSS-REFERENCE TO RELATED APPLICATIONS

This application claims the benefit of priority of U.S. provisional application No. 63/671,855, filed 16 Jul. 2024, the contents of it being hereby incorporated by reference in its entirety for all purposes.

### TECHNICAL INVENTION

The present invention relates generally to the field of bioinformatics. In particular, the present invention relates to the use of Mendelian randomization in the identification of causal relationships.

### BACKGROUND

Cancer is emerging as a leading cause of death in diabetes, which was associated with an up to two-fold increased risk of all-site cancer, except for prostate cancer. The relationship between diabetes and cancer is complex. A joint consensus statement of the American Diabetes Association and the American Cancer Society indicated that it is unclear whether such associations are direct (for example, due to hyperglycemia), indirect (for example, due to diabetes as a marker of underlying biologic factors such as insulin resistance or hyperinsulinemia that alter the risk of cancer), or due to shared risk factors (for example, obesity) or a combination of these.

Anti-diabetic drugs are the most used drugs among 347 million individuals diagnosed with diabetes globally. There had been reports associating anti-diabetic drugs with increased risk of cancer, but these had been largely confounded by the increased risk of cancer in people with diabetes and obesity which frequently coexist.

The contradictory cancer risks associated with use of various anti-diabetic drugs can be attributed, in part, to multiple biases inherent in pharmacoepidemiologic analyses.

Thus, there is an unmet need for a method for identifying causal relationships between a drug used for one disease and a different disease.

### SUMMARY

In one aspect, the present disclosure refers to a method of identifying a causal relationship between a drug and an unrelated disease, the method comprising: a) obtaining genetic instruments for therapeutic targets of the drug; b) identifying genes (or alleles, or variants thereof) associated with the genetic instruments obtained from step a); c) obtaining effect sizes of genetic instruments for targets associated with the unrelated disease; d) identifying genes (or alleles, or variants thereof) associated with the genetic instruments obtained from step c); e) harmonising the genes obtained from step b) and the genes identified from step d); f) identifying overlapping genes in the harmonised genes obtained in step e); and g) performing two-sample Mendelian randomization using the overlapping genes identified in step e), wherein the results obtained from the two-sample Mendelian randomization identify the causal relationship, if present; wherein the drug is a specific drug of a group of structurally or functionally related drugs.

### BRIEF DESCRIPTION OF THE DRAWINGS

The invention will be better understood with reference to the detailed description when considered in conjunction with the non-limiting examples and the accompanying drawings, in which:

FIG. 1 shows the flow chart for the whole study. Step 1. During this stage, two approaches were employed to construct genetic instruments that could serve as proxies for drug targets and facilitate subsequent analytical steps. In the “GTEx Instrument” approach, variants associated with drug targets using data from the GTEx.v8 project were identified. Single nucleotide polymorphisms (SNPs) with the lowest nominal p-values across all tissues were selected as the genetic instruments for further testing of the Mendelian randomization (MR) analysis. In the “GWAS Instruments” approach, instrumental variables (IVs) were chosen for drug targets. This involved utilizing PLINK (an open-source toolset for performing genome-wide association studies (GWAS) and population genetics analyses) to construct instruments by identifying SNPs associated with T2D in the DIAGRAM dataset. Only SNPs that reached genome-wide significance (P<5×10⁻⁸) and were located within the gene encoding each respective drug target or within a range of about 200 kb were included in the analysis. Step 2. In this stage, drug target Mendelian randomization (MR) analyses was conducted for five specific drug targets. Subsequently, the focus was on statistically robust results from the drug target MR analyses and proceeded with PPI-based (protein-protein interaction) MR analyses and all targets-based MR analyses based on the drug target MR analysis results. Step 3 involved conducting colocalization analysis and mediation analysis to assess the MR assumptions. These analyses were performed to ensure the validity of the MR results. In the final stage (Step 4), differential expression analysis (DEGs) and predictive analyses were conducted to evaluate the expression patterns of KCNJ11, PPARG, and potential drug targets in both cancer and normal tissues. Additionally, the causal association between KCNJ11 and PPARG was investigated, accounting for confounding factors.

FIG. 2 shows the results of Mendelian randomization associations of drug targets with 40 cancer risks. The adoption of statistical methods in this study depends on the number of instruments available. If the total number of instruments is greater than two, the IVW random effects model will be employed. On the other hand, if the number of instruments is less than two, the Wald ratio MR method will be employed. (A) shows a heat map of drug target MR analysis to estimate the causal association between drug target and cancer risk (SNPs included in the MR analysis were selected by “GTEx instruments” approach). (B) shows a heat map of drug target MR analysis to estimate the causal association between drug target and cancer risk (SNPs included in the MR analysis were selected by “GTEx instruments” approach).

FIG. 3 shows forest plots of MR causal estimates for drug target on cancer risks using IVW random effects model. (A) shows a forest plot of drug target MR analysis used to estimate the causal association between drug target and cancer risk (SNPs included in the MR analysis were selected by “GTEx instruments” approach). (B) shows a forest plot of drug target MR analysis used to estimate the causal association between drug target and cancer risk (SNPs included in the MR analysis were selected by “GWAS instruments” approach). Dark pink colour represents a strong association (P<0.00125 [0.05/40]). Light pink colour represents a weak association (0.00125<P<0.05). The point size represents the number of SNPs included in the MR analyses. The squares represent causal estimates, and the error bars represent 95% CI. OR odds ratio, CI confidence interval, IVs instrumental variables, nSNP number of single nucleotide polymorphisms, IVW inverse variance weighted, GC gastric cancer, OC oropharynx cancer. (C) shows a forest plot of drug target MR analysis used to estimate the causal association between KCNJ11 and GC risk using 12 MR methods (SNPs included in the MR analysis were selected by “GTEx instruments” approach). (D) shows a forest plot of drug target MR analysis used to estimate the causal association between PPARG and OC risk using 11 MR methods (SNPs included in the MR analysis were selected by “GTEx instruments” approach). Dark colour represents a strong association (P<0.00125 [0.05/40]). Light colour represents a weak association (0.00125<P<0.05). Grey colour represents insignificant association. The squares represent causal estimates, and the error bars represent 95% CI. OR odds ratio, CI confidence interval, IVs instrumental variables, nSNP number of single nucleotide polymorphisms, IVW inverse variance weighted, GC gastric cancer, OC oropharynx cancer.

FIG. 4 shows forest plots of MR causal estimates for all targets on GC and OC risk using up to 10 MR methods. (A) shows aforest plot of drug target MR analysis used to estimate the causal association between all targets and GC risk using 12 MR methods (SNPs included in the MR analysis were selected by “GTEx instruments” approach). (B) Forest plot of drug target MR analysis to estimate the causal association between all targets and OC risk using 11 MR methods (SNPs included in the MR analysis were selected by “GTEx instruments” approach). Dark colour represents a strong association (P<0.00125 [0.05/40]). Light shading represents a weak association (0.00125<P<0.05). Grey colour represents insignificant association. The squares represent causal estimates, and the error bars represent 95% CI. OR odds ratio, CI confidence interval, IVs instrumental variables, nSNP number of single nucleotide polymorphisms, IVW inverse variance weighted, GC gastric cancer, OC oropharynx cancer. (C) shows a forest plot of PPI-based MR analysis used to estimate the causal association between KCNJ11 PPI-based genes and GC risk using 12 MR methods (SNPs included in the MR analysis were selected by “GTEx instruments” approach). (D) shows a forest plot of PPI-based MR analysis used to estimate the causal association between PPARG PPI-based genes and OC risk using 11 MR methods (SNPs included in the MR analysis were selected by “GTEx instruments” approach). Dark shading represents a strong association (P<0.00125 [0.05/40]). Light colour represents a weak association (0.00125<P<0.05). Grey colour represents insignificant association. The squares represent causal estimates, and the error bars represent 95% CI. OR odds ratio, CI confidence interval, IVs instrumental variables, nSNP number of single nucleotide polymorphisms, IVW inverse variance weighted, GC gastric cancer, OC oropharynx cancer.

FIG. 5 shows results of a mediation analysis of the causal effect of genetically proxied activation of KCNJ11 on GC via potential mediators. (A) is a schematic showing the framework for sensitivity analysis. In step 1, IVs were used for genetically proxied activation of KCNJ11 to estimate its causal effect on potential mediators (BMI, HbA1c, fasting glucose, fasting insulin, HDL-C, and LDL-C); in step 2, IVs were used for potential mediators to estimate the causal effect of potential mediators on GC. The term “Direct effect” refers to the effect of genetically proxied expression of KCNJ11 on gastric cancer after adjusting for the mediators. The term “Indirect effect” refers to the effect of genetically proxied expression of KCNJ11 on gastric cancer via the mediator, namely the mediating effect. (B) shows the MR results for the association between genetically proxied KCNJ11 and potential mediators. (C) shows the multivariable MR results for the association between LDL-C and GC risk adjusting BMI, HbA1c, fasting glucose, fasting insulin, and HDL-C. In B and C, the squares represent causal estimates (IVW OR for binary outcomes, IVW Beta for continuous outcomes), and the error bars represent 95% CI. CI confidence interval, IVs instrumental variables, SNP single nucleotide polymorphisms, IVW inverse variance weighted, BMI body mass index, HDL-C High density lipoprotein cholesterol, LDL-C low density lipoprotein cholesterol, GC gastric cancer.

FIG. 6 shows the differentially expressed PPI based genes of KCNJ11 and PPARG. (A) to (B) show the heatmaps of differentially expressed KCNJ11-PPI based genes in the training (38 gastric cancer samples and 38 adjacent normal samples, GSE13911) and (B) validation dataset (96 gastric cancer samples and 12 adjacent non-tumor pair-wised samples, GSE26899). (C) to (D) show AUC graphs demonstrating the classification performance of the KCNJ11-PPI based gene model in the training (GSE13911, as shown in (C)) and validation (GSE26899, as shown in (D)) datasets. (E) to (F) show heatmaps of differentially expressed PPARG-PPI based genes in the training (17 oral squamous cell carcinoma samples and 5 normal tissues from oral cavity, GSE23558, as shown in (E)) and validation datasets (40 male oral squamous cell carcinoma samples and 40 non-tumor pair-wised samples, GSE37991, as shown in (F)). (G) to (H) show AUC graphs demonstrating the classification performance of the PPARG-PPI based genes in the training (GSE23558; (G)) and validation datasets (GSE37991(H)). (I) to (J) show volcano plots of differentially expressed KCNJ11-PPI based genes in training (GSE13911, as shown in (I)) and validation (GSE26899, as shown in (J)) datasets. (M) to (N) show volcano plots of differentially expressed PPARG-PPI based genes in training (GSE23558, as shown in (M)) and validation (GSE37991, as shown in (N)) datasets. (K) to (L) show box plots depicting the expression levels of differentially expressed PPI based genes of KCNJ11 in training (as shown in (K)) and validation (as shown in (L)) datasets. (O) to (P) show box plots depicting the expression levels of differentially expressed PPI based genes of PPARG in training (as shown in (O)) and validation (as shown in (P)) datasets. The volcano plots depict log 2-fold change on the x-axis and False Discovery Rate adjusted p value (q-value) on the y-axis. Single genes are depicted as dots. The volcano plots indicated that transcript levels of 3 KCNJ11-PPI based genes (KCNJ11, ABCC8, and KCNQ1) had higher than 2-fold change in the gastric cancer tissue (Padj<0.05). Three genes (KCNJ11, ABCC8, and KCNQ1) were down-regulated in both training and validation datasets. The transcript levels of 7 PPARG-PPI based genes (PPARG, VEGFA, HDAC5, HSD11B1, AGTR1, TNFRSF1A and TGFB1) had higher than 2-fold change in the cancer tissue (Padj<0.05). Two genes (PPARG and AGTR1) were down-regulated and 3 genes (TNFRSF1A, VEGFA and TGFB1) were up-regulated in both training and validation datasets. The box plots represent the maximum, the 75th percentile, the median, the 25th percentile and the minimum value of data.

FIG. 7 shows a diagram of instrument selection of anti-diabetic drug targets. The selected genetic predictors for the anti-diabetic drug targets were selected based on “GTEx Instruments” approach and listed in Table 2 and a further raw data set (data not shown).

FIG. 8 shows Results of Mendelian randomization associations of 13 anti-diabetic drug targets with cancer risks. The adoption of statistical methods in this study depends on the number of instruments available. If the total number of instruments is greater than two, the IVW random effects model will be employed. On the other hand, if the number of instruments is less than two, the Wald ratio MR method will be used.

FIG. 9 shows a heatmap showing the genetically-proxied gene expression changes in T2D susceptibility for increased KCNJ11 and PPARG expression in 49 GTEx tissues. Statistically significant (P<0.05/(14569*49)).

FIG. 10 shows a heatmap of the results of Mendelian randomization associations of 9 anti-diabetic drug classes with cancer risks. The adoption of statistical methods in this study depends on the number of instruments available. If the total number of instruments is greater than two, the IVW random effects model will be employed. On the other hand, if the number of instruments is less than two, the Wald ratio MR method will be used.

FIG. 11 shows the results of Mendelian randomization associations of KCNJ11 and ABCC8 with cancer risks. The adoption of statistical methods in this study depends on the number of instruments available. If the total number of instruments is greater than two, the IVW random effects model will be employed. On the other hand, if the number of instruments is less than two, the Wald ratio MR method will be used.

FIG. 12 shows a leave-one-out plot for a MR test sensitivity analysis for KCNJ11 (A) and PPARG (B).

FIG. 13 shows forest plots of MR causal estimates for KCNJ11 and PPARG on GC and OC risk using up to 10 MR methods in BMI adjusted population. (A) shows a forest plot of drug target MR analysis to estimate the causal association between KCNJ11 and GC risk using 11 MR methods (SNPs included in the MR analysis were selected by “GTEx instruments” approach) in BMI adjusted population. (B) shows a forest plot of drug target MR analysis to estimate the causal association between PPARG and OC risk using 11 MR methods (SNPs included in the MR analysis were selected by “GTEx instruments” approach in BMI adjusted population. Dark shading represents a strong association (P<0.00125 [0.05/40]). Light colour represents a weak association (0.00125<P<0.05). Grey colour represents insignificant association. The squares represent causal estimates, and the error bars represent 95% CI. OR odds ratio, CI confidence interval, IVs instrumental variables, nSNP number of single nucleotide polymorphisms, IVW inverse variance weighted, GC gastric cancer, OC oropharynx cancer.

FIG. 14 shows forest plots of MR causal estimates for KCNJ11 on GC risk using up to 10 MR methods in East Asian population. In this case, a forest plot of drug target MR analysis to estimate the causal association between KCNJ11 and GC risk using 11 MR methods (SNPs included in the MR analysis were selected by “GTEx instruments” approach) in BMI adjusted population. Dark shading represents a strong association (P<0.00125 [0.05/40]). Light shading represents a week association (0.00125<P<0.05). Grey represents insignificant association. The squares represent causal estimates, and the error bars represent 95% CI. OR odds ratio, CI confidence interval, IVs instrumental variables, nSNP number of single nucleotide polymorphisms, IVW inverse variance weighted, GC gastric cancer.

FIG. 15 shows forest plots of MR causal estimates for anti-diabetic drug targets on pan cancer risk.

FIG. 16 shows the results of the identification of modules linked to gastric cancer by WGCNA and pathway enrichment analysis of top 100 genes by Enrichr. Cluster dendrogram of co-expressed genes in GC. (A) displays a cluster dendrogram representing the hierarchical clustering of co-expressed genes in GC. It provides insights into the relationships and patterns of gene expression within the GC dataset. Module-trait relationships of GC risk and 9 modules. (B) shows a heatmap diagram where each row represents a module, while each column represents risk of GC. The heatmap allows for the identification of modules that exhibit strong associations with GC. Scatter plot of gene significance (GS) score for GC vs. module membership (MM) score in turquoise module. (C) shows a scatterplot, where the GS score reflects the association between a gene and GC risk, while the MM score quantifies how well a gene fits into the module. This plot helps identify highly significant genes within the module and assess their membership strength. Pathway enrichment analysis of top 100 genes which have highest GS score, and MM score clustered in turquoise module. (D) shows a bar graph with the results of examination of the enrichment of biological pathways associated with these genes using the KEGG database. This analysis provides insights into the potential biological mechanisms and pathways related to the genes within the turquoise module. GC gastric cancer.

FIG. 17 shows a graphic abstract and summary of the method described herein.

FIG. 18 shows a line graph showing the cumulative incidence of cancer in the population with different risk factors from a published study.

FIG. 19 shows a diagram of the relationship between decreased levels of insulin and cancer risk from a published study.

FIG. 20 shows a schematic outlining the three core, instrumental variable assumptions underlying Mendelian randomization, which have to be considered true (i.e., valid): 1. The genetic variant(s) being used as an instrument for the exposure is associated with the exposure. This is known as the “relevance” assumption. 2. There are no common causes (i.e. confounders) of the genetic variant(s) and the outcome of interest. This is known as the “independence” or “exchangeability” assumption. 3. There is no independent pathway between the genetic variant(s) and the outcome other than through the exposure. This is known as the “exclusion restriction” or “no horizontal pleiotropy” assumption.

FIG. 21 shows an example of the presentation of harmonised data.

FIG. 22 shows another example of the presentation of harmonised data.

FIG. 23 shows an exemplary table showing data related to an “exposure dataset”.

FIG. 24 shows an exemplary table showing data related to an “outcome dataset”.

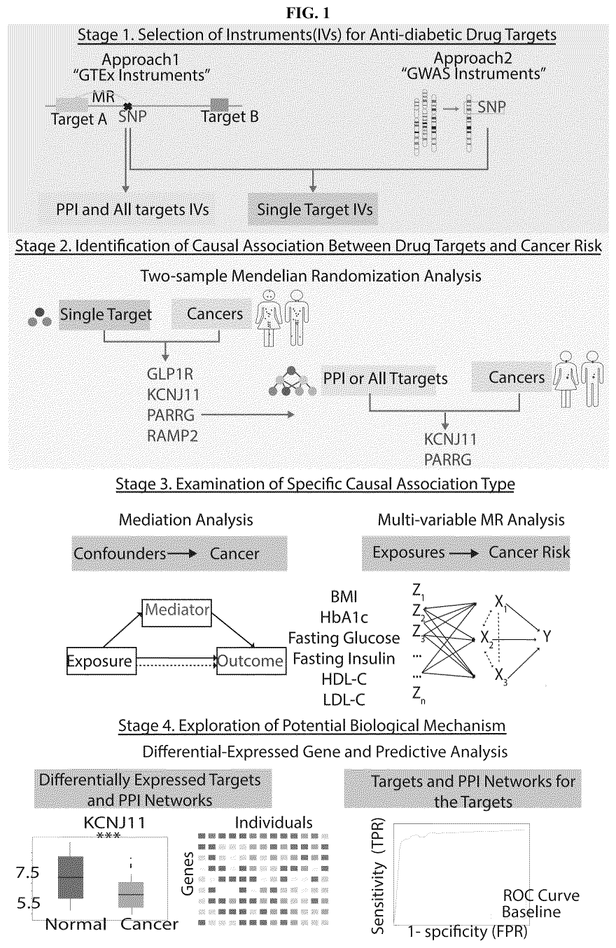

### DEFINITIONS

As used herein, the term “genetic instrument” refers to a genetic variant, or multiple genetic variants, that is used as a proxy to determine the causal relationship between modifiable exposures (like to proxy and health outcomes). One non-limiting example of a modifiable exposure is the use of a drug or compound to treat a disease that it was not intended to be treated (also referred to herein as an “unrelated” disease, in respect of the intended treatment use of the drug or compound). For example, the use of anti-diabetic drugs in the treatment of cancer (an unrelated disease). To be considered a valid genetic instrument, the genetic variant should satisfy at least the following criteria: 1. The genetic variant should be robustly associated with the exposure of interest. 2. The genetic variant must not be associated with confounders that affect both the exposure and the outcome. 3. The genetic variant should influence the outcome only through its effect on the exposure, not via other pathways (also referred to as the exclusion-restriction criterion).

Examples of a genetic instrument can be, but are not limited to, one or more single nucleotide polymorphisms (SNPs), protein levels, physiological features (for example, but not limited to, blood pressure, temperature, body mass index (BMI)), as well as social or socio-economical features (such as, but not limited to, education level, income, working stability, living area).

As used herein, the term “unrelated disease” refers to a disease that is not treated by the drug in question. For example, in the context of the present disclosure, if the drug is an anti-diabetic drug, the “unrelated disease” is cancer.

As used herein, the term “drug” refers to any substance that affects the structure or functioning of a living organism. Drugs are widely used for the prevention, diagnosis, treatment of diseases, and for the relief of symptoms.

As used herein, the term “genetically-proxied activation” refers to the process of activating certain biological functions, for example, drug target expression, through the manipulation of its genetic makeup. This can involve, for example, but is not limited to, altering specific genes to induce a desired biomedical mechanism change in the human.

As used herein, the term “effect size” refers to a value that measures the strength of the relationship between two variables on a numeric scale. In other words, an effect size indicates how meaningful the relationship between variables or the difference between two groups is and is independent of the sample size. An effect size therefore indicates the practical significance of a specific outcome or finding. For example, for two independent groups, effect size can be measured as the difference between two means divided by a standard deviation for the data. This is also known as Cohen's d, the equation for which is shown here:

$$d = \frac{{\overline{x}}_{1} - {\overline{x}}_{2}}{s}$$

whereby d is Cohen's d, x₁ is the mean of group, x₂ is the mean of group 2, and s is the standard deviation.

Exemplary methods of determining/calculating effect sizes between groups are, but are not limited to odds ratio (OR), relative risk. or risk ration (RR). Exemplary methods of determining/calculating effect sizes as measures of association are, but are not limited to, Pearson's r correlation and r2 coefficient of determination, Cohen's d, and Hedges' d.

As used herein, the term “data harmonisation” refers to one or more procedures used in statistics with the aim of achieving, or at least improving, the comparability of data obtained from different surveys and previously performed measurements. Harmonisation can refer to input harmonisation or output harmonisation. Input harmonisation is a situation where the data is harmonised through standardisation of definitions, indicators, classifications, training, and technical requirements prior to the analysis being performed). In contrast, output harmonisation is the situation where previously obtained data (for example, using non-standardised methods) is harmonised by mapping the data to a unified measurement scheme. Harmonisation is different from standardisation, whereby the former involves a reduction in variation of standards, while the latter entails moving towards the eradication of any variation with the adoption of a single standard. In one example, data harmonisation includes steps of, for example, matching genetic instruments, extracting effect sizes, and aligning the direction of previously identified alleles with exposure or outcome datasets, thereby allowing for a valid Mendelian randomization analysis.

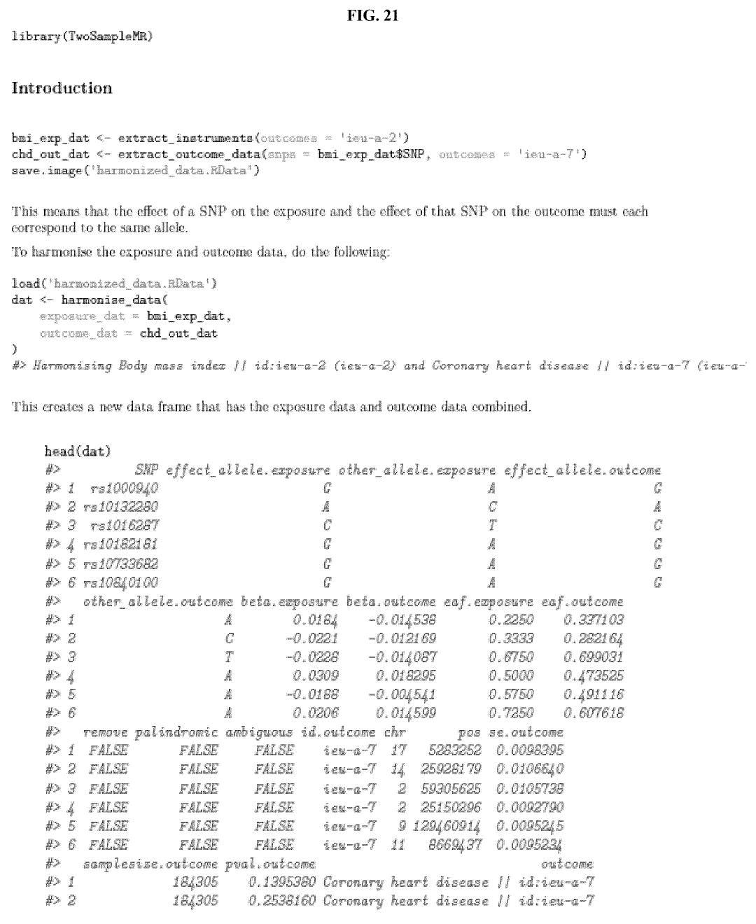

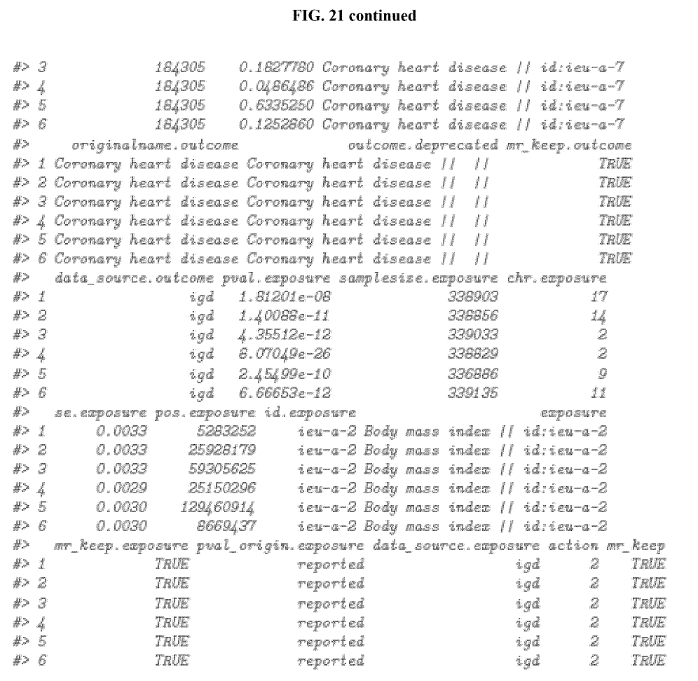

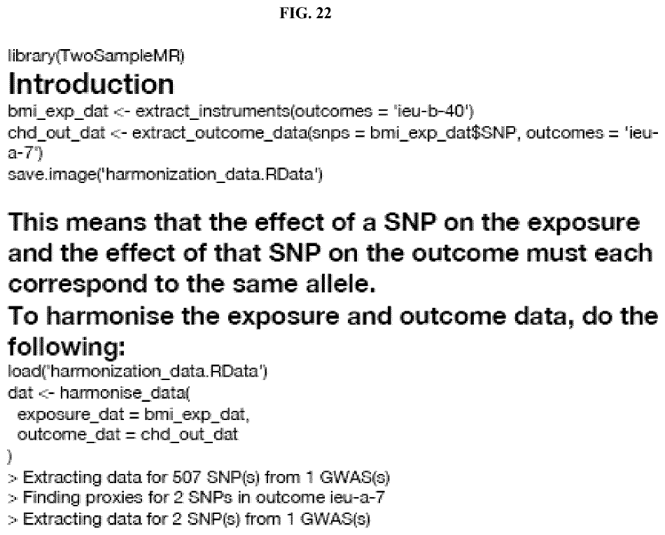

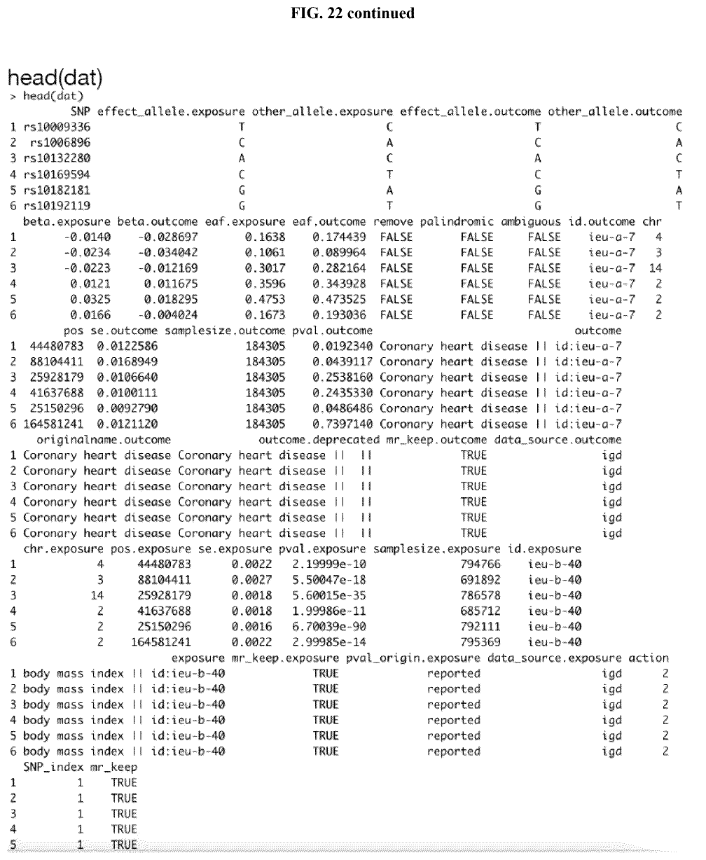

An example of the presentation of harmonised data is provided in FIGS. 21 and 22. The raw data used to obtain these examples is shown in Tables 33 and 34 below.

As used herein, the term “valid” refers to how likely a specific feature is to correspond accurately to the same scenario in a real-world application. This can also be referred to as statistical conclusion validity, which indicates which conclusions about a relationship between variables based on the data obtained are correct.

As used herein, the term “Mendelian randomization” (also abbreviated herein as MR) refers to a method that uses measured variation in genes to examine a causal effect of an exposure on an outcome. In summary, Mendelian randomization (MR) is fundamentally an instrumental variants estimation method. The method uses the properties of germline genetic variation (usually in the form of single nucleotide polymorphisms or SNPs) strongly associated with a putative exposure as a “proxy” or “instrument” (also referred to as a “genetic instrument”) for that exposure to test for and estimate a causal effect of the exposure on an outcome of interest from observational data. The genetic variation used will have either well-understood effects on exposure patterns (for example, propensity to smoke heavily) or effects that mimic those produced by modifiable exposures (for example, raised blood cholesterol). Importantly, the genotype must only affect the disease status indirectly via its effect on the exposure of interest.

As genotypes are assigned randomly when passed from parents to offspring during meiosis, then groups of individuals defined by genetic variation associated with an exposure at a population level should be largely unrelated to the confounding factors that typically plague observational epidemiology studies. Germline genetic variation (that is, a variation which can be inherited) is also temporarily fixed at conception and is not modified by the onset of any outcome or disease, thereby precluding reverse causation. Additionally, given the advancement in methodology, measurement error and systematic misclassification is often low with genetic data. In this regard, Mendelian randomization can be thought of as analogous to “nature's randomized controlled trial”.

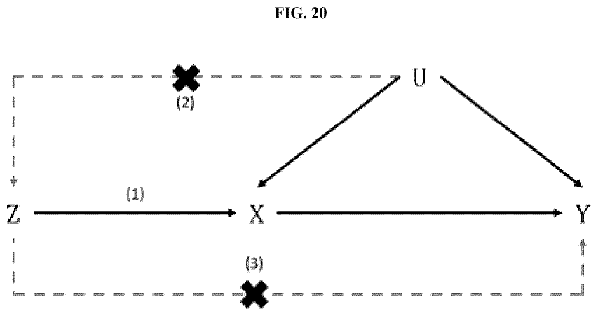

Mendelian randomization requires three core, instrumental variable assumptions to be considered true (i.e., valid): 1. The genetic variant(s) being used as an instrument for the exposure is associated with the exposure. This is known as the “relevance” assumption. 2. There are no common causes (i.e., confounders) of the genetic variant(s) and the outcome of interest. This is known as the “independence” or “exchangeability” assumption. 3. There is no independent pathway between the genetic variant(s) and the outcome other than through the exposure. This is known as the “exclusion restriction” or “no horizontal pleiotropy” assumption. A schematic outlining this concept underlying Mendelian randomization is provided in FIG. 20.

To ensure that the first core assumption is validated, Mendelian randomization requires distinct associations between genetic variation and exposures of interest. These are usually obtained from genome-wide association studies (GWAS) but can also be obtained from candidate gene studies. The second assumption relies on there being no population substructure (for example, but not limited to, geographical factors that induce an association between the genotype and outcome), mate choice that is not associated with genotype (i.e., random mating or panmixia) and no dynastic effects (i.e., the situation where the expression of parental genotype in the parental phenotype directly affects the offspring phenotype). A Mendelian randomization design, using certain assumptions, has been shown to reduce both reverse causation and confounding, both of which can often impede or mislead interpretation of results from, for example, epidemiological studies.

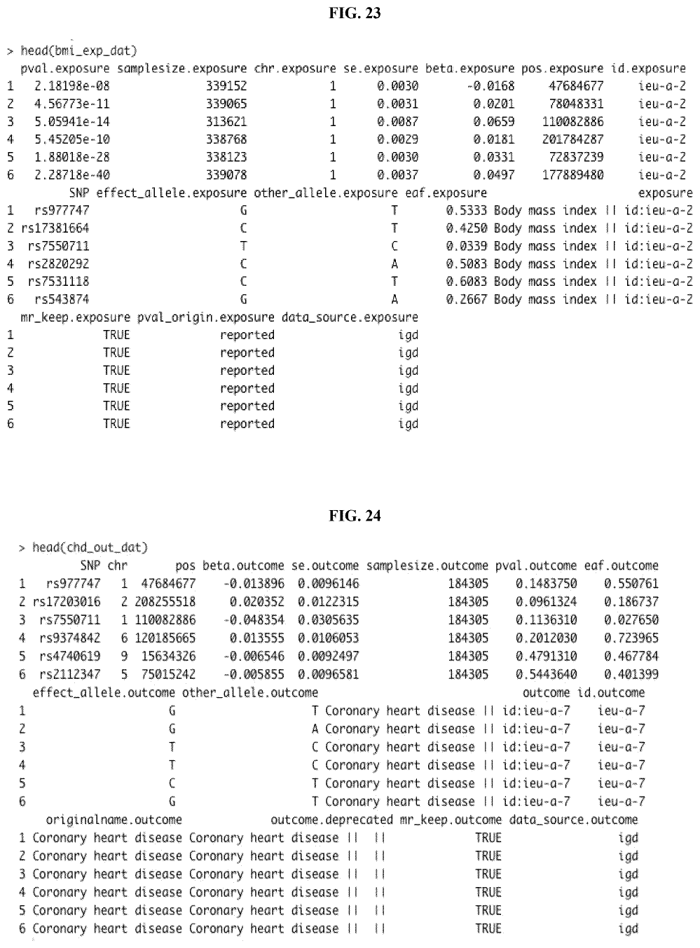

As used herein, the term “exposure dataset” refers to a dataset containing information related to the exposure variable of interest, such as, but not limited to, genetic variants or markers. In the exposure dataset, each row represents a unique genetic variant or marker. The columns of the dataset include details such as, but not limited to, variant ID, allele coding, effect sizes (e.g., beta coefficients or odds ratios), standard errors, p-values, and other relevant statistical data. Additional columns may also be present in the dataset containing information on allele frequencies, sample sizes, or any other pertinent variables deemed to be important to, or associated with, the exposure. An exemplary table showing data related to an “exposure dataset” is provided in FIG. 23. Briefly, from the dataset shows in FIG. 23, information of IVs (pval.exposure, samplesize.exposure, chr.exposure, se.exposure, beta.exposure, pos.exposure, id.exposure, SNP, effect_allele.exposure, other_allele.exposure, eaf.exposure, exposure, pval_origin.exposure, and data_source.exposure) and the remainder can be identified in performing the claimed Mendelian randomization analysis (mr_keep.exposure).

As used herein, the term “outcome dataset” refers to a dataset containing data relevant to the outcome variable being studied. Similar to the exposure dataset as outlined above, each row in the outcome dataset represents, but is not limited to, a distinct genetic variant or marker. The columns of the outcome dataset include information such as, but not limited to, variant ID, allele coding, effect sizes, standard errors, p-values, and other relevant statistics. Also similar to the exposure dataset, additional columns may be included, providing information on sample sizes, allele frequencies, or any other relevant variables associated with the outcome. An exemplary table of an outcome dataset is provided in FIG. 24, wherein the outcome under analysis is that of coronary heart disease.

### DETAILED DESCRIPTION

Understanding the effect of anti-diabetic drugs on cancer risk allows clinicians to make informed decisions when prescribing these medications for treating cancer, for example, given the close associations between glycaemic control and cancer development or progression. To date, no large randomization trials have examined the effects of anti-diabetic drugs on the risk of cancer in patients with type 2 diabetes, for example.

Given the possible causal relationship between hyperglycemia and diabetes and cancer, the aim of the present disclosure was to ascertain as to what, if any, potential anti-diabetic drugs have in reducing the risk of cancer. Without being bound by theory, it was thought that this could be achieved by improving the metabolic milieu or modulating pathways implicated in both hyperglycemia and cancer.

Observational studies suggested that use of some classes of anti-diabetic medications, including metformin, sulfonylureas (SU), and thiazolidinediones (TZD) were associated with reduced risk of cancer. Notably, metformin, an insulin sensitizer widely used as the first-line therapy for type-2 diabetes (T2D), has been found to reduce cell proliferation, induce apoptosis, and cause cell cycle arrest. For example, thiazolidinediones are selective agonists of the peroxisome proliferator-activated receptor gamma (PPARG) with insulin-sensitizing actions increased cellular differentiation, reduced cellular proliferation, and induced apoptosis in certain cell lines. In vitro studies also reported potential antiproliferative effects of glucagon-like peptide-1 receptor agonists (GLP1RA) on various cancer cell types.

Taken together, without being bound by theory, it is thought that certain anti-diabetic drugs, such as metformin, sulfonylureas, thiazolidinediones, and glucagon-like peptide-1 receptor agonists can have an effect on the prevention and treatment of cancer through reducing cell proliferation, inducing apoptosis, and promoting cellular differentiation.

Beside identifying the causal relationship between diabetes-associated pathways and cancer risk, the approach disclosed herein allows for the tailoring of treatment strategies and the identification of other therapeutic approaches for cancer management.

In the field of biomedical research, identifying causal relationships between exposures and outcomes is often crucial for developing effective therapeutic interventions. Confounding variables often pose challenges in establishing such relationships. Here, Mendelian randomization (MR) analysis, a robust analytical method, was used to investigate the presence of causal pathways and inform drug repositioning strategies.

Mendelian randomization was thus employed to investigate causal relationships between exposures and outcomes. This approach leverages genetic variants as unconfounded instrumental variants (IVs). By using germline genetic variants that are randomly assorted during meiosis, Mendelian randomization analysis mitigates the conventional issues of confounding and enhance the reliability of the findings. Mendelian randomization analysis can therefore mimic the pharmacological modulation of a drug target in clinical trials, emulates the genetically-proxied impact of anti-diabetic drugs, and has the advantage of allowing evaluation of the effects of long-term modulation of drug targets on the disease. The methodology disclosed herein has been employed to predict both clinical benefits and adverse effects of therapeutic interventions for drug repositioning based on causal pathways, whereby the method disclosed herein allows for the identification of causal associations at the genetic level and significantly enhances the reliability and validity of the results.

The instrumental variants identified herein can then be used to provide unbiased estimates of the causal effect of drug usage on specified outcomes. This is because genetic variants are randomly assigned at conception and are not influenced by factors that may confound the drug-outcome relationship.

The advantages of the Mendelian randomization analysis disclosed herein can be summarised as follows: 1. Mitigating Confounding: Mendelian randomization analysis employs germline genetic variants as instrumental variants, ensuring that exposure-outcome associations are not confounded by other factors. This eliminates biases commonly encountered in conventional observational studies. 2. Pharmacological Mimicry: Mendelian randomization analysis emulates the pharmacological modulation of drug targets in clinical trials. This enables the evaluation of long-term effects of modulating specific targets, providing insights into clinical benefits and adverse effects. 3. Informing Drug Repositioning: By elucidating causal pathways, Mendelian randomization analysis enables the anticipation of therapeutic benefits and adverse effects associated with drug repositioning strategies.

Thus, in one example, the genetic instruments from the method disclosed herein are obtained using at least two different methods. In a further example, genetic instruments from step a are obtained using at least two different methods. In another example, the genetic instruments shown to be statistically significant in both of the at least two different methods are selected. In yet another example, the at least two different methods are GTEx instruments and GWAS instruments. In a further example, the genetic instruments have a weak linkage disequilibrium with each other. In another example, the genetic instruments are obtained from a database selected from the group consisting of the GWAS Catalogue, Integrative Epidemiology Unit (IEU) Open GWAS, FinnGen Consortium, the Breast Cancer Association Consortium, and combinations thereof. In one example, the genetic instruments are obtained from DrugBank.

The method described herein has been shown to reduce the time-consuming drug development stage, while providing treatments for cancers through the application of precision medicine. By incorporating Mendelian Randomization (MR) analysis, researchers and clinicians can efficiently identify therapeutic targets, evaluate their long-term effects, and make informed decisions regarding drug repositioning, all based on causal pathways.

Mendelian Randomization analysis was performed to explore the causal association between genetically-proxied expression of single drug target and cancer risk. The impact of activating drug targets, modulated by commonly prescribed anti-diabetic drugs, including, but not limited to, biguanides (metformin), thiazolidinediones, ATP-sensitive potassium (KATP) channel blockers (for example, sulfonylureas and meglitinide analogues), dipeptidyl peptidase-4 inhibitors (DPP-4i), alpha-glucosidase inhibitor (AGI), sodium glucose cotransporter 2 inhibitors (SGLT2i), glucagon-like peptide-1 receptor agonists (GLP1RA), as well as insulin and amylin analogues, on the risk of developing 40 site-specific cancers were investigated.

The disclosed method combines, for example, genome-wide association studies (GWAS) and expression quantitative trait loci (eQTL) data with Mendelian randomization analysis to assess the effect of anti-diabetic drug classes on cancer risk. In other words, drug-target Mendelian Randomization (using genetic instruments) is used to evaluate the causal association between changes in target expression (genetically proxied) and cancer risks. Protein-protein interaction (PPI)-based Mendelian randomization (which assesses the interaction between drug targets and other proteins) and all-target-based Mendelian randomization analyses (which mimics the combined effect of multiple drugs by evaluating the impact of all drug targets collectively on a single target, for example, the collective impact of anti-diabetic drugs on cancer treatment) were employed for result validation and identification of previously unidentified drug targets. This approach allows the assessment of the combined effect of different drug classes, such as, but not limited to, statins, nonsteroidal anti-inflammatory drugs (NSAIDs), and anti-diabetic drugs, providing insights into their synergistic capabilities in, for example, cancer treatment.

PPI-based gene identification was also employed to identify protein-protein interactions associated with each anti-diabetic target. This allowed the identification of PPI-based genes that play a role in the functioning of these targets. Subsequently, the anti-diabetic and anti-cancer effects of the identified PPI-based genes were investigated by performing differential-expression genes (DEGs) analysis and co-expression network analysis.

Thus, in one example, the therapeutic target of the drug of the method disclosed herein is a drug target gene or a protein affected by the drug. In another example, the drug target gene is identified using protein-protein-interaction (PPI)-based gene identification. In yet another example, method further comprises a step of validating the results obtained therein. In one example, the validation step can be, but is not limited to, in vitro assays, in vivo assays, and in silico assays.

The method disclosed herein has applications in the fields of drug discovery, personalized medicine, and precision oncology, as well as allowing the (re-)evaluation of drug targets and their effects on, for example, diabetes and cancer risk. By combining genome-wide association studies (GWAS), expression quantitative trait loci (eQTL) data, and Mendelian randomization analysis, previously unidentified anti-cancer drug targets were identified and evaluated, their impact on cancer risk assessed, and targeted therapies developed. By identifying PPI-based genes associated with anti-diabetic targets and evaluating their effects on both diabetes and cancer, therapeutic candidates for targeted interventions were identified.

Lastly, the mechanism linking anti-diabetic drugs and cancer treatment remains unclear. To address this, mediation analysis was employed to identify mediators thought to explain the observed association. This approach allowed exploration and elucidation of the pathways or biological factors that mediate the relationship between anti-diabetic drugs and their effects on cancer treatment.

A method has been described herein that identifies drug targets and protein-protein interaction-based genes using expression quantitative trait loci (eQTL), genome-wide association studies (GWAS), and protein-protein-interaction network dataset. This method further allows for the execution of drug-target Mendelian randomization analysis, protein-protein-interaction-based Mendelian randomization analysis, and all-targets-based Mendelian randomization analysis, requiring only minor adjustments to the genome-wide association studies summary data format. Additionally, this method also outlines the step-by-step procedures for conducting differential-expression gene analysis and co-expression network analysis.

Thus, in one example, the present disclosure describes a method of identifying drug targets and assessing their impact on diabetes and cancer risk. In another example, the target associated with the unrelated disease can be, but is not limited to, a gene, a protein, a mutant gene, a mutant protein, a dysregulated gene, or a dysregulated a protein.

In another example, the present disclosure describes a method of identifying a causal relationship between a drug and an unrelated disease. In other words, the term unrelated disease, as defined above, refers to a disease that was not intended to be treated with the drug in question. In another example, the method comprises a) obtaining genetic instruments for therapeutic targets of the drug; b) identifying genes (or alleles, or variants thereof) associated with the genetic instruments obtained from step a); c) obtaining effect sizes of genetic instruments for targets associated with the unrelated disease; d) identifying genes (or alleles, or variants thereof) associated with the genetic instruments obtained from step c); e) harmonising the genes obtained from step b) and the genes identified from step d); f) identifying overlapping genes in the harmonised genes obtained in step e); and g) performing two-sample Mendelian randomisation using the overlapping genes identified in step e), wherein the results obtained from the two-sample Mendelian randomisation identify the causal relationship, if present. In another example, the drug is a specific drug of a group of structurally or functionally related drugs.

The present disclosure and the method disclosed herein uses a combination of analytical techniques, including, but not limited to, genome-wide association studies (GWAS), expression quantitative trait loci (eQTL), Mendelian randomization analysis, and protein-protein-interaction-based Mendelian randomization analysis, to provide a comprehensive evaluation of drug targets. This approach enhances the accuracy and reliability of the results, this in turn allowing the results to be applied in drug development and personalised medicine. By considering individual genetic variations and their influence on drug targets, the method described herein allows healthcare providers to tailor treatment plans to specific patients, thereby increasing the likelihood of therapeutic success.

Using the “GTEx Instruments” method as described herein, statistically significant single-nucleotide polymorphisms (SNPs) linked to 21 anti-diabetic drug targets were identified from eQTL data. These SNPs were validated as instruments imitating anti-diabetic drug effects by assessing the correlation between target expression changes and type 2 diabetes (n=251,509) using a two-sample Mendelian randomization (MR) method. All SNPs with a P value of less than 0.05 were recorded. Furthermore, a post-hoc sensitivity analysis was conducted, including SNPs in the 21 drug targets that showed a genome-wide significant association (P<5×10-8) with type 2 diabetes (“GWAS Instruments”). Subsequently, a drug-target Mendelian randomization analysis was performed to explore the causal association between genetically-proxied expression of drug targets and cancer risk. Only consistent results from these two methods were carried forward to be validated by Protein-Protein Interaction (PPI)-based Mendelian randomization and all-targets-based Mendelian randomization analyses. The roles of metabolic traits (such as, but not limited to, BMI, HbA1c, fasting glucose (FG), fasting insulin (FI), high-density lipoprotein cholesterol (HDL-C), and low-density lipoprotein cholesterol (LDL-C)) implicated in cancer risk were investigated by performing mediation and multi-variable Mendelian randomization (MVMR) analyses.

Thus, in one example, the method disclosed herein further comprises a step of validating the genetic instruments. In another example, the genetic instruments can be, but are not limited to, single nucleotide polymorphisms (SNPs), proteins, protein-protein interactions, gene expression, and protein expression.

Shown below is an exemplary workflow of the data harmonisation steps in Mendelian randomization.

The harmonisation step in the TwoSampleMR package mentioned herein refers to a process used in two-sample Mendelian randomization (MR) analysis to ensure the compatibility of genetic variants across different datasets or studies. In one example, the harmonisation data process used in such a TwoSampleMR package can comprise the following steps:
i. Data Extraction: Retrieve the relevant exposure and outcome datasets from the appropriate sources. These sources can include genome-wide association studies (GWAS), consortia databases, or publicly available repositories.
ii. Variant Matching: Identify and align the genetic variants across the exposure and outcome datasets. This step ensures that the same variants are being analysed in both datasets, allowing for a consistent comparison.
iii. Allele harmonisation: Check and harmonize the allele coding for the genetic variants in both the exposure and outcome datasets. This step ensures that the alleles are coded consistently to maintain accurate associations between the exposure and outcome.
iv. Effect Direction Alignment: Verify and align the effect directions of the genetic variants in both datasets. This step is crucial for ensuring that the genetic variants' effects on the exposure and outcome are consistent and compatible.
v. Summary Statistic harmonisation: Apply any necessary transformations or adjustments to the summary statistics, such as standardization or scaling, to ensure comparability between the exposure and outcome datasets.
vi. Quality Control: Perform quality control checks to identify and handle any outliers, missing data, or data inconsistencies. This step helps ensure the reliability and integrity of the harmonized data.
vii. Data Filtering: Apply additional filters or exclusions based on pre-defined criteria to remove any variants or data points that may introduce bias or confounding factors.
viii. Data Integration: Combine the harmonized exposure and outcome datasets into a single dataset suitable for further analysis, for example, using the TwoSampleMR package/framework. Due to the steps performed above, the resulting integrated dataset would contain the necessary information for conducting Mendelian randomization analysis.

Provided below is an exemplary write up of how to perform the method as described herein.

### 1. Two-Sample Mendelian Randomization (2SMR) Step-by-Step Implementation:

(1) Instrumental variable (IV) Selection
   Genome-wide significant SNPs (p<5×10⁻⁸) from GWAS of the exposure trait.
   Clumping (r2<0.01, 250 kb window) using 1000 Genomes reference.
(2) Harmonization
   Align effect alleles between exposure/outcome datasets.
   Exclude palindromic SNPs with intermediate allele frequencies.
(3) Effect Estimation
   Inverse-variance weighted (IVW) as primary method.
(4) Pleiotropy Assessment
   MR-Egger intercept test (p>0.05 indicates validity).
   Leave-one-out analysis.

### 2. Protein-Protein Interaction (PPI) Mendelian Randomization

### Novel Additions to 2SMR:

(1) PPI Network Integration
   Extract interacting proteins from STRING DB (confidence score>0.7);
   Apply diffusion algorithm to prioritize causal pathways.
(2) Multi-IV Construction
   Refer to the 2SMR (1).

### 3. All-Target Mendelian Randomization

### Novel Additions to 2SMR

(1) All target identification
   Collecting all the anti-diabetic drug targets, ignoring the drug class.

Thus, in one example the harmonisation comprises aligning the direction of alleles identified with an exposure dataset and aligning the direction of alleles identified with an outcome dataset. In yet another example, the harmonisation of step e) comprises aligning the direction of alleles identified in step b) with an exposure dataset and aligning the direction of alleles identified in step d) with an outcome dataset.

The method disclosed herein has the following advantageous effects. One example concerns the technical implementation of the method described herein with a customized workflow. Regarding target Identification Module, the presently described method is based on an automated R-based pipeline that integrates DrugBank through custom functions, enabling high-efficiency target identification for anti-diabetic drugs. This approach results in a reduced target extraction time from more than 12 hours (manual curation) to less than 2 hours (automated processing); achieves a 83%-time savings while maintaining 100/6 data accuracy through batch query optimization. This is illustrated in the information shown in Supplement Code 1 provided below.

Supplement Code 1

```
###
# 1. define function 1 to extract drug targets from drug-bank data
###
clean_drugbank <-function( ) {
  vocab <-read_csv(″~/drugbank_vocabulary.csv″)
vocab$substance <-paste0(vocab$‘Common name‘,″ | ″,vocab$Synonyms)
vocab <-vocab[,c(″DrugBank ID″,″substance″)]
colnames(vocab) <-c(″drugbank_id″,″substance″)
# replace | as ;
vocab$substance <-gsub(″ \\| ″,″;″,vocab$substance)
vocab <-separate_rows(vocab,substance,sep = ″;″)
# read drug active file
active <-read_csv(″drug_target_identifiers_all_pharmacologically_active_v5.1.9.csv″)
active <-active[,c(″Gene Name″,″Drug IDs″)]
colnames(active) <-c(″gene″,″drugbank_id″)
  active <-separate_rows(active, drugbank_id)
df <-merge(vocab,active,by = c(″drugbank_id″),all.x =TRUE)
df <-df[df$substance!=″NA″,]
df$substance <-tolower(df$substance)
df <-unique(df)
return(df)
}
```

The described method also allows for the development of an automated Mendelian Randomization pipeline for drug repurposing. An automated MR analysis framework was developed that systematically evaluates causal relationships between, for example,
Inputs: Genetically proxied targets of 9 anti-diabetic drugs (via DrugBank)
Outputs: Effect estimates for 40 site-specific cancers (ICD-10 classified)

This provides advantages, such as a unified workflow from target input to cancer risk estimation (as shown in Supplement Code 2 below), and eliminated manual data handling between analytical steps

Supplement Code 2

```
###
# 2. define function 2 to extract anti-diabetic drug targets
###
clean_bnf <-function( ) {
# Load BNF data =============================================
df <-read_csv(“exposure.csv”)
# Format dataframe ==========================================
df <-df[,c(“Class of antidiabetic medication (route of administration)”,“Representative agents”)]
colnames(df) <-c(“drug”,“substance”)
# Remove polypharmacy medines ================================
df <-df[!grepl(“AND”,df$drug,ignore.case =FALSE),]
# Tidy drug substance information ================================
df$drug <-tolower(df$drug)
df$substance <-tolower(df$substance)
df <-df[!is.na(df$substance),]
df <-df[!grepl(“/”,df$substance),]
df <-unique(df)
# Format drug names =========================================
df$drug <-ifelse(df$drug==“alpha glucosidase inhibitors”, “Alpha glucosidase inhibitors”,df$drug)
df$drug <-ifelse(df$drug==“amylin analog”,
  “Amylin analog”,df$drug)
df$drug <-ifelse(df$drug==“biguanide”,
  “Biguanide”,df$drug)
df$drug <-ifelse(df$drug==“dipeptidyl peptidase 4 (dpp-iv) inhibitor”,
  “Dipeptidyl peptidase 4 (dpp-iv) inhibitor”,df$drug)
df$drug <-ifelse(df$drug==“GLP-1 agonists”,
  “Glucagon-like peptide 1 agonists”,df$drug)
df$drug <-ifelse(df$drug==“meglitinides”,
  “Meglitinides”,df$drug)
df$drug <-ifelse(df$drug==“insulins”,
  “Insulins”,df$drug)
df$drug <-ifelse(df$drug==“sodium-glucose cotransporter (SGLT2)inhibitor”,
  “Sodium-glucose cotransporter (SGLT2)inhibitor”,df$drug)
df$drug <-ifelse(df$drug==“sulfonylureas”,
  “Sulfonylureas”,df$drug)
df$drug <-ifelse(df$drug==“thiazolidinediones”,
  “Thiazolidinediones”,df$drug)
return(df)
}
```

The method described herein also allowed for a population-level clinical validation. In this context, a nested case-control analysis was performed within the Women's Health Initiative (WHI) cohort, comprising 143,184 postmenopausal women. The analytical sample included 8,400 participants with prevalent diabetes at baseline, among whom 5,921 received sulfonylurea (SU) treatment targeting KCNJ11. The summarized findings so far are, pertaining to the diabetes status and gastric cancer (GC) risk, that participants with diabetes showed significantly elevated gastric cancer incidence in unadjusted models (hazard ratio=1.75, 95% CI 1.12-2.71, P=0.014). Regarding the sulfonylurea (SU) effect, a protective trend was observed among sulfonylurea users, though statistical power was limited by low case numbers (n=43 gastric cancer events) (Crude hazards ratio=0.58 (95% CI 0.24-1.43, P=0.23) More information can be found in Table 32 below.

### Primary Analysis in Cancer Endpoints

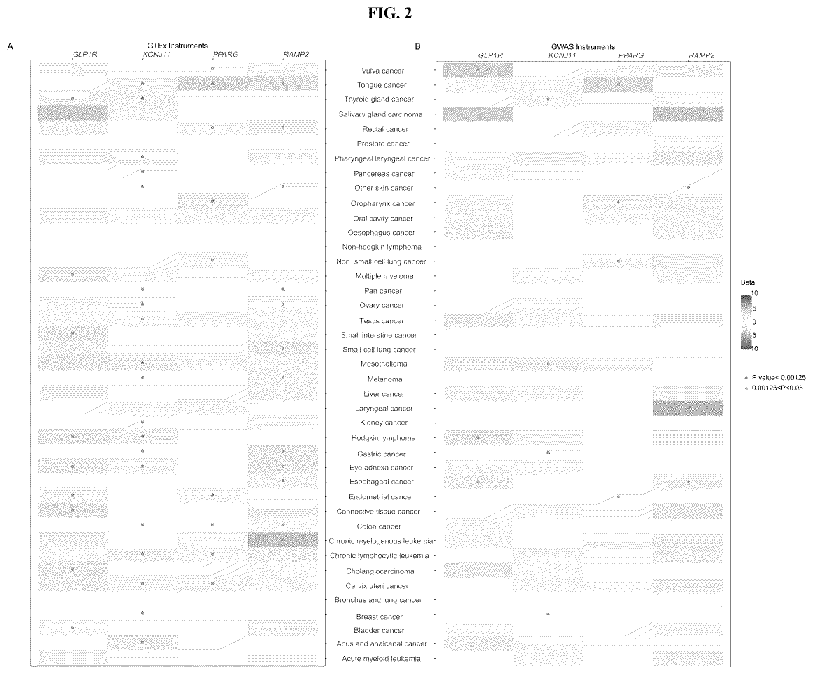

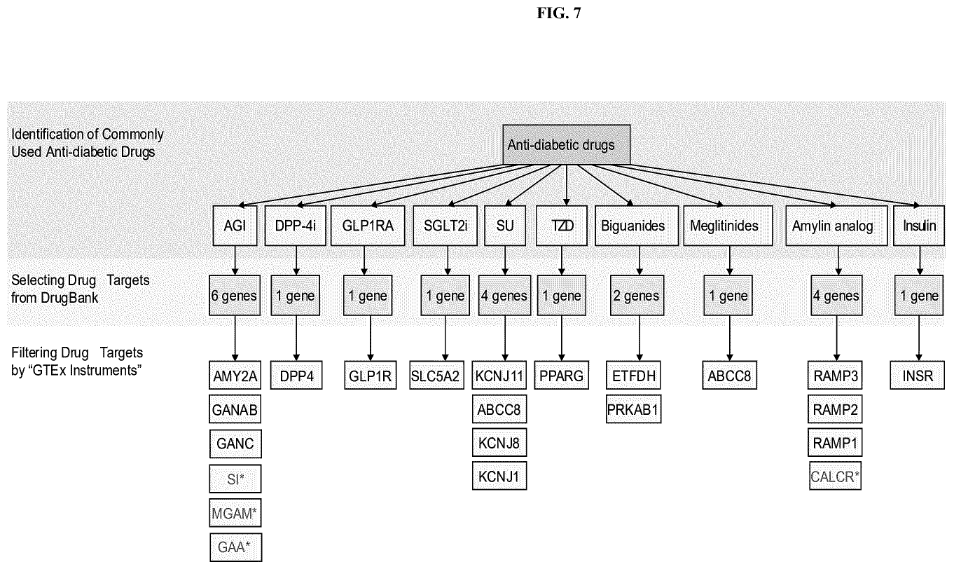

Twenty-one (21) anti-diabetic drug targets (MGAM, GANAB, AMY2A, SI, GANC, GAA, DPP4, ABCC8, KCNJ1, KCNJ8, INSR, ETFDH, PRKAB1, RAMP3, RAMP1, CALCR, KCNJ11, GLP1R, PPARG, RAMP2, and SLC5A2) were identified in DrugBank for commonly prescribed anti-diabetic drugs (refer to Table 2 and FIG. 7). Of associations between 21 targets and 40 cancers, 4 drug targets (KCNJ11, GLP1R, PPARG, and RAMP2) and 7 cancers that exhibited consistent association across both the “GTEx Instruments” (FIG. 2A) and “GWAS Instruments” approaches (FIG. 2B). Specific

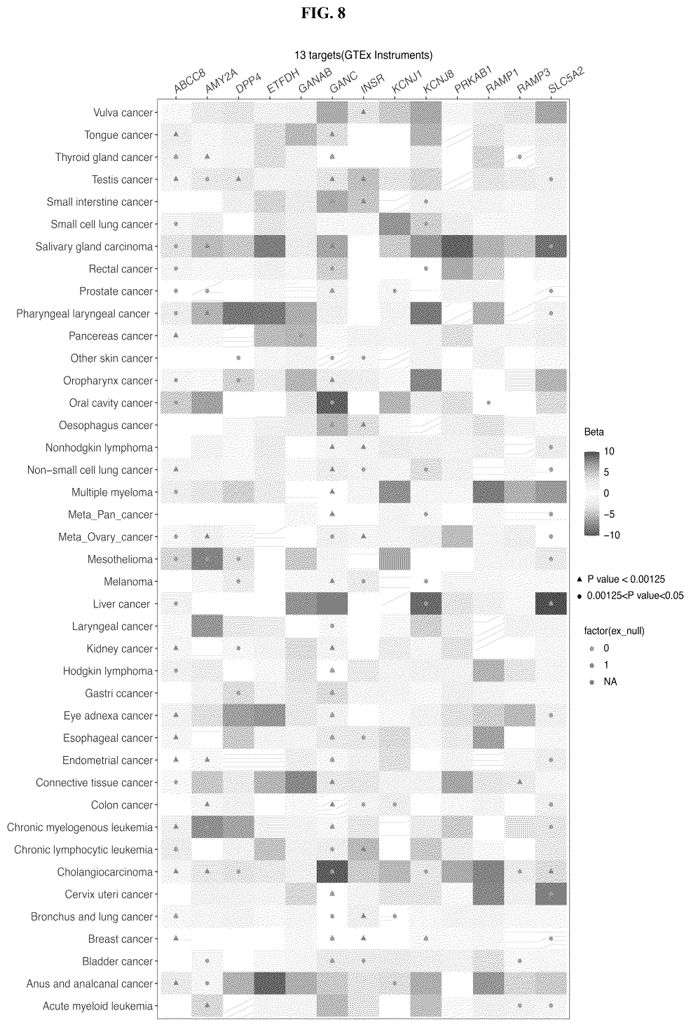

For the remaining 17 targets, the “GTEx Instruments” approach was employed to select instrumental variants (IVs), and 13 targets (ABCC8, KCNJ1, KCNJ8, GANAB, AMY2A, DPP4, GANC, ETFDH, PRKAB1, INSR, RAMP1, SLC5A2 and RAMP3) showed statistically significant associations after applying the “GTEx Instruments” approach. Subsequently, drug-target Mendelian randomization analysis was conducted to investigate the causal relationship between the 13 targets and 40 cancers (FIG. 8).

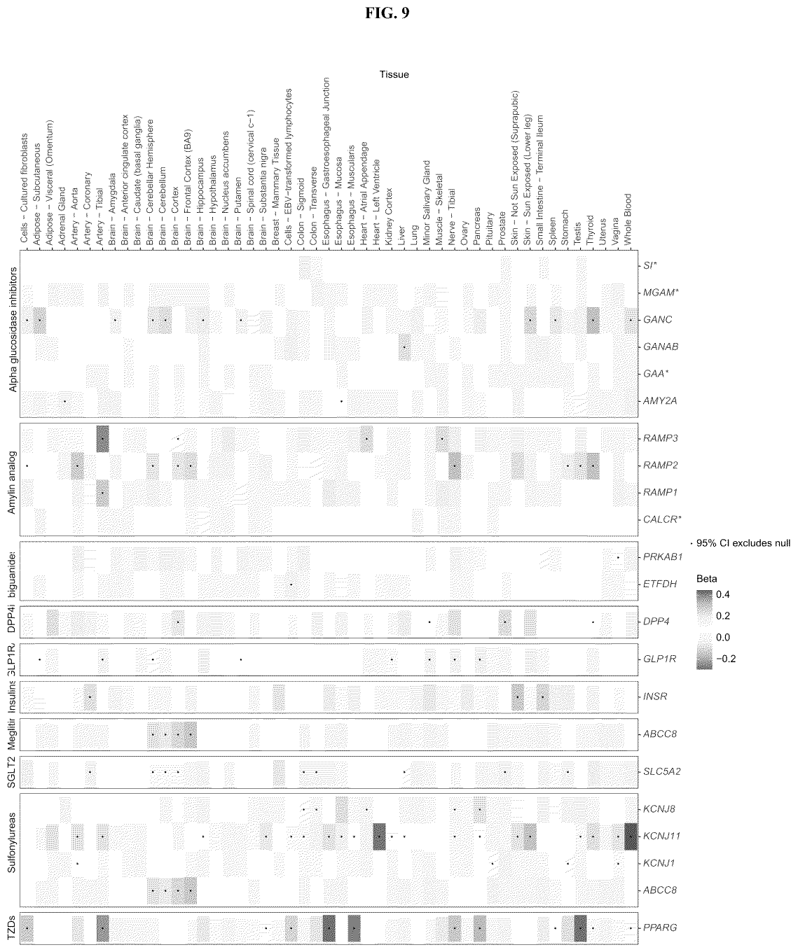

A total of 16 single nucleotide polymorphisms (SNPs) were employed in Potassium Inwardly Rectifying Channel Subfamily J Member 11 (KCNJ11) as instrumental variants to proxy the effects of sulfonylurea. Similarly, 8 SNPs in Glucagon-Like Peptide 1 Receptor (GLPIR) were utilized to proxy the effects of Glucagon-Like Peptide 1 Receptor Agonist (GLP1RA). For thiazolidinedione, 10 SNPs were employed in Peroxisome Proliferator-Activated Receptor Gamma (PPARG) as instrumental variants. Additionally, while 8 SNPs in Receptor Activity Modifying Protein 2 (RAMP2) were used to proxy the effects of amylin analogues. Further details regarding the effects of these SNPs can be found in Table 1. Heatmapping was used to explore the direction of the proxied effects of anti-diabetic drugs on targets expression (FIG. 9). This heatmap offers an overview of the directionality of the effects and provides insights into the relationship between the anti-diabetic drugs and targets expression.

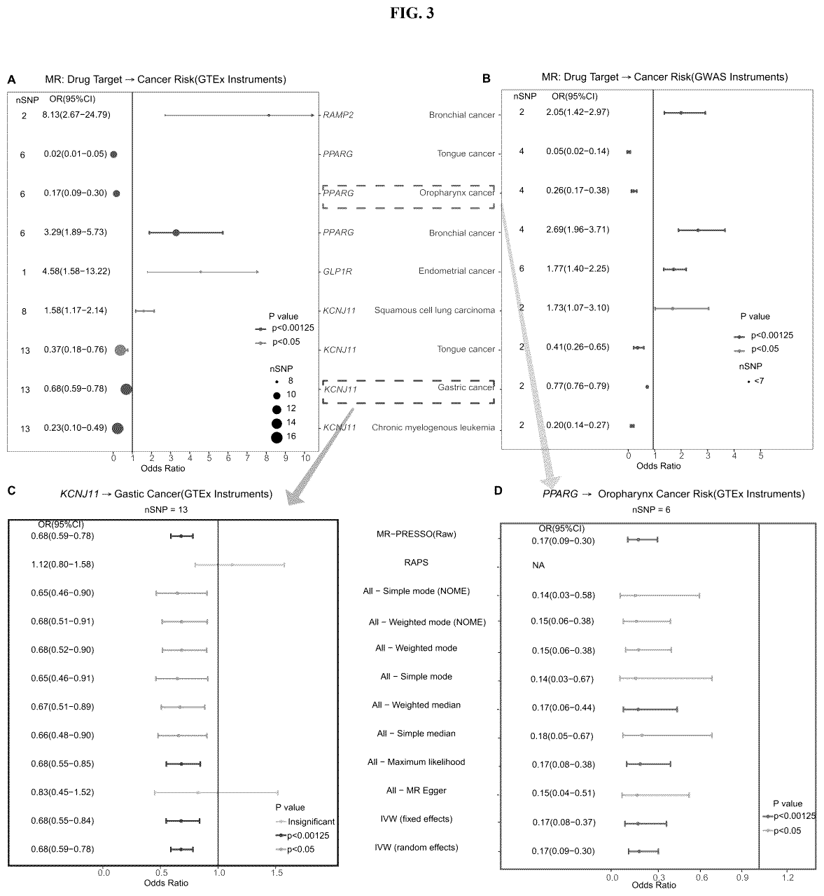

The results showed a correlation between genetically-proxied activation of KCNJ11 on reduced risk of chronic myelogenous leukemia (odds ratio inverse-variance-weighted 0.23; 95% confidence interval (CI) 0.10-0.49., P=1.82×10⁻⁴, FIG. 3A and Table 7), and gastric cancer (GC) (odds ratio inverse-variance-weighted (OR IVW) 0.68; 95% confidence interval (CI) 0.59-0.78., P=7.37×10⁻⁸, FIG. 3A and Table 7). A weak correlation was observed for genetically-activation of KCNJ11 on the decreased risk of tongue cancer (odds ratio inverse-variance-weighted 0.37; 95% confidence interval (CI) 0.18-0.76; FIG. 3A and Table 7). A correlation between genetically-proxied activation of PPARG on the decreased risk of oropharynx cancer (odds ratio inverse-variance-weighted 0.17; 95% confidence interval 0.09-0.30, P=1.94×10⁻⁹, FIG. 3A and Table 7), tongue cancer (odds ratio inverse-variance-weighted 0.02; 95% confidence interval 0.01-0.05, P=3.95×10⁻¹⁸, FIG. 3A and Table 7) and the increased risk of bronchial cancer (odds ratio inverse-variance-weighted 3.29; 95% confidence interval 1.89-5.73; P=2.68×10⁻⁵; FIG. 3A and Table 7) were shown. Similar results were observed using a standard two-sample Mendelian Randomization with IVs passing the genomic significance (P<5×10⁻⁸) (FIG. 3B and Table 8). In the analysis of genetically-proxied expression of GLP1R and RAMP2, the direction of cancer risk was with wide 95% confidence interval (CI), hence, the next analysis to explore the association between genetically-proxied protein-protein interactions and cancer risk (Table 9) was not undertaken. The results obtained from studies with limited sample sizes were consolidated, specifically those with a case sample size of less than 500 and statistical power below 80% (Table 10). In following analyses, the focus was on the causal association between genetically-proxied activation of KCNJ11 and gastric cancer, as well as the genetically-proxied activation of PPARG and oropharynx cancer.

The Mendelian randomization analyses provided insights into the genetically-proxied activation of KCNJ11 and its impact on the risk of gastric cancer. Through an array of assessments employing twelve different Mendelian randomization methods (FIG. 3C and Table 11), results were found consistently confirming that genetically-proxied activation of KCNJ11 decreased the risk of gastric cancer.

Similar confirmation was obtained for the causal association between genetically-proxied activation of PPARG and oropharynx cancer. Results from eleven distinct Mendelian randomization methods (FIG. 3D, further and Table 11) consistently indicated that the genetic activation of PPARG led to decreased risk of Mendelian randomization. This study had 80% power with a high likelihood of detecting odd ratios (ORs) of less than 0.81 in the KCNJ11 analyses and less than 0.63 in the PPARG analyses (Table 10).

### Secondary Analyses in Cancer Endpoints

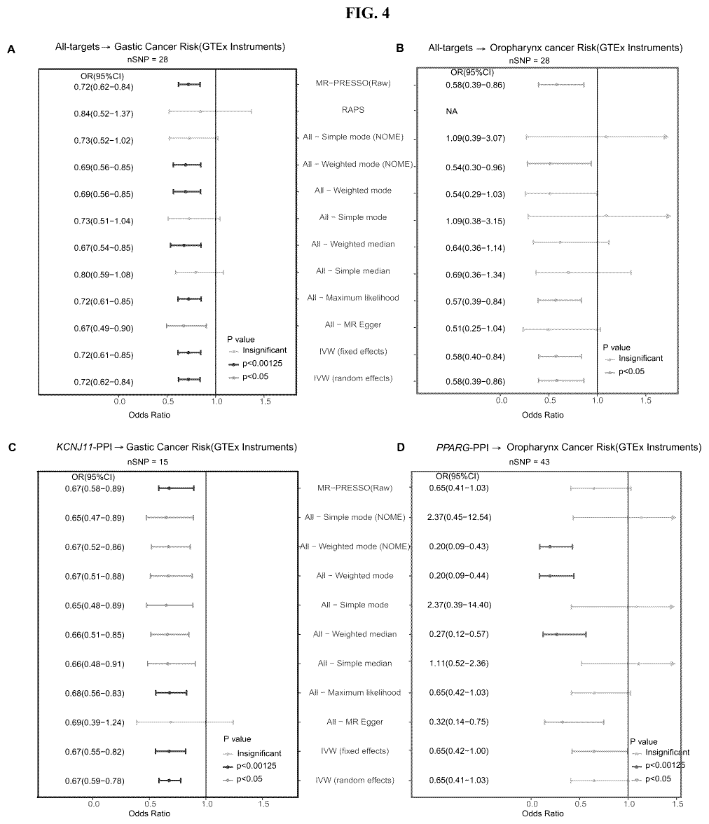

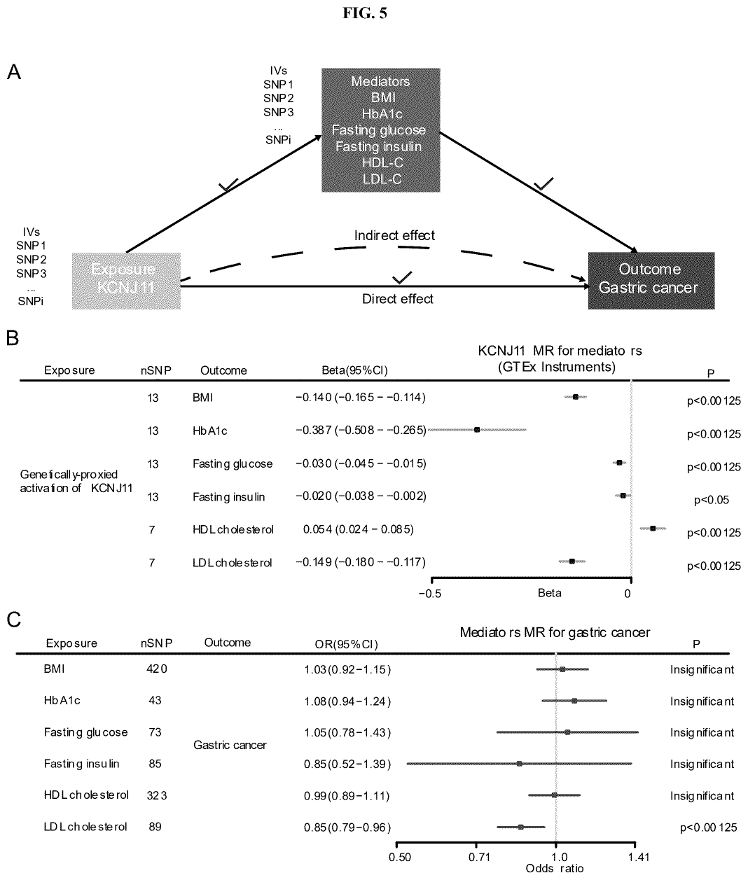

The results of combined effect of all drug targets indicated a decreased risk of gastric cancer (odds ratio inverse-variance-weighted (OR IVW) 0.72, 95% confidence interval (CI) 0.62-0.84, P=2.89×10⁻⁵, FIG. 4A and Table 12). Causal association was identified between genetically-proxied all-targets-based and oropharynx cancer risk (odds ratio 0.58; 95% confidence interval 0.39-0.86, P=6.82×10⁻³, FIG. 4B and Table 12). In the protein-protein interaction-based Mendelian randomization analysis, 6 protein-protein interaction (PPI)-based genes were identified for KCNJ11 (Table 5), and 30 protein-protein interaction-based genes were identified for PPARG (Table 6). The results of protein-protein interaction-based Mendelian randomization analysis showed a similar result for the effect of genetically-proxied of KCNJ11-PPI on gastric cancer (odds ratio inverse-variance-weighted (OR IVW) 0.67; 95% confidence interval (CI) 0.59-0.78, P=5.87×10⁻⁸, FIG. 4C and Table 13). Results for the causal association between genetically-proxied PPARG-PPI on oropharynx cancer were shown in FIG. 4D and Table 13 (odds ratio inverse-variance-weighted 0.65; 95% confidence interval (CI) 0.41-1.03, P=0.066).

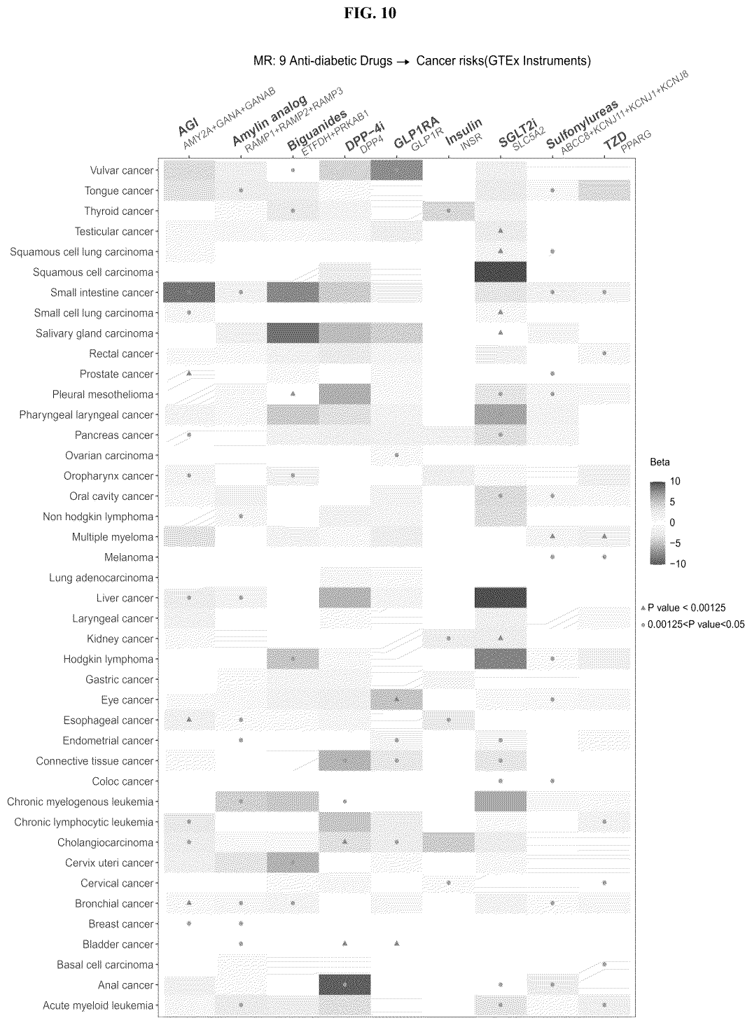

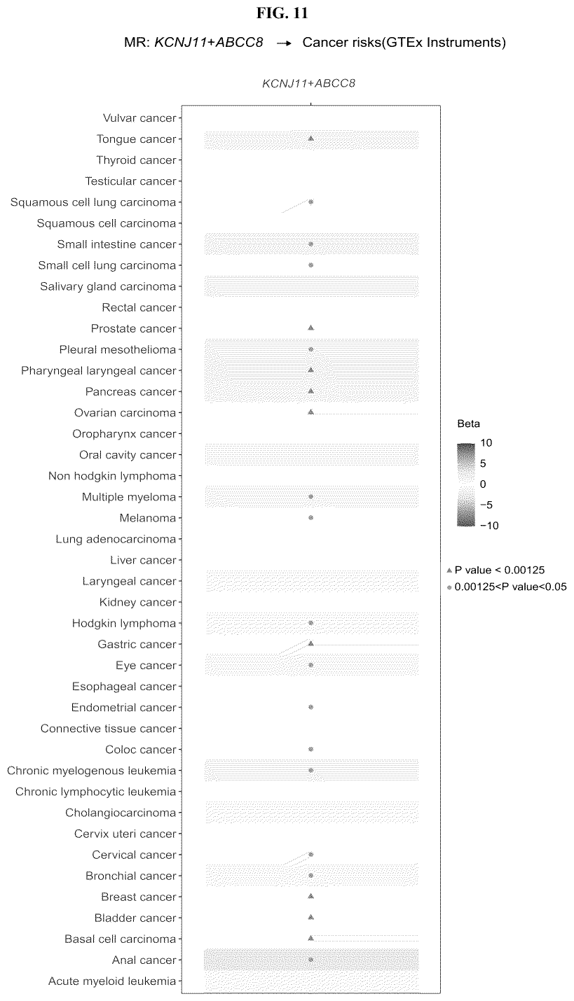

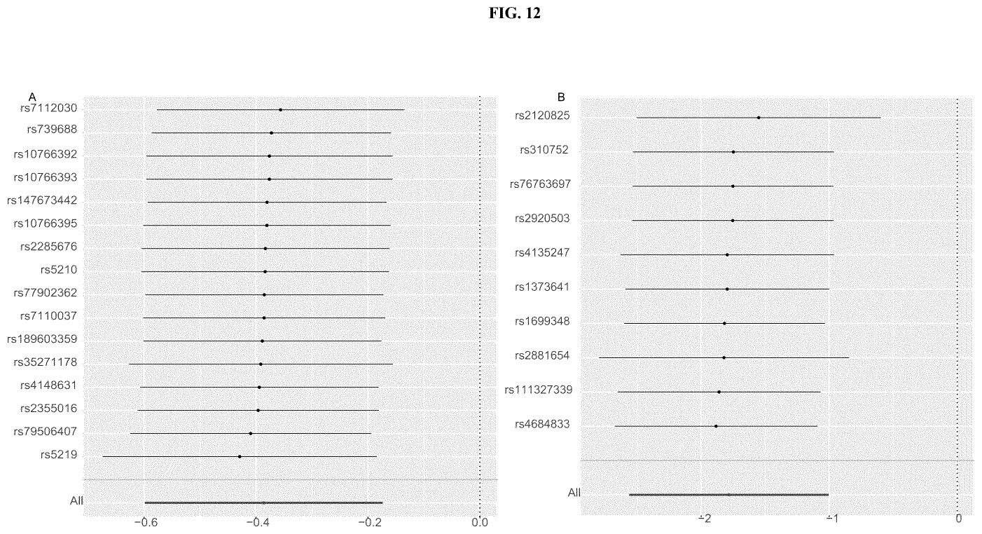

The causal association between various anti-diabetic drugs, including metformin, thiazolidinediones, sulfonylurea, dipeptidyl peptidase 4 inhibitors (DPP-4i), alpha-glucosidase inhibitors (AGIs), sodium-glucose cotransporter inhibitors (SGLT2i), glucagon-like peptide 1 receptor agonist (GLP1RA), as well as insulin and amylin analogs, and their impact on the 40 different types of cancers were also investigated. This data showed that certain anti-diabetic drugs, such as sulfonylurea and thiazolidinedione, demonstrated a potential reduction in the risk of gastric cancer (odds ratio inverse-variance-weighted (OR IVW) 0.66, 95% confidence interval (CI) 0.57-0.76, P=5.37×10⁻⁵) and oropharynx cancer (odds ratio inverse-variance-weighted 0.17, 95% confidence interval 0.09-0.30, P=1.94×10⁻⁹, Table 14), respectively. The results of the Mendelian randomization analysis for 9 anti-diabetic drugs and their association with 40 cancers can be found in FIG. 10 (further data not shown). The combined effect of KCNJ11 and ABCC8 indicated a protective effect on gastric cancer risk (odds ratio inverse-variance-weighted 0.66, 95% confidence interval 0.58-0.75, P=3.03×10⁻¹⁰, Table 15). The results of Mendelian randomization analysis for the effect of combined KCNJ11 and ABCC8 on 40 cancers can be found in FIG. 11 (further data not shown).

### Instrument Validation

Genetically-proxied activation of KCNJ11 (β IVW −0.14, 95% confidence interval −0.17-−0.11, P=1.70×10⁻²⁶), and GLP1R (β MR-Egger 0.73, 95% confidence interval 0.14-1.34, P=0.04, Table 14) were associated with lower BMI (Table 16). This indicated an association of genetically-proxied expression of RAMP2 (β IVW 0.22 95% confidence interval 0.002-0.45, P=0.043, Table 16) with lower HbA1c. Genetically-proxied peroxisome proliferator-activated receptor gamma (PPARG) activation was associated with lower alanine aminotransferase (ALT) (β IVW 0.44, 95% confidence interval 0.37-0.51, P=2.56×10⁻³², Table 16) and AST (β IVW 0.30, 95% confidence interval 0.21-0.39, P=9.75×10⁻¹¹, Table 16).

### Sensitivity and Mediation Analysis

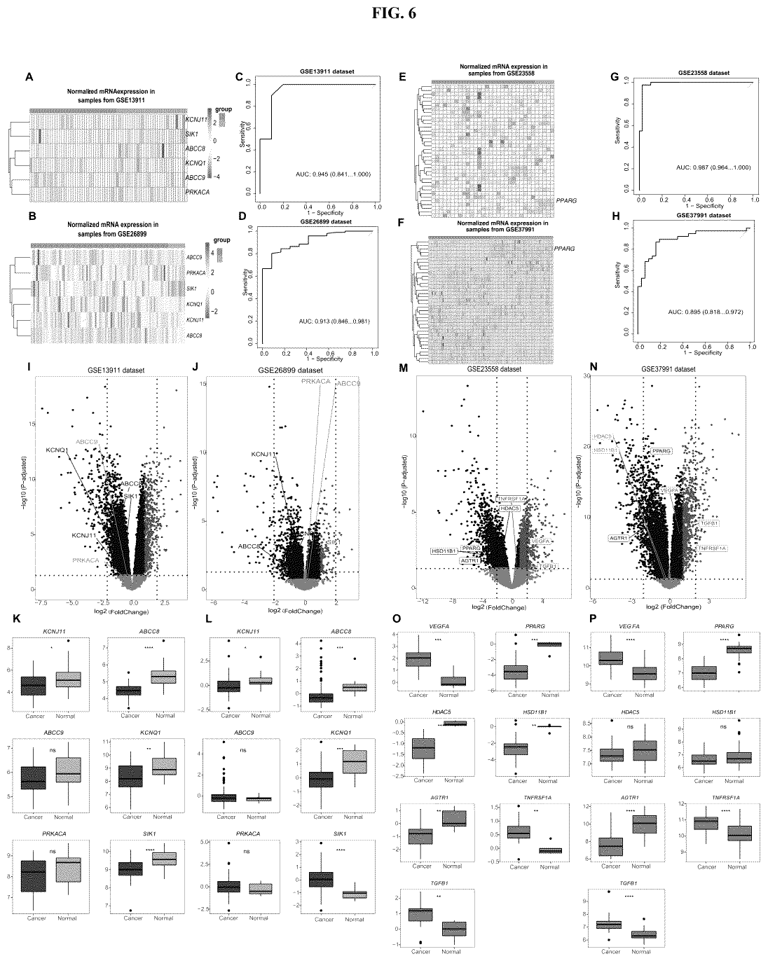

There was limited data suggesting bias of the instrumental variants (F-statistics>10). The proportion of variance in the phenotype (R²) explained by the genetic instruments ranged from 0.024 to 0.34 (Table 1). There was limited evidence of heterogeneity in the SNP effect estimates for inverse-variance-weighted (IVW) and Mendelian randomization-Egger regression for KCNJ11 and PPARG (Table 17). Mendelian randomization-Egger intercept also indicated limited evidence of pleiotropy (Table 17). There were no (individual) outliers in leave-one-out plots (FIG. 6) and MR-PRESSO (Table 9). The heterogeneity test and pleiotropy test for all-targets-based Mendelian randomization analysis and protein-protein interaction-based Mendelian randomization analysis indicated that there were no potential heterogeneity and pleiotropy (Table 18).

Co-localization analysis was performed to investigate whether the significant findings for KCNJ11 and PPARG were due to violation of the exclusion restriction assumption. Co-localization analysis suggested that KCNJ11 and gastric cancer associations had an 81.53% posterior probability of sharing a causal variant (rs2074310) (Table 19). Then, co-localization analysis confirmed that KCNJ11 and low-density lipoprotein cholesterol (LDL-C) had 83.72% posterior probability of sharing a causal variant (rs4148646). The SNP rs4148646 exhibited a high level of concordance with rs2074311, characterized by a D′ value of 0.996 and an R² value of 0.991. Notably, both eQTLs served as proxies for rs5215, an instrument to proxy KCNJ11 in the Mendelian randomization analysis. A subsequent two-step Mendelian randomization analysis showed the presence of a causal effect between lower LDL-C and gastric cancer risk (odds ratio inverse-variance-weighted (OR IVW) 0.87, 95% confidence interval (CI) 0.79-0.97, P=8.60×10⁻³, Table 20). The mediation analysis indicated LDL-C accounts for a 4.81% (95% confidence interval 3.81%-5.80%) proportion of the causal association between genetically-proxied activation of KCNJ11 and gastric cancer (FIG. 5 and Table 21).

Other risk factors, except for LDL-C, were included in a multi-variable Mendelian randomization (MVMR) analysis, the results of which indicated that genetically-proxied activation of KCNJ11 could reduce the risk of gastric cancer after adjusting for potential confounders (odds ratio inverse-variance-weighted (OR IVW) 0.43, 95% confidence interval 0.28-0.65, P=1.26×10⁻³, Table 22). No data was found indicating that genetically-proxied activation of PPARG and oropharynx cancer shared a causal variant (posterior probability: 0.312%, Table 23). A MVMR analysis including putative risk factors showed no causal association between genetically-proxied activation of PPARG and reduced oropharynx cancer risk after adjusting for potential confounders (OR IVW 0.35, 95% confidence interval 0.13-0.97, P=0.18, Table 24).

### Validation Analysis of Causal Association Between Anti-Diabetic Drugs and Cancer Risks

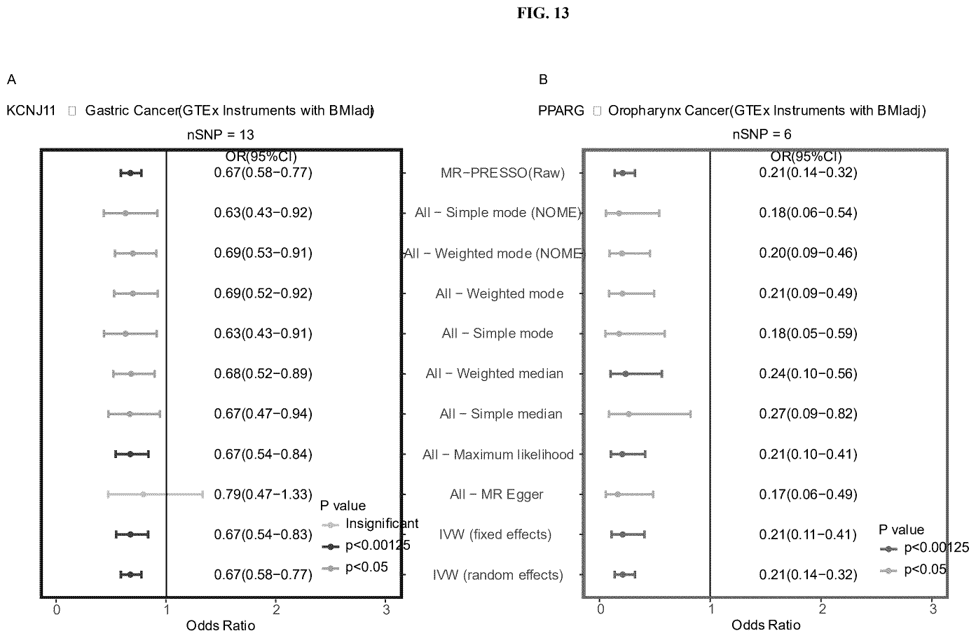

Drug-target Mendelian randomization analyses were replicated in a body mass index (BMI)-adjusted European ancestry. The results revealed a similar association where genetically-proxied activation of KCNJ11 and PPARG would decrease the risk of gastric cancer (odds ratio inverse-variance-weighted 0.67, 95% confidence interval 0.58-0.77, P=1.80×10⁻⁸) and oropharynx cancer, respectively (odds ratio inverse-variance-weighted 0.21, 95% confidence interval 0.14-0.32, P=5.22×10⁻¹³, Table 25). In an independent, East Asian population, it was confirmed that genetically-proxied activation of KCNJ11 could reduce gastric cancer risk (odds ratio inverse-variance-weighted 0.75, 95% confidence interval 0.67-0.85, P=1.87×10⁻⁶, FIG. 13 and Table 26).

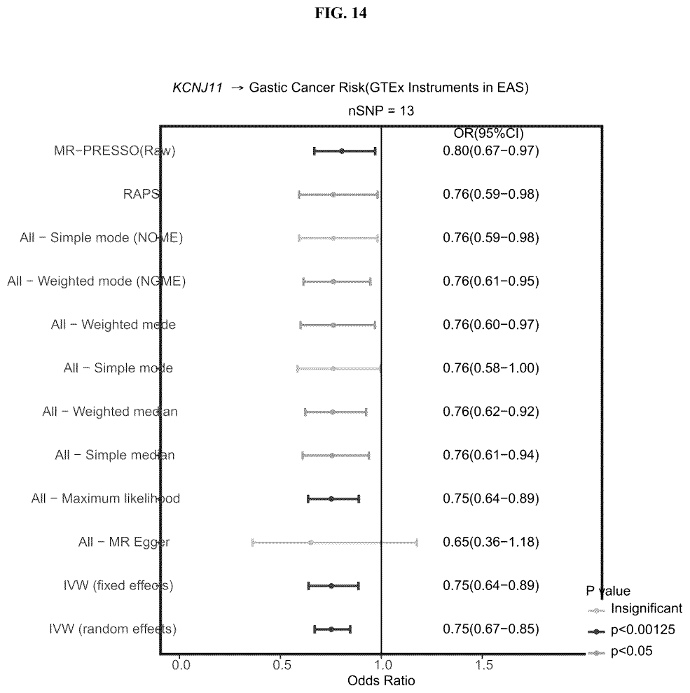

A causal association was identified between genetically-proxied effect of all-targets (odds ratio inverse-variance-weighted 0.99, 95% confidence interval 0.98-1.00, P=0.022) and sulfonylurea (odds ratio MR-Egger 0.97, 95% confidence interval 0.95-1.00, P=0.048, Pleiotropy=0.02) on the reduced risk of pan cancer (FIG. 14 and Table 27).

### Bio-Informatics Analysis

The heatmap of the KCNJ11 and PPARG-PPI genes was shown in FIG. 6(A/B/E/F). These results indicated that 3 KCNJ11-PPI genes (ABCC8, KCNQ1, and SIK1) were differentially expressed between gastric cancer and control tissues in training and validation datasets (FIG. 6I/J/K/L, Table 28). The area under the Receiver Operating Characteristic (ROC) curve (AUC) of the model based on all PPI genes of KCNJ11 was 0.945 in the training cohort (GSE13911) indicating performance in classification of gastric cancer samples and healthy controls (FIG. 6C). A separate dataset (GSE26899) was used to verify the effectiveness of the constructed classification score model. The AUC verification result of Random Forest (RF) model was 0.913 (FIG. 6D).

Four (4) genes were found to be interacting with PPARG (AGTR1, VEGFA, TNFRSF1A, and TGFB1) which were differentially expressed between oropharynx cancer and healthy tissues in training and validation datasets (FIG. 6M/N/O/P, Table 29). The AUC of the model based on all PPI genes of PPARG was 0.987 in the training cohort (GSE23558) with classification performance for oropharynx cancer samples and healthy controls (FIG. 6G). In the validation dataset (GSE73991), the AUC verification result of Random Forest (RF) predictive model was 0.895 (FIG. 6H).

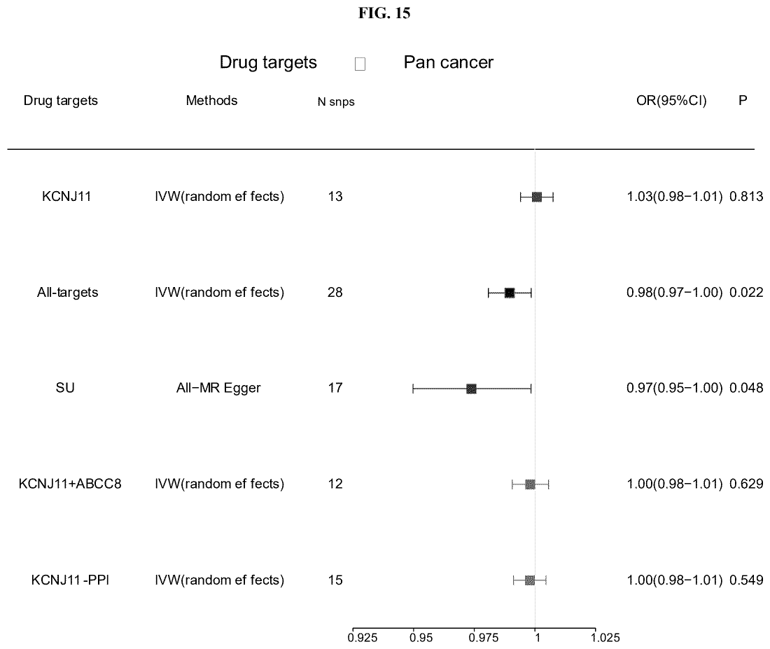

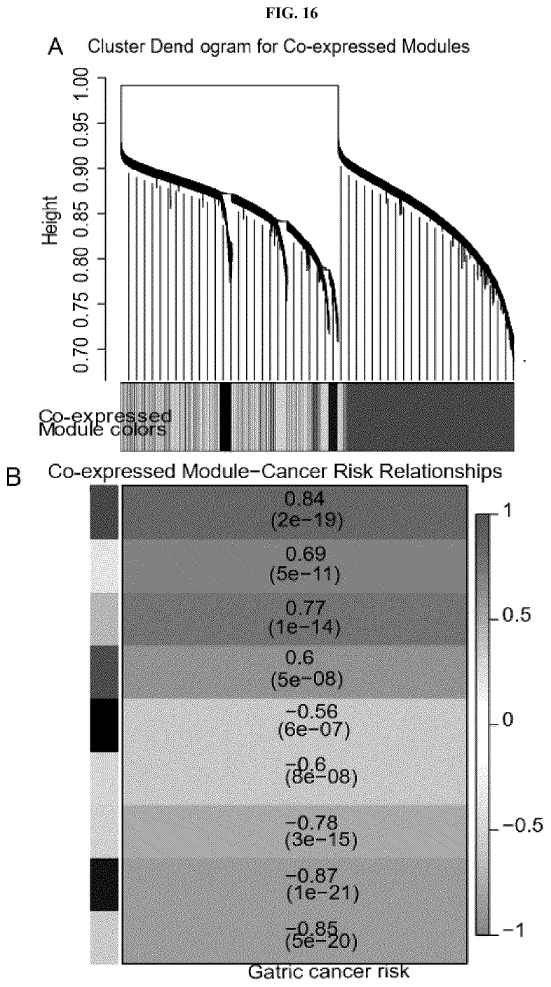

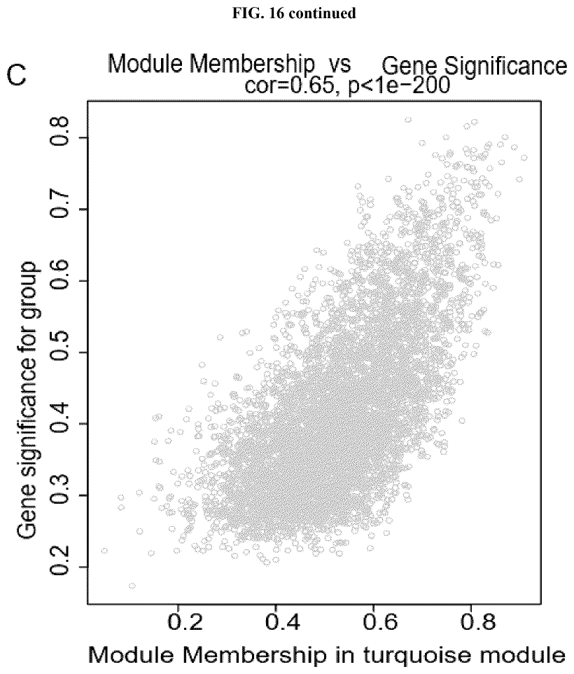

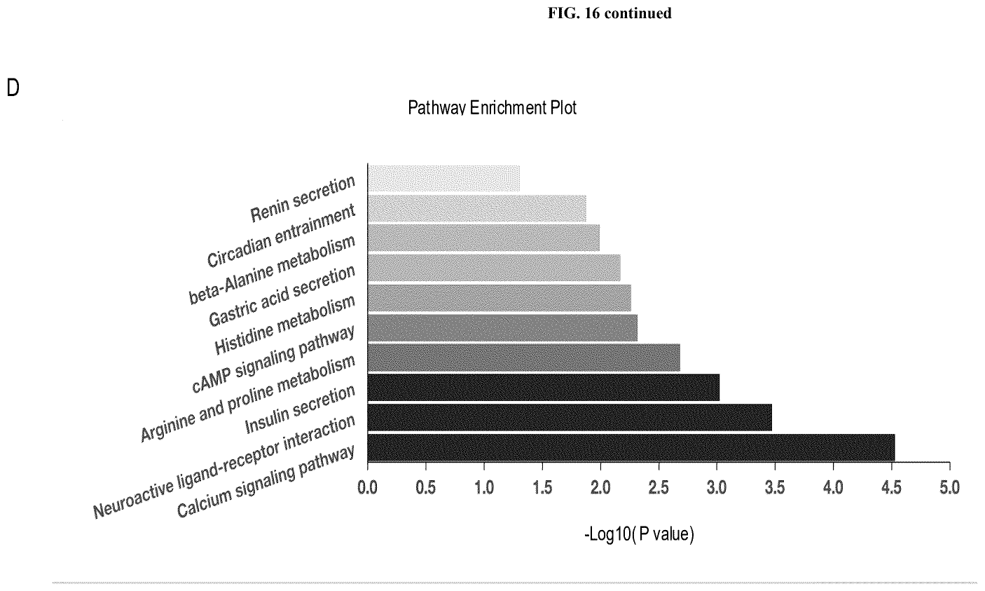

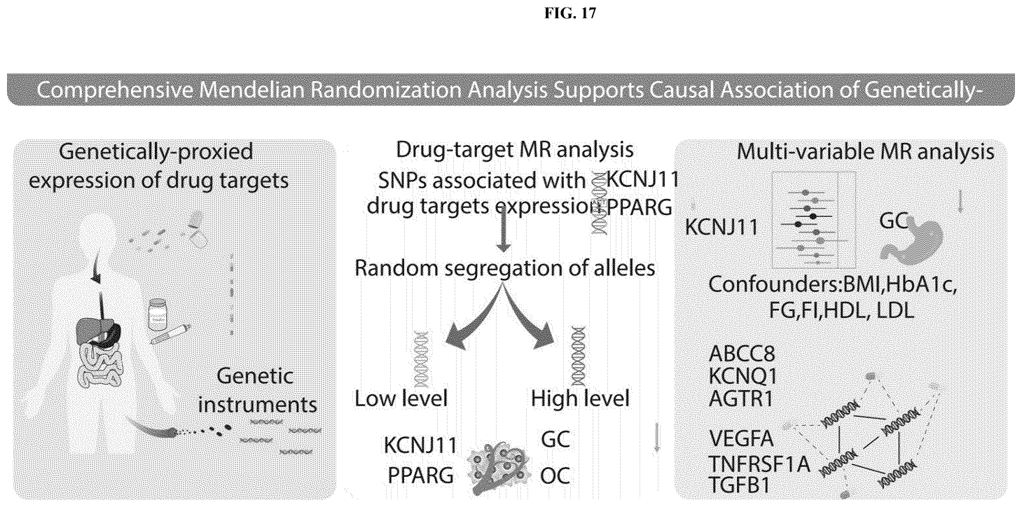

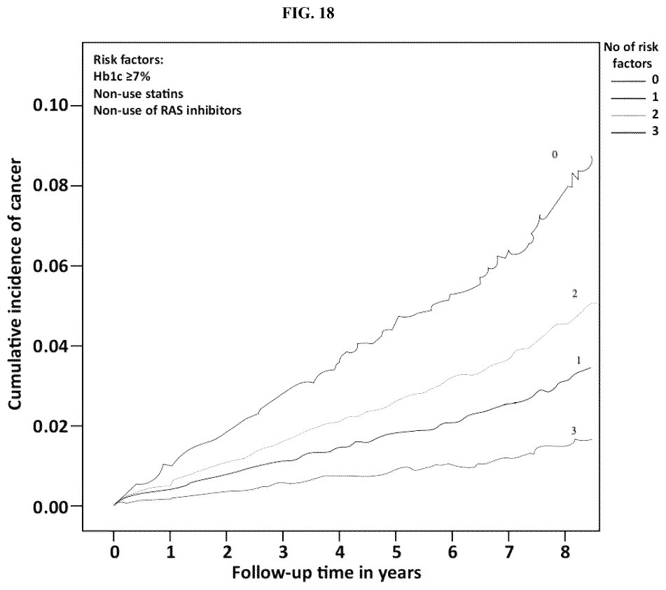

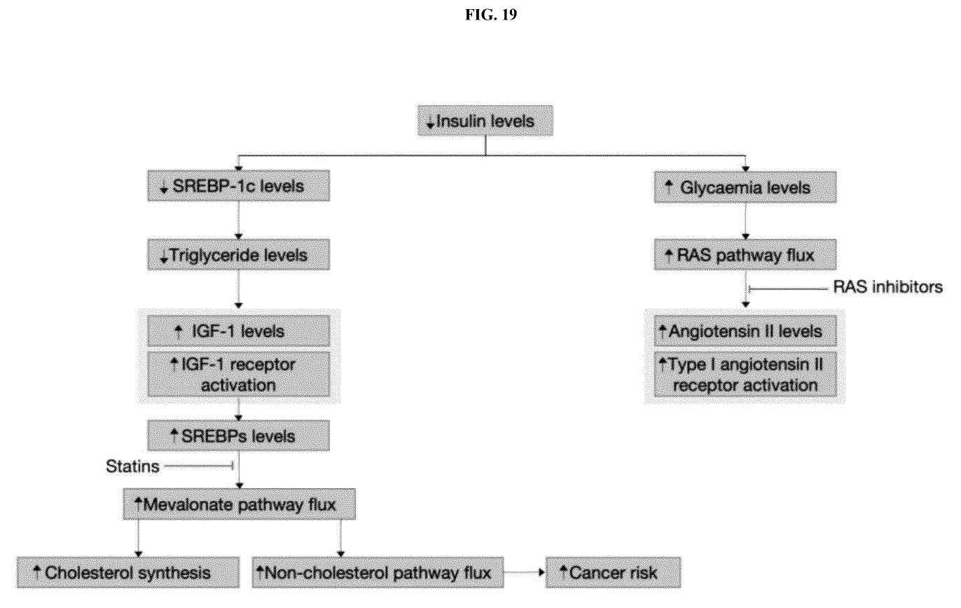

Eight (8) modules were identified in GSE13911 by WGCNA, and the KCNJ11 and ABCC8 clustering in turquoise module which has a negative association with gastric cancer (r=−0.78, P<3×10⁻¹⁵, genes=4801, FIG. 15). The pathway enrichment analysis indicated that top 100 co-expression genes in turquoise module enriched in calcium signalling pathway, insulin secretion, cAMP signalling pathway and gastric acid secretion (Table 30).

Using the inverse-variance weighted (IVW) Mendelian randomization method, data supporting the association between genetically-proxied activation of KCNJ11 and reduced risk of gastric cancer (odds ratio (OR) inverse-variance-weighted (IVW) 0.68; 95% confidence interval (95% CI) 0.59-0.78, P=7.37×10⁻⁸) was obtained. The results of combined effects of all-targets-based (OR IVW 0.72, 95% confidence interval 0.62-0.84, P=2.89×10⁻⁵) and KCNJ11-PPI-based Mendelian randomization analyses (OR IVW 0.67; 95% confidence interval 0.59-0.78, P=5.87×10⁻⁸) also indicated decreased risk of gastric cancer. A statistically significant association between genetically-proxied activation of PPARG and reduced risk of oropharynx cancer (OR IVW 0.17; 95% confidence interval 0.09-0.30, P=1.94×10⁻⁹) was observed, supported by all-targets-based Mendelian randomization analysis (OR 0.58; 95% confidence interval 0.39-0.86, P=6.82×10⁻³). Mediation analysis indicates that LDL-C accounts for 4.81% proportion underlying the association between genetically-proxied activation of KCNJ11 and gastric cancer risk. The causal association between genetically-proxied activation of KCNJ11 and gastric cancer was attenuated after adjusting for confounders in the MVMR analysis.

In this study, the potential of anti-diabetic drugs repurposing for cancer prevention was explored. By integrating publicly available GWAS and eQTL data, two approaches were employed to select SNPs as genetic instrumental variants to proxy expression of drug targets and to explore the causal association between genetically-proxied expression of drug targets and cancers susceptibility in a comprehensive MR analysis. Results were obtained indicating that therapeutic modulation of KCNJ11 can reduce the risk of GC. These findings were supported by estimates from PPI-based MR analysis, all-targets-based MR analysis, and MVMR analysis. In a series of sensitivity analyses, the beneficial effect of genetically-proxied activation of KCNJ11 on decreased risk of GC was observed to be partly mediated by lowering LDL-C. These combined results highlight the potential role of KCNJ11 activation in reducing GC risk and suggest that the mechanism of action may involve LDL-C modulation. Analysis did not reveal any causal association between genetically-proxied activation of PPARG and OC after adjusting for confounding factors.

KCNJ11, a member of the potassium channel gene family, is located at 11p15.1 and does not have any intron. This gene encodes an inward-rectifier potassium ion channel (Kir6.2), which forms the KATP channels. Defects in KCNJ11 may alter the charges of the ATP-binding region and decrease its sensitivity to ATP. The latter plays a key role in the bio-energetic metabolism of all cellular compartments that form the tumour microenvironment (TME). Potassium channels have been implicated in regulating cancer cell proliferation and apoptosis, making them potential targets for cancer therapy. However, the roles of KCNJ11 in cancers susceptibility have not been comprehensively studied. A case-control study of 2,011 colorectal cancer cases and 6,049 controls nested in the multi-ethnic cohort as part of the Population Architecture using Genomics and Epidemiology (PAGE) initiative have reported mutations of KCNJ11 (rs5219) was associated with colorectal cancer risk among males (OR 1.18; 95% CI: 1.05-1.31) and the association remains significant (OR 1.15; 95% CI: 1.031-1.28) after adjustment of genetic ancestry (i.e. adjustment for the leading principal components that can distinguish between African, Asian, European, Latino, and Native Hawaiian ancestry). Nevertheless, a study employing loss-of-function and gain-of-function approaches reported that elevated KCNJ11 expression is associated with cell proliferation, apoptosis, and invasion. Researchers observed an up-regulation of KCNJ11 mRNA levels in tumour tissues (such as HCC and lung cancer) compared to their corresponding non-tumour tissues. Several GEO human cohorts were mined and DEG analysis was conducted to detect differences in KCNJ11 expression between GC and healthy tissues. The data suggested higher expression of KCNJ11 in healthy tissues and lower expression of KCNJ11 in GC tissue (FIG. 6. I/J). It was also found that the IVs of KCNJ11 were associated with up-regulated expression of KCNJ11 in several tissues including whole blood, pancreas, liver, oesophagus, colon, kidney cortex, hippocampus and testis using GTEx v8 data (FIG. 7 and Table 31). These findings suggested that anti-diabetic drugs targeting KCNJ11, e.g., glipizide and glimepiride, might effectively lower blood glucose levels and potentially reduce risk of GC by restoring the mRNA expression of KCNJ11. Furthermore, clinical studies conducted in two independent cohorts of Chinese T2D patients (cohort 1: n=661, cohort 2: n=607) treated with glipizide demonstrated that decreased mRNA expression associated with missense SNPs in KCNJ11 could be effectively rescued by treatment with glipizide in cohort 1. Further, a distinct clinical trial conducted in Europe, involving 44 patients, demonstrated the efficacy and safety of SU therapy for short-term use in patients with diabetes caused by KCNJ11 mutations. This consistent evidence suggested SU which lowers blood glucose by activating the expression of KCNJ11 holds promise as a preventive or therapeutic agent for GC.

Proteins typically implement their functions by regulating other molecules and rarely act alone. PPI offer insights into the relationship between drug targets and other proteins which can influence pathophysiological processes such as signal transduction, cell proliferation, growth, differentiation, and apoptosis. As shown herein, 3 KCNJ11-PPI genes (ABCC8, KCNQ1, and SIK1) were identified differentially expressed in GC and control tissues. The association between genetic variant mutation located in ABCC8 (ATP binding cassette subfamily C member 8) and its effect on dysfunction of sulfonylurea receptor 1 (SUR1) protein in T2D has well been investigated. KCNQ1 (potassium voltage-gated channel subfamily Q member 1) has been identified as susceptibility gene of T2D in different ethnic groups. Studies focused on the function of SIK1 (salt inducible kinase 1) and glucose metabolism indicated that SIK1 knockout animals were strikingly with both increased plasma insulin and enhanced peripheral insulin sensitivity especially in obese mice. These findings provide insights into the therapeutic potential of KCNQ1 and SIK1 in disease management. In the present study, the PPI-based MR analysis confirmed that the genetically-proxied effect of KCNJ11-PPI also decreased the risk of GC, suggesting these proteins may also have potential as an anti-diabetic drug target with anti-cancer properties. However, designing small molecule for PPI interface is not without challenge. PPIs occur at specific interfaces, often without grooves or pockets, hindering small molecule binding. The binding of amino acid residues involved in PPIs can be continuous or discontinuous which complicates drug design for interference. Additionally, PPIs lack endogenous small molecule references available in traditional list of drug targets. Despite these challenges, the analytical approach shown herein demonstrates the value of using PPI-based MR analysis to discover novel drug targets, such as KCNQ1 and SIK1, with anti-diabetic and anti-cancer potential.

As two of the most common diseases in the world, diabetes and cancer have attracted the attention of a great number of investigators with the intention of their epidemiological connections. A European cohort study involved 68,076 participants found that diabetes correlated with increased risk of gastrointestinal cancers (HR 1.5; 95% CI 1.3-1.7) even after applying a 1-year lag period to adjust for detection bias. One meta-analysis reported an increased GC risk in patients with T2D (RR 1.19; 95% CI 1.08-1.31) which persisted in population of European and Asian ancestry. Another meta-analysis utilizing individual-level data from 14 studies within the ‘Stomach Cancer Pooling (StoP) Project’, including 5,592 gastric cancer cases and 12,477 controls, there was no risk association between T2D and GC. However, when stratified by cancer subsite, an elevated risk of GC was observed specifically among patients with cardia tumours. Consistent association between T2D and increased cardia cancer was reported by other researchers. It is still not clear what might be responsible for the difference being observed in the cardia and noncardiac gastric cancer risk, but a recent hypothesis suggests that the answer might lie in the distinct cancer etiopathogenetic mechanisms for the two stomach subsites. Obesity (particularly severe type) and gastroesophageal reflux disease (GERD) are risk factors that are almost unique to cardia cancers, which are different with non-cardiac gastric cancers. The combined effects of diabetes and obesity were responsible for an estimated 804,100 new cases of cancer worldwide. The elevated risk of cardia cancer observed in individuals with T2D may be attributed to their heightened susceptibility to obesity, leading to increased levels of reactive oxygen species (ROS) and subsequent DNA damage and mutations. The accumulation of mutations in cells that escape apoptosis can ultimately lead to cancer. Additionally, this obesity-associated condition may also lead to increased expression and activity of protein tyrosine phosphatases (PTPs), enzymes that dephosphorylate protein tyrosine residues. These alterations can disrupt insulin signalling, ultimately resulting in insulin resistance and hyperinsulinemia. Hyperinsulinemia can give rise to enhanced insulin binding to insulin receptors (IRs), thereby triggering escalated activation of mitogen-activated protein kinase (MAPK) pathways and the phosphoinositide 3-kinase (PI3K) pathways. These signalling cascades subsequently facilitate cell proliferation. Furthermore, using prospective data from a register with extensive phenotypes, our group had reported the interactive association of glycaemic variability and obesity with all-site cancer and related death. Based these pieces of evidence, without being bound by theory, it was thought that the combination of anti-diabetic medications holds promise in reducing the risk of cancer by improving blood glucose control and BMI management. The hypothesis has been supported by a study, which demonstrated that the combined administration of insulin and GLP1RA can effectively improve glycaemic control while promoting weight loss. Additionally, it may reduce the interactive effect between hyperglycaemia and obesity, thereby decreasing the cancer risk in patients with T2D. In line with these epidemiological findings, the MR analyses shown herein provide additional support for the hypothesis that the use of combination anti-diabetic medications to achieve optimal blood glucose levels and BMI can potentially reduce the risk of developing specific subtypes of cancer, e.g. GC, and pan cancer. These findings reinforce the causal relationship between the interactive effects of hyperglycaemia and obesity and certain cancer subtypes, while also highlighting the potential of leveraging genetic variants for precision treatment of diabetes and cancer prevention.

Despite the robust results from the MR and bioinformatics analysis disclosed herein, the biological mechanism underlying the reduced GC risk and genetically-proxied activation of KCNJ11 requires further elucidation. Previous cohort studies indicated that patients with GC group had higher LDL-C than control group, which aligned with the MR analysis suggesting lower LDL-C levels and reduced GC risk. In support of these findings, nonlinear relationships between lipids and cancer risks have been reported. The risk association of cancer with LDL-C was V-shaped, with both LDL-C levels of <2.80 mmol/l and ≥3.80 mmol/l being associated with elevated risks of cancer. Reduced risk of cancer was observed in T2D patients who were exposed to statins and/or renin angiotensin system (RAS) inhibitors after adjustment for drug use indications and demographic and lifestyle covariates.

There are complex inter-relationships amongst lipid, glucose metabolism and cancer risk. In a review, it is highlighted herein that insulin insufficiency could lead to dysregulation of triglyceride synthesis accompanied by activation of the insulin-like growth factor-1(IGF1), RAS and 3-hydroxy-3-methyl-glutaryl-coenzyme-A-reductase (HMGCR) pathway resulting in increased production of oncogenes. Thus, by correcting the defective insulin insufficiency, it is thought that drugs such as SU may reduce cancer risk by improving the metabolic environment or through other direct mechanisms in cancer cells.

These clinical observations suggest close links between perturbation of lipid and glucose metabolism with cancer risks. To this end, the mediation analysis also suggested low LDL-C account for 4.81% proportion of anti-cancer effects of KCNJ11 activation.

In conclusion, this study provides evidence that genetically-proxied activation of KCNJ11 is associated with reduced risk of GC when considered a single drug target, PPI-based drug targets, and combined anti-diabetic drug targets, partly mediated by lowering of LDL-C levels.

The invention illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

As used in this application, the singular form “a,” “an,” and “the” include plural references unless the context clearly dictates otherwise. For example, the term “a genetic marker” includes a plurality of genetic markers, including mixtures and combinations thereof.

As used herein, the term “about”, in the context of concentrations of components of the formulations, typically means+/−5% of the stated value, more typically +/−4% of the stated value, more typically +/−3% of the stated value, more typically, +/−2% of the stated value, even more typically +/−1% of the stated value, and even more typically +/−0.5% of the stated value.

Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Certain embodiments may also be described broadly and generically herein. Each of the narrower species and sub-generic groupings falling within the generic disclosure also form part of the disclosure. This includes the generic description of the embodiments with a proviso or negative limitation removing any subject matter from the genus, regardless of whether or not the excised material is specifically recited herein.

The invention has been described broadly and generically herein. Each of the narrower species and sub-generic groupings falling within the generic disclosure also form part of the invention. This includes the generic description of the invention with a proviso or negative limitation removing any subject matter from the genus, regardless of whether or not the excised material is specifically recited herein.

Other embodiments are within the following claims and non-limiting examples. In addition, where features or aspects of the invention are described in terms of Markush groups, those skilled in the art will recognize that the invention is also thereby described in terms of any individual member or subgroup of members of the Markush group.

### Acronyms Used Herein

AUC (Area Under the ROC Curve), AGI (Alpha-glucosidase Inhibitor), DEGs (Differential-Expressed Genes), ALT (Alanine Aminotransferase), AST (Aspartate Aminotransferase), BMI (Body Mass Index), DPP-4i (Dipeptidyl Peptidase 4 Inhibitors), eQTL (Expression Quantitative Trait Loci), FG (Fasting Glucose), FI (Fasting Insulin), GC (Gastric Cancer), GEO (National Center for Biotechnology Information-Gene Expression Omnibus), GERD (Gastroesophageal Reflux Disease), GLP1RA (Glucagon-Like Peptide 1 Receptor Agonist), GRCh37 (Genome Reference Consortium Human Build 37), GS (Gene Significance), GTEx (Genotype-Tissue Expression), GWAS (Genome-Wide Association Studies), HDL-C (High-Density Lipoprotein Cholesterol), HMGCR (3-hydroxy-3-methyl-glutaryl-coenzyme-A-reductase), IEU (Integrative Epidemiology Unit), IGF1 (Insulin-like Growth Factor-1), IRs (Insulin Receptors), IVW (Inverse-Variance-Weighted), IVs (Instrumental Variants), KATP (ATP-sensitive Potassium), KCNJ11 (Potassium Inwardly Rectifying Channel Subfamily J Member 11), KCNQ1 (Potassium Voltage-gated Channel Subfamily Q Member 1), LD (Linkage Disequilibrium), LDL-C (Low-Density Lipoprotein Cholesterol), MAPK (Mitogen-activated Protein Kinase), MM (Scores and Module Membership), ML (Maximum Likelihood), MR (Mendelian Randomization), MR-PRESSO (Mendelian Randomization Pleiotropy RESidual Sum and Outlier), MVMR (Multi-variable MR), NOME (NO Measurement Error), OC (Oropharynx Cancer), OOB (Out-of-bag), OR (Odds Ratio), PAGE (Population Architecture using Genomics and Epidemiology), PI3K (Phosphoinositide 3-kinase), PPARG (Peroxisome Proliferator-Activated Receptor Gamma), PPI (Protein-protein Network), PTPs (Protein Tyrosine Phosphatases), RAMP2 (Receptor Activity Modifying Protein 2), RAPS (Robust Adjusted Profile Score), RAS (Renin Angiotensin System), RF (Random Forest), ROS (Reactive Oxygen Species), R2 (Proportion of Variance Explained), SD (Standard Deviation), SGLT2i (Sodium-Glucose Cotransporter Inhibitors), SIK1 (Salt Inducible Kinase 1), SLC5A2 (Solute Carrier Family 5 Member 2), SNPs (Single Nucleotide Polymorphisms), StoP (Stomach Cancer Pooling), SU (Sulfonylurea), SUR1 (Sulfonylurea Receptor 1), T2D (Type 2 Diabetes), TME (Tumor Microenvironment), TZD (Thiazolidinedione)

### EXPERIMENTAL SECTION

### Data Collection

Datasets were collected from multiple sources, including the GWAS Catalog, Integrative Epidemiology Unit (IEU) Open GWAS, FinnGen Consortium, and Breast Cancer Association Consortium, which collectively provide a comprehensive list of 40 distinct types of cancer. All data used in this study were sourced from publicly accessible databases which specifically included individuals of European ancestry. The data were categorized into nine cancer groups, following the classification of cancers by anatomical location or system provided by the National Cancer Institute (Table 1). All studies contributing data to these analyses were approved by relevant institutional review board from each country conducted in accordance with the Declaration of Helsinki and all participants had provided informed consent.

### Data Availability

Several summary statistics of GWAS used in this study are publicly available on the MRC Integrative Epidemiology Unit (IEU) Open GWAS project (https://gwas.mrcieu.ac.uk/), GWAS Catalog (https://www.ebi.ac.uk/gwas/), DIAGRAM Consortium (http://diagram-consortium.org/downloads.html). Summary genetic association data from the Finngen Consortium can be accessed by visiting https://www.finngen.fi/en/access_results. Breast cancer GWAS from the BCAC—Breast Cancer Association Consortium, Michailidou et al. Tissue-derived gene expression eQTL data is available from the GTEx project via https://www.gtexportal.org/home/. The data used for the analyses described in this manuscript were obtained from the GTEx on Jan. 10, 2022. Whole blood eQTL data is available from https://www.egtlgen.org/cis-eqtls.html.

### Instrument Selection and Pre-Processing

All drug names and their corresponding identification numbers can be accessed online from DrugBank (version 5.1.10) at https://go.drugbank.com/. The available drug categories include AGI, amylin analogs, biguanides (metformin), DPP-4i, GLP1RA, SGLT2i, KATP channels blockers (SU and meglitinide), TZD, and insulin. Pharmacologically approved and active protein targets in each class of anti-diabetic drugs were defined in DrugBank where data on relevant targets were extracted (Table 2).

Two approaches (“GTEx Instruments” and “GWAS Instruments”) were implemented to construct genetic instruments that serve as proxies for drug targets. Only the targets that demonstrated statistical significance in both “GTEx Instruments” approach and “GWAS Instruments” approach were further investigated.

In “GTEx Instruments” approach, significant variants associated with anti-diabetic drug targets were identified using data from the Genotype-Tissue Expression version eight (GTEx.v8) project. SNPs with the lowest P values across all tissues were considered as the most promising genetic instruments for MR analysis. Associations between SNPs and gene expression, as well as between SNPs and T2D, were extracted and harmonized to identify effect alleles corresponding to the changes of targets expression and risk of T2D. To validate these SNPs as instruments that mimic the effects of anti-diabetic drugs, the association between expression changes of drug targets and T2D was estimated using a two-sample MR method. SNPs with a nominal P value below 0.05 were listed, regardless of the tissue in which the SNP was identified in the main analysis (Table 1). To assess the causal effect of genetically-proxied activation of anti-diabetic drugs targets on the risk of type 2 diabetes, the Wald ratio was applied for each single SNP and utilized in the inverse variance weighted (IVW) method that involved multiple SNPs.

To conduct an additional post-hoc sensitivity analysis, the “GWAS Instruments” approach was employed to generate instruments to proxy anti-diabetic drug targets. This involved constructing instruments using PLINK by identifying SNPs associated with T2D in the DIAGRAM dataset (cases=74,124 and controls=824,006, European ancestry). Only SNPs that reached genome-wide significance (P<5×10-8) and were located within a 200 kb window range of the gene encoding each drug target were considered (as listed in Table 3).

To enhance the strength of the instruments and maximize the proportion of variance explained in each respective drug target, the SNPs used as instruments have weak linkage disequilibrium (LD) (r2<0.2) with each other. Population specific correlations among variants were estimated from the 1000 Genomes Project Phase 3 (1000G).

In a separate population the UK Biobank cohort study, the association of SNPs selected by the two approaches with HbA1c levels was evaluated to minimize winner's curse bias. SNP with directional effect on HbA1c opposite to that on T2D were removed from the instrument, since these inconsistencies likely represent pleiotropic mechanisms.

To prioritize the primary instrument for proxying drug targets, the proportion of variance explained (R2) for each respective instrument were compared. The “GTEx Instruments” approach was given priority as the primary instrument's selection and construction for the PPI networks and all-targets.

Instrument Selection of all-Targets, PPI Networks, Drug Classes, and Combined KCNJ11+ABCC8

Since anti-diabetic drugs are often used in combination to lower blood glucose, all IVs for the all the drug targets were combined into the analyses performed (Table 4). To enhance the results of drug-target MR analysis for identifying potential drug targets and to explore the potential causal association between pathways and cancer risk, PPI-based MR analysis was conducted. PPI networks of drug targets were generated by STRING (https://string-db.org, version 11.0b). Genes located in tier 1 cluster (Table 5) were selected as potential drug targets and genetically-proxied as the PPI networks.

Due to KCNJ11 and ABCC8 proximity on chromosome 11p15.1 and their combined formation of the KATP channel, the impact of both KCNJ11 and ABCC8 genes together on 40 cancer risks. Furthermore, the causal association between various classes of anti-diabetic drugs, e.g., metformin, TZDs, SU, DPP-4i, AGI, SGLT2i, GLP1RA, as well as insulin and amylin analogs and their potential impact on the risk of 40 cancers were explored.

“GTEx Instruments” approach was applied to selected SNPs mapping to genes for all-targets, PPI-based genes, as well as single anti-diabetic class. Stringent LD clumping was employed using the clump_data (r2<0.001, 100 kb window, 1000G reference panel) function to generate an independent set of these IVs.

### Instrument Validation

To validate the instrumental variants for drug targets, their associations were investigated with relevant clinical phenotype. Specifically, the association between genetically-proxied KCNJ11 and GLP1R with BMI (N=461,460, European ancestry) was examined. Additionally, the association between RAMP2 and SLC5A2 with fasting glucose (FG) levels (N=200,622 European ancestry) was assessed. Lastly, the association between PPARG and levels of alanine aminotransferase (ALT) (N=389,733) and aspartate aminotransferase (AST) levels (N=388,490) in a population of European ancestry was examined.

### Two-Sample Mendelian Randomization

In the primary analysis, drug-target Mendelian randomization was applied to investigate the association of genetically-proxied expression of all anti-diabetic drug targets on the 40 different type cancers. Only findings that exhibited consistency in both the “GTEx Instruments” and “GWAS Instruments” approaches were further investigated. Subsequently, a PPI-based MR analysis and all-targets-based MR analysis were performed using the consistent and robust results in the previous step. All analysis was performed in R using the “TwoSampleMR” package with Genome Reference Consortium Human Build 37 (GRCh37), assembly Hg19.

In the primary drug-target Mendelian randomization analysis, the random-effects inverse-variance weighted (IVW) model was employed to obtain the MR estimates. The random-effects IVW model (Function 1) can provide an unbiased effect in the absence of horizontal pleiotropy or when horizontal pleiotropy is balanced. To evaluate the potential for unbalanced horizontal pleiotropy, where genetic variants influence multiple traits through independent biological pathways, sensitivity analyses were conducted. In the secondary analysis, the TwoSampleMR package was employed to implement up to 10 additional MR methods. These methods included IVW (fixed-effects model), MR Egger, simple mode, simple median, weighted median, weighted mode, simple mode (with the assumption of NO Measurement Error, NOME), weighted mode (NOME), MR-PRESSO (Mendelian Randomization Pleiotropy RESidual Sum and Outlier), RAPS (Robust Adjusted Profile Score), and maximum likelihood (ML).

$$\hat{β} = \frac{{\sum }_{t = 1}^{t} {β}_{T 2 D} {β}_{cancer} {σ}_{{β}_{T 2 D}}^{- 2}}{{\sum }_{t = 1}^{t} {β}_{T 2 D}^{2} {σ}_{{β}_{cancer}}^{- 2}} , s e ( \hat{β} ) = \sqrt{\frac{1}{{\sum }_{t = 1}^{t} {β}_{T 2 D}^{2} {σ}_{{β}_{cancer}}^{- 2}}} \qquad ( Function 1 )$$

To enhance the robustness of these findings, additional analyses were conducted to evaluate the presence of heterogeneity in the individual SNP estimates. This was accomplished by using the Cochran Q-statistic, which can indicate the existence of invalid instruments, possibly due to horizontal pleiotropy. Iterative leave-one-out analysis was performed by removing one SNP at a time from instruments to examine whether finding showing nominal evidence of association were driven by a single influential SNP. MR-PRESSO was also employed to detect and correct for potential outliers, thus ensuring the reliability of the MR analysis by addressing any concerns related to pleiotropic effects.

To account for multiple testing across primary drug target analyses, a Bonferroni correction was used to establish a P value threshold of <0.00125 (0.05/40 statistical tests [40 cancer endpoints]), which was used as a heuristic to define “strong evidence”, with findings between P≥0.00125 and P<0.05 defined as “weak evidence, with findings P≥0.05 defined as “insignificant”.

### Mendelian Randomization Assumption Test

Mendelian randomization analyses assume that the genetic IV (i) is associated with the drug target (“relevance”); (ii) does not associate with confounders of the risk factor-outcome association as a common cause with the outcome (“independence”); and (iii) affects the outcome only through the drug target (“exclusion restriction”).

The “relevance” assumption was tested by generating estimates of the proportion of variance of each drug target explained by the instrument (R²) and F-statistics. F-statistics can be used to examine whether results are likely to be influenced by weak instrument bias. As a convention, an F-statistic of at least 10 is indicative of minimal weak instrument bias.

The “exclusion restriction” assumption was evaluated by performing co-localization to examine whether drug targets and cancer endpoints showing nominal evidence of an association in Mendelian randomization analyses share the same causal variant at a given locus. This analysis allowed exploration of whether the drug targets and cancer outcomes were influenced by different causal variants in linkage disequilibrium, indicating the presence of horizontal pleiotropy. Horizontal pleiotropy refers to an instrumental variable influencing an outcome through pathways that are independent of the exposure, which violates the exclusion restriction assumption. Co-localization analysis was performed by generating ±200 kb windows from the top SNP used to proxy each respective drug target. As a convention, a posterior probability of ≥0.80 was used to indicate support for a configuration tested.

For analyses showing evidence of co-localization across drug target and cancer endpoint signals, it was examined whether there was evidence of an association of genetically-proxied expression of that target with putative risk factors (i.e., BMI, HbA1c, FG, FI, HDL-C and LDL-C) for the relevant cancer endpoint. If there was evidence for an association between a genetically-proxied drug target and traits (P<0.05) related to previous reported risk factors, this was thought to reflect vertical pleiotropy (i.e., “mediated pleiotropy” where an instrument has an effect on 2 or more traits that influence an outcome via the same biological pathway). Two-step Mendelian randomization analysis was then applied to estimate the exposure-mediator, exposure-outcome, and mediator-outcome effects separately. The mediation analysis employs a two-step Mendelian randomization (MR) approach to assess whether an intermediate factor (mediator, M) explains part or all of the causal effect between an exposure (X) and an outcome (Y). First, the effect of X on M is estimated using genetic instruments (e.g., SNPs associated with X), followed by estimating the effect of M on Y while adjusting for X to avoid confounding. The indirect effect (X→M→Y) is calculated by multiplying these two estimates, while the total effect (X→Y) is derived from standard MR. The proportion of the total effect mediated by M is then computed as the ratio of the indirect to total effect, expressed as a percentage (see. Mendelian randomisation for mediation analysis: current methods and challenges for implementation. Eur J Epidemiol. 2021 May; 36(5):465-478. doi: 10.1007/s10654-021-00757-1).

If there was evidence only for an association between a genetically-proxied expression of drug target and previously reported risk factor, this was thought to reflect horizontal pleiotropy. In the presence of an association with a previously reported risk factor, to account for horizontal pleiotropy, the IVW results were compared with three MR sensitivity analyses using MR-Egger, weighted median, and weighted mode. MR-Egger can provide unbiased estimates even when all SNPs in an instrument violate the exclusion restriction assumption. Lastly, a MVMR analysis was adopted to adjust for the effect of previously reported confounders on cancer risk.

### Validation Analyses of Causal Association Between Anti-Diabetic Drugs and Cancer Risks

Drug-target Mendelian randomization was applied and detected potential causal association between genetically-proxied activation of KCNJ11 and PPARG and gastric cancer and oropharynx cancer risk in a BMI adjusted European population (74,124 cases and 824,006 controls), respectively. To validate the results specifically for gastric cancer, which has a higher prevalence in Asian populations, summary statistics were obtained for type 2 diabetes from DIAGRAM with East Asian ancestry (N=139,782). Additionally, gastric cancer GWAS summary statistics were extracted from an East Asian ancestry (7,921 cases and 159,201 controls). Subsequently, drug-target Mendelian randomization was applied to explore the potential causal association between genetically-proxied activation of KCNJ11 and gastric cancer risk.

Moreover, drug-target Mendelian randomization was performed to explore the association between genetically-proxied effect of anti-diabetic drugs targets on pan cancer risk (27,483 cases, 372,016 controls) (https://doi.org/10.5523/bris.aed0u12w0ede20olb0m77p4b9).

### Bio-Informatics Analysis

### Data Acquisition

Transcriptome expression data of the gastric cancer cohorts (GSE13911 and GSE79973) and the oropharynx cancer cohorts (GSE37991 and GSE23558) were downloaded from the National Center for Biotechnology Information-Gene Expression Omnibus (GEO, https://www.ncbi.nlm.nih.gov/geo/). The clinical and pathological information of these patients was obtained from the GEO Database.

### Identification of Differential-Expressed Genes (DEGs)

In this analysis, the initial focus was on extracting the expression levels of genes associated with KCNJ11-PPI and PPARG-PPI. Next, differential-expressed genes analysis was conducted to identify differential-expressed genes between normal samples and tumour samples based on these KCNJ11-PPI and PPARG-PPI genes with a cutoff value of |log 2FC|>2 and adjusted P<0.05 by using limma package. To provide an overview of the expression profiles of the KCNJ11-PPI and PPARG-PPI-based genes and to identify the distinct expression patterns among the samples, a heatmap was generated using the pheatmap package. Furthermore, boxplots were employed to highlight the differences between normal and tumour samples for the KCNJ11-PPI and PPARG-PPI-based genes. Genes having |log 2FC|>2 and adjusted P<0.05 were highlighted and labelled in the volcano plot.

### Random Forest (RF) Classification and Cancer Classification Model

To construct a predictive model, the randomForest package was utilised to build a random forest model using the differential-expressed KCNJ11-PPI and PPARG-PPI genes as input features. The effective estimation of our RF prediction error based on the out-of-bag (OOB) error was established. The latter was used to optimize the parameters in this model. The constitution of a classification model of gastric cancer and oropharynx cancer depends on the differential-expressed PPI-based genes information within the three hidden layers as model parameters. The model results of three-fold cross-validation were calculated using the confusion matrix function. The validation results of area under the curve (AUC) classification performance were calculated using the pROC package. A higher AUC value indicates better discriminative power and overall classification performance.

### Weighted Gene Co-Expression Network Analysis (WGCNA) and Pathway Enrichment Analysis

A weighted gene co-expression network analysis was conducted to identify genes that exhibit similar expression patterns to KCNJ11 based on GSE13911 dataset. Following the construction of gene co-expression networks for gastric cancer using the WGCNA package, pathway enrichment analysis was performed on the top 100 genes exhibiting higher gene significance (GS) scores and module membership (MM) scores within the same module. This analysis aimed to uncover the underlying biological mechanisms associated with these genes. To conduct the pathway enrichment analysis, the Enrichr tool and the KEGG database were applied. Pathway enrichment analysis was specifically focused on the genes identified within a single WGCNA module.

### Code Availability

All code for data cleaning and analysis is available at GitHub (https://github.com/Jaycie1024/MR_Antidiabtic_Cancers).

### Tables

TABLE 1
Data Summary for Primary, Secondary, Sensitivity, and Additional Analyses. This table
provides a summary of the data used in the primary, secondary, sensitivity, and additional
analyses conducted in the study. The table includes information such as the source
of the data, sample size, variables, and any relevant preprocessing steps.

| | Cancer ClassB2: | | |
|---|---|---|---|
| | G34 A1B2: B2: G43 | Cancer | Data ID |
| Outcomes | Gastrointestinal | Anal Cancer | GCST90043915 |
| | Cancers | Cholangiocarcinoma | GCST90018803 |
| | | Colon Cancer | ukb-b-20145 |
| | | Esophageal Cancer | GCST003739 |
| | | Gastric Cancer | GCST90011807/GCST90018849 |
| | | Liver Cancer | GCST90041812 |
| | | Pancreatic Cancer | GCST90011815 |
| | | Rectal Cancer | GCST90011810 |
| | | Small Intestine | GCST90041816 |
| | | Cancer | |
| | Endocrine Cancer | Thyroid Cancer | GCST90011813 |
| | Eye Cancer | Eye Cancer | GCST90041860 |
| | Genitourinary Cancers | Bladder Cancer | ukb-b-8193 |
| | | Kidney Cancer | GCST90011818 |
| | | Prostate Cancer | GCST90043894 |
| | | Testicular Cancer | GCST90041906 |
| | Gynecologic Cancers | Breast Cancer | https://bcac.ccge.medschl.cam.ac.uk/ |
| | | | bcacdata/oncoarray/oncoarray-and- |
| | | | combined-summary-result/gwas-summary- |
| | | | results-breast-cancer-risk-2017/ |
| | | Endometrial Cancer | GCST006464 |
| | | Ovarian Cancer | ieu-a-1120 |
| | | Vulvar Cancer | GCST90043931 |
| | | Cervix Uteri Cancer | GCST90043891 |
| | Head and Neck Cancers | Laryngeal Cancer | GCST90041800 |
| | | Oral Cavity Cancer | GCST012238 |
| | | Oropharynx cancer | GCST90011806 |
| | | Salivary Gland | GCST90041792 |
| | | Carcinoma | |
| | | Cancer of tongue | GCST90041791 |
| | Hematologic Cancers | Acute Myeloid | GCST90042758 |
| | | Leukemia | |
| | | Chronic Lymphocytic | GCST90042757 |
| | | Leukemia | |
| | | Chronic Myelogenous | GCST90043913 |
| | | Leukemia | |
| | | Hodgkin Lymphoma | GCST90042738 |
| | | Non-Hodgkin | GCST90042741 |
| | | Lymphoma | |
| | | Multiple myeloma | GCST90043910 |
| | | Malignant neoplasm | GCST90041825 |
| | | of connective tissue | |
| | Respiratory/Thoracic | Lung adenocarcinoma | GCST004744 |
| | | Small cell lung | GCST004746 |
| | | carcinoma | |
| | | Bronchial Cancer | GCST90041821 |
| | | Malignant | GCST90043881 |
| | | Mesothelioma | |
| | | Squamous cell | GCST004750 |
| | | lung cancer | |
| | Skin | Melanoma | ukb-b-2750 |
| | | Squamous cell | GCST90041917 |
| | | carcinoma | |
| | | Basal cell carcinoma | GCST90041916 |
| | All-sites | Pan cancer | ieu-b-4966 |

| | Cancer ClassB2: | | No. | No. |
|---|---|---|---|---|
| | G34 A1B2: B2: G43 | PubMed ID | cases | controls |
| Outcomes | Gastrointestinal | 34737426 | 107 | 456,241 |
| | Cancers | 34594039 | 832 | 475,259 |
| | | | 1,494 | 461,439 |
| | | 27527254 | 4,112 | 17,159 |
| | | 32887889 | 1,091 | 410,350 |
| | | 34737426 | 128 | 456,220 |
| | | 32887889 | 1,896 | 1,939 |
| | | 32887889 | 2,091 | 410,350 |
| | | 34737426 | 172 | 456,176 |
| | Endocrine Cancer | 32887889 | 762 | 410,35 |
| | Eye Cancer | 34737426 | 183 | 456,165 |
| | Genitourinary Cancers | | 1,101 | 461,832 |
| | | 32887889 | 1,338 | 410,350 |
| | | 34737426 | 7,769 | 201,039 |
| | | 34737426 | 797 | 207,971 |
| | Gynecologic Cancers | 5798588 | 14,910 | 17,588 |
| | | 30093612 | 12,906 | 108,979 |
| | | 28346442 | 25,509 | 40,941 |
| | | 34737426 | 113 | 247,427 |
| | | 34737426 | 372 | 247,168 |
| | Head and Neck Cancers | 34737426 | 269 | 456,079 |
| | | 27749845 | 1,135 | 2,329 |
| | | 32887889 | 1,223 | 410,350 |
| | | 34737426 | 105 | 456,243 |
| | | 34737426 | 322 | 56,026 |
| | Hematologic Cancers | 34737426 | 312 | 456,036 |
| | | 34737426 | 356 | 455,992 |
| | | 34737426 | 110 | 456,238 |
| | | 34737426 | 215 | 456,133 |
| | | 34737426 | 1,395 | 454,953 |
| | | 34737426 | 488 | 455,860 |
| | | 34737426 | 319 | 456,029 |
| | Respiratory/Thoracic | 28604730 | 11,273 | 55,483 |
| | | 28604730 | 2,664 | 21,444 |
| | | 34737426 | 2,120 | 454,228 |
| | | 34737426 | 210 | 456,138 |
| | | 28604730 | 7,426 | 55,627 |
| | Skin | | 1,058 | 461,952 |
| | | 34737426 | 557 | 455,719 |
| | | 34737426 | 4,257 | 452,019 |
| | All-sites | https://doi.org/10.5523/ | 70,223 | 372,016 |
| | | bris.aed0u12w0ede20olb0m77p4b9 | | |

| Analytic stage | Phenotypes | Source | PMID | Sample size |
|---|---|---|---|---|
| Instruments | GTEx V8 tissues | https://gtexportal.org/home/downloads/adult-gtex | | |
| selection | Type 2 diabetes | https://diagram-consortium.org/ | | |
| Instruments | BMI | MRC-IEU consortium | | 461,460 |
| validation | Fasting glucose | / | 34059833 | 200,622 |
| | Fasting insulin | / | 34059833 | 151,013 |
| | Alanine | UKB data | 34017140 | 389,733 |
| | aminotransferase | | | |
| | Aspartate | UKB data | 34017140 | 388,490 |
| | aminotransferase | | | |
| MR validation | T2D BMI adjusted | DIAGRAM consortium | 28566273 | 159,208 |
| analysis | ancestry | | | |
| | T2D East Asia | DIAGRAM consortium | 35551307 | 380,528 |
| | ancestry | | | |
| | Gastric cancer | | 34594039 | 167,122 |
| | East Asia ancestry | | | |
| Sensitivity | HbA1c | Within family | | 45,734 |
| analysis | | GWAS consortium | | |
| | HDL-C | GLGC consortium | 24097068 | 187,167 |
| | LDL-C | GLGC consortium | 24097068 | 173,082 |
| Additional | Gastric cancer | GEO | GSE13911 | 69 |
| analysis | | | GSE79973 | 20 |
| | Oropharynx cancer | | GSE23558 | 32 |
| | | | GSE37991 | 80 |

TABLE 2
A selection of information of anti-diabetic drugs targets in DrugBank

| Substance | Drugs | DrugBank ID | Targets | Class |
|---|---|---|---|---|
| Acarbose | Alpha | DB00284 | MGAM | Oral anti-diabetic drugs |
| Voglibose | glucosidase | DB04878 | MGAM | Oral anti-diabetic drugs |
| Miglitol | inhibitors (AGI) | DB00491 | MGAM | Oral anti-diabetic drugs |
| Miglitol | | DB00491 | GANAB | Oral anti-diabetic drugs |
| Acarbose | | DB00284 | AMY2A | Oral anti-diabetic drugs |
| Acarbose | | DB00284 | SI | Oral anti-diabetic drugs |
| Miglitol | | DB00491 | GANC | Oral anti-diabetic drugs |
| Miglitol | | DB00491 | GAA | Oral anti-diabetic drugs |
| Alogliptin | Dipeptidyl | DB06203 | DPP4 | Oral anti-diabetic drugs |
| Gemigliptin | peptidase 4 | DB12412 | | Oral anti-diabetic drugs |
| Linagliptin | inhibitor | DB08882 | DPP4 | Oral anti-diabetic drugs |
| Saxagliptin | (DPP-4i) | DB06335 | DPP4 | Oral anti-diabetic drugs |
| Sitagliptin | | DB01261 | DPP4 | Oral anti-diabetic drugs |
| Trelagliptin | | DB15323 | | Oral anti-diabetic drugs |
| Vildagliptin | | DB04876 | DPP4 | Oral anti-diabetic drugs |
| Dulaglutide | Glucagon-like | DB09045 | GLP1R | Oral anti-diabetic drugs |
| Exenatide | peptide-1 | DB01276 | GLP1R | Oral anti-diabetic drugs |
| Liraglutide | receptor agonists | DB06655 | GLP1R | Oral anti-diabetic drugs |
| Lixisenatide | (GLP1RA) | DB09265 | GLP1R | Oral anti-diabetic drugs |
| Semaglutide | | DB13928 | GLP1R | Oral anti-diabetic drugs |
| Tirzepatide | | DB15171 | | Oral anti-diabetic drugs |
| Canagliflozin | Sodium-glucose | DB08907 | SLC5A2 | Oral anti-diabetic drugs |
| Dapagliflozin | cotransporter | DB06292 | SLC5A2 | Oral anti-diabetic drugs |
| Empagliflozin | inhibitor | DB09038 | SLC5A2 | Oral anti-diabetic drugs |
| Ertugliflozin | (SGLT2i) | DB11827 | SLC5A2 | Oral anti-diabetic drugs |
| Luseogliflozin | | DB12214 | | Oral anti-diabetic drugs |
| Gliclazide | Sulfonylureas | DB01120 | ABCC8 | Oral anti-diabetic drugs |
| Glimepiride | (SU) | DB00222 | ABCC8 | Oral anti-diabetic drugs |
| Glipizide | | DB01067 | ABCC8 | Oral anti-diabetic drugs |
| Gliquidone | | DB01251 | ABCC8 | Oral anti-diabetic drugs |
| Tolazamide | | DB00839 | ABCC8 | Oral anti-diabetic drugs |
| Tolbutamide | | DB01124 | ABCC8 | Oral anti-diabetic drugs |
| Tolazamide | | DB00839 | KCNJ11 | Oral anti-diabetic drugs |
| Glimepiride | | DB00222 | KCNJ11 | Oral anti-diabetic drugs |
| Glimepiride | | DB00222 | KCNJ1 | Oral anti-diabetic drugs |
| Gliquidone | | DB01251 | KCNJ8 | Oral anti-diabetic drugs |
| Insulin aspart | Insulins | DB01306 | INSR | Injection |
| Insulin | | DB09564 | INSR | Injection |
| degludec | | | | |
| Insulin | | DB01307 | INSR | Injection |
| detemir | | | | |
| Insulin | | DB00047 | INSR | Injection |
| glargine | | | | |
| Insulin | | DB01309 | INSR | Injection |
| glulisine | | | | |
| Insulin human | | DB00030 | INSR | Injection |
| Insulin lispro | | DB00046 | INSR | Injection |
| Metformin | Biguanides | DB00331 | ETFDH | Oral anti-diabetic drugs |
| Metformin | | DB00331 | PRKAB1 | Oral anti-diabetic drugs |
| Mitiglinide | Meglitinides | DB01252 | ABCC8 | Oral anti-diabetic drugs |
| Nateglinide | | DB00731 | ABCC8 | Oral anti-diabetic drugs |
| Repaglinide | | DB00912 | ABCC8 | Oral anti-diabetic drugs |
| Pramlintide | Amylin analog | DB01278 | RAMP3 | Injeciton |
| Pramlintide | | DB01278 | RAMP2 | Injeciton |
| Pramlintide | | DB01278 | RAMP1 | Injeciton |
| Pramlintide | | DB01278 | CALCR | Injeciton |
| Pioglitazone | Thiazolidinedione | DB01132 | PPARG | Oral anti-diabetic drugs |
| Rosiglitazone | (TZD) | DB00412 | PPARG | Oral anti-diabetic drugs |

TABLE 3
Characteristics of Single Nucleotide Polymorphisms (SNPs) Used as Instruments
to Proxy Single Targets (Using GWAS Instruments). This table presents
the characteristics of the SNPs used as instruments to proxy all drug
targets, utilizing data from the T2D Instruments. The table includes
information such as SNP identifiers, and allele information.

| | | | | | | |
|---|---|---|---|---|---|---|
| | | Effect | | P | F | R2 |
| SNP | EA/NEA | (GWAS) | SE | value | value | explained |

DeABCC8

| | | | | | | |
|---|---|---|---|---|---|---|
| rs214086 | C/G | 0.0358 | 0.005 | 9.92E−15 | 51.2656 | 0.006686696 |
| rs5215 | T/C | −0.0743 | 0.005 | 1.27E−54 | 220.8196 | 0.013444785 |
| rs7935408 | T/C | 0.0377 | 0.0058 | 3.43E−12 | 42.25 | 0.006011594 |

GLP1R

| | | | | | | |
|---|---|---|---|---|---|---|
| rs1929899 | A/G | −0.0438 | 0.0084 | 2.27E−09 | 27.18877551 | 0.005163377 |
| rs10305420 | T/C | −0.0288 | 0.0057 | 2.87E−08 | 25.52908587 | 0.004793831 |
| rs9366994 | T/C | −0.0392 | 0.0067 | 5.96E−15 | 34.2312319 | 0.006742407 |
| rs34179517 | A/C | −0.0421 | 0.0071 | 1.38E−08 | 35.15988891 | 0.004903328 |
| rs34247110 | A/G | 0.0429 | 0.0049 | 3.37E−21 | 76.65181175 | 0.008164129 |
| rs9471070 | T/C | −0.0572 | 0.0096 | 1.60E−11 | 35.50173611 | 0.005821114 |

KCNJ11

| | | | | | | |
|---|---|---|---|---|---|---|
| rs214086 | C/G | 0.0358 | 0.005 | 9.92E−15 | 51.2656 | 0.006686696 |
| rs5215 | T/C | −0.0743 | 0.005 | 1.27E−54 | 220.8196 | 0.013444785 |
| rs7935408 | T/C | 0.0377 | 0.0058 | 3.43E−12 | 42.25 | 0.006011594 |

KCNJ8

| | | | | | | |
|---|---|---|---|---|---|---|
| rs10841890 | T/C | 0.034 | 0.006 | 4.22E−08 | 32.11111111 | 0.004735455 |

PPARG

| | | | | | | |
|---|---|---|---|---|---|---|
| rs308958 | A/T | −0.0429 | 0.0071 | 1.02E−09 | 36.50882761 | 0.005274951 |
| rs7637403 | A/G | −0.0658 | 0.0078 | 1.48E−19 | 71.16436555 | 0.007814648 |
| rs2920502 | C/G | 0.0305 | 0.0054 | 2.60E−08 | 31.9015775 | 0.004808643 |
| rs17036160 | T/C | −0.1059 | 0.0086 | 2.88E−38 | 151.6334505 | 0.011173365 |
| rs116219174 | A/G | −0.1254 | 0.0208 | 2.01E−09 | 36.34698595 | 0.005180728 |
| rs4518111 | A/C | 0.0392 | 0.0051 | 4.91E−16 | 59.07881584 | 0.007009304 |

EA: effect allele, NEA: alter effect allele. Range of R2 and F-statistics correspond to estimates of these metrics across instruments constructed using independent (r2 <0.001) and weakly correlated (r2 <0.20) SNPs.

TABLE 4
Characteristics of Single Nucleotide Polymorphisms (SNPs) Used as
Instruments to Proxy All Drug Targets (Using GTEx Instruments)

| SNP | Target | EA | NEA | Beta | SE | P value | F value |
|---|---|---|---|---|---|---|---|
| rs10305525 | GLP1R | C | A | 0.0158 | 0.0068 | 0.0207 | 5.3988 |
| rs10766392 | KCNJ11 | G | T | 0.0384 | 0.0054 | 0.0000 | 50.5679 |
| rs10766393 | KCNJ11 | G | A | 0.0381 | 0.0054 | 0.0000 | 49.7809 |
| rs10766395 | KCNJ11 | T | C | 0.0399 | 0.0049 | 0.0000 | 66.3061 |
| rs1078523 | RAMP2 | A | G | 0.0160 | 0.0051 | 0.0019 | 9.8424 |
| rs10841887 | KCNJ8 | T | C | 0.0340 | 0.0060 | 0.0000 | 32.1111 |
| rs111327339 | PPARG | T | C | 0.0662 | 0.0240 | 0.0057 | 7.6084 |
| rs11150624 | SLC5A2 | T | C | 0.0122 | 0.0050 | 0.0155 | 5.9536 |
| rs111917515 | GANC | T | C | 0.0271 | 0.0103 | 0.0085 | 6.9225 |
| rs112898001 | GANC | A | G | 0.0251 | 0.0106 | 0.0183 | 5.6071 |
| rs112968754 | GANC | T | C | 0.0246 | 0.0102 | 0.0158 | 5.8166 |
| rs113126535 | GANC | T | C | 0.0250 | 0.0106 | 0.0187 | 5.5625 |
| rs11654396 | RAMP2 | T | G | 0.0136 | 0.0058 | 0.0195 | 5.4982 |
| rs116842927 | KCNJ1 | C | T | 0.0459 | 0.0230 | 0.0455 | 3.9826 |
| rs11772021 | RAMP3 | C | T | 0.0483 | 0.0084 | 0.0000 | 33.0625 |
| rs117973841 | KCNJ8 | G | T | 0.0302 | 0.0150 | 0.0441 | 4.0535 |
| rs11865835 | SLC5A2 | T | C | 0.0115 | 0.0056 | 0.0399 | 4.2172 |
| rs11869741 | RAMP2 | C | T | 0.0359 | 0.0156 | 0.0211 | 5.2959 |
| rs11963172 | GLP1R | A | G | 0.0180 | 0.0071 | 0.0107 | 6.4273 |
| rs12215108 | GLP1R | G | T | 0.0193 | 0.0093 | 0.0378 | 4.3067 |
| rs12448775 | SLC5A2 | T | C | 0.0312 | 0.0141 | 0.0270 | 4.8963 |
| rs12603201 | RAMP2 | C | T | 0.0247 | 0.0050 | 0.0000 | 24.4036 |
| rs12816749 | KCNJ8 | A | G | 0.0169 | 0.0063 | 0.0070 | 7.1960 |
| rs12941945 | RAMP2 | G | A | 0.0229 | 0.0063 | 0.0003 | 13.2126 |
| rs1373641 | PPARG | T | C | 0.0222 | 0.0057 | 0.0001 | 15.1690 |
| rs142133957 | SLC5A2 | A | G | 0.0613 | 0.0281 | 0.0292 | 4.7589 |
| rs142878626 | GLP1R | G | A | 0.0434 | 0.0180 | 0.0161 | 5.8135 |
| rs144057856 | INSR | T | G | 0.0901 | 0.0374 | 0.0160 | 5.8037 |
| rs147673442 | KCNJ11 | C | T | 0.0386 | 0.0118 | 0.0010 | 10.7007 |
| rs150243609 | ETFDH | T | G | 0.0700 | 0.0312 | 0.0250 | 5.0337 |
| rs1659215 | GANC | C | T | 0.0185 | 0.0085 | 0.0297 | 4.7370 |
| rs1699348 | PPARG | C | T | 0.0125 | 0.0054 | 0.0200 | 5.3584 |
| rs174587 | GANAB | C | T | 0.0179 | 0.0067 | 0.0077 | 7.1377 |
| rs17485664 | PRKAB1 | C | T | 0.0269 | 0.0128 | 0.0351 | 4.4166 |
| rs188581353 | DPP4 | A | G | 0.0799 | 0.0365 | 0.0286 | 4.7919 |
| rs189603359 | KCNJ11 | A | G | 0.0514 | 0.0262 | 0.0498 | 3.8488 |
| rs2074312 | ABCC8 | G | A | 0.0370 | 0.0054 | 0.0000 | 46.9479 |
| rs2080714 | DPP4 | G | T | 0.0154 | 0.0064 | 0.0158 | 5.7900 |
| rs2120825 | PPARG | T | G | 0.0889 | 0.0090 | 0.0000 | 97.5705 |
| rs2285676 | KCNJ11 | A | G | 0.0410 | 0.0049 | 0.0000 | 70.0125 |
| rs228817 | GLP1R | C | T | 0.0129 | 0.0055 | 0.0187 | 5.5012 |
| rs2355016 | KCNJ11 | G | A | 0.0360 | 0.0065 | 0.0000 | 30.6746 |
| rs28360624 | GLP1R | A | G | 0.0167 | 0.0078 | 0.0331 | 4.5840 |
| rs2881654 | PPARG | G | A | 0.0887 | 0.0077 | 0.0000 | 132.6984 |
| rs2920503 | PPARG | T | C | 0.0172 | 0.0054 | 0.0014 | 10.1454 |
| rs310752 | PPARG | A | G | 0.0130 | 0.0050 | 0.0099 | 6.7600 |
| rs34359922 | GANC | G | A | 0.0236 | 0.0099 | 0.0166 | 5.6827 |
| rs34497199 | SLC5A2 | C | T | 0.0129 | 0.0054 | 0.0164 | 5.7068 |
| rs35271178 | KCNJ11 | C | T | 0.0645 | 0.0050 | 0.0000 | 166.4100 |
| rs4031066 | INSR | T | G | 0.0146 | 0.0060 | 0.0157 | 5.9211 |
| rs4135247 | PPARG | G | A | 0.0373 | 0.0050 | 0.0000 | 55.6516 |
| rs4148631 | KCNJ11 | A | G | 0.0143 | 0.0059 | 0.0160 | 5.8745 |
| rs4233648 | DPP4 | T | C | 0.0124 | 0.0054 | 0.0210 | 5.2730 |
| rs4561482 | SLC5A2 | A | G | 0.0117 | 0.0050 | 0.0202 | 5.4756 |
| rs4663804 | RAMP1 | C | T | 0.0102 | 0.0051 | 0.0476 | 4.0000 |
| rs4684833 | PPARG | T | C | 0.0228 | 0.0062 | 0.0002 | 13.5234 |
| rs4724382 | RAMP3 | A | C | 0.0111 | 0.0053 | 0.0349 | 4.3863 |
| rs4924660 | GANC | A | G | 0.0174 | 0.0065 | 0.0074 | 7.1659 |
| rs4937311 | KCNJ1 | C | A | 0.0126 | 0.0053 | 0.0166 | 5.6518 |
| rs5210 | KCNJ11 | G | A | 0.0406 | 0.0049 | 0.0000 | 68.6531 |
| rs5219 | KCNJ11 | T | C | 0.0743 | 0.0051 | 0.0000 | 212.2449 |
| rs59406438 | KCNJ8 | T | C | 0.0473 | 0.0161 | 0.0033 | 8.6312 |
| rs59795094 | RAMP2 | C | T | 0.0130 | 0.0050 | 0.0099 | 6.7600 |
| rs7110037 | KCNJ11 | T | C | 0.0380 | 0.0063 | 0.0000 | 36.3820 |
| rs7110898 | KCNJ1 | A | G | 0.0235 | 0.0092 | 0.0105 | 6.5247 |
| rs7112030 | KCNJ11 | G | A | 0.0403 | 0.0049 | 0.0000 | 67.6422 |
| rs72681706 | AMY2A | A | G | 0.0427 | 0.0195 | 0.0284 | 4.7950 |
| rs72692396 | AMY2A | A | G | 0.0501 | 0.0216 | 0.0204 | 5.3798 |
| rs73111954 | RAMP3 | C | T | 0.0235 | 0.0062 | 0.0001 | 14.3665 |
| rs73402785 | GANC | C | T | 0.0237 | 0.0099 | 0.0161 | 5.7309 |
| rs739688 | KCNJ11 | C | T | 0.0167 | 0.0051 | 0.0012 | 10.7224 |
| rs74732083 | KCNJ8 | G | A | 0.0446 | 0.0184 | 0.0151 | 5.8754 |
| rs75390434 | RAMP3 | C | T | 0.0117 | 0.0057 | 0.0404 | 4.2133 |
| rs75850673 | DPP4 | G | T | 0.0161 | 0.0068 | 0.0184 | 5.6058 |
| rs76763697 | PPARG | T | C | 0.0224 | 0.0091 | 0.0135 | 6.0592 |
| rs77023203 | ABCC8 | G | A | 0.0428 | 0.0050 | 0.0000 | 73.2736 |
| rs77902362 | KCNJ11 | C | T | 0.0308 | 0.0138 | 0.0253 | 4.9813 |
| rs7940894 | KCNJ1 | A | G | 0.0460 | 0.0232 | 0.0472 | 3.9313 |
| rs79506407 | KCNJ11 | G | A | 0.0411 | 0.0107 | 0.0001 | 14.7542 |
| rs8037100 | GANC | C | T | 0.0195 | 0.0083 | 0.0186 | 5.5197 |
| rs8054784 | SLC5A2 | T | C | 0.0111 | 0.0051 | 0.0311 | 4.7370 |
| rs8057029 | SLC5A2 | A | C | 0.0116 | 0.0051 | 0.0243 | 5.1734 |
| rs8057326 | SLC5A2 | T | C | 0.0105 | 0.0050 | 0.0371 | 4.4100 |
| rs880347 | GLP1R | G | A | 0.0209 | 0.0054 | 0.0001 | 14.9798 |
| rs9462535 | GLP1R | A | C | 0.0155 | 0.0050 | 0.0021 | 9.6100 |
| rs9892728 | RAMP2 | C | T | 0.0152 | 0.0050 | 0.0026 | 9.2416 |
| rs9905939 | RAMP2 | C | T | 0.0154 | 0.0050 | 0.0022 | 9.4864 |

TABLE 5
Characteristics of Single Nucleotide Polymorphisms (SNPs) Used as Instruments for 6 KCNJ11-PPI of Drug Targets (Using
GTEx Instruments). This table presents the characteristics of the SNPs used as instruments for assessing the 6
PPARG-PPI (KCNJ11, ABCC8, ABCC9, KCNQ1, SIK1, and PRKACA) of drug targets, utilizing data from the Genotype-Tissue
Expression (GTEx) project. The table includes information such as SNP identifiers, and allele information.

| SNP | | | | | | | | |
|---|---|---|---|---|---|---|---|---|
| KCNJ11 PPI | | | | | P | F | R2 | |
| (GTEx-based) | Target | EA/NEA | Beta | SE | value | value | expl. | Tissue |
| Rs10766392 | KCNJ11 | T/G | −0.0384 | 0.0054 | 6E−15 | 50.5679 | 0.1581 | Vagina |
| Rs10766393 | KCNJ11 | A/G | −0.0381 | 0.0054 | 1.2E−14 | 49.7809 | 0.1569 | Esophagus_Mucosa |
| Rs10766395 | KCNJ11 | C/T | −0.0399 | 0.0049 | 0 | 66.3061 | 0.1807 | Skin_Not_Sun_Exposed_Suprapubic |
| Rs147673442 | KCNJ11 | T/C | −0.0386 | 0.0118 | 0.002926099 | 10.7007 | 0.0731 | Brain_Substantia_nigra |
| Rs189603359 | KCNJ11 | A/G | 0.0514 | 0.0262 | 0.288553863 | 3.8488 | 0.0439 | Brain_Hippocampus |
| rs2074312 | ABCC8 | A/G | −0.037 | 0.0054 | 8.2E−14 | 46.9479 | 0.1524 | Brain_Cerebellar_Hemisphere |
| rs2285676 | KCNJ11 | G/A | −0.041 | 0.0049 | 0 | 70.0125 | 0.1856 | Artery_Aorta |
| rs2355016 | KCNJ11 | A/G | 0.036 | 0.0065 | 2.57E−08 | 30.6746 | 0.1234 | Esophagus_Muscularis |
| rs35095853 | PRKACA | G/A | −0.0177 | 0.0076 | 0.118535629 | 5.4240 | 0.0521 | Esophagus_Mucosa |
| rs35271178 | KCNJ11 | T/C | 0.0645 | 0.005 | 0 | 166.4100 | 0.2827 | Whole_Blood |
| rs4148631 | KCNJ11 | A/G | 0.0143 | 0.0059 | 0.038175997 | 5.8745 | 0.0542 | Nerve_Tibial |
| rs5210 | KCNJ11 | A/G | −0.0406 | 0.0049 | 0 | 68.6531 | 0.1838 | Esophagus_Gastroesophageal_Junction |
| rs5219 | KCNJ11 | C/T | −0.0743 | 0.0051 | 0 | 212.2449 | 0.3175 | Skin_Sun_Exposed_Lower_leg |
| rs61928469 | ABCC9 | C/T | −0.053 | 0.0162 | 0.000302708 | 10.7034 | 0.0731 | Kidney_Cortex |
| rs7110037 | KCNJ11 | C/T | 0.038 | 0.0063 | 1.91E−09 | 36.3820 | 0.1343 | Colon_Sigmoid |
| rs7112030 | KCNQ1 | A/G | 0.0403 | 0.0049 | 0 | 67.6422 | 0.1825 | Testis |
| rs739688 | KCNJ11 | C/T | 0.0167 | 0.0051 | 0.00059763 | 10.7224 | 0.0732 | Liver |
| rs77023203 | ABCC8 | A/G | −0.0428 | 0.005 | 0 | 73.2736 | 0.1898 | Brain_Frontal_Cortex_BA9 |
| rs77902362 | KCNJ11 | C/T | 0.0308 | 0.0138 | 0.100412889 | 4.9813 | 0.0499 | Kidney_Cortex |
| rs78953484 | SIK1 | T/G | −0.04 | 0.0181 | 0.021517185 | 4.8839 | 0.0449 | Uterus |
| rs79506407 | KCNQ1 | A/G | 0.0411 | 0.0107 | 0.000135134 | 14.7542 | 0.0858 | Cells_EBV-transformed_lymphocytes |

EA: effect allele, NEA: alter effect allele; R2 expl: R2 explained.

TABLE 6
Characteristics of Single Nucleotide Polymorphisms (SNPs) Used as Instruments for 30 PPARG Protein-Protein
Interaction (PPI) of Drug Targets (Using GTEx Instruments). This table presents the characteristics of the
SNPs used as instruments for assessing the 30 PPARG-PPI-based targets (NFKB1, RXRB, NOS3, CDK5, MAPK8, NCOR2,
MTOR, PPARG, IL6, VEGFA, TP53, HDAC5, CDK19, SL2A4, ADRB3, ICAM1, MMP9, AGTR1, TNF, HDAC1, CDK8, TGFB1, EGFR,
HDAC2, RXRG, NFKB1, ABCA1, REN, HDAC7, HSD11B1, and HDAC3), utilizing data from the Genotype-Tissue Expression
(GTEx) project. The table includes information such as SNP identifiers, and allele information.

| SNP | | | | | | | | |
|---|---|---|---|---|---|---|---|---|
| PPARG PPI | | | | | P | F | R2 | |
| (GTEx-based) | Target | EA/NEA | Beta | SE | value | value | expl | Tissues |
| rs10014230 | NFKB1 | G/A | 0.0157 | 0.0065 | 0.03140662 | 5.8341 | 0.0031 | Brain_Cerebellar_Hemisphere |
| rs1009616 | RXRB | T/G | 0.0127 | 0.0056 | 0.006804363 | 5.1432 | 0.0039 | Brain_Cerebellum |
| rs10236214 | NOS3 | T/C | 0.0241 | 0.0063 | 0.000681472 | 14.6337 | 0.0048 | Skin_Not_Sun_Exposed_Suprapubic |
| rs10278673 | CDK5 | A/G | 0.0283 | 0.0054 | 4.27E−08 | 27.4654 | 0.0078 | Skin_Sun_Exposed_Lower_leg |
| rs10437448 | MAPK8 | C/T | 0.0098 | 0.0049 | 0.224407866 | 4.0000 | 0.0017 | Ovary |
| rs10456417 | RXRB | C/T | 0.0519 | 0.0148 | 0.000414958 | 12.2973 | 0.0050 | Uterus |
| rs10773050 | NCOR2 | G/A | 0.0223 | 0.0062 | 0.001497569 | 12.9368 | 0.0045 | Whole_Blood |
| rs10857566 | MAPK8 | A/G | 0.0109 | 0.0049 | 0.143711885 | 4.9484 | 0.0021 | Whole_Blood |
| rs11057692 | NCOR2 | G/A | 0.0148 | 0.0059 | 0.002115149 | 6.2924 | 0.0044 | Esophagus_Muscularis |
| rs11101325 | MAPK8 | T/C | 0.0129 | 0.0049 | 0.033118513 | 6.9309 | 0.0030 | Esophagus_Muscularis |
| rs11121704 | MTOR | T/C | 0.0249 | 0.006 | 6.01E−05 | 17.2225 | 0.0057 | Muscle_Skeletal |
| rs111327339 | PPARG | T/C | 0.0662 | 0.024 | 0.040901902 | 7.6084 | 0.0029 | Spleen |
| rs113525358 | IL6 | G/T | 0.0402 | 0.0151 | 0.045860192 | 7.0876 | 0.0028 | Adrenal_Gland |
| rs114227078 | RXRB | A/G | 0.0629 | 0.0243 | 0.077198794 | 6.7002 | 0.0025 | Heart_Left_Ventricle |
| rs11571999 | VEGFA | C/A | 0.0308 | 0.0151 | 0.048911257 | 4.1605 | 0.0028 | Adipose_Visceral_Omentum |
| rs117135784 | TP53 | A/G | 0.0442 | 0.0221 | 0.016335713 | 4.0000 | 0.0034 | Prostate |
| rs117274551 | HDAC5 | C/ | 0.0593 | 0.0193 | 0.013734432 | 9.4405 | 0.0035 | Brain_Nucleus_accumbens_basal_ganglia |
| rs117557561 | TP53 | A/G | 0.0607 | 0.0226 | 0.006088894 | 7.2137 | 0.0039 | Pituitary |
| rs11758099 | CDK19 | G/A | 0.0144 | 0.0064 | 0.117195516 | 5.0625 | 0.0022 | Skin_Not_Sun_Exposed_Suprapubic |
| rs117643180 | SLC2A4 | A/C | 0.0539 | 0.019 | 0.014099333 | 8.0477 | 0.0035 | Muscle_Skeletal |
| rs11774114 | ADRB3 | C/T | 0.012 | 0.0049 | 0.11019599 | 5.9975 | 0.0023 | Brain_Anterior_cingulate_cortex_BA24 |
| rs11776404 | ADRB3 | G/A | 0.0193 | 0.0077 | 0.015884594 | 6.2825 | 0.0034 | Stomach |
| rs118115488 | ICAM1 | T/C | 0.0386 | 0.0121 | 0.003291524 | 10.1766 | 0.0042 | Pituitary |
| rs11907381 | MMP9 | T/C | 0.0129 | 0.0059 | 0.043896002 | 4.7805 | 0.0029 | Brain_Anterior_cingulate_cortex_BA24 |
| rs11926270 | AGTR1 | C/T | 0.0139 | 0.0067 | 0.121086853 | 4.3041 | 0.0022 | Brain_Cerebellum |
| rs12453401 | HDAC5 | G/A | 0.0188 | 0.0081 | 0.084730256 | 5.3870 | 0.0025 | Thyroid |
| rs12525616 | TNF | G/T | 0.0314 | 0.009 | 0.002398201 | 12.1723 | 0.0043 | Heart_Atrial_Appendage |
| rs12567940 | HDAC1 | A/C | 0.0413 | 0.0104 | 0.000546176 | 15.7701 | 0.0049 | Brain_Hypothalamus |
| rs12864131 | CDK8 | G/A | 0.01 | 0.0051 | 0.044702556 | 3.8447 | 0.0029 | Artery_Aorta |
| rs12983775 | TGFB1 | A/G | 0.0109 | 0.0051 | 0.153499796 | 4.5679 | 0.0020 | Spleen |
| rs13238083 | EGFR | G/A | 0.046 | 0.0218 | 0.050420748 | 4.4525 | 0.0028 | Uterus |
| rs1373641 | PPARG | T/C | 0.0222 | 0.0057 | 0.001238905 | 15.1690 | 0.0046 | Artery_Tibial |
| rs139072136 | HDAC5 | A/C | 0.0541 | 0.0243 | 0.084645237 | 4.9566 | 0.0025 | Spleen |
| rs140897096 | VEGFA | C/T | 0.0862 | 0.0369 | 0.069406329 | 5.4571 | 0.0026 | Brain_Anterior_cingulate_cortex_BA24 |
| rs142433590 | HDAC1 | A/G | 0.0896 | 0.0331 | 0.02882321 | 7.3276 | 0.0031 | Spleen |
| rs148721283 | MAPK8 | T/C | 0.0558 | 0.0256 | 0.113653085 | 4.7510 | 0.0023 | Kidney_Cortex |
| rs149634409 | NCOR2 | T/C | 0.0515 | 0.0261 | 0.322556844 | 3.8934 | 0.0014 | Adrenal_Gland |
| rs1575050 | HDAC2 | T/C | 0.0108 | 0.0053 | 0.046653191 | 4.1524 | 0.0028 | Brain_Nucleus_a_ccumbens_basal_ganglia |
| rs1594570 | RXRG | T/G | 0.0115 | 0.0056 | 0.160029096 | 4.2172 | 0.0020 | Pancreas |
| rs1699348 | PPARG | C/T | 0.0125 | 0.0054 | 0.157849107 | 5.3584 | 0.0020 | Whole_Blood |
| rs17033014 | NFKB1 | A/G | 0.0114 | 0.0051 | 0.011056686 | 4.9965 | 0.0036 | Breast_Mammary_Tissue |
| rs17344810 | MMP9 | G/A | 0.02 | 0.0088 | 0.002674011 | 5.1653 | 0.0043 | Lung |
| rs17583407 | EGFR | C/T | 0.0099 | 0.0049 | 0.040964504 | 4.0820 | 0.0029 | Colon_Transverse |
| rs1800781 | NOS3 | G/A | 0.0264 | 0.0076 | 0.003619905 | 12.0665 | 0.0041 | Whole_Blood |
| rs1811376 | ICAM1 | G/A | 0.0272 | 0.0134 | 0.086058638 | 4.1203 | 0.0024 | Artery_Aorta |
| rs1883965 | MTOR | G/A | 0.0261 | 0.006 | 1.56E−05 | 18.9225 | 0.0062 | Esophagus_Mucosa |
| rs189732367 | NCOR2 | G/A | 0.0703 | 0.0301 | 0.111158625 | 5.4548 | 0.0023 | Prostate |
| rs1924825 | CDK8 | T/C | 0.0261 | 0.0067 | 1.09E−05 | 15.1751 | 0.0063 | Brain_Nucleus_accumbens_basal_ganglia |
| rs2120825 | PPARG | T/G | 0.0889 | 0.009 | 5.00E−25 | 97.5705 | 0.0147 | Testis |
| rs2149076 | CDK8 | C/A | 0.0203 | 0.0053 | 1.04E−05 | 14.6703 | 0.0063 | Heart_Left_Ventricle |
| rs222853 | SLC2A4 | A/G | 0.0239 | 0.0092 | 0.017554946 | 6.7487 | 0.0034 | Small_Intestine_Terminal_Ileum |
| rs2231258 | RXRB | G/A | 0.0498 | 0.0254 | 0.071229976 | 3.8441 | 0.0026 | Skin_Not_Sun_Exposed_Suprapubic |
| rs2231648 | HDAC5 | C/T | 0.0418 | 0.0113 | 0.000770353 | 13.6835 | 0.0048 | Heart_Atrial_Appendage |
| rs2244278 | ABCA1 | C/A | 0.0147 | 0.0069 | 0.110634482 | 4.5388 | 0.0023 | Cells_Cultured_fibroblasts |
| rs2269423 | TNF | C/A | 0.0145 | 0.0053 | 2.71E−06 | 7.4849 | 0.0067 | Kidney_Cortex |
| rs2277127 | VEGFA | T/C | 0.0164 | 0.0082 | 0.221969497 | 4.0000 | 0.0017 | Vagina |
| rs2317131 | TGFB1 | T/C | 0.0203 | 0.0049 | 0.000341858 | 17.1633 | 0.0051 | Esophagus_Mucosa |
| rs2472491 | ABCA1 | A/G | 0.0198 | 0.0055 | 0.002206049 | 12.9600 | 0.0044 | Lung |
| rs2487151 | NOS3 | G/A | 0.014 | 0.0063 | 0.083039431 | 4.9383 | 0.0025 | Brain_Spinal_cord_cervical_c-1 |
| rs2515602 | ABCA1 | A/G | 0.0182 | 0.0054 | 0.000673905 | 11.3594 | 0.0048 | Brain_Putamen_basal_ganglia |
| rs2530714 | TNF | G/A | 0.0203 | 0.0058 | 0.001004209 | 12.2500 | 0.0047 | Brain_Frontal_Cortex_BA9 |
| rs2744820 | MTOR | G/A | 0.02 | 0.0057 | 0.000141784 | 12.3115 | 0.0054 | Colon_Transverse |
| rs2777795 | ABCA1 | G/A | 0.0161 | 0.0074 | 0.040031028 | 4.7336 | 0.0029 | Muscle_Skeletal |
| rs2788543 | MTOR | T/C | 0.0198 | 0.0057 | 0.000234396 | 12.0665 | 0.0052 | Brain_Cortex |
| rs2791656 | MTOR | G/A | 0.0286 | 0.0065 | 1.76E−05 | 19.3600 | 0.0061 | Skin_Sun_Exposed_Lower_leg |
| rs281436 | ICAM1 | A/G | 0.0148 | 0.0059 | 0.04679777 | 6.2924 | 0.0028 | Whole_Blood |
| rs28507274 | REN | A/G | 0.0277 | 0.0097 | 0.000321474 | 8.1549 | 0.0051 | Artery_Tibial |
| rs2881654 | PPARG | G/A | 0.0887 | 0.0077 | 2.98E−36 | 132.6984 | 0.0179 | Esophagus_Gastroesophageal_Junction |
| rs2920503 | PPARG | T/C | 0.0172 | 0.0054 | 0.001501756 | 10.1454 | 0.0045 | Nerve_Tibial |
| rs3025000 | VEGFA | T/C | 0.0141 | 0.0054 | 0.070255216 | 6.8179 | 0.0026 | Thyroid |
| rs3094006 | TNF | C/T | 0.0312 | 0.0056 | 7.72E−09 | 31.0408 | 0.0082 | Cells_EBV-transformed_lymphocytes |
| rs310752 | PPARG | A/G | 0.013 | 0.005 | 0.025471437 | 6.7600 | 0.0032 | Cells_Cultured_fibroblasts |
| rs3199966 | ABCA1 | T/C | 0.0223 | 0.0092 | 0.052257643 | 5.8754 | 0.0028 | Small_Intestine_Terminal_Ileum |
| rs34137317 | TNF | C/T | 0.0774 | 0.0253 | 0.015055077 | 9.3592 | 0.0035 | Artery_Coronary |
| rs34186648 | VEGFA | G/A | 0.0133 | 0.0058 | 0.061327874 | 5.2583 | 0.0027 | Brain_Putamen_basal_ganglia |
| rs34238147 | CDK8 | G/A | 0.045 | 0.0056 | 1.55E−17 | 64.5727 | 0.0121 | Adrenal_Gland |
| rs34945449 | TGFB1 | T/C | 0.0124 | 0.0059 | 0.242790849 | 4.4171 | 0.0017 | Adrenal_Gland |
| rs3730305 | CDK5 | C/A | 0.0207 | 0.0091 | 0.139536649 | 5.1744 | 0.0021 | Brain_Cerebellar_Hemisphere |
| rs373494 | RXRG | G/A | 0.013 | 0.0065 | 0.014057843 | 4.0000 | 0.0035 | Thyroid |
| rs3793341 | CDK5 | A/G | 0.0257 | 0.0069 | 0.001510891 | 13.8729 | 0.0045 | Brain_Putamen_basal_ganglia |
| rs3815138 | HDAC7 | T/C | 0.0129 | 0.0065 | 0.241925239 | 3.9387 | 0.0017 | Brain_Hypothalamus |
| rs382454 | HDAC1 | A/G | 0.0428 | 0.0091 | 1.42E−05 | 22.1210 | 0.0062 | Esophagus_Gastroesophageal_Junction |
| rs3827681 | MAPK8 | C/T | 0.0109 | 0.0049 | 0.148919582 | 4.9484 | 0.0021 | Colon_Transverse |
| rs3910433 | CDK8 | C/T | 0.066 | 0.014 | 1.65E−05 | 22.2245 | 0.0061 | Brain_Caudate_basal_ganglia |
| rs3950310 | MAPK8 | C/T | 0.0099 | 0.0049 | 0.285099907 | 4.0820 | 0.0015 | Pituitary |
| rs4135247 | PPARG | G/A | 0.0373 | 0.005 | 2.26E−14 | 55.6516 | 0.0109 | Esophagus_Muscularis |
| rs4648052 | NFKB1 | G/T | 0.01 | 0.0049 | 0.014728618 | 4.1649 | 0.0035 | Testis |
| rs4684833 | PPARG | T/C | 0.0228 | 0.0062 | 3.14E−05 | 13.5234 | 0.0059 | Thyroid |
| rs4791840 | TP53 | C/A | 0.0117 | 0.0058 | 0.112851317 | 4.0693 | 0.0023 | Vagina |
| rs4810482 | MMP9 | T/C | 0.0158 | 0.0051 | 0.015522766 | 9.5978 | 0.0034 | Testis |
| rs4838593 | MAPK8 | C/T | 0.01 | 0.005 | 0.043219167 | 4.0000 | 0.0029 | Colon_Sigmoid |
| rs536109 | RXRG | C/A | 0.0118 | 0.005 | 0.005574455 | 5.5696 | 0.0040 | Brain_Spinal_cord_cervical_c-1 |
| rs547632 | CDK8 | C/T | 0.0286 | 0.0051 | 5.81E−09 | 31.4479 | 0.0083 | Nerve_Tibial |
| rs56310407 | CDK8 | T/C | 0.0516 | 0.0176 | 0.013819438 | 8.5956 | 0.0035 | Brain_Cortex |
| rs58477215 | NFKB1 | C/T | 0.0241 | 0.0063 | 9.73E−06 | 14.6337 | 0.0063 | Skin_Sun_Exposed_Lower_leg |
| rs6017556 | MMP9 | C/T | 0.0198 | 0.0085 | 0.137305391 | 5.4262 | 0.0021 | Uterus |
| rs6017721 | MMP9 | A/G | 0.0154 | 0.0051 | 0.010758065 | 9.1180 | 0.0036 | Cells_Cultured_fibroblasts |
| rs6130997 | MMP9 | A/G | 0.0163 | 0.005 | 0.008277382 | 10.6276 | 0.0038 | Minor_Salivary_Gland |
| rs62421524 | HDAC2 | C/T | 0.0117 | 0.0058 | 0.293910116 | 4.0693 | 0.0015 | Skin_Not_Sun_Exposed_Suprapubic |
| rs628300 | HSD11B1 | T/C | 0.0112 | 0.0054 | 0.163849304 | 4.3018 | 0.0020 | Adipose_Subcutaneous |
| rs6503062 | SLC2A4 | G/A | 0.0117 | 0.005 | 0.032714225 | 5.4756 | 0.0030 | Spleen |
| rs6691635 | REN | T/C | 0.0259 | 0.0109 | 0.115371095 | 5.6461 | 0.0022 | Brain_Caudate_basal_ganglia |
| rs67511749 | TGFB1 | A/G | 0.0125 | 0.0059 | 0.227325781 | 4.4887 | 0.0017 | Cells_Cultured_fibroblasts |
| rs6913605 | RXRB | G/A | 0.0261 | 0.0053 | 1.81E−06 | 24.2510 | 0.0068 | Stomach |
| rs72668700 | NFKB1 | G/A | 0.0555 | 0.0223 | 0.004203379 | 6.1941 | 0.0041 | Brain_Cortex |
| rs72790024 | HDAC3 | G/A | 0.0217 | 0.0109 | 0.049607035 | 3.9634 | 0.0028 | Muscle_Skeletal |
| rs72828635 | SLC2A4 | G/A | 0.0527 | 0.0233 | 0.0586308 | 5.1158 | 0.0027 | Heart_Left_Ventricle |
| rs735286 | VEGFA | T/C | 0.0139 | 0.0054 | 0.077273833 | 6.6259 | 0.0025 | Adrenal _Gland |
| rs742594 | MMP9 | C/T | 0.0129 | 0.0055 | 0.02941899 | 5.5012 | 0.0031 | Brain_Nucleus_accumbens_basal_ganglia |
| rs75359196 | RXRB | T/C | 0.0213 | 0.0104 | 0.046100868 | 4.1946 | 0.0028 | Brain_Hypothalamus |
| rs76178978 | CDK19 | T/C | 0.0148 | 0.0066 | 0.128644507 | 5.0285 | 0.0022 | Minor_Salivary_Gland |
| rs76316010 | MAPK8 | C/T | 0.02 | 0.0091 | 0.035495373 | 4.8303 | 0.0030 | Liver |
| rs7638700 | AGTR1 | C/A | 0.0213 | 0.0083 | 0.015946534 | 6.5857 | 0.0034 | Pancreas |
| rs76432155 | SLC2A4 | T/C | 0.0831 | 0.0119 | 1.95E−13 | 48.7650 | 0.0105 | Brain_Spinal_cord_cervical_c-1 |
| rs76498519 | NFKB1 | C/T | 0.0343 | 0.0158 | 0.187307444 | 4.7127 | 0.0019 | Thyroid |
| rs76593531 | TP53 | C/T | 0.0678 | 0.0113 | 2.60E−09 | 36.0000 | 0.0085 | Nerve_Tibial |
| rs7674212 | NFKB1 | G/T | 0.0199 | 0.005 | 6.96E−07 | 15.8404 | 0.0071 | Pituitary |
| rs76763697 | PPARG | T/C | 0.0224 | 0.0091 | 0.044006911 | 6.0592 | 0.0029 | Brain_Substantia_nigra |
| rs77161475 | SLC2A4 | G/A | 0.0458 | 0.0198 | 0.06021273 | 5.3506 | 0.0027 | Artery_Tibial |
| rs77307957 | HDAC5 | G/A | 0.0603 | 0.0271 | 0.150981428 | 4.9510 | 0.0020 | Brain_Cortex |
| rs79862595 | HDAC2 | T/C | 0.0156 | 0.0063 | 0.000229267 | 6.1315 | 0.0053 | Brain_Spinal_cord_cervical_c-1 |
| rs7989124 | CDK8 | G/A | 0.0102 | 0.005 | 0.042486854 | 4.1616 | 0.0029 | Heart_Atrial_Appendage |
| rs833069 | VEGFA | C/T | 0.0164 | 0.0051 | 0.003121839 | 10.3406 | 0.0042 | Esophagus_Muscularis |
| rs9262172 | TNF | C/T | 0.0164 | 0.0062 | 0.007268348 | 6.9969 | 0.0038 | Lung |
| rs9277895 | RXRB | A/G | 0.0229 | 0.0054 | 0.000138715 | 17.9839 | 0.0054 | Brain_Cerebellar_Hemisphere |
| rs9295981 | TNF | A/G | 0.0192 | 0.0054 | 0.003675419 | 12.6420 | 0.0041 | Liver |
| rs9469444 | RXRB | A/G | 0.0682 | 0.019 | 0.002612181 | 12.8843 | 0.0043 | Adipose_Subcutaneous |
| rs9970244 | RXRG | G/T | 0.0111 | 0.0051 | 0.00571998 | 4.7370 | 0.0039 | Spleen |

EA: effect allele, NEA: alter effect allele, R2 expl: R2 explained.

TABLE 7
Drug-Target Mendelian Randomization (MR) Estimates Examining the Association of
Genetically Proxied Perturbation of Single Drug Targets with Cancer Risk (Using
GTEx Instruments). This table provides MR estimates for the association between
genetically proxied perturbation of single drug targets and cancer risk using instrumental
variables derived from GWAS instruments. The analysis investigates the causal effect
of perturbing individual drug targets on the risk of developing cancer. The table
includes effect sizes (odds ratios), confidence intervals, and p-values, providing
insights into the strength and statistical significance of the observed associations.
The estimates include effect sizes (odds ratios, OR), 95% low confidence intervals
(95% LCI), 95% high confidence intervals (95% UCI) and P value, indicating the
strength and significance of the observed associations.

| Exposure | Outcome | nSNP | SNP | OR | 95% LCI | 95% UCI | P value |
|---|---|---|---|---|---|---|---|
| KCNJ11 | Tongue | 16 | IVW | 0.3686 | 0.1791 | 0.7589 | 0.006747663 |
| | cancer | | (random | | | | |
| | | | effects) | | | | |
| KCNJ11 | Tongue | 16 | IVW | 0.3686 | 0.1256 | 1.0818 | 0.069236133 |
| | cancer | | (fixed | | | | |
| | | | effects) | | | | |
| KCNJ11 | Tongue | 16 | All - | 0.2095 | 0.0107 | 4.1052 | 0.32060895 |
| | cancer | | MR | | | | |
| | | | Egger | | | | |
| KCNJ11 | Chronic | 16 | IVW | 0.2250 | 0.1030 | 0.4914 | 0.000182216 |
| | myelogenous | | (random | | | | |
| | leukemia | | effects) | | | | |
| KCNJ11 | Chronic | 16 | IVW | 0.2250 | 0.0356 | 1.4200 | 0.112520911 |
| | myelogenous | | (fixed | | | | |
| | leukemia | | effects) | | | | |
| KCNJ11 | Chronic | 16 | All - | 0.2765 | 0.0017 | 44.8915 | 0.628215584 |
| | myelogenous | | MR | | | | |
| | leukemia | | Egger | | | | |
| KCNJ11 | Gastric | 16 | IVW | 0.6796 | 0.5904 | 0.7822 | 7.37E−08 |
| | cancer | | (random | | | | |
| | | | effects) | | | | |
| KCNJ11 | Gastric | 16 | IVW | 0.6796 | 0.5495 | 0.8404 | 0.000364285 |
| | cancer | | (fixed | | | | |
| | | | effects) | | | | |
| KCNJ11 | Gastric | 16 | All - | 0.8253 | 0.4483 | 1.5193 | 0.54730662 |
| | cancer | | MR | | | | |
| | | | Egger | | | | |
| KCNJ11 | Squamous | 8 | IVW | 1.5836 | 1.1706 | 2.1422 | 0.002862773 |
| | cell lung | | (random | | | | |
| | carcinoma | | effects) | | | | |
| KCNJ11 | Squamous | 8 | IVW | 1.5836 | 1.0490 | 2.3906 | 0.028697199 |
| | cell lung | | (fixed | | | | |
| | carcinoma | | effects | | | | |
| KCNJ11 | Squamous | 8 | All - | 1.9992 | 0.8935 | 4.4729 | 0.142766314 |
| | cell lung | | MR | | | | |
| | carcinoma | | Egger | | | | |
| GLP1R | Endometrial | 8 | IVW | 4.5751 | 1.5831 | 13.2213 | 0.00497726 |
| | cancer | | (random | | | | |
| | | | effects) | | | | |
| GLP1R | Endometrial | 8 | IVW | 4.5751 | 2.1374 | 9.7927 | 0.0000899 |
| | cancer | | (fixed | | | | |
| | | | effects) | | | | |
| GLP1R | Endometrial | 8 | All - | 3.8558 | 0.0244 | 608.3625 | 0.619951533 |
| | cancer | | MR | | | | |
| | | | Egger | | | | |
| PPARG | Bronchial | 10 | IVW | 3.2866 | 1.8859 | 5.7276 | 0.0000268 |
| | cancer | | (random | | | | |
| | | | effects) | | | | |
| PPARG | Bronchial | 10 | IVW | 3.2866 | 1.7659 | 6.1170 | 0.000173965 |
| | cancer | | (fixed | | | | |
| | | | effects) | | | | |
| PPARG | Bronchial | 10 | All - | 4.5554 | 1.6937 | 12.2524 | 0.016974204 |
| | cancer | | MR | | | | |
| | | | Egger | | | | |
| PPARG | Oropharynx | 10 | IVW | 0.1680 | 0.0938 | 0.3008 | 1.94E−09 |
| | cancer | | (random | | | | |
| | | | effects) | | | | |
| PPARG | Oropharynx | 10 | IVW | 0.1680 | 0.0772 | 0.3658 | 0.00000703 |
| | cancer | | (fixed | | | | |
| | | | effects) | | | | |
| PPARG | Oropharynx | 10 | All - | 0.1483 | 0.0432 | 0.5090 | 0.016225456 |
| | cancer | | MR | | | | |
| | | | Egger | | | | |
| PPARG | Tongue | 10 | IVW | 0.0219 | 0.0092 | 0.0518 | 3.94662E−18 |
| | cancer | | (random | | | | |
| | | | effects) | | | | |
| PPARG | Tongue | 10 | IVW | 0.0219 | 0.0044 | 0.1080 | 2.70473E−06 |
| | cancer | | (fixed | | | | |
| | | | effects) | | | | |
| PPARG | Tongue | 10 | All - | 0.0097 | 0.0008 | 0.1232 | 0.00726947 |
| | cancer | | MR | | | | |
| | | | Egger | | | | |
| RAMP2 | Bronchial | 8 | IVW | 8.1341 | 2.6685 | 24.7947 | 0.000227802 |
| | cancer | | (random | | | | |
| | | | effects) | | | | |
| RAMP2 | Bronchial | 8 | IVW | 8.1341 | 2.1755 | 30.4127 | 0.001838262 |
| | cancer | | (fixed | | | | |
| | | | effects) | | | | |
| RAMP2 | Bronchial | 8 | All - | 2.1316 | 0.0191 | 237.3660 | 0.763595527 |
| | cancer | | MR | | | | |
| | | | Egger | | | | |
| All | Gastric | 86 | IVW | 0.7199 | 0.6172 | 0.8398 | 0.0000289 |
| targets | cancer | | (random | | | | |
| | | | effects) | | | | |
| All | Gastric | 86 | IVW | 0.7199 | 0.6121 | 0.8467 | 7.15E−05 |
| targets | cancer | | (fixed | | | | |
| | | | effects) | | | | |
| All | Gastric | 86 | All - | 0.6678 | 0.4936 | 0.9036 | 0.010513794 |
| targets | cancer | | MR | | | | |
| | | | Egger | | | | |
| All | Oropharynx | 86 | IVW | 0.5818 | 0.3930 | 0.8614 | 0.006823232 |
| targets | cancer | | (random | | | | |
| | | | effects) | | | | |
| All | Oropharynx | 86 | IVW | 0.5818 | 0.4016 | 0.8428 | 0.004178641 |
| targets | cancer | | (fixed | | | | |
| | | | effects) | | | | |
| All | Oropharynx | 86 | All - | 0.5096 | 0.2497 | 1.0400 | 0.067504734 |
| targets | cancer | | MR | | | | |
| | | | Egger | | | | |

TABLE 8
Drug-Target Mendelian Randomization (MR) Estimates Examining the Association of Genetically Proxied
Perturbation of Single Drug Targets with Cancer Risk (Using GWAS Instruments). This table provides
MR estimates for the association between genetically proxied perturbation of single drug targets
and cancer risk using instrumental variables derived from GWAS instruments. The analysis investigates
the causal effect of perturbing individual drug targets on the risk of developing cancer. The table
includes effect sizes (odds ratios), confidence intervals, and p-values, providing insights into
the strength and statistical significance of the observed associations. The estimates include effect
sizes (odds ratios, OR), 95% low confidence intervals (95% LCI), 95% high confidence intervals (95%
UCI) and P value, indicating the strength and significance of the observed associations.

| Exposure | Outcome | nSNP | SNP | OR | 95% LCI | 95% UCI | P value |
|---|---|---|---|---|---|---|---|
| ABCC8/ | Gastric cancer | 2 | IVW (random | 0.773629213 | 0.761619073 | 0.785828744 | 8.24E−227 |
| KCNJ11 | | | effects) | | | | |
| ABCC8/ | Gastric cancer | 2 | IVW (fixed | 0.7736 | 0.5194 | 1.1523 | 2.07E−01 |
| KCNJ11 | | | effects) | | | | |
| ABCC8/ | Tongue cancer | 2 | IVW (random | 0.4110 | 0.2619 | 0.6451 | 0.000110595 |
| KCNJ11 | | | effects) | | | | |
| ABCC8/ | Tongue cancer | 2 | IVW (fixed | 0.4110 | 0.0578 | 2.9217 | 0.374261718 |
| KCNJ11 | | | effects) | | | | |
| ABCC8/ | Chronic | 2 | IVW (random | 0.1965 | 0.1428 | 0.2703 | 1.62E−23 |
| KCNJ11 | myelogenous | | effects) | | | | |
| | leukemia | | | | | | |
| ABCC8/ | Chronic | 2 | IVW (fixed | 0.1965 | 0.0068 | 5.6398 | 3.42E−01 |
| KCNJ11 | myelogenous | | effects) | | | | |
| | leukemia | | | | | | |
| ABCC8/ | Squamous cell | 2 | IVW (random | 1.7340887 | 1.07132131 | 3.095848 | 0.04627 |
| KCNJ11 | lung carcinoma | | effects) | | | | |
| ABCC8/ | Squamous cell | 2 | IVW (fixed | 1.734088684 | 1.093442878 | 2.750087477 | 0.019300303 |
| KCNJ11 | lung carcinoma | | effects) | | | | |
| GLP1R | Endometrial | 6 | IVW (random | 1.7721 | 1.3977 | 2.2467 | 2.29E−06 |
| | cancer | | effects) | | | | |
| GLP1R | Endometrial | 6 | IVW (fixed | 1.7721 | 1.2006 | 2.6156 | 0.003971853 |
| | cancer | | effects) | | | | |
| GLP1R | Endometrial | 6 | All - MR | 3.2116 | 0.4240 | 24.3278 | 0.321868887 |
| | cancer | | Egger | | | | |
| PPARG | Bronchial | 4 | IVW (random | 2.6949 | 1.9561 | 3.7128 | 1.33E−09 |
| | cancer | | effects) | | | | |
| PPARG | Bronchial | 4 | IVW (fixed | 2.6949 | 1.4347 | 5.0618 | 0.002053528 |
| | cancer | | effects) | | | | |
| PPARG | Bronchial | 4 | All - MR | 3.1955 | 0.7189 | 14.2044 | 0.266455701 |
| | cancer | | Egger | | | | |
| PPARG | Oropharynx | 4 | IVW (random | 0.2565 | 0.1737 | 0.3789 | 8.07E−12 |
| | cancer | | effects) | | | | |
| PPARG | Oropharynx | 4 | IVW (fixed | 0.2565 | 0.1153 | 0.5710 | 0.000860527 |
| | cancer | | effects) | | | | |
| PPARG | Oropharynx | 4 | All - MR | 0.3529 | 0.0523 | 2.3824 | 0.396987706 |
| | cancer | | Egger | | | | |
| PPARG | Tongue cancer | 4 | IVW (random | 0.0488 | 0.0175 | 0.1363 | 8.27E−09 |
| | | | effects) | | | | |
| PPARG | Tongue cancer | 4 | IVW (fixed | 0.0488 | 0.0097 | 0.2458 | 0.000251177 |
| | | | effects) | | | | |
| PPARG | Tongue cancer | 4 | All - MR | 0.0227 | 0.0005 | 1.0372 | 0.191731718 |
| | | | Egger | | | | |
| RAMP2 | Bronchial | 2 | IVW (random | 2.0547 | 1.4210 | 2.9711 | 0.000129444 |
| | cancer | | effects | | | | |
| RAMP2 | Bronchial | 2 | IVW (fixed | 2.0547 | 0.6254 | 6.7504 | 2.35E−01 |
| | cancer | | effects) | | | | |
| All | Gastric cancer | 7 | IVW (random | 0.7870 | 0.5811 | 1.0658 | 0.121540938 |
| targets | | | effects) | | | | |
| All | Gastric cancer | 7 | IVW (fixed | 0.7870 | 0.5954 | 1.0402 | 0.092411085 |
| targets | | | effects) | | | | |
| All | Gastric cancer | 7 | All - MR | 0.8393 | 0.3275 | 2.1507 | 0.730107188 |
| targets | | | Egger | | | | |
| All | Oropharynx | 7 | IVW (random | 0.7494 | 0.3540 | 1.5866 | 0.45095562 |
| targets | cancer | | effects) | | | | |
| All | Oropharynx | 7 | IVW (fixed | 0.7494 | 0.3992 | 1.4069 | 0.369352558 |
| targets | cancer | | effects) | | | | |
| All | Oropharynx | 7 | All - MR | 0.1682 | 0.0335 | 0.8432 | 0.082421733 |
| targets | cancer | | Egger | | | | |

TABLE 9
Detailed drug-target MR estimates examining the association of genetically proxied perturbation
of other significant drug targets with cancer risk (GTEx instruments & GWAS instruments).

| Exposure | Outcome | nSNP | SNP | OR | 95% LCI | 95% UCI | P value | Method |
|---|---|---|---|---|---|---|---|---|
| KCNJ11 | TC | 16 | IVW | 0.3686 | 0.1791 | 0.7589 | 0.006747663 | GTEx |
| | | | (random | | | | | |
| | | | effects) | | | | | |
| KCNJ11 | TC | 16 | IVW (fixed | 0.3686 | 0.1256 | 1.0818 | 0.069236133 | GTEx |
| | | | effects) | | | | | |
| KCNJ11 | TC | 16 | All - | 0.3717 | 0.1259 | 1.0976 | 0.073225702 | GTEx |
| | | | Maximum | | | | | |
| | | | likelihood | | | | | |
| KCNJ11 | TC | 16 | All - | 0.6990 | 0.1397 | 3.4981 | 0.66292064 | GTEx |
| | | | Simple | | | | | |
| | | | median | | | | | |
| KCNJ11 | TC | 16 | All - | 0.3096 | 0.0783 | 1.2243 | 0.094638371 | GTEx |
| | | | Weighted | | | | | |
| | | | median | | | | | |
| KCNJ11 | TC | 16 | All - | 0.5709 | 0.0829 | 3.9298 | 0.577475523 | GTEx |
| | | | Simple | | | | | |
| | | | mode | | | | | |
| KCNJ11 | TC | 16 | All - | 0.2710 | 0.0611 | 1.2023 | 0.106437586 | GTEx |
| | | | Weighted | | | | | |
| | | | mode | | | | | |
| KCNJ11 | TC | 16 | All - | 0.2616 | 0.0577 | 1.1863 | 0.102592293 | GTEx |
| | | | Weighted | | | | | |
| | | | mode | | | | | |
| | | | (NOME) | | | | | |
| KCNJ11 | TC | 16 | All - | 0.5709 | 0.0931 | 3.5028 | 0.553853932 | GTEx |
| | | | Simple | | | | | |
| | | | mode | | | | | |
| | | | (NOME) | | | | | |
| KCNJ11 | TC | 16 | RAPS | | | | | GTEx |
| KCNJ11 | TC | 16 | MR- | | | | 0.01616278 | GTEx |
| | | | PRESSO | | | | | |
| | | | (Raw) | | | | | |
| KCNJ11 | SCLC | 8 | IVW | 1.5836 | 1.1706 | 2.1422 | 0.002862773 | GTEx |
| | | | (random | | | | | |
| | | | effects) | | | | | |
| KCNJ11 | SCLC | 8 | IVW (fixed | 1.5836 | 1.0490 | 2.3906 | 0.028697199 | GTEx |
| | | | effects) | | | | | |
| KCNJ11 | SCLC | 8 | All - MR | 1.9992 | 0.8935 | 4.4729 | 0.142766314 | GTEx |
| | | | Egger | | | | | |
| KCNJ11 | SCLC | 8 | All - | 1.5961 | 1.0507 | 2.4246 | 0.02839495 | GTEx |
| | | | Maximum | | | | | |
| | | | likelihood | | | | | |
| KCNJ11 | SCLC | 8 | All - | 1.3358 | 0.5960 | 2.9943 | 0.481964304 | GTEx |
| | | | Simple | | | | | |
| | | | median | | | | | |
| KCNJ11 | SCLC | 8 | All - | 1.5617 | 0.9607 | 2.5388 | 0.072160436 | GTEx |
| | | | Weighted | | | | | |
| | | | median | | | | | |
| KCNJ11 | SCLC | 8 | All - | 1.6152 | 0.6679 | 3.9061 | 0.322574103 | GTEx |
| | | | Simple | | | | | |
| | | | mode | | | | | |
| KCNJ11 | SCLC | 8 | All - | 1.6001 | 0.9984 | 2.5646 | 0.091737131 | GTEx |
| | | | Weighted | | | | | |
| | | | mode | | | | | |
| KCNJ11 | SCLC | 8 | All - | 1.6001 | 0.9947 | 2.5742 | 0.093824484 | GTEx |
| | | | Weighted | | | | | |
| | | | mode | | | | | |
| | | | (NOME) | | | | | |
| KCNJ11 | SCLC | 8 | All - | 1.6152 | 0.6730 | 3.8767 | 0.318711177 | GTEx |
| | | | Simple | | | | | |
| | | | mode | | | | | |
| | | | (NOME) | | | | | |
| KCNJ11 | SCLC | 8 | RAPS | | | | 0.8414154 | GTEx |
| KCNJ11 | SCLC | 8 | MR- | | | | | GTEx |
| | | | PRESSO | | | | | |
| | | | (Raw) | | | | | |
| KCNJ11 | Eye | 16 | IVW | 2.4994 | 1.1039 | 5.6589 | 0.028007857 | GTEx |
| | cancer | | (random | | | | | |
| | | | effects) | | | | | |
| KCNJ11 | Eye | 16 | IVW (fixed | 2.4994 | 0.5998 | 10.4149 | 0.208368049 | GTEx |
| | cancer | | effects) | | | | | |
| KCNJ11 | Eye | 16 | All - MR | 1.1323 | 0.0219 | 58.4419 | 0.951633251 | GTEx |
| | cancer | | Egger | | | | | |
| KCNJ11 | Eye | 16 | All - | 2.5646 | 0.6110 | 10.7636 | 0.198131933 | GTEx |
| | cancer | | Maximum | | | | | |
| | | | likelihood | | | | | |
| KCNJ11 | Eye | 16 | All - | 3.5023 | 0.5142 | 23.8542 | 0.200371713 | GTEx |
| | cancer | | Simple | | | | | |
| | | | median | | | | | |
| KCNJ11 | Eye | 16 | All - | 3.1922 | 0.5397 | 18.8798 | 0.200564485 | GTEx |
| | cancer | | Weighted | | | | | |
| | | | median | | | | | |
| KCNJ11 | Eye | 16 | All - | 3.4943 | 0.3444 | 35.4511 | 0.306640701 | GTEx |
| | cancer | | Simple | | | | | |
| | | | mode | | | | | |
| KCNJ11 | Eye | 16 | All - | 3.3718 | 0.4715 | 24.1130 | 0.244651054 | GTEx |
| | cancer | | Weighted | | | | | |
| | | | mode | | | | | |
| KCNJ11 | Eye | 16 | All - | 3.3718 | 0.4615 | 24.6374 | 0.249562378 | GTEx |
| | cancer | | Weighted | | | | | |
| | | | mode | | | | | |
| | | | (NOME) | | | | | |
| KCNJ11 | Eye | 16 | All - | 3.4943 | 0.3315 | 36.8290 | 0.314266307 | GTEx |
| | cancer | | Simple | | | | | |
| | | | mode | | | | | |
| | | | (NOME) | | | | | |
| KCNJ11 | Eye | 16 | RAPS | | | | | GTEx |
| | cancer | | | | | | | |
| KCNJ11 | Eye | 16 | MR- | | | | 0.04413272 | GTEx |
| | cancer | | PRESSO | | | | | |
| | | | (Raw) | | | | | |
| KCNJ11 | CML | 16 | All - | 0.2301 | 0.0362 | 1.4616 | 0.11932383 | GTEx |
| | | | Maximum | | | | | |
| | | | likelihood | | | | | |
| KCNJ11 | CML | 16 | All - | 0.2328 | 0.0151 | 3.5881 | 0.296284616 | GTEx |
| | | | Simple | | | | | |
| | | | median | | | | | |
| KCNJ11 | CML | 16 | All - | 0.2413 | 0.0244 | 2.3900 | 0.224297972 | GTEx |
| | | | Weighted | | | | | |
| | | | median | | | | | |
| KCNJ11 | CML | 16 | All - | 0.2030 | 0.0091 | 4.5135 | 0.329631417 | GTEx |
| | | | Simple | | | | | |
| | | | mode | | | | | |
| KCNJ11 | CML | 16 | All - | 0.2433 | 0.0181 | 3.2645 | 0.302883493 | GTEx |
| | | | Weighted | | | | | |
| | | | mode | | | | | |
| KCNJ11 | CML | 16 | All - | 0.2433 | 0.0225 | 2.6363 | 0.263149587 | GTEx |
| | | | Weighted | | | | | |
| | | | mode | | | | | |
| | | | (NOME) | | | | | |
| KCNJ11 | CML | 16 | All - | 0.2030 | 0.0102 | 4.0241 | 0.31198678 | GTEx |
| | | | Simple | | | | | |
| | | | mode | | | | | |
| | | | (NOME) | | | | | |
| KCNJ11 | CML | 16 | RAPS | | | | | GTEx |
| KCNJ11 | CML | 16 | MR- | | | | 0.001960919 | GTEx |
| | | | PRESSO | | | | | |
| | | | (Raw) | | | | | |
| KCNJ11 | GC | 16 | All - | 0.6829 | 0.5511 | 0.8463 | 0.000491435 | GTEx |
| | | | Maximum | | | | | |
| | | | likelihood | | | | | |
| KCNJ11 | GO | 16 | All - | 0.6576 | 0.4786 | 0.9036 | 0.009728083 | GTEx |
| | | | Simple | | | | | |
| | | | median | | | | | |
| KCNJ11 | GC | 16 | All - | 0.6698 | 0.5067 | 0.8855 | 0.004893962 | GTEX |
| | | | Weighted | | | | | |
| | | | median | | | | | |
| KCNJ11 | GC | 16 | All - | 0.6471 | 0.4594 | 0.9116 | 0.025016034 | GTEx |
| | | | Simple | | | | | |
| | | | mode | | | | | |
| KCNJ11 | GC | 16 | All - | 0.6831 | 0.5175 | 0.9016 | 0.016747059 | GTEx |
| | | | Weighted | | | | | |
| | | | mode | | | | | |
| KCNJ11 | GC | 16 | All - | 0.6831 | 0.5143 | 0.9073 | 0.018854857 | GTEx |
| | | | Weighted | | | | | |
| | | | mode | | | | | |
| | | | (NOME) | | | | | |
| KCNJ11 | GC | 16 | All - | 0.6471 | 0.4632 | 0.9041 | 0.022160594 | GTEx |
| | | | Simple | | | | | |
| | | | mode | | | | | |
| | | | (NOME) | | | | | |
| KCNJ11 | GC | 16 | RAPS | | | | 0.50026 | GTEx |
| KCNJ11 | GC | 16 | MR- | | | | 7.62E−05 | GTEx |
| | | | PRESSO | | | | | |
| | | | (Raw) | | | | | |
| GLP1R | EC | 8 | All - | 5.1561 | 2.1605 | 12.3051 | 0.000219159 | GTEx |
| | | | Maximum | | | | | |
| | | | likelihood | | | | | |
| GLP1R | EC | 8 | All - | 2.0979 | 0.5954 | 7.3914 | 0.248864374 | GTEx |
| | | | Simple | | | | | |
| | | | median | | | | | |
| GLP1R | EC | 8 | All - | 2.0632 | 0.6330 | 6.7249 | 0.229592042 | GTEx |
| | | | Weighted | | | | | |
| | | | median | | | | | |
| GLP1R | EC | 8 | All - | 1.5189 | 0.3377 | 6.8326 | 0.602790745 | GTEx |
| | | | Simple | | | | | |
| | | | mode | | | | | |
| GLP1R | EC | 8 | All - | 1.7287 | 0.4432 | 6.7429 | 0.45645244 | GTEx |
| | | | Weighted | | | | | |
| | | | mode | | | | | |
| GLP1R | EC | 8 | All - | 1.9048 | 0.4297 | 8.4428 | 0.4243621 | GTEx |
| | | | Weighted | | | | | |
| | | | mode | | | | | |
| | | | (NOME) | | | | | |
| GLP1R | EC | 8 | All - | 1.5189 | 0.3090 | 7.4657 | 0.622739186 | GTEx |
| | | | Simple | | | | | |
| | | | mode | | | | | |
| | | | (NOME) | | | | | |
| GLP1R | EC | 8 | RAPS | | | | | GTEx |
| GLP1R | EC | | MR- | | | | 0.02620224 | GTEx |
| | | | PRESSO | | | | | |
| | | | (Raw) | | | | | |
| SLC5A2 | Pancreas | 9 | IVW | 43.3850 | 3.4257 | 549.4514 | 0.003607456 | GTEx |
| | cancer | | (random | | | | | |
| | | | effects) | | | | | |
| SLC5A2 | Pancreas | 9 | IVW (fixed | 43.3850 | 2.0622 | 912.7493 | 0.01528024 | GTEx |
| | cancer | | effects) | | | | | |
| SLC5A2 | Pancreas | 9 | All - MR | 3.1187 | 0.0016 | 6007.8466 | 0.776729592 | GTEx |
| | cancer | | Egger | | | | | |
| SLC5A2 | Pancreas | 9 | All - | 66.3447 | 2.0081 | 2191.8808 | 0.018738097 | GTEx |
| | cancer | | Maximum | | | | | |
| | | | likelihood | | | | | |
| SLC5A2 | Pancreas | 9 | All - | 14.1015 | 0.1428 | 1392.5487 | 0.258746525 | GTEx |
| | cancer | | Simple | | | | | |
| | | | median | | | | | |
| SLC5A2 | Pancreas | 9 | All - | 7.5269 | 0.0984 | 575.6540 | 0.361665413 | GTEx |
| | cancer | | Weighted | | | | | |
| | | | median | | | | | |
| SLC5A2 | Pancreas | 9 | All - | 10.9928 | 0.0292 | 4138.1351 | 0.451072055 | GTEx |
| | cancer | | Simple | | | | | |
| | | | mode | | | | | |
| SLC5A2 | Pancreas | 9 | All - | 6.6261 | 0.0320 | 1370.4583 | 0.50665291 | GTEx |
| | cancer | | Weighted | | | | | |
| | | | mode | | | | | |
| SLC5A2 | Pancreas | 9 | All - | 7.2859 | 0.0267 | 1985.1245 | 0.507242131 | GTEx |
| | cancer | | Weighted | | | | | |
| | | | mode | | | | | |
| | | | (NOME) | | | | | |
| SLC5A2 | Pancreas | 9 | All - | 10.9928 | 0.0257 | 4710.8833 | 0.460466715 | GTEx |
| | cancer | | Simple | | | | | |
| | | | mode | | | | | |
| | | | (NOME) | | | | | |
| SLC5A2 | Pancreas | | RAPS | | | | | GTEx |
| | cancer | | | | | | | |
| SLC5A2 | Pancreas | | MR- | | | | 0.01957136 | GTEx |
| | cancer | | PRESSO | | | | | |
| | | | (Raw) | | | | | |
| SLC5A2 | NHL | 9 | IVW | 0.0262 | 0.0044 | 0.1579 | 7.02E−05 | GTEx |
| | | | (random | | | | | |
| | | | effects) | | | | | |
| SLC5A2 | NHL | 9 | IVW (fixed | 0.0262 | 0.0033 | 0.2089 | 0.000582927 | GTEx |
| | | | effects) | | | | | |
| SLC5A2 | NHL | 9 | All - MR | 5.0453 | 0.0324 | 784.5221 | 0.549577141 | GTEx |
| | | | Egger | | | | | |
| SLC5A2 | NHL | 9 | All - | 0.0243 | 0.0021 | 0.2814 | 0.002929011 | GTEx |
| | | | Maximum | | | | | |
| | | | likelihood | | | | | |
| SLC5A2 | NHL | 9 | All - | 0.0112 | 0.0004 | 0.2802 | 0.006254112 | GTEx |
| | | | Simple | | | | | |
| | | | median | | | | | |
| SLC5A2 | NHL | 9 | All - | 0.0122 | 0.0004 | 0.3410 | 0.009494761 | GTEx |
| | | | Weighted | | | | | |
| | | | median | | | | | |
| SLC5A2 | NHL | 9 | All - | 0.0072 | 0.0000 | 1.1422 | 0.092740696 | GTEx |
| | | | Simple | | | | | |
| | | | mode | | | | | |
| SLC5A2 | NHL | 9 | All - | 0.0076 | 0.0000 | 3.5331 | 0.158057351 | GTEx |
| | | | Weighted | | | | | |
| | | | mode | | | | | |
| SLC5A2 | NHL | 9 | All - | 0.0072 | 0.0000 | 1.1830 | 0.094639299 | GTEx |
| | | | Weighted | | | | | |
| | | | mode | | | | | |
| | | | (NOME) | | | | | |
| SLC5A2 | NHL | 9 | All - | 0.0072 | 0.0001 | 0.5471 | 0.056011101 | GTEx |
| | | | Simple | | | | | |
| | | | mode | | | | | |
| | | | (NOME) | | | | | |
| SLC5A2 | NHL | | RAPS | | | | | GTEx |
| SLC5A2 | NHL | | MR- | | | | 0.004086879 | GTEx |
| | | | PRESSO | | | | | |
| | | | (Raw) | | | | | |
| PPARG | BC | 10 | All - | 3.3775 | 1.7776 | 6.4173 | 0.000201895 | GTEx |
| | | | Maximum | | | | | |
| | | | likelihood | | | | | |
| PPARG | BC | 10 | All - | 4.1032 | 1.4893 | 11.3043 | 0.006325725 | GTEx |
| | | | Simple | | | | | |
| | | | median | | | | | |
| PPARG | BC | 10 | All - | 3.9060 | 1.7246 | 8.8467 | 0.001088251 | GTEx |
| | | | Weighted | | | | | |
| | | | median | | | | | |
| PPARG | BC | 10 | All - | 5.0245 | 1.5824 | 15.9538 | 0.022901279 | GTEx |
| | | | Simple | | | | | |
| | | | mode | | | | | |
| PPARG | BC | 10 | All - | 3.8231 | 1.7773 | 8.2237 | 0.007488492 | GTEx |
| | | | Weighted | | | | | |
| | | | mode | | | | | |
| PPARG | BC | 10 | All - | 3.8985 | 1.7170 | 8.8516 | 0.009964056 | GTEx |
| | | | Weighted | | | | | |
| | | | mode | | | | | |
| | | | (NOME) | | | | | |
| PPARG | BC | 10 | All - | 5.0245 | 1.5357 | 16.4384 | 0.025646875 | GTEx |
| | | | Simple | | | | | |
| | | | mode | | | | | |
| | | | (NOME) | | | | | |
| PPARG | BC | 10 | RAPS | | | | | GTEx |
| PPARG | BC | 10 | MR- | | | | 0.002311234 | GTEx |
| | | | PRESSO | | | | | |
| | | | (Raw) | | | | | |
| PPARG | OC | 10 | All - | 0.1694 | 0.0755 | 0.3801 | 0.0000166 | GTEx |
| | | | Maximum | | | | | |
| | | | likelihood | | | | | |
| PPARG | OC | 10 | All - | 0.1812 | 0.0492 | 0.6678 | 0.01026574 | GTEx |
| | | | Simple | | | | | |
| | | | median | | | | | |
| PPARG | OC | 10 | All - | 0.1658 | 0.0630 | 0.4365 | 0.000273649 | GTEx |
| | | | Weighted | | | | | |
| | | | median | | | | | |
| PPARG | OC | 10 | All - | 0.1377 | 0.0284 | 0.6686 | 0.036192002 | GTEx |
| | | | Simple | | | | | |
| | | | mode | | | | | |
| PPARG | OC | 10 | All - | 0.1513 | 0.0605 | 0.3783 | 0.00293246 | GTEx |
| | | | Weighted | | | | | |
| | | | mode | | | | | |
| PPARG | OC | 10 | All - | 0.1478 | 0.0568 | 0.3842 | 0.003497908 | GTEx |
| | | | Weighted | | | | | |
| | | | mode | | | | | |
| | | | (NOME) | | | | | |
| PPARG | 0C | 10 | All - | 0.1377 | 0.0325 | 0.5832 | 0.024712602 | GTEx |
| | | | Simple | | | | | |
| | | | mode | | | | | |
| | | | (NOME) | | | | | |
| PPARG | OC | 10 | RAPS | | | | | GTEx |
| PPARG | OC | 10 | MR- | | | | 0.000201734 | GTEx |
| | | | PRESSO | | | | | |
| | | | (Raw) | | | | | |
| PPARG | TC | 10 | All - | 0.0214 | 0.0041 | 0.1122 | 5.41E−06 | GTEx |
| | | | Maximum | | | | | |
| | | | likelihood | | | | | |
| PPARG | TC | 10 | All - | 0.0472 | 0.0028 | 0.7872 | 0.033445602 | GTEx |
| | | | Simple | | | | | |
| | | | median | | | | | |
| PPARG | TC | 10 | All - | 0.0145 | 0.0019 | 0.1127 | 5.18E−05 | GTEx |
| | | | Weighted | | | | | |
| | | | median | | | | | |
| PPARG | TC | 10 | All - | 0.0270 | 0.0012 | 0.6355 | 0.051724469 | GTEx |
| | | | Simple | | | | | |
| | | | mode | | | | | |
| PPARG | TC | 10 | All - | 0.0122 | 0.0014 | 0.1047 | 0.003016311 | GTEx |
| | | | Weighted | | | | | |
| | | | mode | | | | | |
| PPARG | TC | 10 | All - | 0.0120 | 0.0019 | 0.0771 | 0.001190662 | GTEx |
| | | | Weighted | | | | | |
| | | | mode | | | | | |
| | | | (NOME) | | | | | |
| PPARG | TC | 10 | All - | 0.0270 | 0.0013 | 0.5745 | 0.045828579 | GTEx |
| | | | Simple | | | | | |
| | | | mode | | | | | |
| | | | (NOME) | | | | | |
| PPARG | TC | | RAPS | | | | | GTEx |
| PPARG | TC | | MR- | | | | 1.15E−05 | GTEx |
| | | | PRESSO | | | | | |
| | | | (Raw) | | | | | |
| ABCC8/ | CML | 2 | IVW | 0.1965 | 0.1428 | 0.2703 | 1.62E−23 | GWAS |
| KCNJ11 | | | (random | | | | | |
| | | | effects) | | | | | |
| ABCC8/ | CML | 2 | IVW (fixed | 0.1965 | 0.0068 | 5.6398 | 3.42E−01 | GWAS |
| KCNJ11 | | | effects) | | | | | |
| ABCC8/ | CML | 2 | All - MR | NA | NA | NA | NA | GWAS |
| KCNJ11 | | | Egger | | | | | |
| ABCC8/ | CML | 2 | All - | 0.1965 | 0.0068 | 5.6719 | 3.43E−01 | GWAS |
| KCNJ11 | | | Maximum | | | | | |
| | | | likelihood | | | | | |
| ABCC8/ | CML | 2 | All - | NA | NA | NA | NA | GWAS |
| KCNJ11 | | | Simple | | | | | |
| | | | median | | | | | |
| ABCC8/ | CML | 2 | All - | NA | NA | NA | NA | GWAS |
| KCNJ11 | | | Weighted | | | | | |
| | | | median | | | | | |
| ABCC8/ | CML | 2 | All - | NA | NA | NA | NA | GWAS |
| KCNJ11 | | | Simple | | | | | |
| | | | mode | | | | | |
| ABCC8/ | CML | 2 | All - | NA | NA | NA | NA | GWAS |
| KCNJ11 | | | Weighted | | | | | |
| | | | mode | | | | | |
| ABCC8/ | CML | 2 | All - | NA | NA | NA | NA | GWAS |
| KCNJ11 | | | Weighted | | | | | |
| | | | mode | | | | | |
| | | | (NOME) | | | | | |
| ABCC8/ | CML | 2 | All - | NA | NA | NA | NA | GWAS |
| KCNJ11 | | | Simple | | | | | |
| | | | mode | | | | | |
| | | | (NOME) | | | | | |
| ABCC8/ | CML | 2 | RAPS | | | | | GWAS |
| KCNJ11 | | | | | | | | |
| ABCC8/ | CML | 2 | MR- | | | | | GWAS |
| KCNJ11 | | | PRESSO | | | | | |
| | | | (Raw) | | | | | |
| ABCC8/ | CTC | 2 | IVW | 0.4514 | 0.4032 | 0.5054 | 2.52E−43 | GWAS |
| KCNJ11 | | | (random | | | | | |
| | | | effects) | | | | | |
| ABCC8/ | CTC | 2 | IVW (fixed | 0.4514 | 0.0629 | 3.2403 | 0.429008882 | GWAS |
| KCNJ11 | | | effects) | | | | | |
| ABCC8/ | CTC | 2 | All - MR | NA | NA | NA | NA | GWAS |
| KCNJ11 | | | Egger | | | | | |
| ABCC8/ | CTC | 2 | All - | | 0.0627 | 3.2478 | 0.429553237 | GWAS |
| KCNJ11 | | | Maximum | | | | | |
| | | | likelihood | | | | | |
| ABCC8/ | CTC | 2 | All - | NA | NA | NA | NA | GWAS |
| KCNJ11 | | | Simple | | | | | |
| | | | median | | | | | |
| ABCC8/ | CTC | 2 | All - | NA | NA | NA | NA | GWAS |
| KCNJ11 | | | Weighted | | | | | |
| | | | median | | | | | |
| ABCC8/ | CTC | 2 | All - | NA | NA | NA | NA | GWAS |
| KCNJ11 | | | Simple | | | | | |
| | | | mode | | | | | |
| ABCC8/ | CTC | 2 | All - | NA | NA | NA | NA | GWAS |
| KCNJ11 | | | Weighted | | | | | |
| | | | mode | | | | | |
| ABCC8/ | CTC | 2 | All - | NA | NA | NA | NA | GWAS |
| KCNJ11 | | | Weighted | | | | | |
| | | | mode | | | | | |
| | | | (NOME) | | | | | |
| ABCC8/ | CTC | 2 | All - | NA | NA | NA | NA | GWAS |
| KCNJ11 | | | Simple | | | | | |
| | | | mode | | | | | |
| | | | (NOME) | | | | | |
| ABCC8/ | CTC | 2 | RAPS | | | | 0.9999137 | GWAS |
| KCNJ11 | | | | | | | | |
| ABCC8/ | CTC | 2 | MR- | | | | | GWAS |
| KCNJ11 | | | PRESSO | | | | | |
| | | | (Raw) | | | | | |
| ABCC8/ | GC | 2 | All - | 0.7736 | 0.5188 | 1.1537 | 2.08E−01 | GWAS |
| KCNJ11 | | | Maximum | | | | | |
| | | | likelihood | | | | | |
| ABCC8/ | GC | 2 | All - | NA | NA | NA | NA | GWAS |
| KCNJ11 | | | Simple | | | | | |
| | | | median | | | | | |
| ABCC8/ | GC | 2 | All - | NA | NA | NA | NA | GWAS |
| KCNJ11 | | | Weighted | | | | | |
| | | | median | | | | | |
| ABCC8/ | GC | 2 | All - | NA | NA | NA | NA | GWAS |
| KCNJ11 | | | Simple | | | | | |
| | | | mode | | | | | |
| ABCC8/ | GC | 2 | All - | NA | NA | NA | NA | GWAS |
| KCNJ11 | | | Weighted | | | | | |
| | | | mode | | | | | |
| ABCC8/ | GC | 2 | All - | NA | NA | NA | NA | GWAS |
| KCNJ11 | | | Weighted | | | | | |
| | | | mode | | | | | |
| | | | (NOME) | | | | | |
| ABCC8/ | GC | 2 | All - | NA | NA | NA | NA | GWAS |
| KCNJ11 | | | Simple | | | | | |
| | | | mode | | | | | |
| | | | (NOME) | | | | | |
| ABCC8/ | GC | 2 | RAPS | | | | 9.66E−01 | GWAS |
| KCNJ11 | | | | | | | | |
| ABCC8/ | GC | 2 | MR- | | | | | GWAS |
| KCNJ11 | | | PRESSO | | | | | |
| | | | (Raw) | | | | | |
| ABCC8/ | SCLC | 2 | All - | 1.7397 | 1.0906 | 2.7752 | 0.02012932 | GWAS |
| KCNJ11 | | | Maximum | | | | | |
| | | | likelihood | | | | | |
| ABCC8/ | SCLC | 2 | All - | NA | NA | NA | NA | GWAS |
| KCNJ11 | | | Simple | | | | | |
| | | | median | | | | | |
| ABCC8/ | SCLC | 2 | All - | NA | NA | NA | NA | GWAS |
| KCNJ11 | | | Weighted | | | | | |
| | | | median | | | | | |
| ABCC8/ | SCLC | 2 | All - | NA | NA | NA | NA | GWAS |
| KCNJ11 | | | Simple | | | | | |
| | | | mode | | | | | |
| ABCC8/ | SCLC | 2 | All - | NA | NA | NA | NA | GWAS |
| KCNJ11 | | | Weighted | | | | | |
| | | | mode | | | | | |
| ABCC8/ | SCLC | 2 | All - | NA | NA | NA | NA | GWAS |
| KCNJ11 | | | Weighted | | | | | |
| | | | mode | | | | | |
| | | | (NOME) | | | | | |
| ABCC8/ | SCLC | 2 | All - | NA | NA | NA | NA | GWAS |
| KCNJ11 | | | Simple | | | | | |
| | | | mode | | | | | |
| | | | (NOME) | | | | | |
| ABCC8/ | SCLC | 2 | RAPS | | | | 0.9984602 | GWAS |
| KCNJ11 | | | | | | | | |
| ABCC8/ | SCLC | 2 | MR- | | | | | GWAS |
| KCNJ11 | | | PRESSO | | | | | |
| | | | (Raw) | | | | | |
| SLC5A2 | NHL | 2 | IVW | 7.2198 | 3.9978 | 13.0385 | 5.56E−11 | GWAS |
| | | | (random | | | | | |
| | | | effects) | | | | | |
| SLC5A2 | NHL | 2 | IVW (fixed | 7.2198 | 0.9242 | 56.4026 | 5.95E−02 | GWAS |
| | | | effects) | | | | | |
| SLC5A2 | NHL | 2 | All - MR | NA | NA | NA | NA | GWAS |
| | | | Egger | | | | | |
| SLC5A2 | NHL | 2 | All | 7.2083 | 0.8797 | 59.0669 | 6.57E−02 | GWAS |
| | | | Maximum | | | | | |
| | | | likelihood | | | | | |
| SLC5A2 | NHL | 2 | All - | NA | NA | NA | NA | GWAS |
| | | | Simple | | | | | |
| | | | median | | | | | |
| SLC5A2 | NHL | 2 | All - | NA | NA | NA | NA | GWAS |
| | | | Weighted | | | | | |
| | | | median | | | | | |
| SLC5A2 | NHL | 2 | All - | NA | NA | NA | NA | GWAS |
| | | | Simple | | | | | |
| | | | mode | | | | | |
| SLC5A2 | NHL | 2 | All - | NA | NA | NA | NA | GWAS |
| | | | Weighted | | | | | |
| | | | mode | | | | | |
| SLC5A2 | NHL | 2 | All - | NA | NA | NA | NA | GWAS |
| | | | Weighted | | | | | |
| | | | mode | | | | | |
| | | | (NOME) | | | | | |
| SLC5A2 | NHL | 2 | All - | NA | NA | NA | NA | GWAS |
| | | | Simple | | | | | |
| | | | mode | | | | | |
| | | | (NOME) | | | | | |
| SLC5A2 | NHL | 2 | RAPS | | | | | GWAS |
| SLC5A2 | NHL | 2 | MR- | | | | | GWAS |
| | | | PRESSO | | | | | |
| | | | (Raw) | | | | | |
| SLC5A2 | PC | 2 | IVW | 0.2439 | 0.1752 | 0.3395 | 6.00E−17 | GWAS |
| | | | (random | | | | | |
| | | | effects) | | | | | |
| SLC5A2 | PC | 2 | IVW (fixed | 0.2439 | 0.0116 | 5.1179 | 3.64E−01 | GWAS |
| | | | effects) | | | | | |
| SLC5A2 | PC | 2 | All - MR | NA | NA | NA | NA | GWAS |
| | | | Egger | | | | | |
| SLC5A2 | PC | 2 | All - | 0.2440 | 0.0114 | 5.2067 | 3.66E−01 | GWAS |
| | | | Maximum | | | | | |
| | | | likelihood | | | | | |
| SLC5A2 | PC | 2 | All - | NA | NA | NA | NA | GWAS |
| | | | Simple | | | | | |
| | | | median | | | | | |
| SLC5A2 | PC | 2 | All - | NA | NA | NA | NA | GWAS |
| | | | Weighted | | | | | |
| | | | median | | | | | |
| SLC5A2 | PC | 2 | All - | NA | NA | NA | NA | GWAS |
| | | | Simple | | | | | |
| | | | mode | | | | | |
| SLC5A2 | PC | 2 | All - | NA | NA | NA | NA | GWAS |
| | | | Weighted | | | | | |
| | | | mode | | | | | |
| SLC5A2 | PC | 2 | All - | NA | NA | NA | NA | GWAS |
| | | | Weighted | | | | | |
| | | | mode | | | | | |
| | | | (NOME) | | | | | |
| SLC5A2 | PC | 2 | All - | NA | NA | NA | NA | GWAS |
| | | | Simple | | | | | |
| | | | mode | | | | | |
| | | | (NOME) | | | | | |
| SLC5A2 | PC | 2 | RAPS | | | | | GWAS |
| SLC5A2 | PC | 2 | MR- | | | | | GWAS |
| | | | PRESSO | | | | | |
| | | | (Raw) | | | | | |
| PPARG | OC | 4 | All - | 0.2558 | 0.1132 | 0.5782 | 0.001049234 | GWAS |
| | | | Maximum | | | | | |
| | | | likelihood | | | | | |
| PPARG | OC | 4 | All - | 0.2776 | 0.1035 | 0.7445 | 0.01089429 | GWAS |
| | | | Simple | | | | | |
| | | | median | | | | | |
| PPARG | OC | 4 | All - | 0.2573 | 0.1011 | 0.6552 | 0.004417845 | GWAS |
| | | | Weighted | | | | | |
| | | | median | | | | | |
| PPARG | OC | 4 | All - | 0.3188 | 0.0950 | 1.0701 | 0.161367172 | GWAS |
| | | | Simple | | | | | |
| | | | mode | | | | | |
| PPARG | 0C | 4 | All - | 0.2478 | 0.0866 | 0.7096 | 0.080423937 | GWAS |
| | | | Weighted | | | | | |
| | | | mode | | | | | |
| PPARG | OC | 4 | All - | 0.2464 | 0.0884 | 0.6868 | 0.075148844 | GWAS |
| | | | Weighted | | | | | |
| | | | mode | | | | | |
| | | | (NOME) | | | | | |
| PPARG | OC | 4 | All - | 0.3188 | 0.0895 | 1.1356 | 0.175951397 | GWAS |
| | | | Simple | | | | | |
| | | | mode | | | | | |
| | | | (NOME) | | | | | |
| PPARG | OC | 4 | RAPS | | | | 9.57E−01 | GWAS |
| PPARG | OC | 4 | MR- | | | | 6.40E−03 | GWAS |
| | | | PRESSO | | | | | |
| | | | (Raw) | | | | | |
| PPARG | TC | 4 | All - | 0.0485 | 0.0093 | 0.2526 | 0.000325739 | GWAS |
| | | | Maximum | | | | | |
| | | | likelihood | | | | | |
| PPARG | TC | 4 | All - | 0.0444 | 0.0057 | 0.3479 | 0.003021455 | GWAS |
| | | | Simple | | | | | |
| | | | median | | | | | |
| PPARG | TC | 4 | All - | 0.0355 | 0.0052 | 0.2421 | 0.00065562 | GWAS |
| | | | Weighted | | | | | |
| | | | median | | | | | |
| PPARG | TC | 4 | All - | 0.0382 | 0.0027 | 0.5497 | 0.095875599 | GWAS |
| | | | Simple | | | | | |
| | | | mode | | | | | |
| PPARG | TC | 4 | All - | 0.0291 | 0.0032 | 0.2628 | 0.051256753 | GWAS |
| | | | Weighted | | | | | |
| | | | mode | | | | | |
| PPARG | TC | 4 | All - | 0.0291 | 0.0036 | 0.2384 | 0.04585857 | GWAS |
| | | | Weighted | | | | | |
| | | | mode | | | | | |
| | | | (NOME) | | | | | |
| PPARG | TC | 4 | All - | 0.0382 | 0.0027 | 0.5354 | 0.093843234 | GWAS |
| | | | Simple | | | | | |
| | | | mode | | | | | |
| | | | (NOME) | | | | | |
| PPARG | TC | 4 | RAPS | | | | | GWAS |
| PPARG | TC | 4 | MR- | | | | 0.01038449 | GWAS |
| | | | PRESSO | | | | | |
| | | | (Raw) | | | | | |
| All targets | GC | 513 | All - | 0.8617 | 0.8049 | 0.9227 | 0.0000196 | GWAS |
| | | | Inverse | | | | | |
| | | | variance | | | | | |
| | | | weighted | | | | | |
| | | | (multipli- | | | | | |
| | | | cative | | | | | |
| | | | random | | | | | |
| | | | effects) | | | | | |
| All targets | GC | 513 | All - MR | 0.8476 | 0.7380 | 0.9735 | 0.02129231 | GWAS |
| | | | Egger | | | | | |

SCLC: Squamous cell lung carcinoma; CML: Chronic myelogenous leukemia; GC: Gastric cancer; EC: Endometrial cancer; NHL: Non hodgkin lymphoma; BC: Bronchial cancer; TC: Tongue cancer; OC: Oropharynx cancer; CTC: Connective tissue cancer. Method: GTEx or GWAS instruments.

TABLE 10
Estimates of the smallest detectable odds ratio per unit change in drug
target-mediated inverse rank-normal transformed HbA1c reduction (mmol/mol)
with 80% power to detect an effect (α = 0.05). This table presents
estimates of the smallest detectable odds ratio (OR) per unit change
in drug target-mediated inverse rank-normal transformed HbA1c reduction
(in mmol/mol), assuming 80% power to detect an effect at a significance
level (α) of 0.05. The estimates provide insights into the minimum
effect size that can be reliably detected in the analysis.

| Outcome | KCNJ11 | GLP1R | SLC5A2 | PPARG | RAMP2 |
|---|---|---|---|---|---|
| Tongue cancer | 0.32 (sample | — | — | — | — |
| | size) | | | | |
| Squamous cell lung | 3.08 (low | | | | |
| carcinoma | power) | | | | |
| Chronic | 0.002 (sample | | | | |
| myelogenous | size) | | | | |
| leukemia | | | | | |
| Gastric cancer | 0.82 | | | | |
| Endometrial cancer | | 1.35(wide | | | |
| | | range) | | | |
| Pancreas cancer | | | 26.5 | | |
| | | | (reverse | | |
| | | | direction) | | |
| Non hodgkin | | | 0.09 | | |
| lymphoma | | | (reverse | | |
| | | | direction) | | |
| Bronchial cancer | | | | 6.29 (low | |
| | | | | power) | |
| Oropharynx cancer | | | | 0.63 | |
| Tongue cancer | | | | 0.0034 | |
| | | | | (sample size) | |
| Bronchial cancer | | | | | 7.01 (wide |
| | | | | | range) |

TABLE 11
Detailed drug-target MR estimates examining the association of genetically proxied
perturbation of KCNJ11 and PPARG with cancer risk (GTEx instruments). This table
provides detailed MR estimates for the single drug target analysis, investigating
the association between genetically proxied perturbation single drug target with
cancer risk. The analysis utilizes genetic instruments derived from the Genotype-
Tissue Expression (GTEx) project. The estimates include effect sizes (odds ratios,
OR), 95% low confidence intervals (95% LCI), 95% high confidence intervals (95% UCI)
and P value, indicating the strength and significance of the observed associations.

| | | | | | 95% | 95% | |
|---|---|---|---|---|---|---|---|
| Exposure | Outcome | nSNP | SNP | OR | LCI | UCI | P value |
| KCNJ11 | Gastric | 16 | IVW (random | 0.6796 | 0.5904 | 0.7822 | 7.37E−08 |
| | cancer | | effects) | | | | |
| KCNJ11 | Gastric | 16 | IVW (fixed | 0.6796 | 0.5495 | 0.8404 | 0.000364285 |
| | cancer | | effects) | | | | |
| KCNJ11 | Gastric | 16 | All - MR | 0.8253 | 0.4483 | 1.5193 | 0.54730662 |
| | cancer | | Egger | | | | |
| KCNJ11 | Gastric | 16 | All - Maximum | 0.6829 | 0.5511 | 0.8463 | 0.000491435 |
| | cancer | | likelihood | | | | |
| KCNJ11 | Gastric | 16 | All - Simple | 0.6576 | 0.4786 | 0.9036 | 0.009728083 |
| | cancer | | median | | | | |
| KCNJ11 | Gastric | 16 | All - Weighted | 0.6698 | 0.5067 | 0.8855 | 0.004893962 |
| | cancer | | median | | | | |
| KCNJ11 | Gastric | 16 | All - Simple | 0.6471 | 0.4594 | 0.9116 | 0.025016034 |
| | cancer | | mode | | | | |
| KCNJ11 | Gastric | 16 | All - Weighted | 0.6831 | 0.5175 | 0.9016 | 0.016747059 |
| | cancer | | mode | | | | |
| KCNJ11 | Gastric | 16 | All - Weighted | 0.6831 | 0.5143 | 0.9073 | 0.018854857 |
| | cancer | | mode (NOME) | | | | |
| KCNJ11 | Gastric | 16 | All - Simple | 0.6471 | 0.4632 | 0.9041 | 0.022160594 |
| | cancer | | mode (NOME) | | | | |
| KCNJ11 | Gastric | 16 | RAPS | | | | 0.50026 |
| | cancer | | | | | | |
| KCNJ11 | Gastric | 16 | MR- | | | | 7.62E−05 |
| | cancer | | PRESSO(Raw) | | | | |
| PPARG | Oropharynx | 10 | IVW (random | 0.1680 | 0.0938 | 0.3008 | 1.94E−09 |
| | cancer | | effects) | | | | |
| PPARG | Oropharynx | 10 | IVW (fixed | 0.1680 | 0.0772 | 0.3658 | 0.00000703 |
| | cancer | | effects) | | | | |
| PPARG | Oropharynx | 10 | All - MR | 0.1483 | 0.0432 | 0.5090 | 0.016225456 |
| | cancer | | Egger | | | | |
| PPARG | Oropharynx | 10 | All - Maximum | 0.1694 | 0.0755 | 0.3801 | 0.0000166 |
| | cancer | | likelihood | | | | |
| PPARG | Oropharynx | 10 | All - Simple | 0.1812 | 0.0492 | 0.6678 | 0.01026574 |
| | cancer | | median | | | | |
| PPARG | Oropharynx | 10 | All - Weighted | 0.1658 | 0.0630 | 0.4365 | 0.000273649 |
| | cancer | | median | | | | |
| PPARG | Oropharynx | 10 | All - Simple | 0.1377 | 0.0284 | 0.6686 | 0.036192002 |
| | cancer | | mode | | | | |
| PPARG | Oropharynx | 10 | All - Weighted | 0.1513 | 0.0605 | 0.3783 | 0.00293246 |
| | cancer | | mode | | | | |
| PPARG | Oropharynx | 10 | All - Weighted | 0.1478 | 0.0568 | 0.3842 | 0.003497908 |
| | cancer | | mode (NOME) | | | | |
| PPARG | Oropharynx | 10 | All - Simple | 0.1377 | 0.0325 | 0.5832 | 0.024712602 |
| | cancer | | mode (NOME) | | | | |
| PPARG | Oropharynx | 10 | RAPS | | | | |
| | cancer | | | | | | |
| PPARG | Oropharynx | 10 | MR- | | | | 0.000201734 |
| | cancer | | PRESSO(Raw) | | | | |

MR-PRESSO: Mendelian Randomization Pleiotropy RESidual Sum and Outlier.
RAPS: Robust Adjusted Profile Score.

TABLE 12
Detailed MR estimates for all targets-based analysis examining the association
of genetically proxied perturbation of KCNJ11 PPI and PPARG PPI with cancer
risk (using GTEx instruments). This table provides detailed MR estimates for
the all targets-based analysis, investigating the association between genetically
proxied perturbation all targets with cancer risk. The analysis utilizes genetic
instruments derived from the Genotype-Tissue Expression (GTEx) project. The
estimates include effect sizes (odds ratios, OR), 95% low confidence intervals
(95% LCI), 95% high confidence intervals (95% UCI) and P value, indicating
the strength and significance of the observed associations.

| | | | | | 95% | 95% | |
|---|---|---|---|---|---|---|---|
| Exposure | Outcome | nSNP | SNP | OR | LCI | UCI | P value |
| All targets | Gastric | 86 | IVW (random | 0.7199 | 0.6172 | 0.8398 | 2.89E−05 |
| | cancer | | effects) | | | | |
| All targets | Gastric | 86 | IVW (fixed | 0.7199 | 0.6121 | 0.8467 | 7.15E−05 |
| | cancer | | effects) | | | | |
| All targets | Gastric | 86 | All - MR | 0.6678 | 0.4936 | 0.9036 | 0.010513794 |
| | cancer | | Egger | | | | |
| All targets | Gastric | 86 | All - Maximum | 0.7219 | 0.6122 | 0.8512 | 0.000105686 |
| | cancer | | likelihood | | | | |
| All targets | Gastric | 86 | All - Simple | 0.7959 | 0.5860 | 1.0811 | 0.144051257 |
| | cancer | | median | | | | |
| All targets | Gastric | 86 | All - Weighted | 0.6743 | 0.5356 | 0.8491 | 0.000803522 |
| | cancer | | median | | | | |
| All targets | Gastric | 86 | All - Simple | 0.7297 | 0.5098 | 1.0446 | 0.088774205 |
| | cancer | | mode | | | | |
| All targets | Gastric | 86 | All - Weighted | 0.6908 | 0.5645 | 0.8452 | 0.000546829 |
| | cancer | | mode | | | | |
| All targets | Gastric | 86 | All - Weighted | 0.6908 | 0.5633 | 0.8471 | 0.000623138 |
| | cancer | | mode (NOME) | | | | |
| All targets | Gastric | 86 | All - Simple | 0.7297 | 0.5207 | 1.0226 | 0.070708266 |
| | cancer | | mode (NOME) | | | | |
| All targets | Gastric | 86 | RAPS | 0.8443 | 0.5207 | 1.3689 | 0.715651 |
| | cancer | | | | | | |
| All targets | Gastric | 86 | MR_PRESSO | 0.7199 | 0.6172 | 0.8398 | 7.01E−05 |
| | cancer | | | | | | |
| All targets | Oropharynx | 86 | IVW (random | 0.5818 | 0.3930 | 0.8614 | 0.006823232 |
| | cancer | | effects) | | | | |
| All targets | Oropharynx | 86 | IVW (fixed | 0.5818 | 0.4016 | 0.8428 | 0.004178641 |
| | cancer | | effects) | | | | |
| All targets | Oropharynx | 86 | All - MR | 0.5096 | 0.2497 | 1.0400 | 0.067504734 |
| | cancer | | Egger | | | | |
| All targets | Oropharynx | 86 | All - Maximum | 0.5734 | 0.3918 | 0.8392 | 0.004210857 |
| | cancer | | likelihood | | | | |
| All targets | Oropharynx | 86 | All - Simple | 0.6930 | 0.3584 | 1.3402 | 0.275818557 |
| | cancer | | median | | | | |
| All targets | Oropharynx | 86 | All - Weighted | 0.6360 | 0.3557 | 1.1373 | 0.126981095 |
| | cancer | | median | | | | |
| All targets | Oropharynx | 86 | All - Simple | 1.0914 | 0.3786 | 3.1465 | 0.871756598 |
| | cancer | | mode | | | | |
| All targets | Oropharynx | 86 | All - Weighted | 0.5421 | 0.2855 | 1.0292 | 0.06465185 |
| | cancer | | mode | | | | |
| All targets | Oropharynx | 86 | All - Weighted | 0.5421 | 0.3046 | 0.9647 | 0.040320329 |
| | cancer | | mode (NOME) | | | | |
| All targets | Oropharynx | 86 | All - Simple | 1.0914 | 0.3879 | 3.0711 | 0.868777482 |
| | cancer | | mode (NOME) | | | | |
| All targets | Oropharynx | 86 | RAPS | | | | |
| | cancer | | | | | | |
| All targets | Oropharynx | 86 | MR_PRESSO | | | | 0.008242899 |
| | cancer | | | | | | |

MR-PRESSO: Mendelian Randomization Pleiotropy RESidual Sum and Outlier.
RAPS: Robust Adjusted Profile Score.

TABLE 13
Detailed PPI-based Mendelian Randomization (MR) estimates examining the association
of genetically proxied perturbation of KCNJ11 protein-protein interaction (PPI)
and PPARG PPI with cancer risk (using GTEx instruments). This table provides detailed
MR estimates for the PPI-based analysis, investigating the association between
genetically proxied perturbation of KCNJ11 PPI and PPARG PPI with cancer risk.
The analysis utilizes genetic instruments derived from the Genotype-Tissue Expression
(GTEx) project. The estimates include effect sizes (odds ratios, OR), 95% low confidence
intervals (95% LCI), 95% high confidence intervals (95% UCI) and P value, indicating
the strength and significance of the observed associations.

| | | | | | 95% | 95% | |
|---|---|---|---|---|---|---|---|
| Exposure | Outcome | nSNP | SNP | OR | LCI | UCI | P value |
| KCNJ11_PPI | Gastric | 22 | IVW (random | 0.6745 | 0.5851 | 0.7777 | 5.87E−08 |
| | cancer | | effects) | | | | |
| KCNJ11_PPI | Gastric | 22 | IVW (fixed | 0.6745 | 0.5539 | 0.8215 | 9.04E−05 |
| | cancer | | effects) | | | | |
| KCNJ11_PPI | Gastric | 22 | All - MR | 0.6921 | 0.3860 | 1.2411 | 0.23115183 |
| | cancer | | Egger | | | | |
| KCNJ11_PPI | Gastric | 22 | All - Maximum | 0.6793 | 0.5566 | 0.8290 | 0.000141735 |
| | cancer | | likelihood | | | | |
| KCNJ11_PPI | Gastric | 22 | All - Simple | 0.6623 | 0.4840 | 0.9062 | 0.009998517 |
| | cancer | | median | | | | |
| KCNJ11_PPI | Gastric | 22 | All - Weighted | 0.6600 | 0.5127 | 0.8497 | 0.001263952 |
| | cancer | | median | | | | |
| KCNJ11_PPI | Gastric | 22 | All - Simple | 0.6485 | 0.4750 | 0.8853 | 0.012640729 |
| | cancer | | mode | | | | |
| KCNJ11_PPI | Gastric | 22 | All - Weighted | 0.6682 | 0.5082 | 0.8784 | 0.008786591 |
| | cancer | | mode | | | | |
| KCNJ11_PPI | Gastric | 22 | All - Weighted | 0.6682 | 0.5183 | 0.8613 | 0.005269576 |
| | cancer | | mode (NOME) | | | | |
| KCNJ11_PPI | Gastric | 22 | All - Simple | 0.6485 | 0.4731 | 0.8889 | 0.013662106 |
| | cancer | | mode (NOME) | | | | |
| KCNJ11_PPI | Gastric | 22 | RAPS | | | | |
| | cancer | | | | | | |
| KCNJ11_PPI | Gastric | 22 | MR- | | | | 3.52E−05 |
| | cancer | | PRESSO(Raw) | | | | |
| PPARG_PPI | Oropharynx | 123 | IVW (random | 0.6486 | 0.4088 | 1.0289 | 0.065940834 |
| | cancer | | effects) | | | | |
| PPARG_PPI | Oropharynx | 123 | IVW (fixed | 0.6486 | 0.4220 | 0.9969 | 0.048354227 |
| | cancer | | effects) | | | | |
| PPARG_PPI | Oropharynx | 123 | All - MR | 0.3233 | 0.1395 | 0.7493 | 0.009573323 |
| | cancer | | Egger | | | | |
| PPARG_PPI | Oropharynx | 123 | All - Maximum | 0.6528 | 0.4152 | 1.0264 | 0.064698636 |
| | cancer | | likelihood | | | | |
| PPARG_PPI | Oropharynx | 123 | All - Simple | 1.1091 | 0.5208 | 2.3621 | 0.78825417 |
| | cancer | | median | | | | |
| PPARG_PPI | Oropharynx | 123 | All - Weighted | 0.2651 | 0.1241 | 0.5663 | 0.00060734 |
| | cancer | | median | | | | |
| PPARG_PPI | Oropharynx | 123 | All - Simple | 2.3734 | 0.3912 | 14.4002 | 0.349267638 |
| | cancer | | mode | | | | |
| PPARG_PPI | Oropharynx | 123 | All - Weighted | 0.1950 | 0.0861 | 0.4415 | 0.000146077 |
| | cancer | | mode | | | | |
| PPARG_PPI | Oropharynx | 123 | All - Weighted | 0.1950 | 0.0892 | 0.4265 | 7.65E−05 |
| | cancer | | mode (NOME) | | | | |
| PPARG_PPI | Oropharynx | 123 | All - Simple | 2.3734 | 0.4491 | 12.5436 | 0.310917446 |
| | cancer | | mode (NOME) | | | | |
| PPARG_PPI | Oropharynx | 123 | MR- | 0.6486 | 0.4088 | 1.0289 | 0.06837271 |
| | cancer | | PRESSO(Raw) | | | | |

MR-PRESSO: Mendelian Randomization Pleiotropy RESidual Sum and Outlier.
RAPS: Robust Adjusted Profile Score.

TABLE 14
Mendelian Randomization (MR) estimates examining the association of genetically proxied perturbation
of sulfonylurea (SU) and thiazolidinedione (TZD) with cancer risk (using GTEx instruments). This
table provides MR estimates for the SU and TZD anti-diabetic drugs, investigating the association
between genetically proxied perturbation of SU and TZD with cancer risk. The analysis utilizes genetic
instruments derived from the Genotype-Tissue Expression (GTEx) project. The estimates include effect
sizes (odds ratios, OR), 95% low confidence intervals (95% LCI), 95% high confidence intervals (95%
UCI) and P value, indicating the strength and significance of the observed associations.

| Exposure | Outcome | nSNP | SNP | OR | 95% LCI | 95% UCI | P value |
|---|---|---|---|---|---|---|---|
| Sulfonylureas | Gastric | 20 | IVW | 0.662925562 | 0.585131104 | 0.751062963 | 1.08307E−10 |
| | cancer | | (random | | | | |
| | | | effects) | | | | |
| Sulfonylureas | Gastric | 20 | IVW | 0.662925562 | 0.546477032 | 0.804188054 | 3.03076E−05 |
| | cancer | | (fixed | | | | |
| | | | effects) | | | | |
| Sulfonylureas | Gastric | 20 | All - MR | 0.770896526 | 0.45945831 | 1.293439343 | 0.334217554 |
| | cancer | | Egger | | | | |
| TZD | Oropharynx | 10 | IVW | 0.167999363 | 0.093836461 | 0.300776325 | 1.936E−09 |
| | cancer | | (random | | | | |
| | | | effects) | | | | |
| TZD | Oropharynx | 10 | IVW | 0.167999363 | 0.077152959 | 0.365815987 | 7.02609E−06 |
| | cancer | | (fixed | | | | |
| | | | effects) | | | | |
| TZD | Oropharynx | 10 | All - MR | 0.148275817 | 0.04319812 | 0.508950806 | 0.016225456 |
| | cancer | | Egger | | | | |

SU: sulfonylurea;
TDZ: thiazolidinedione.

TABLE 15
Mendelian Randomization (MR) estimates examining the association of genetically proxied
perturbation of KCNJ11 and ABCC8 with cancer risk (using GTEx instruments). This table
provides MR estimates for the KCNJ11 and ABCC8, investigating the association between
genetically proxied perturbation of KCNJ11 and ABCC8 activation with cancer risk.
The analysis utilizes genetic instruments derived from the Genotype-Tissue Expression
(GTEx) project. The estimates include effect sizes (odds ratios, OR), 95% low confidence
intervals (95% LCI), 95% high confidence intervals (95% UCI) and P value, indicating
the strength and significance of the observed associations.

| Exposure | Outcome | nSNP | SNP | OR | 95% LCI | 95% UCI | P value |
|---|---|---|---|---|---|---|---|
| KCNJ11 + | Gastric | 18 | IVW (random | 0.6637842 | 0.5842954 | 0.7540869 | 3.03E−10 |
| ABCC8 | cancer | | effects) | | | | |
| KCNJ11 + | Gastric | 18 | IVW (fixed | 0.6637842 | 0.5445361 | 0.8091466 | 4.99E−05 |
| ABCC8 | cancer | | effects) | | | | |
| KCNJ11 + | Gastric | 18 | All - MR | 0.8208701 | 0.4470043 | 1.5074299 | 0.5334277 |
| ABCC8 | cancer | | Egger | | | | |

TABLE 16
Drug-Target (KCNJ11 and PPARG) MR Estimates Examining the Association of Genetically Proxied
Perturbation of Single Drug Targets with Potential Risk Factors (Stage 1 MR). This table
presents the results of the first stage of the two-stage MR analysis investigating the
association between genetically proxied perturbation of single drug targets (KCNJ11 and
PPARG) and potential risk factors. The first stage MR analysis utilizes genetic variants
as instrumental variables to estimate the causal effect of perturbing each drug target
on the potential risk factors. The estimates include effect sizes (odds ratios, OR),
95% low confidence intervals (95% LCI), 95% high confidence intervals (95% UCI) and P
value, indicating the strength and significance of the observed associations.

| | | | | | | | Sample | |
|---|---|---|---|---|---|---|---|---|
| Exp | Out-come | nSNP | SNP | Beta | SE | P value | size | id.outcome |
| KCNJ11 | Body mass | 16 | IVW | −0.1396 | 0.0131 | 1.69521E−26 | 461460 | ukb-b-19953 |
| | index | | (random | | | | | |
| | | | effects) | | | | | |
| KCNJ11 | Body mass | 16 | All - MR | −0.1366 | 0.0331 | 0.001028593 | 461460 | ukb-b-19953 |
| | index | | Egger | | | | | |
| GLP1R | Body mass | 8 | IVW | 0.0729 | 0.0897 | 0.42 | 461460 | ukb-b-19953 |
| | index | | (random | | | | | |
| | | | effects) | | | | | |
| GLP1R | Body mass | 8 | All - MR | 0.7363 | 0.3058 | 0.04 | 461460 | ukb-b-19953 |
| | index | | Egger | | | | | |
| SLC5A2 | HbA1c | 9 | IVW | 0.4815 | 0.1237 | 9.91241E−05 | 45734 | ieu-b-4842 |
| | | | (random | | | | | |
| | | | effects) | | | | | |
| SLC5A2 | HbA1c | 9 | All - MR | 0.9403 | 0.5295 | 0.126137 | 45734 | ieu-b-4842 |
| | | | Egger | | | | | |
| RAMP2 | HbA1c | 8 | IVW | 0.2232 | 0.1152 | 0.04279687 | 45734 | ieu-b-4842 |
| | | | (random | | | | | |
| | | | effects) | | | | | |
| RAMP2 | HbA1c | 8 | All - MR | 0.2466 | 0.5268 | 0.65938521 | 45734 | ieu-b-4842 |
| | | | Egger | | | | | |
| PPARG | AAL | 10 | IVW | 0.4381 | 0.0370 | 2.56496E−32 | 389733 | ebi-a- |
| | | | (random | | | | | GCST90013992 |
| | | | effects) | | | | | |
| PPARG | AAL | 10 | All - MR | 0.3751 | 0.0557 | 0.000146634 | 389733 | ebi-a- |
| | | | Egger | | | | | GCST90013992 |
| PPARG | AAL | 10 | IVW | 0.2989 | 0.0462 | 9.75061E−11 | 388490 | ebi-a- |
| | | | (random | | | | | GCST90013996 |
| | | | effects) | | | | | |
| PPARG | AAL | 10 | All - MR | 0.3254 | 0.0772 | 0.002929342 | 388490 | ebi-a- |
| | | | Egger | | | | | GCST90013996 |
| KCNJ11 | HbA1c | 16 | IVW | 0.3869 | 0.0620 | 4.43E−10 | 44337 | ieu-b-4842 |
| | | | (random | | | | | |
| | | | effects) | | | | | |
| KCNJ11 | HbA1c | 16 | All - MR | 0.2706 | 0.1496 | 0.091966468 | 44337 | ieu-b-4842 |
| | | | Egger | | | | | |
| KCNJ11 | Fasting | 16 | IVW | 0.0296 | 0.0077 | 0.000119462 | 200622 | ebi-a- |
| | glucose | | (random | | | | | GCST90002232 |
| | | | effects) | | | | | |
| KCNJ11 | Fasting | 16 | All - MR | −0.0041 | 0.0288 | 0.889330712 | 200622 | ebi-a- |
| | glucose | | Egger | | | | | GCST90002232 |
| KCNJ11 | Fasting | 16 | IVW | −0.0203 | 0.0091 | 0.026448844 | 151013 | ebi-a- |
| | insulin | | (random | | | | | GCST90002238 |
| | | | effects) | | | | | |
| KCNJ11 | Fasting | 16 | All - MR | −0.0084 | 0.0323 | 0.798832489 | 151013 | ebi-a- |
| | insulin | | Egger | | | | | GCST90002238 |
| KCNJ11 | HDL | 7 | IVW | 0.0545 | 0.0157 | 0.000537174 | 94310 | ieu-a-299 |
| | cholesterol | | (random | | | | | |
| | | | effects) | | | | | |
| KCNJ11 | HDL | 7 | All - MR | 0.0182 | 0.1202 | 0.885347338 | 94310 | ieu-a-299 |
| | cholesterol | | Egger | | | | | |
| KCNJ11 | LDL | 7 | IVW | 0.1485 | 0.0162 | 4.01E−20 | 89887 | ieu-a-300 |
| | cholesterol | | (random | | | | | |
| | | | effects) | | | | | |
| KCNJ11 | LDL | 7 | All - MR | 0.1209 | 0.1283 | 0.389128128 | 89887 | ieu-a-300 |
| | cholesterol | | Egger | | | | | |
| PPARG | Body mass | 10 | IVW | −0.1517 | 0.0331 | 4.55E−06 | 461460 | ukb-b- 19953 |
| | index | | (random | | | | | |
| | | | effects) | | | | | |
| PPARG | Body mass | 10 | All - MR | −0.1305 | 0.0567 | 0.050312697 | 461460 | ukb-b- 19953 |
| | index | | Egger | | | | | |
| PPARG | HbA1c | 10 | IVW | 0.2949 | 0.1160 | 0.011046561 | 41889 | ieu-b- 4842 |
| | | | (random | | | | | |
| | | | effects) | | | | | |
| PPARG | HbA1c | 10 | All - MR | −0.0265 | 0.1393 | 0.854046624 | 41889 | ieu-b- 4842 |
| | | | Egger | | | | | |
| PPARG | Fasting | 10 | IVW | 0.0471 | 0.0115 | 4.45E−05 | 200622 | ebi-a- |
| | glucose | | (random | | | | | GCST90002232 |
| | | | effects) | | | | | |
| PPARG | Fasting | 10 | All - MR | 0.0801 | 0.0227 | 0.00771626 | 200622 | ebi-a- |
| | glucose | | Egger | | | | | GCST90002232 |
| PPARG | Fasting | 10 | IVW | 0.1921 | 0.0185 | 3.01E−25 | 151013 | ebi-a- |
| | insulin | | (random | | | | | GCST90002238 |
| | | | effects) | | | | | |
| PPARG | Fasting | 10 | All - MR | 0.2367 | 0.0262 | 1.79E−05 | 151013 | ebi-a- |
| | insulin | | Egger | | | | | GCST90002238 |
| PPARG | HDL | 8 | IVW | −0.0492 | 0.0772 | 0.524230953 | 186953 | ieu-a-299 |
| | cholesterol | | (random | | | | | |
| | | | effects) | | | | | |
| PPARG | HDL | 8 | All - MR | −0.2148 | 0.0984 | 0.071747625 | 186953 | ieu-a-299 |
| | cholesterol | | Egger | | | | | |
| PPARG | LDL | 8 | IVW | 0.1523 | 0.1244 | 0.220816435 | 172879 | ieu-a-300 |
| | cholesterol | | (random | | | | | |
| | | | effects) | | | | | |
| PPARG | LDL | 8 | All - MR | −0.1702 | 0.1264 | 0.2267448 | 172879 | ieu-a-300 |
| | cholesterol | | Egger | | | | | |

AAL: Alanine aminotransferase levels;
exp: Exposure.

TABLE 17
Heterogeneity and pleiotropy test for drug target Mendelian Randomization (MR) using GTEx
instruments. This table presents the results of the heterogeneity and pleiotropy tests
conducted for PPI-based MR analysis using genetic instruments from the Genotype-Tissue
Expression (GTEx) project. The heterogeneity analysis assesses the presence of variability
in causal estimates across individual genetic variants, while the pleiotropy test examines
the potential influence of pleiotropic effects (i.e., when a genetic variant affects multiple
traits or outcomes) on the MR analysis. The table includes test statistics such as Q statistics
and Egger statistics. The Q statistic evaluates heterogeneity by comparing the observed
variance to the expected variance under the assumption of homogeneity. Significantly high
Q values indicate heterogeneity, suggesting potential effect modification or subgroup-specific
effects. Additionally, the Egger statistic tests for directional pleiotropy, which occurs
when pleiotropic effects introduce bias in the MR analysis. Deviation from zero in the
Egger statistic suggests the presence of directional pleiotropy.

| | | | | Q_{—} | Q_{—} | Egger_{—} | | P |
|---|---|---|---|---|---|---|---|---|
| Outcome | Exposure | Method | Q | df | pval | intercept | SE | value |
| Gastric | KCNJ11 | MR Egger | 6.1374 | 14 | 0.9629 | −0.0092 | 0.0138 | 0.5164 |
| cancer | | | | | | | | |
| Gastric | KCNJ11 | Inverse | 6.5805 | 15 | 0.9683 | | | |
| cancer | | variance | | | | | | |
| | | weighted | | | | | | |
| Endometrial | GLP1R | MR Egger | 13.6013 | 6 | 0.0344 | 0.0031 | 0.0459 | 0.9480 |
| cancer | | | | | | | | |
| Endometrial | GLP1R | Inverse | 13.6118 | 7 | 0.0585 | | | |
| cancer | | variance | | | | | | |
| | | weighted | | | | | | |
| Bronchial | PPARG | MR Egger | 6.5046 | 8 | 0.5909 | −0.0161 | 0.0194 | 0.4301 |
| cancer | | | | | | | | |
| Bronchial | PPARG | Inverse | 7.1950 | 9 | 0.6168 | | | |
| cancer | | variance | | | | | | |
| | | weighted | | | | | | |
| Oropharynx | PPARG | MR Egger | 4.9759 | 8 | 0.7602 | 0.0064 | 0.0252 | 0.8045 |
| cancer | | | | | | | | |
| Oropharynx | PPARG | Inverse | 5.0413 | 9 | 0.8307 | | | |
| cancer | | variance | | | | | | |
| | | weighted | | | | | | |
| Bronchial | RAMP2 | MR Egger | 4.6633 | 6 | 0.5877 | 0.0254 | 0.0438 | 0.5829 |
| cancer | | | | | | | | |
| Bronchial | RAMP2 | Inverse | 4.9998 | 7 | 0.6600 | | | |
| cancer | | variance | | | | | | |
| | | weighted | | | | | | |

TABLE 18
Heterogeneity and pleiotropy test for protein-protein interaction (PPI)-Based and all targets-
based Mendelian Randomization (MR) using GTEx instruments. This table presents the results
of the heterogeneity and pleiotropy tests conducted for PPI-based MR analysis using genetic
instruments from the Genotype-Tissue Expression (GTEx) project. The heterogeneity analysis
assesses the presence of variability in causal estimates across individual genetic variants,
while the pleiotropy test examines the potential influence of pleiotropic effects (i.e.,
when a genetic variant affects multiple traits or outcomes) on the MR analysis. The table
includes test statistics such as Q statistics and Egger statistics. The Q statistic evaluates
heterogeneity by comparing the observed variance to the expected variance under the assumption
of homogeneity. Significantly high Q values indicate heterogeneity, suggesting potential
effect modification or subgroup-specific effects.

| | | | | Q_{—} | Q_{—} | Egger_{—} | | |
|---|---|---|---|---|---|---|---|---|
| Outcome | Exposure | Method | Q | df | pval | intercept | SE | P value |
| Gastric | KCNJ11- | MR | 10.9361 | 19 | 0.9260 | −0.0012 | 0.0128 | 0.9292 |
| cancer | PPI | Egger | | | | | | |
| Gastric | KCNJ11- | Inverse | 10.9442 | 20 | 0.9477 | | | |
| cancer | PPI | variance | | | | | | |
| | | weighted | | | | | | |
| Oropharynx | PPARG_PPI | MR | 136.4178 | 121 | 0.1600 | 0.0190 | 0.0099 | 0.0556 |
| cancer | | Egger | | | | | | |
| Oropharynx | PPARG_PPI | Inverse | 140.6305 | 122 | 0.1192 | | | |
| cancer | | variance | | | | | | |
| | | weighted | | | | | | |
| Gastric | All targets | MR | 148.7451 | 100 | 0.0011 | | | |
| cancer | | Egger | | | | | | |
| Gastric | All targets | Inverse | 148.8529 | 101 | 0.0014 | | | |
| cancer | | variance | | | | | | |
| | | weighted | | | | | | |
| Oropharynx | All targets | MR | 142.6679 | 100 | 0.0011 | | | |
| cancer | | Egger | | | | | | |
| Oropharynx | All targets | Inverse | 142.7456 | 101 | 0.0014 | | | |
| cancer | | variance | | | | | | |
| | | weighted | | | | | | |

TABLE 19
Colocalization analysis of posterior probabilities under differing
hypotheses relating to the associations between T2D variants in
or within proximity to the KCNK11 locus and gastric cancer.
GTEx KCNJ11 on GC

| Configuration | H0 | H1 | H2 | H3 | H4 | PP.H4 |
|---|---|---|---|---|---|---|
| | 1.61E−240 | 9.69E−01 | 3.70E−242 | 2.22E−02 | 8.98E−03 | 0.8153 |
| rs2074310 | pvalues.df1 | 4.83E−248 | | | | |

“PP abf for shared variant: 0.898%”
GTEx KCNJ11 on LDL

| Configuration | H0 | H1 | H2 | H3 | H4 | PP.H4 |
|---|---|---|---|---|---|---|
| | 1.45E−149 | 9.74E−01 | 1.11E−151 | 7.44E−03 | 1.84E−02 | 0.8372 |
| rs4148646 | pvalues.df1 | 8.75E−157 | | | | |

“PP abf for shared variant: 1.84%”

| SNP1 | SNP2 | D′ | R2 |
|---|---|---|---|
| rs5215 | rs2074310 | 0.965 | 0.919 |
| rs2074310 | rs4148646 | 0.996 | 0.991 |
| rs5215 | rs4148646 | 0.969 | 0.927 |

TABLE 20
Drug-target (KCNJ11 and PPARG) Mendelian Randomization (MR) estimates examining the association
of potential risk factors with cancer risk (Stage 2 MR). This table presents the results
of the second stage of the two-stage MR analysis investigating the association between
potential risk factors and cancer risk, using KCNJ11 and PPARG as drug targets. The
two-stage MR approach utilizes genetic variants as instrumental variables to assess
causal relationships. The estimates include effect sizes (odds ratios, OR), 95% low
confidence intervals (95% LCI), 95% high confidence intervals (95% UCI) and P value,
indicating the strength and significance of the observed associations.

| | | | | | 95% | 95% | P | |
|---|---|---|---|---|---|---|---|---|
| Exposure | Outcome | nSNP | SNP | OR | LCI | UCI | value | Targets |
| BMI | OC | 378 | IVW | 1.0685 | 0.8280 | 1.3789 | 0.610465268 | GTEx_PPARG |
| | | | (random | | | | | |
| | | | effects) | | | | | |
| BMI | OC | 378 | All - MR | 0.6137 | 0.3092 | 1.2179 | 0.163472432 | GTEx_PPARG |
| | | | Egger | | | | | |
| HbA1c | OC | 29 | IVW | 1.4266 | 1.1541 | 1.7636 | 0.001021349 | GTEx_PPARG |
| | | | (random | | | | | |
| | | | effects) | | | | | |
| HbA1c | OC | 29 | All - MR | 1.6021 | 1.0047 | 2.5547 | 0.058832969 | GTEx_PPARG |
| | | | Egger | | | | | |
| Fasting | OC | 67 | IVW | 1.2462 | 0.6787 | 2.2882 | 0.477828674 | GTEx_PPARG |
| glucose | | | (random | | | | | |
| | | | effects) | | | | | |
| Fasting | OC | 67 | All - MR | 2.3866 | 0.7029 | 8.1028 | 0.168487998 | GTEx_PPARG |
| glucose | | | Egger | | | | | |
| Fasting | OC | 31 | IVW | 0.9397 | 0.3272 | 2.6984 | 0.9080045 | GTEx_PPARG |
| insulin | | | (random | | | | | |
| | | | effects) | | | | | |
| Fasting | OC | 31 | All - MR | 0.0141 | 0.0007 | 0.2913 | 0.009394587 | GTEx_PPARG |
| insulin | | | Egger | | | | | |
| HDL | OC | 296 | IVW | 0.9228 | 0.7432 | 1.1457 | 0.466535763 | GTEx_PPARG |
| cholesterol | | | (random | | | | | |
| | | | effects) | | | | | |
| HDL | OC | 296 | All - MR | 1.2814 | 0.8449 | 1.9432 | 0.246542332 | GTEx_PPARG |
| cholesterol | | | Egger | | | | | |
| LDL | OC | 79 | IVW | 0.8972 | 0.7263 | 1.1082 | 0.313990443 | GTEx_PPARG |
| cholesterol | | | (random | | | | | |
| | | | effects) | | | | | |
| LDL | OC | 79 | All - MR | 1.0640 | 0.7834 | 1.4452 | 0.692397304 | GTEx_PPARG |
| cholesterol | | | Egger | | | | | |
| BMI | Gastric | 407 | IVW | 1.0291 | 0.9217 | 1.1492 | 0.609706927 | GTEx_KCNJ11 |
| | cancer | | (random | | | | | |
| | | | effects) | | | | | |
| BMI | Gastric | 407 | All - MR | 0.8385 | 0.6222 | 1.1301 | 0.247962892 | GTEx_KCNJ11 |
| | cancer | | Egger | | | | | |
| HbA1c | Gastric | 30 | IVW | 1.0837 | 0.9441 | 1.2440 | 0.253161965 | GTEx_KCNJ11 |
| | cancer | | (random | | | | | |
| | | | effects) | | | | | |
| HbA1c | Gastric | 30 | All - MR | 1.2189 | 0.8056 | 1.8442 | 0.357436659 | GTEx_KCNJ11 |
| | cancer | | Egger | | | | | |
| Fasting | Gastric | 60 | IVW | 1.0518 | 0.7754 | 1.4268 | 0.745416985 | GTEx_KCNJ11 |
| glucose | cancer | | (random | | | | | |
| | | | effects) | | | | | |
| Fasting | Gastric | 60 | All - MR | 1.1987 | 0.6922 | 2.0757 | 0.520252096 | GTEx_KCNJ11 |
| glucose | cancer | | Egger | | | | | |
| Fasting | Gastric | 72 | IVW | 0.8529 | 0.5247 | 1.3864 | 0.520897967 | GTEx_KCNJ11 |
| insulin | cancer | | (random | | | | | |
| | | | effects) | | | | | |
| Fasting | Gastric | 72 | All - MR | 0.2749 | 0.0610 | 1.2397 | 0.101544106 | GTEx_KCNJ11 |
| insulin | cancer | | Egger | | | | | |
| HDL | Gastric | 310 | IVW | 0.9934 | 0.8903 | 1.1085 | 0.905620912 | GTEx_KCNJ11 |
| cholesterol | cancer | | (random | | | | | |
| | | | effects) | | | | | |
| HDL | Gastric | 310 | All - MR | 0.8267 | 0.6748 | 1.0127 | 0.069672345 | GTEx_KCNJ11 |
| cholesterol | cancer | | Egger | | | | | |
| LDL | Gastric | 75 | IVW | 0.8731 | 0.7890 | 0.9661 | 0.008602559 | GTEx_KCNJ11 |
| cholesterol | cancer | | (random | | | | | |
| | | | effects) | | | | | |
| LDL | Gastric | 75 | All - MR | 0.8762 | 0.7515 | 1.0216 | 0.095633811 | GTEx_KCNJ11 |
| cholesterol | cancer | | Egger | | | | | |

OC: Oropharynx cancer.

TABLE 21
Mediation effect of LDL-C on the causal effect of genetically proxied
activation of KCNJ11 on gastric cancer (GC) risk. This table presents
the results of mediation analysis examining the potential role of
LDL-C in mediating the causal effect between genetically proxied
activation of KCNJ11 and the risk of gastric cancer (GC). The analysis
investigates whether LDL-C acts as a mediator in the biological
mechanism underlying the observed association.

| | Total | Direct | Indirect | Mediation |
|---|---|---|---|---|
| Mediator | effect (β) | effect | effect (β) | proportion |
| LDL-C | −0.167774243 | −0.159020485 | −0.008753758 | 4.81% |

TABLE 22
Multivariable Mendelian Randomization (MR) results for genetically proxied activation
of KCNJ11 on gastric cancer. This table presents the results of multivariable MR
analysis examining the association between genetically proxied inhibition of KCNJ11
and the risk of gastric cancer. The multivariable MR approach accounts for potential
confounding factors and adjusts for relevant covariates to provide a more robust
estimation of the causal effect. The estimates include effect sizes (odds ratios,
OR), 95% low confidence intervals (95% LCI), 95% high confidence intervals (95% UCI)
and P value, indicating the strength and significance of the observed associations.

| Exposure | Outcome | Confounders | P value | OR | 95% LCI | 95% UCI |
|---|---|---|---|---|---|---|
| KCNJ11 | Gastric | All | 0.001264842 | 0.4266 | 0.2805 | 0.6487 |
| | cancer | BMI | 0.000363618 | 0.4262 | 0.2979 | 0.6098 |
| | | HbA1c | 0.24963006 | 0.8713 | 0.6958 | 1.0910 |
| | | Fasting glucose | 0.000332191 | 0.7307 | 0.6414 | 0.8325 |
| | | FI | 0.001117873 | 0.6887 | 0.5758 | 0.8237 |
| | | HDL | 0.005292711 | 0.6695 | 0.5274 | 0.8498 |
| | | BMI, HbA1c | 0.1102047 | 0.5788 | 0.3098 | 1.0815 |
| | | BMI, FG | 0.1219569 | 0.5373 | 0.2574 | 1.1216 |
| | | BMI, FI | 0.000546575 | 0.4303 | 0.2992 | 0.6188 |
| | | BMI, HDL | 0.000115634 | 0.4011 | 0.2883 | 0.5578 |
| | | HbA1c, FG | 0.1767691 | 0.7980 | 13.1173 | 0.0485 |
| | | HbA1c, FI | 0.2645424 | 0.8758 | 0.7009 | 1.0945 |
| | | HbA1c, HDL | 0.34462049 | 0.8654 | 0.6483 | 1.1552 |
| | | FG, FI | 0.002161116 | 0.7334 | 0.6252 | 0.8602 |
| | | FG, HDL | 0.02069107 | 0.7461 | 0.5999 | 0.9279 |
| | | FI, HDL | 0.008110723 | 0.6731 | 0.5250 | 0.8631 |
| | | BMI, HbA1c, FG | 0.146073 | 0.5542 | 0.2633 | 1.1666 |
| | | BMI, HbA1c, FI | 0.3443684 | 0.6182 | 0.2373 | 1.6107 |
| | | BMI, HbA1c, HDL | 0.03762161 | 0.4222 | 0.2048 | 0.8703 |
| | | BMI, HbA1c, FG, FI | 0.3550152 | 0.6120 | 0.2259 | 1.6581 |
| | | BMI, HbA1c, FG, HDL | 0.005906897 | 0.1749 | 0.0641 | 0.4776 |
| | | BMI, HbA1c, FI, HDL | 0.1335096 | 0.4125 | 0.1413 | 1.2042 |
| | | BMI, FG, FI | 0.1168159 | 0.4946 | 0.2186 | 1.1190 |
| | | BMI, FG, HDL | 0.008746339 | 0.1849 | 0.0642 | 0.5327 |
| | | BMI, FG, FI, HDL | 0.01015351 | 0.1715 | 0.0562 | 0.5234 |
| | | BMI, FI, HDL | 0.000241986 | 0.4027 | 0.2848 | 0.5693 |
| | | HbA1c, FG, FI | 0.5487175 | 0.8791 | 0.5837 | 1.3238 |
| | | HbA1c, FG, HDL | 0.2387201 | 0.8040 | 0.5696 | 1.1350 |
| | | HbA1c, FG, FI, HDL | 0.8444065 | 0.9489 | 0.5690 | 1.5826 |
| | | HbA1c, FI, HDL | 0.61431602 | 0.9233 | 0.6824 | 1.2493 |

SE: standard error;
BMI: body mass index;
FG: fasting glucose;
FI: fasting insulin;
HDL-C: high-density lipoprotein cholesterol;
LDL-C: low-density lipoprotein cholesterol.

TABLE 23
Colocalization analysis of posterior probabilities under differing
hypotheses relating to the associations between T2D variants in
or within proximity to the PPARG locus and oropharynx cancer. The
hypotheses are as follows: H0 is that neither T2D nor BMI has a
genetic association in the region, H1 is that only T2D has a genetic
association in the region, H2 is that only BMI has a genetic association
in the region, H3 is that both T2D and BMI are associated but have
different causal variants, and H4 is that both T2D and BMI are
associated and share a single causal variant.

| Configuration | H0 | H1 | H2 | H3 | H4 |
|---|---|---|---|---|---|
| | 0.838 | 0.112 | 0.0411 | 0.00543 | 0.00312 |

TABLE 24
Multivariable Mendelian Randomization (MR) results for genetically proxied activation
of PPARG on oropharynx cancer. This table presents the results of multivariable MR
analysis examining the association between genetically proxied inhibition of PPARG
and the risk of oropharynx cancer. The multivariable MR approach accounts for potential
confounding factors and adjusts for relevant covariates to provide a more robust estimation
of the causal effect. The estimates include effect sizes (odds ratios, OR), 95% low
confidence intervals (95% LCI), 95% high confidence intervals (95% UCI) and P value,
indicating the strength and significance of the observed associations.

| Exposure | Outcome | Confounders | P value | OR | 95% LCI | 95% UCI |
|---|---|---|---|---|---|---|
| PPARG | Oropharynx | All | 0.1799838 | 0.3517 | 0.1280 | 0.9664 |
| | cancer | BMI | 0.1021578 | 0.1666 | 0.0828 | 0.3352 |
| | | HbA1c | 0.000394032 | 0.1618 | 0.0877 | 0.2987 |
| | | FG | 0.05110608 | 0.2387 | 0.1016 | 0.5612 |
| | | FI | 0.05308281 | 0.2116 | 0.0811 | 0.5518 |
| | | HDL | 0.3204483 | 0.3380 | 0.2082 | 0.5486 |
| | | LDL | 0.000435475 | 0.1570 | 0.0933 | 0.2644 |
| | | BMI, HbA1c | 0.001021578 | 0.1666 | 0.0828 | 0.3352 |
| | | BMI, FG | 0.01637684 | 0.2334 | 0.0941 | 0.5788 |
| | | BMI, FI | 0.02769549 | 0.2314 | 0.0822 | 0.6519 |
| | | BMI, HDL | 0.003027534 | 0.2658 | 0.1480 | 0.4774 |
| | | BMI, LDL | 0.00363005 | 0.1690 | 0.0858 | 0.3327 |
| | | HbA1c, FG | 0.01445502 | 0.2315 | 0.0952 | 0.5625 |
| | | HbA1c, FI | 0.03282881 | 0.2606 | 0.0965 | 0.7040 |
| | | HbA1c, HDL | 0.002143497 | 0.2784 | 0.1621 | 0.4781 |
| | | HbA1c, LDL | 0.002401885 | 0.1866 | 0.1043 | 0.3339 |
| | | FG, FI | 0.02365515 | 0.2310 | 0.0852 | 0.6263 |
| | | FG, HDL | 0.003769606 | 0.2206 | 0.1099 | 0.4425 |
| | | FG, LDL | 0.02178362 | 0.3042 | 0.1496 | 0.6185 |
| | | FI, HDL | 0.01189657 | 0.2791 | 0.1329 | 0.5861 |
| | | FI, LDL | 0.02608224 | 0.3246 | 0.1603 | 0.6573 |
| | | HDL, LDL | 0.004106898 | 0.2843 | 0.1736 | 0.4655 |
| | | BMI, HbA1c, FG | 0.02461594 | 0.2320 | 0.0887 | 0.6063 |
| | | BMI, HbA1c, FI | 0.05111101 | 0.2623 | 0.0891 | 0.7717 |
| | | BMI, HbA1c, HDL | 0.006383925 | 0.2781 | 0.1507 | 0.5130 |
| | | BMI, HbA1c, LDL | 0.009210031 | 0.1847 | 0.0915 | 0.3728 |
| | | BMI, HbA1c, FG, FI | 0.07630758 | 0.2623 | 0.0808 | 0.8511 |
| | | BMI, HbA1c, FG, HDL | 0.005335077 | 0.2285 | 0.1234 | 0.4229 |
| | | BMI, HbA1c, FG, LDL | 0.06982441 | 0.3107 | 0.1357 | 0.7114 |
| | | BMI, HbA1c, FG, FI, | 0.02345605 | 0.2474 | 0.1148 | 0.5330 |
| | | HDL | | | | |
| | | BMI, HbA1c, FG, FI, | 0.1291464 | 0.3657 | 0.1665 | 0.8033 |
| | | LDL | | | | |
| | | BMI, HbA1c, FG, FI, | 0.4739103 | 0.5287 | 0.1673 | 1.6709 |
| | | HDL, LDL | | | | |
| | | BMI, HbA1c, FI, HDL | 0.01760606 | 0.2698 | 0.1223 | 0.5955 |
| | | BMI, HbA1c, FI, LDL | 0.03951947 | 0.3508 | 0.1774 | 0.6938 |
| | | BMI, HbA1c, FI, HDL, | 0.07783883 | 0.3684 | 0.1754 | 0.7738 |
| | | LDL | | | | |
| | | BMI, FG, FI | 0.04370756 | 0.2426 | 0.0816 | 0.7217 |
| | | BMI, FG, HDL | 0.004379888 | 0.2276 | 0.1184 | 0.4375 |
| | | BMI, FG, FI, HDL | 0.003769606 | 0.2206 | 0.1099 | 0.4425 |
| | | BMI, FG, FI, LDL | 0.057135069 | 0.5078 | 0.0582 | 4.4318 |
| | | BMI, FG, FI, HDL, LDL | 0.06397396 | 0.3314 | 0.1559 | 0.7043 |
| | | HbA1c, FG, FI | 0.209109901 | 0.0887 | 0.0030 | 2.5926 |
| | | HbA1c, FG, HDL | 0.70501302 | 2.7999 | 0.0174 | 450.7501 |
| | | HbA1c, FG, LDL | 0.05183737 | 0.2995 | 0.1346 | 0.6667 |
| | | HbA1c, FG, FI, HDL | 0.08785655 | 0.3295 | 0.1379 | 0.7873 |
| | | HbA1c, FG, FI, LDL | 0.45231036 | 0.1016 | 0.0006 | 18.4510 |
| | | HbA1c, FG, FI, HDL, | 0.61803029 | 0.3153 | 0.0048 | 20.8547 |
| | | LDL | | | | |
| | | FG, FI, HDL | 0.01220674 | 0.2784 | 0.1372 | 0.5651 |
| | | FG, FI, LDL | 0.24112826 | 0.5345 | 0.0457 | 6.2512 |
| | | FG, FI, HDL, LDL | 0.68592234 | 2.5196 | 0.0434 | 146.4359 |
| | | FI, HDL, LDL | 0.68592234 | 4.5664 | 1.9109 | 10.9118 |

SE: standard error;
BMI: body mass index;
FG: fasting glucose;
FI: fasting insulin;
HDL-C: high-density lipoprotein cholesterol;
LDL-C: low-density lipoprotein cholesterol.

TABLE 25
Drug-Target Mendelian Randomization estimates examining the association of genetically
proxied inhibition of KCNJ11 and PPARG with cancer risk (adjusted for body mass index
(BMI)). This table presents the Mendelian Randomization (MR) estimates investigating
the association between genetically proxied inhibition of KCNJ11 and PPARG and the risk
of cancer. The MR analysis utilizes genetic variants as instrumental variables and adjusts
for the confounding effects of BMI. The estimates include effect sizes (odds ratios,
OR), 95% low confidence intervals (95% LCI), 95% high confidence intervals (95% UCI)
and P value, indicating the strength and significance of the observed associations.

| Exposure | Outcome | nSNP | SNP | OR | 95% LCI | 95% UCI | P value |
|---|---|---|---|---|---|---|---|
| PPARG | Oropharynx | 10 | IVW (random | 0.2096 | 0.1371 | 0.3203 | 5.22E−13 |
| | cancer | | effects) | | | | |
| PPARG | Oropharynx | 10 | IVW (fixed | 0.2096 | 0.1083 | 0.4056 | 3.51E−06 |
| | cancer | | effects) | | | | |
| PPARG | Oropharynx | 10 | All - MR | 0.1659 | 0.0567 | 0.4854 | 0.011194903 |
| | cancer | | Egger | | | | |
| PPARG | Oropharynx | 10 | All - | 0.2058 | 0.1029 | 0.4118 | 7.91E−06 |
| | cancer | | Maximum | | | | |
| | | | likelihood | | | | |
| PPARG | Oropharynx | 10 | All - Simple | 0.2655 | 0.0857 | 0.8229 | 0.021577866 |
| | cancer | | median | | | | |
| PPARG | Oropharynx | 10 | All - Weighted | 0.2366 | 0.0994 | 0.5630 | 0.0011187 |
| | cancer | | median | | | | |
| PPARG | Oropharynx | 10 | All - Simple | 0.1777 | 0.0536 | 0.5899 | 0.019968711 |
| | cancer | | mode | | | | |
| PPARG | Oropharynx | 10 | All - Weighted | 0.2081 | 0.0876 | 0.4943 | 0.006156007 |
| | cancer | | mode | | | | |
| PPARG | Oropharynx | 10 | All - Weighted | 0.2040 | 0.0912 | 0.4566 | 0.003805576 |
| | cancer | | mode (NOME) | | | | |
| PPARG | Oropharynx | 10 | All - Simple | 0.1777 | 0.0584 | 0.5406 | 0.01392708 |
| | cancer | | mode (NOME) | | | | |
| PPARG | Oropharynx | 10 | MR- | 0.2096 | 0.1371 | 0.3203 | 4.98E−05 |
| | cancer | | PRESSO(Raw) | | | | |
| KCNJ11 | Gastric | 16 | IVW (random | 0.6714 | 0.5845 | 0.7713 | 1.80E−08 |
| | cancer | | effects) | | | | |
| KCNJ11 | Gastric | 16 | IVW (fixed | 0.6714 | 0.5411 | 0.8331 | 0.000296465 |
| | cancer | | effects) | | | | |
| KCNJ11 | Gastric | 16 | All - MR | 0.7907 | 0.4696 | 1.3315 | 0.392100427 |
| | cancer | | Egger | | | | |
| KCNJ11 | Gastric | 16 | All - | 0.6697 | 0.5370 | 0.8352 | 0.000374124 |
| | cancer | | Maximum | | | | |
| | | | likelihood | | | | |
| KCNJ11 | Gastric | 16 | All - Simple | 0.6655 | 0.4709 | 0.9407 | 0.021102621 |
| | cancer | | median | | | | |
| KCNJ11 | Gastric | 16 | All - Weighted | 0.6788 | 0.5159 | 0.8930 | 0.005625791 |
| | cancer | | median | | | | |
| KCNJ11 | Gastric | 16 | All - Simple | 0.6274 | 0.4315 | 0.9121 | 0.027493559 |
| | cancer | | mode | | | | |
| KCNJ11 | Gastric | 16 | All - Weighted | 0.6941 | 0.5237 | 0.9201 | 0.022680251 |
| | cancer | | mode | | | | |
| KCNJ11 | Gastric | 16 | All - Weighted | 0.6941 | 0.5307 | 0.9080 | 0.017660933 |
| | cancer | | mode (NOME) | | | | |
| KCNJ11 | Gastric | 16 | All - Simple | 0.6274 | 0.4288 | 0.9178 | 0.029731494 |
| | cancer | | mode (NOME) | | | | |
| KCNJ11 | Gastric | 16 | MR- | 0.6714 | 0.5845 | 0.7713 | 4.79E−05 |
| | cancer | | PRESSO(Raw) | | | | |

TABLE 26
Drug-Target Mendelian Randomization (MR) estimates examining the association of genetically
proxied inhibition of KCNJ11 with gastric cancer risk (ESA Ancestry). This table provides
the MR estimates examining the association between genetically proxied inhibition of KCNJ11
and the risk of gastric cancer. MR analysis utilizes genetic variants as instrumental variables
to assess causal relationships. The estimates include effect sizes (odds ratios, OR), 95%
low confidence intervals (95% LCI), 95% high confidence intervals (95% UCI) and p values,
indicating the strength and significance of the observed associations. The analysis specifically
focuses on individuals of European South Asian (ESA) ancestry.

| Exposure | Outcome | nSNP | SNP | OR | 95% LCI | 95% UCI | P value |
|---|---|---|---|---|---|---|---|
| KCNJ11 | Gastric | 14 | IVW (random | 0.7528 | 0.6699 | 0.8461 | 1.87E−06 |
| | cancer (EAS) | | effects) | | | | |
| KCNJ11 | Gastric | 14 | IVW (fixed | 0.7528 | 0.6393 | 0.8865 | 0.000661978 |
| | cancer (EAS) | | effects) | | | | |
| KCNJ11 | Gastric | 14 | All - MR Egger | 0.6514 | 0.3607 | 1.1764 | 0.180660313 |
| | cancer (EAS) | | | | | | |
| KCNJ11 | Gastric | 14 | All - Maximum | 0.7524 | 0.6373 | 0.8883 | 0.000786014 |
| | cancer (EAS) | | likelihood | | | | |
| KCNJ11 | Gastric | 14 | All - Simple | 0.7564 | 0.6099 | 0.9380 | 0.011003833 |
| | cancer (EAS) | | median | | | | |
| KCNJ11 | Gastric | 14 | All - Weighted | 0.7589 | 0.6228 | 0.9248 | 0.006230404 |
| | cancer (EAS) | | median | | | | |
| KCNJ11 | Gastric | 14 | All - Simple | 0.7622 | 0.5839 | 0.9950 | 0.067199527 |
| | cancer (EAS) | | mode | | | | |
| KCNJ11 | Gastric | 14 | All - Weighted | 0.7622 | 0.5999 | 0.9684 | 0.04459468 |
| | cancer (EAS) | | mode | | | | |
| KCNJ11 | Gastric | 14 | All - Weighted | 0.7622 | 0.6139 | 0.9463 | 0.028679345 |
| | cancer (EAS) | | mode (NOME) | | | | |
| KCNJ11 | Gastric | 14 | All - Simple | 0.7622 | 0.5920 | 0.9814 | 0.055243009 |
| | cancer (EAS) | | mode (NOME) | | | | |
| KCNJ11 | Gastric | 14 | RAPS | 0.7622 | 0.6139 | 0.9463 | 0.02220763 |
| | cancer (EAS) | | | | | | |
| KCNJ11 | Gastric | 14 | MR - PRESSO | 0.7622 | 0.5999 | 0.9950 | 8.36E−10 |
| | cancer (EAS) | | | | | | |

TABLE 27
Mendelian Randomization (MR) estimates examining the association of genetically proxied
perturbation of anti-diabetic drugs with gastric cancer risk (Using GTEx Instruments).
This table provides MR estimates for the anti-diabetic drugs, investigating the association
between genetically proxied perturbation of anti-diabetic drug targets with gastric
cancer risk. The analysis utilizes genetic instruments derived from the Genotype-Tissue
Expression (GTEx) project. The estimates include effect sizes (odds ratios, OR), 95%
low confidence intervals (95% LCI), 95% high confidence intervals (95% UCI) and P value,
indicating the strength and significance of the observed associations.

| Exposure | Outcome | nSNP | SNP | OR | 95% LCI | 95% UCI | P value |
|---|---|---|---|---|---|---|---|
| KCNJ11 | Gastric | 16 | IVW | 1.03125331 | 0.981283 | 1.010002 | 0.8133245 |
| | cancer | | (random | | | | |
| | | | effects) | | | | |
| KCNJ11 + | Gastric | 18 | IVW (fixed | 1.00202341 | 0.982337 | 1.010203 | 0.6294343 |
| ABCC8 | cancer | | effects) | | | | |
| KCNJ11 − | Gastric | 22 | All - MR | 1.02202301 | 0.984532 | 1.010314 | 0.5493233 |
| PPI | cancer | | Egger | | | | |
| SU | Gastric | 27 | IVW | 0.97087234 | 0.967342 | 0.998222 | 0.0483422 |
| | cancer | | (random | | | | |
| | | | effects) | | | | |
| All-targets | Gastric | 87 | IVW (fixed | 0.97866532 | 0.952313 | 0.999124 | 0.0223234 |
| | cancer | | effects) | | | | |

SU: Sulfonylurea.

TABLE 28
Differential expression analysis of KCNJ11 PPI-based genes using the limma package. In
this table, logFC represents the log2 fold change, which indicates the relative change
in gene expression between the compared groups. A positive logFC indicates upregulation,
while a negative logFC indicates downregulation. Beta refers to the estimated effect
size of the gene expression difference between groups. A positive beta value indicates
increased expression, while a negative beta value indicates decreased expression. P value
represents the p value associated with each gene, indicating the statistical significance
of the differential expression. Adjusted P may be reported when multiple testing corrections
have been applied, such as the false discovery rate (FDR) correction.

| logFC | AveExpr | t | P value | Adjusted P | Beta | Targets | Dataset ID |
|---|---|---|---|---|---|---|---|
| −0.8849 | 4.8704 | −6.8010 | 2.81E−09 | 1.04E−07 | 10.9531 | ABCC8 | GSE13911 |
| −0.5671 | 9.2512 | −4.2627 | 6.16E−05 | 0.000457533 | 1.2987 | SIK1 | GSE13911 |
| −0.8235 | 8.6923 | −3.6086 | 0.000571922 | 0.002780735 | −0.8065 | KCNQ1 | GSE13911 |
| −0.6844 | 4.9106 | −2.5178 | 0.014087193 | 0.037297656 | −3.7450 | KCNJ11 | GSE13911 |
| −0.3783 | 8.2059 | −1.9111 | 0.060067665 | 0.11852434 | −5.0003 | PRKACA | GSE13911 |
| −0.2592 | 5.8980 | −1.5573 | 0.123878784 | 0.208217914 | −5.5890 | ABCC9 | GSE13911 |
| −1.1822 | 7.4006 | −3.6346 | 0.001610771 | 0.031757533 | −1.1769 | KCNQ1 | GSE79973 |
| −0.8844 | 3.7415 | −2.1599 | 0.042860555 | 0.18340983 | −4.1648 | ABCC8 | GSE79973 |
| −0.5100 | 8.5836 | −2.1313 | 0.045415973 | 0.189145047 | −4.2151 | SIK1 | GSE79973 |
| 0.4143 | 5.1412 | 1.4866 | 0.15241625 | 0.373347202 | −5.2241 | ABCC9 | GSE79973 |
| −0.2972 | 6.5039 | −1.1686 | 0.256065216 | 0.495864148 | −5.6163 | PRKACA | GSE79973 |
| −0.5801 | 3.9194 | −0.7578 | 0.457269907 | 0.680219113 | −5.9985 | KCNJ11 | GSE79973 |

AveExpr: Average expression.

TABLE 29
Differential expression analysis of PPARG PPI-based genes using the limma package.
In this table, logFC represents the log2 fold change, which indicates the relative
change in gene expression between the compared groups. A positive logFC indicates
upregulation, while a negative logFC indicates downregulation. Beta refers to
the estimated effect size of the gene expression difference between groups. A
positive beta value indicates increased expression, while a negative beta value
indicates decreased expression. P value represents the p value associated with
each gene, indicating the statistical significance of the differential expression.
Adjusted P may be reported when multiple testing corrections have been applied,
such as the false discovery rate (FDR) correction.

| logFC | AveExpr | t | P value | Adjusted P | Beta | Targets | Dataset ID |
|---|---|---|---|---|---|---|---|
| 1.7549 | 8.1228 | 4.9493 | 1.10E−05 | 0.000517349 | 3.2102 | MMP9 | GSE23558 |
| −0.3045 | 4.5407 | −3.9585 | 0.000267376 | 0.005168319 | 0.1925 | PPARG | GSE23558 |
| 0.5051 | 4.8802 | 3.7074 | 0.000575605 | 0.008983562 | −0.5260 | ABCG1 | GSE23558 |
| 0.2430 | 6.9988 | 3.1906 | 0.002597867 | 0.026341094 | −1.9234 | CDK5 | GSE23558 |
| −0.1829 | 7.1899 | −3.1485 | 0.002922522 | 0.028444587 | −2.0315 | CDK19 | GSE23558 |
| 0.7232 | 7.0933 | 3.1328 | 0.00305337 | 0.029385454 | −2.0717 | EGFR | GSE23558 |
| −3.1285 | −2.9259 | −4.4345 | 9.35E−05 | 0.001784884 | 1.1755 | PPARG | GSE37991 |
| 1.4154 | 1.3048 | 2.5725 | 0.014687565 | 0.070595417 | −3.5706 | EGFR | GSE37991 |
| 1.7517 | 1.8268 | 2.5413 | 0.015830547 | 0.074374346 | −3.6381 | MMP9 | GSE37991 |
| −0.6020 | −0.0757 | −1.6854 | 0.101165274 | 0.265385312 | −5.2433 | CDK19 | GSE37991 |
| 0.1677 | 0.0905 | 0.6778 | 0.502549194 | 0.687274339 | −6.3912 | CDK5 | GSE37991 |
| 0.0488 | −0.1160 | 0.1085 | 0.914242928 | 0.955930303 | −6.6161 | ABCG1 | GSE37991 |

AveExpr: Average expression.

TABLE 30
Pathway enrichment analysis of top 100 genes clustered in the Turquoise
module using the KEGG database. The table presents the results of pathway
enrichment analysis conducted using the Enrichr tool on the top 100
genes clustered within the turquoise module. The turquoise module was
identified using the WGCNA (Weighted Gene Co-expression Network Analysis)
approach. The pathway enrichment analysis was performed specifically
using the KEGG (Kyoto Encyclopedia of Genes and Genomes) database.

| | | Adjusted | | Combined |
|---|---|---|---|---|
| Name | P-value | p-value | Odds Ratio | Score |
| Calcium signaling pathway | 0.00002961 | 0.002428 | 7.29 | 76.04 |
| cAMP signaling pathway | 0.0003357 | 0.01376 | 5.05 | 40.43 |
| Insulin secretion | 0.0009473 | 0.02589 | 9.97 | 69.38 |
| Gastric acid secretion | 0.002062 | 0.04228 | 12.93 | 79.96 |
| Neuroactive ligand-receptor interaction | 0.004804 | 0.07463 | 4.86 | 25.94 |
| Histidine metabolism | 0.005461 | 0.07463 | 20.08 | 104.62 |
| Arginine and proline metabolism | 0.00673 | 0.07884 | 8.31 | 41.58 |
| beta-Alanine metabolism | 0.01002 | 0.1027 | 14.34 | 66 |
| Circadian entrainment | 0.01308 | 0.1192 | 6.45 | 27.97 |
| Renin secretion | 0.0476 | 0.3903 | 5.98 | 18.21 |

TABLE 31
SNPs associated with regulation of anti-diabetic drug target expression in GTEx tissues using Mendelian Randomization
(MR) methods. SNP refers to the unique identifier for each genetic variant, and the Beta represents the estimated
effect size of the SNP on the expression of the corresponding anti-diabetic drug target. P value indicates
the statistical significance of the association between the SNP and drug target expression. GTEx tissues
refer to the specific tissues obtained from the Genotype-Tissue Expression (GTEx v8) project.

| | | | | | 95% | 95% | | P | | | Drug |
|---|---|---|---|---|---|---|---|---|---|---|---|
| Tissue | SNP | Mthd | nsnp | Beta | LCI | UCI | SE | value | Subst. | Drug | Bank ID |
| KC | rs77902362 | Wald | 1 | 0.0368 | 0.0045 | 0.0691 | 0.0165 | 0.025622627 | G. | SU | DB00222 |
| | | ratio | | | | | | | | | |
| P | rs4148631 | Wald | 1 | 0.0500 | 0.0096 | 0.0905 | 0.0206 | 0.015362072 | G. | SU | DB00222 |
| | | ratio | | | | | | | | | |
| Br. H | rs189603359 | Wald | 1 | 0.0846 | 0.0001 | 0.1691 | 0.0431 | 0.04978204 | G. | SU | DB00222 |
| | | ratio | | | | | | | | | |
| Liver | rs739688 | Wald | 1 | 0.0577 | 0.0232 | 0.0923 | 0.0176 | 0.001058455 | G. | SU | DB00222 |
| | | ratio | | | | | | | | | |
| EM | rs2355016 | Wald | 1 | 0.0622 | 0.0402 | 0.0842 | 0.0112 | 3.05E−08 | G. | SU | DB00222 |
| | | ratio | | | | | | | | | |
| CS | rs7110037 | Wald | 1 | 0.1250 | 0.0844 | 0.1656 | 0.0207 | 1.62E−09 | G. | SU | DB00222 |
| | | ratio | | | | | | | | | |
| EBV | rs79506407 | Wald | 1 | 0.0217 | 0.0106 | 0.0328 | 0.0056 | 0.000122473 | G. | SU | DB00222 |
| | | ratio | | | | | | | | | |
| Testis | rs7112030 | Wald | 1 | 0.1229 | 0.0936 | 0.1522 | 0.0149 | 1.96E−16 | G. | SU | DB00222 |
| | | ratio | | | | | | | | | |
| WB | rs35271178 | Wald | 1 | 0.4456 | 0.3779 | 0.5133 | 0.0345 | 4.50E−38 | G. | SU | DB00222 |
| | | ratio | | | | | | | | | |

G.: glimepiride, SU: Sulfonylureas; Subst.: Subtance; EM: Esophagus Muscularis; EBV: Cells EBV-transformed lymphocytes; Br. H: Brain Hippo-campus; WB: Whole Blood: CS: Colon Sigmoid; P: Pancreas; KC: kidney cortex; Mthd: Method.

TABLE 32
Prevalent diabetes and hazard ratios for gastric cancer in the Women's
Health Initiative (WHI) cohort: comparison of Sulfonylureas (SU) treatment.

| | | | | Participants with | | |
|---|---|---|---|---|---|---|
| | All participants | | | diabetes at baseline | | |
| | HR | Wald. | P | HR | Wald. | P |
| Characteristics | (95% CI) | Test | values | (95% CI) | Test | values |
| Diabetes at | 1.75(1.12- | 6 | 0.014 | / | / | / |
| baseline | 2.71) | | | | | |
| SU treatment | / | / | / | 0.58(0.24- | 1.40 | 0.23 |
| | | | | 1.43) | | |

TABLE 33
Raw data set 1. “Remove” is “FALSE” for all SNPs shown
in the list below. “id.outcome” is “ieu-a-7” for all SNPs shown in the list below.

| SNP | EAE | OAE | EAO | OAO | B ex | B Out | Eaf Ex | Eaf Out | palindromic | ambiguous | chr | pos |
|---|---|---|---|---|---|---|---|---|---|---|---|---|
| rs10009336 | T | C | T | C | −0.014 | −0.0287 | 0.1638 | 0.174439 | FALSE | FALSE | 4 | 44480783 |
| rs1006896 | C | A | C | A | −0.0234 | −0.03404 | 0.1061 | 0.089964 | FALSE | FALSE | 3 | 88104411 |
| rs10132280 | A | C | A | C | −0.0223 | −0.01217 | 0.3017 | 0.282164 | FALSE | FALSE | 14 | 25928179 |
| rs10169594 | C | T | C | T | 0.0121 | 0.011675 | 0.3596 | 0.343928 | FALSE | FALSE | 2 | 41637688 |
| rs10182181 | G | A | G | A | 0.0325 | 0.018295 | 0.4753 | 0.473525 | FALSE | FALSE | 2 | 25150296 |
| rs10192119 | G | T | G | T | 0.0166 | −0.00402 | 0.1673 | 0.193036 | FALSE | FALSE | 2 | 164581241 |
| rs10197031 | C | T | C | T | 0.0166 | 0.009969 | 0.2834 | 0.302131 | FALSE | FALSE | 2 | 105454590 |
| rs10243319 | C | T | C | T | −0.0107 | 0.015015 | 0.3939 | 0.408549 | FALSE | FALSE | 7 | 147674678 |
| rs10247983 | A | G | A | G | 0.0201 | 0.005051 | 0.9213 | 0.867948 | FALSE | FALSE | 7 | 114590228 |
| rs10248136 | T | C | T | C | −0.0097 | −0.01885 | 0.5142 | 0.49961 | FALSE | FALSE | 7 | 39077397 |
| rs10269783 | A | G | A | G | 0.0133 | 0.008184 | 0.3896 | 0.410277 | FALSE | FALSE | 7 | 49616203 |
| rs10408324 | T | C | T | C | −0.0124 | 0.002421 | 0.2744 | 0.241525 | FALSE | FALSE | 19 | 51774806 |
| rs10478110 | C | A | C | A | 0.01 | −0.00822 | 0.4348 | 0.445298 | FALSE | FALSE | 5 | 112445734 |
| rs1048932 | A | C | A | C | −0.016 | −0.0022 | 0.4162 | 0.416976 | FALSE | FALSE | 11 | 115044850 |
| rs10492229 | T | C | T | C | 0.0142 | −0.01416 | 0.2268 | 0.193631 | FALSE | FALSE | 12 | 110602173 |
| rs10510419 | T | G | T | G | −0.0177 | 0.014995 | 0.1416 | 0.143865 | FALSE | FALSE | 3 | 12426936 |
| rs10518694 | A | C | A | C | 0.0146 | −0.00931 | 0.1424 | 0.13965 | FALSE | FALSE | 15 | 53072673 |
| rs1064213 | A | G | A | G | 0.012 | 0.005176 | 0.492 | 0.481135 | FALSE | FALSE | 2 | 198950240 |
| rs10733051 | G | A | G | A | −0.0097 | 0.00234 | 0.4802 | 0.489356 | FALSE | FALSE | 1 | 167280354 |
| rs10742752 | C | T | C | T | 0.0124 | −0.00667 | 0.6159 | 0.614415 | FALSE | FALSE | 11 | 45438374 |
| rs10747488 | A | C | A | C | −0.0123 | −0.01839 | 0.7601 | 0.731258 | FALSE | FALSE | 1 | 98299475 |
| rs10750215 | T | G | T | G | 0.0108 | 0.018226 | 0.3883 | 0.3891 | FALSE | FALSE | 11 | 122505344 |
| rs1075901 | C | T | C | T | 0.0121 | 0.006822 | 0.5639 | 0.527617 | FALSE | FALSE | 17 | 15943910 |
| rs10768994 | C | T | C | T | −0.0114 | −0.00589 | 0.4337 | 0.44021 | FALSE | FALSE | 11 | 43936945 |
| rs10795422 | G | A | G | A | 0.0139 | −0.0112 | 0.6905 | 0.69582 | FALSE | FALSE | 10 | 16759312 |
| rs10811871 | G | A | G | A | −0.0108 | −0.0185 | 0.3829 | 0.375246 | FALSE | FALSE | 9 | 23200766 |
| rs10832778 | G | C | G | C | 0.0125 | −0.01247 | 0.6222 | 0.610211 | TRUE | FALSE | 11 | 17394073 |
| rs10858334 | G | C | G | C | 0.0143 | −0.01544 | 0.1415 | 0.133425 | TRUE | FALSE | 9 | 137989785 |
| rs10867256 | T | C | T | C | −0.0118 | −0.00221 | 0.553 | 0.505155 | FALSE | FALSE | 9 | 81367391 |
| rs10878946 | T | C | T | C | −0.0141 | −0.01097 | 0.714 | 0.705235 | FALSE | FALSE | 12 | 69642315 |
| rs10887578 | C | G | C | G | 0.0128 | −0.00142 | 0.4896 | 0.44479 | TRUE | TRUE | 10 | 88096047 |
| rs10914462 | G | A | G | A | −0.0112 | 0.002172 | 0.4255 | 0.415275 | FALSE | FALSE | 1 | 32125943 |
| rs10915840 | A | G | A | G | −0.0118 | −0.00757 | 0.283 | 0.248402 | FALSE | FALSE | 1 | 225668524 |
| rs10920678 | G | A | G | A | −0.0155 | −0.02619 | 0.5709 | 0.578396 | FALSE | FALSE | 1 | 190239907 |
| rs10938397 | G | A | G | A | 0.0324 | 0.030606 | 0.4317 | 0.416076 | FALSE | FALSE | 4 | 45182527 |
| rs10942267 | G | A | G | A | −0.0156 | −0.0004 | 0.3088 | 0.295189 | FALSE | FALSE | 5 | 80841914 |
| rs10953740 | G | A | G | A | −0.0153 | 0.004248 | 0.5534 | 0.507778 | FALSE | FALSE | 7 | 113460282 |
| rs10962550 | C | G | C | G | 0.0182 | 0.023454 | 0.1801 | 0.196347 | TRUE | FALSE | 9 | 16720329 |
| rs10968114 | C | A | C | A | −0.0113 | −0.00667 | 0.4681 | 0.467867 | FALSE | FALSE | 9 | 27800007 |
| rs10971709 | T | C | T | C | 0.0132 | −0.00986 | 0.2062 | 0.223395 | FALSE | FALSE | 9 | 33804813 |
| rs10984756 | G | C | G | C | 0.0174 | −0.01982 | 0.1048 | 0.08825 | TRUE | FALSE | 9 | 122651784 |
| rs11030618 | T | C | T | C | 0.011 | 0.011263 | 0.5679 | 0.572882 | FALSE | FALSE | 11 | 29243293 |
| rs11066188 | A | G | A | G | −0.012 | 0.063162 | 0.4181 | 0.347216 | FALSE | FALSE | 12 | 112610714 |
| rs11084553 | G | A | G | A | −0.021 | −0.02391 | 0.1518 | 0.122823 | FALSE | FALSE | 19 | 31019780 |
| rs11105839 | A | T | A | T | −0.0109 | −0.00978 | 0.3799 | 0.393378 | TRUE | FALSE | 12 | 91237920 |
| rs11115176 | C | T | C | T | −0.0121 | −0.01183 | 0.2399 | 0.220657 | FALSE | FALSE | 12 | 82465797 |
| rs11118308 | G | A | G | A | −0.0101 | 0.013406 | 0.4703 | 0.449185 | FALSE | FALSE | 1 | 219633869 |
| rs1112613 | A | G | A | G | −0.0133 | 0.005066 | 0.1762 | 0.180575 | FALSE | FALSE | 13 | 53651850 |
| rs11150911 | C | A | C | A | −0.0133 | −0.00128 | 0.7191 | 0.675297 | FALSE | FALSE | 18 | 73498528 |
| rs11165643 | T | C | T | C | 0.0206 | 0.00515 | 0.5828 | 0.553284 | FALSE | FALSE | 1 | 96924097 |
| rs11170468 | C | A | C | A | −0.0123 | 0.005351 | 0.2326 | 0.196627 | FALSE | FALSE | 12 | 39430048 |
| rs11173522 | A | C | A | C | 0.0128 | 0.006541 | 0.2078 | 0.227692 | FALSE | FALSE | 12 | 60953472 |
| rs11185111 | A | G | A | G | −0.0129 | 0.007014 | 0.3042 | 0.314106 | FALSE | FALSE | 1 | 107962328 |
| rs11251352 | G | A | G | A | 0.0109 | −0.00664 | 0.5988 | 0.539224 | FALSE | FALSE | 10 | 2585792 |
| rs1144387 | C | G | C | G | 0.0098 | 0.002204 | 0.5714 | 0.523079 | TRUE | TRUE | 13 | 78365190 |
| rs11496125 | T | C | T | C | 0.0169 | 0.010233 | 0.4212 | 0.442857 | FALSE | FALSE | 7 | 103417557 |
| rs11505821 | T | A | T | A | 0.0311 | 0.035485 | 0.0601 | 0.081514 | TRUE | FALSE | 7 | 76818677 |
| rs11538 | G | A | G | A | 0.0135 | 0.001531 | 0.1805 | 0.155212 | FALSE | FALSE | 22 | 18220831 |
| rs1158805 | A | C | A | C | −0.0137 | 0.002731 | 0.3766 | 0.382096 | FALSE | FALSE | 18 | 40736590 |
| rs11609659 | C | T | C | T | −0.0154 | −0.01237 | 0.2371 | 0.203901 | FALSE | FALSE | 12 | 108296260 |
| rs11611246 | T | G | T | G | 0.024 | 0.007423 | 0.21 | 0.214494 | FALSE | FALSE | 12 | 939480 |
| rs11615578 | T | C | T | C | 0.013 | −0.00678 | 0.2474 | 0.222076 | FALSE | FALSE | 12 | 121714935 |
| rs11656076 | A | G | A | G | −0.0142 | −0.01876 | 0.2254 | 0.244584 | FALSE | FALSE | 17 | 31464270 |
| rs11672660 | T | C | T | C | −0.034 | −0.03897 | 0.2049 | 0.195245 | FALSE | FALSE | 19 | 46180184 |
| rs11713193 | A | G | A | G | 0.0239 | 0.030121 | 0.5073 | 0.438401 | FALSE | FALSE | 3 | 49924424 |
| rs11736228 | T | A | T | A | −0.0139 | −0.01461 | 0.2587 | 0.273441 | TRUE | FALSE | 4 | 147376805 |
| rs11738695 | A | C | A | C | 0.0097 | 0.012108 | 0.586 | 0.556541 | FALSE | FALSE | 5 | 108699161 |
| rs11739877 | T | C | T | C | 0.0117 | 0.003696 | 0.6118 | 0.609384 | FALSE | FALSE | 5 | 105876806 |
| rs11781699 | C | T | C | T | 0.0132 | 0.010362 | 0.1896 | 0.173711 | FALSE | FALSE | 8 | 118863061 |
| rs11855853 | T | C | T | C | −0.0145 | −0.03429 | 0.2649 | 0.224087 | FALSE | FALSE | 15 | 78012618 |
| rs1187352 | C | T | C | T | 0.0119 | −0.0106 | 0.6518 | 0.659805 | FALSE | FALSE | 9 | 87293457 |
| rs11880870 | G | A | G | A | −0.0189 | −0.01577 | 0.4801 | 0.501665 | FALSE | FALSE | 19 | 18830704 |
| rs11889536 | G | A | G | A | −0.0189 | −0.02657 | 0.1493 | 0.150425 | FALSE | FALSE | 2 | 220163543 |
| rs11908637 | A | G | A | G | −0.012 | 0.002013 | 0.236 | 0.200731 | FALSE | FALSE | 20 | 47428485 |
| rs11945861 | A | G | A | G | −0.0148 | 0.004684 | 0.2369 | 0.259601 | FALSE | FALSE | 4 | 65700865 |
| rs11951673 | T | C | T | C | −0.0123 | −0.01807 | 0.3941 | 0.406307 | FALSE | FALSE | 5 | 95861012 |
| rs12033257 | G | A | G | A | −0.0146 | 0.014582 | 0.3835 | 0.398445 | FALSE | FALSE | 1 | 112318484 |
| rs12041258 | C | T | C | T | −0.0146 | −0.02051 | 0.2287 | 0.222743 | FALSE | FALSE | 1 | 195047936 |
| rs12044597 | G | A | G | A | 0.0143 | −0.01346 | 0.5029 | 0.464531 | FALSE | FALSE | 1 | 1708801 |
| rs12049202 | T | C | T | C | 0.024 | 0.0273 | 0.203 | 0.198168 | FALSE | FALSE | 1 | 77967523 |
| rs12098284 | T | C | T | C | 0.0178 | 0.039945 | 0.1241 | 0.124815 | FALSE | FALSE | 10 | 76047464 |
| rs12150665 | C | T | C | T | −0.0162 | −0.00512 | 0.4058 | 0.383889 | FALSE | FALSE | 17 | 34914787 |
| rs1218822 | A | G | A | G | 0.0168 | 0.006137 | 0.6663 | 0.63806 | FALSE | FALSE | 13 | 28011963 |
| rs12299814 | A | C | A | C | −0.0157 | 0.007157 | 0.2525 | 0.240233 | FALSE | FALSE | 12 | 90216146 |
| rs12328930 | C | T | C | T | 0.0098 | −0.00558 | 0.4235 | 0.394563 | FALSE | FALSE | 2 | 175079125 |
| rs12334877 | A | G | A | G | −0.0144 | −0.01508 | 0.198 | 0.211835 | FALSE | FALSE | 8 | 67194171 |
| rs12364470 | G | T | G | T | 0.0178 | 0.002392 | 0.1626 | 0.159457 | FALSE | FALSE | 11 | 134601012 |
| rs12369179 | T | C | T | C | −0.0359 | −0.01012 | 0.08782 | 0.072994 | FALSE | FALSE | 12 | 122963550 |
| rs12416812 | A | G | A | G | 0.0111 | 0.000116 | 0.5088 | 0.461789 | FALSE | FALSE | 11 | 888632 |
| rs1241986 | A | G | A | G | −0.0139 | −0.00779 | 0.8479 | 0.789395 | FALSE | FALSE | 18 | 6873954 |
| rs12422552 | C | G | C | G | −0.0134 | −0.01299 | 0.2663 | 0.276211 | TRUE | FALSE | 12 | 14413931 |
| rs12429545 | A | G | A | G | 0.0316 | 0.010619 | 0.1248 | 0.144688 | FALSE | FALSE | 13 | 54102206 |
| rs12448257 | A | G | A | G | 0.0184 | 0.009661 | 0.218 | 0.224463 | FALSE | FALSE | 16 | 3599655 |
| rs12546578 | A | T | A | T | 0.0146 | 0.002714 | 0.7246 | 0.707695 | TRUE | FALSE | 8 | 85085268 |
| rs12564992 | G | A | G | A | 0.0196 | 0.00206 | 0.1144 | 0.126785 | FALSE | FALSE | 1 | 174478100 |
| rs12593036 | G | A | G | A | −0.0154 | −0.01024 | 0.2993 | 0.298044 | FALSE | FALSE | 15 | 81058652 |
| rs12602912 | T | C | T | C | 0.0176 | 0.019844 | 0.2048 | 0.222578 | FALSE | FALSE | 17 | 65870073 |
| rs1260326 | C | T | C | T | 0.0105 | 0.003257 | 0.5973 | 0.609766 | FALSE | FALSE | 2 | 27730940 |
| rs12629015 | G | A | G | A | −0.0135 | −0.00981 | 0.1852 | 0.224677 | FALSE | FALSE | 3 | 119618053 |
| rs1266874 | G | A | G | A | 0.014 | 0.003749 | 0.3558 | 0.407422 | FALSE | FALSE | 6 | 51779638 |
| rs12675063 | T | A | T | A | 0.0156 | −0.00786 | 0.1131 | 0.134045 | TRUE | FALSE | 8 | 132879047 |
| rs1268065 | A | G | A | G | −0.0102 | −0.00258 | 0.4794 | 0.489992 | FALSE | FALSE | 6 | 126042783 |
| rs12680842 | G | A | G | A | −0.0133 | 0.009908 | 0.3205 | 0.343107 | FALSE | FALSE | 8 | 95582606 |
| rs12718572 | T | C | T | C | −0.0117 | −0.00572 | 0.4024 | 0.354827 | FALSE | FALSE | 7 | 50573325 |
| rs12762034 | C | T | C | T | 0.024 | 0.003665 | 0.07583 | 0.085812 | FALSE | FALSE | 10 | 33969931 |
| rs12779328 | T | C | T | C | 0.0105 | −0.01116 | 0.2833 | 0.255457 | FALSE | FALSE | 10 | 12943973 |
| rs1285997 | G | C | G | C | 0.0142 | −0.02562 | 0.7153 | 0.70125 | TRUE | FALSE | 14 | 91513029 |
| rs12888545 | G | A | G | A | 0.0136 | 0.018664 | 0.2519 | 0.208202 | FALSE | FALSE | 14 | 88308044 |
| rs12888955 | A | G | A | G | −0.0178 | −0.02015 | 0.6513 | 0.642185 | FALSE | FALSE | 14 | 103256877 |
| rs12905439 | G | C | G | C | −0.0118 | −0.01776 | 0.3393 | 0.299173 | TRUE | FALSE | 15 | 99521883 |
| rs12914489 | A | G | A | G | 0.0165 | 0.036789 | 0.1103 | 0.091715 | FALSE | FALSE | 15 | 74187937 |
| rs12922346 | C | G | C | G | 0.0136 | 0.014031 | 0.2657 | 0.258268 | TRUE | FALSE | 16 | 82438337 |
| rs12933482 | G | A | G | A | 0.0186 | 0.02648 | 0.1048 | 0.084611 | FALSE | FALSE | 16 | 72189604 |
| rs12936083 | G | A | G | A | 0.0139 | 0.000161 | 0.3267 | 0.384867 | FALSE | FALSE | 17 | 4801887 |
| rs12939549 | G | A | G | A | −0.018 | −0.0155 | 0.4335 | 0.407574 | FALSE | FALSE | 17 | 78611724 |
| rs1296328 | C | A | C | A | −0.0179 | 0.002357 | 0.5657 | 0.486722 | FALSE | FALSE | 4 | 137083193 |
| rs12981256 | A | G | A | G | 0.0142 | 0.013851 | 0.5325 | 0.488725 | FALSE | FALSE | 19 | 1865901 |
| rs13021737 | G | A | G | A | 0.0574 | 0.037025 | 0.8319 | 0.803169 | FALSE | FALSE | 2 | 632348 |
| rs13047416 | G | C | G | C | −0.0154 | 0.002307 | 0.3769 | 0.421483 | TRUE | TRUE | 21 | 40309436 |
| rs13069244 | A | G | A | G | 0.0187 | −0.02203 | 0.07747 | 0.062312 | FALSE | FALSE | 3 | 180441172 |
| rs13107325 | T | C | T | C | 0.047 | −0.00667 | 0.07373 | 0.061282 | FALSE | FALSE | 4 | 103188709 |
| rs13110266 | A | G | A | G | −0.0117 | −0.00879 | 0.4065 | 0.419593 | FALSE | FALSE | 4 | 162129844 |
| rs13132853 | G | A | G | A | −0.0142 | 0.010522 | 0.3452 | 0.285622 | FALSE | FALSE | 4 | 38680015 |
| rs13147390 | C | T | C | T | 0.0103 | −0.02193 | 0.3569 | 0.347908 | FALSE | FALSE | 4 | 80712000 |
| rs13174863 | G | A | G | A | 0.0192 | 0.00976 | 0.1548 | 0.136409 | FALSE | FALSE | 5 | 139080745 |
| rs13184896 | T | G | T | G | −0.0133 | −0.00271 | 0.4346 | 0.434327 | FALSE | FALSE | 5 | 122734005 |
| rs13191362 | G | A | G | A | −0.0236 | −0.03668 | 0.1198 | 0.111976 | FALSE | FALSE | 6 | 163033350 |
| rs1320903 | A | G | A | G | 0.0216 | −0.00187 | 0.3174 | 0.297027 | FALSE | FALSE | 3 | 131758077 |
| rs1321432 | C | A | C | A | 0.0201 | −6.60E−05 | 0.6321 | 0.615132 | FALSE | FALSE | 20 | 6614691 |
| rs13240600 | G | A | G | A | −0.0204 | −0.00958 | 0.1552 | 0.210968 | FALSE | FALSE | 7 | 99064466 |
| rs13250058 | T | G | T | G | 0.0112 | 0.009754 | 0.6771 | 0.590623 | FALSE | FALSE | 8 | 112270826 |
| rs13263601 | C | A | C | A | 0.0154 | 0.011541 | 0.3478 | 0.307214 | FALSE | FALSE | 8 | 14095900 |
| rs1327259 | G | A | G | A | −0.0155 | −0.00413 | 0.3872 | 0.4194 | FALSE | FALSE | 6 | 51177811 |
| rs13287131 | C | T | C | T | 0.0123 | −0.01139 | 0.2491 | 0.243733 | FALSE | FALSE | 9 | 92119579 |
| rs1330052 | G | C | G | C | 0.0132 | 0.00076 | 0.3504 | 0.337018 | TRUE | FALSE | 13 | 86536006 |
| rs13329567 | T | C | T | C | −0.0293 | −0.01415 | 0.2308 | 0.256185 | FALSE | FALSE | 15 | 68104367 |
| rs1365466 | T | C | T | C | −0.0137 | 0.01425 | 0.7406 | 0.705284 | FALSE | FALSE | 18 | 36182440 |
| rs1371108 | A | C | A | C | 0.0119 | 0.011656 | 0.3247 | 0.291818 | FALSE | FALSE | 2 | 81816251 |
| rs138289 | T | A | T | A | −0.0103 | 0.010182 | 0.4829 | 0.451715 | TRUE | TRUE | 22 | 32182708 |
| rs1409818 | T | C | T | C | 0.0201 | 0.005516 | 0.1156 | 0.114236 | FALSE | FALSE | 20 | 21381121 |
| rs1412235 | C | G | C | G | 0.0246 | 0.023064 | 0.3175 | 0.2934 | TRUE | FALSE | 9 | 28410996 |
| rs1421334 | C | A | C | A | −0.0125 | −0.01437 | 0.5431 | 0.519261 | FALSE | FALSE | 8 | 30865733 |
| rs1430387 | C | T | C | T | −0.0114 | 0.01036 | 0.4295 | 0.466378 | FALSE | FALSE | 18 | 58227112 |
| rs1431659 | G | A | G | A | −0.0196 | −0.00103 | 0.7344 | 0.698628 | FALSE | FALSE | 8 | 73439070 |
| rs1436344 | C | G | C | G | 0.0141 | 0.007158 | 0.5922 | 0.566776 | TRUE | TRUE | 3 | 104606144 |
| rs1445652 | A | G | A | G | 0.0123 | −0.00585 | 0.1855 | 0.196377 | FALSE | FALSE | 2 | 155668460 |
| rs1452075 | T | C | T | C | 0.0141 | 0.001795 | 0.7277 | 0.698638 | FALSE | FALSE | 3 | 62481063 |
| rs1454687 | G | C | G | C | −0.0202 | −0.00679 | 0.5227 | 0.515069 | TRUE | TRUE | 3 | 94038085 |
| rs1465900 | C | A | C | A | −0.0125 | −0.00866 | 0.2188 | 0.222651 | FALSE | FALSE | 11 | 76473138 |
| rs1472169 | T | C | T | C | −0.0139 | −0.01028 | 0.3772 | 0.355206 | FALSE | FALSE | 9 | 37209396 |
| rs1476322 | A | G | A | G | 0.0101 | 0.018552 | 0.569 | 0.571365 | FALSE | FALSE | 3 | 161446055 |
| rs1477199 | G | A | G | A | 0.0228 | 0.018891 | 0.1451 | 0.136126 | FALSE | FALSE | 16 | 53712135 |
| rs1492767 | T | C | T | C | 0.0094 | 0.005547 | 0.4957 | 0.452594 | FALSE | FALSE | 4 | 55221467 |
| rs1503526 | C | T | C | T | 0.014 | 0.005636 | 0.4838 | 0.508133 | FALSE | FALSE | 5 | 63020706 |
| rs1521527 | C | G | C | G | −0.0121 | −0.00749 | 0.5324 | 0.517368 | TRUE | TRUE | 2 | 165427825 |
| rs1522569 | G | T | G | T | −0.0164 | 0.007955 | 0.1819 | 0.177846 | FALSE | FALSE | 4 | 171632637 |
| rs1528435 | T | C | T | C | 0.0164 | 0.018036 | 0.6331 | 0.62036 | FALSE | FALSE | 2 | 181550962 |
| rs1535660 | C | T | C | T | −0.0147 | 0.001557 | 0.8554 | 0.821902 | FALSE | FALSE | 9 | 10371073 |
| rs1538247 | C | T | C | T | 0.0108 | 0.001939 | 0.3181 | 0.321989 | FALSE | FALSE | 6 | 153395344 |
| rs1552893 | G | A | G | A | −0.0126 | −0.01158 | 0.2777 | 0.329044 | FALSE | FALSE | 3 | 194851700 |
| rs156201 | C | G | C | G | 0.0123 | −3.40E−05 | 0.7606 | 0.708083 | TRUE | FALSE | 6 | 104847441 |
| rs1624134 | C | G | C | G | 0.0101 | −0.00319 | 0.4068 | 0.41809 | TRUE | FALSE | 10 | 34834482 |
| rs1656377 | C | T | C | T | 0.0099 | 0.001876 | 0.5885 | 0.578678 | FALSE | FALSE | 3 | 158285280 |
| rs1681740 | C | A | C | A | −0.0115 | 0.003998 | 0.3933 | 0.376692 | FALSE | FALSE | 10 | 118564313 |
| rs16849710 | G | A | G | A | −0.0116 | −0.0025 | 0.515 | 0.504205 | FALSE | FALSE | 1 | 202106797 |
| rs16851483 | T | G | T | G | 0.0369 | 0.014404 | 0.06928 | 0.08304 | FALSE | FALSE | 3 | 141275436 |
| rs16871902 | A | G | A | G | 0.0125 | 0.005509 | 0.4877 | 0.4731 | FALSE | FALSE | 5 | 3488462 |
| rs16903285 | C | T | C | T | 0.0331 | 0.031088 | 0.1407 | 0.158652 | FALSE | FALSE | 5 | 87978252 |
| rs16953563 | A | G | A | G | −0.0134 | 0.019145 | 0.2516 | 0.260016 | FALSE | FALSE | 15 | 66686770 |
| rs17001561 | A | G | A | G | 0.0151 | 0.01589 | 0.1573 | 0.139334 | FALSE | FALSE | 4 | 77096118 |
| rs17014375 | G | T | G | T | 0.0172 | −0.00752 | 0.1348 | 0.144606 | FALSE | FALSE | 1 | 209543560 |
| rs17033117 | T | C | T | C | 0.0137 | 0.009903 | 0.1872 | 0.208132 | FALSE | FALSE | 3 | 35443653 |
| rs17056301 | C | T | C | T | 0.0118 | 0.013126 | 0.2636 | 0.290602 | FALSE | FALSE | 5 | 158271680 |
| rs17113297 | T | C | T | C | 0.0166 | −0.00073 | 0.2082 | 0.204052 | FALSE | FALSE | 10 | 102395982 |
| rs17119937 | C | T | C | T | 0.0212 | −0.01495 | 0.06905 | 0.060806 | FALSE | FALSE | 8 | 14502274 |
| rs17203016 | G | A | G | A | 0.015 | 0.020352 | 0.196 | 0.186737 | FALSE | FALSE | 2 | 208255518 |
| rs17207196 | T | C | T | C | −0.0221 | −0.01495 | 0.4118 | 0.424756 | FALSE | FALSE | 7 | 75101065 |
| rs17238110 | G | A | G | A | −0.0353 | 0.0085 | 0.1634 | 0.053921 | FALSE | FALSE | 15 | 62150364 |
| rs17311369 | T | C | T | C | −0.0104 | −3.30E−05 | 0.3278 | 0.321999 | FALSE | FALSE | 15 | 47709199 |
| rs17399237 | C | T | C | T | −0.0129 | −0.02107 | 0.5497 | 0.598881 | FALSE | FALSE | 2 | 35471626 |
| rs17405819 | C | T | C | T | −0.0215 | −0.0115 | 0.301 | 0.307757 | FALSE | FALSE | 8 | 76806584 |
| rs17424296 | A | G | A | G | −0.0108 | 0.016094 | 0.3659 | 0.323838 | FALSE | FALSE | 5 | 60838903 |
| rs17425707 | C | T | C | T | 0.0167 | 0.008986 | 0.1003 | 0.099606 | FALSE | FALSE | 1 | 57874879 |
| rs17446257 | A | G | A | G | 0.0153 | 0.015541 | 0.1292 | 0.115342 | FALSE | FALSE | 13 | 40749213 |
| rs17499593 | G | C | G | C | 0.0125 | 0.007632 | 0.1897 | 0.171156 | TRUE | FALSE | 2 | 172649755 |
| rs17513613 | C | T | C | T | 0.0186 | −0.02249 | 0.3236 | 0.295626 | FALSE | FALSE | 19 | 30286822 |
| rs175165 | G | T | G | T | −0.0103 | −0.0062 | 0.3941 | 0.424326 | FALSE | FALSE | 22 | 20116015 |
| rs17535749 | A | G | A | G | 0.015 | 0.032656 | 0.1023 | 0.087257 | FALSE | FALSE | 3 | 10027724 |
| rs17551974 | A | C | A | C | −0.0141 | −0.00604 | 0.1782 | 0.205463 | FALSE | FALSE | 2 | 142293146 |
| rs17636031 | C | T | C | T | 0.016 | 0.002624 | 0.2701 | 0.249102 | FALSE | FALSE | 10 | 126594078 |
| rs17663412 | A | C | A | C | 0.0157 | −0.00217 | 0.1139 | 0.117381 | FALSE | FALSE | 5 | 167595121 |
| rs17710386 | C | T | C | T | 0.0126 | 0.005693 | 0.3319 | 0.362448 | FALSE | FALSE | 18 | 63461201 |
| rs17724992 | G | A | G | A | −0.0183 | −0.0139 | 0.2596 | 0.293771 | FALSE | FALSE | 19 | 18454825 |
| rs17789218 | C | T | C | T | 0.013 | −0.00287 | 0.2392 | 0.205922 | FALSE | FALSE | 6 | 100600097 |
| rs17806379 | T | C | T | C | −0.0258 | −0.00602 | 0.1789 | 0.166425 | FALSE | FALSE | 20 | 51107290 |
| rs1784460 | A | T | A | T | 0.0132 | 0.017895 | 0.4035 | 0.387718 | TRUE | FALSE | 11 | 118938371 |
| rs1804528 | A | G | A | G | 0.0109 | 0.014209 | 0.3507 | 0.326655 | FALSE | FALSE | 4 | 146056320 |
| rs1830074 | C | T | C | T | 0.0115 | 0.000671 | 0.288 | 0.308417 | FALSE | FALSE | 7 | 6718674 |
| rs1836303 | G | A | G | A | 0.0116 | 0.000219 | 0.3873 | 0.374699 | FALSE | FALSE | 15 | 46539116 |
| rs1843328 | A | C | A | C | −0.0099 | 0.010764 | 0.5085 | 0.511865 | FALSE | FALSE | 12 | 17111188 |
| rs1863652 | A | G | A | G | −0.0115 | −0.02651 | 0.3449 | 0.330965 | FALSE | FALSE | 4 | 95991417 |
| rs1884389 | T | C | T | C | −0.0103 | −0.01638 | 0.4289 | 0.443283 | FALSE | FALSE | 20 | 1410582 |
| rs1885728 | A | G | A | G | 0.0108 | 0.015514 | 0.6787 | 0.597901 | FALSE | FALSE | 6 | 5977833 |
| rs1891216 | G | T | G | T | 0.0107 | 0.006178 | 0.3759 | 0.352443 | FALSE | FALSE | 1 | 7728391 |
| rs1896767 | A | G | A | G | −0.0109 | −0.00609 | 0.5376 | 0.538506 | FALSE | FALSE | 16 | 62838304 |
| rs189843 | C | G | C | G | −0.0098 | −0.00303 | 0.5557 | 0.554706 | TRUE | TRUE | 5 | 164600151 |
| rs1927790 | C | T | C | T | 0.0148 | −0.00714 | 0.4109 | 0.423664 | FALSE | FALSE | 13 | 96922191 |
| rs1928295 | C | T | C | T | −0.0141 | −0.00667 | 0.4461 | 0.436964 | FALSE | FALSE | 9 | 120378483 |
| rs1937683 | T | C | T | C | 0.0109 | 0.006461 | 0.6699 | 0.648175 | FALSE | FALSE | 10 | 53679060 |
| rs1948080 | G | T | G | T | −0.0137 | −0.01105 | 0.3749 | 0.338973 | FALSE | FALSE | 9 | 11852043 |
| rs1982441 | T | G | T | G | 0.0175 | −0.00701 | 0.1381 | 0.131611 | FALSE | FALSE | 8 | 28021769 |
| rs1982725 | T | C | T | C | 0.0097 | −0.00662 | 0.4778 | 0.453511 | FALSE | FALSE | 19 | 30618771 |
| rs1993709 | G | A | G | A | 0.0331 | 0.034146 | 0.8177 | 0.814159 | FALSE | FALSE | 1 | 72838529 |
| rs2007231 | T | C | T | C | −0.0104 | −0.01204 | 0.6387 | 0.653989 | FALSE | FALSE | 1 | 115266306 |
| rs200810 | C | T | C | T | −0.0136 | −0.01961 | 0.3716 | 0.395383 | FALSE | FALSE | 6 | 97922184 |
| rs2009416 | T | C | T | C | −0.0121 | −0.01075 | 0.361 | 0.395688 | FALSE | FALSE | 5 | 92415111 |
| rs2033529 | G | A | G | A | 0.0205 | −0.0063 | 0.2936 | 0.26618 | FALSE | FALSE | 6 | 40348653 |
| rs2051559 | C | T | C | T | 0.0176 | 0.01107 | 0.1308 | 0.135019 | FALSE | FALSE | 4 | 3298800 |
| rs2065418 | G | T | G | T | −0.0166 | 0.00433 | 0.3623 | 0.34348 | FALSE | FALSE | 11 | 30422068 |
| rs208015 | C | T | C | T | −0.0356 | −0.02129 | 0.9216 | 0.884774 | FALSE | FALSE | 17 | 46252346 |
| rs2124499 | C | G | C | G | −0.0123 | 0.000872 | 0.3718 | 0.339958 | TRUE | FALSE | 3 | 123093541 |
| rs2143253 | A | G | A | G | −0.0188 | −0.01185 | 0.1189 | 0.159188 | FALSE | FALSE | 20 | 41987392 |
| rs215634 | G | A | G | A | −0.0152 | −0.01929 | 0.6212 | 0.563847 | FALSE | FALSE | 7 | 32369148 |
| rs2162524 | C | T | C | T | 0.0155 | −0.00649 | 0.3321 | 0.313884 | FALSE | FALSE | 2 | 230817437 |
| rs2163188 | C | G | C | G | 0.0131 | −0.00654 | 0.474 | 0.467371 | TRUE | TRUE | 10 | 65314711 |
| rs2174307 | C | G | C | G | 0.0121 | 0.024677 | 0.4067 | 0.428053 | TRUE | TRUE | 9 | 73791849 |
| rs217671 | G | A | G | A | 0.0144 | 0.008219 | 0.2719 | 0.287208 | FALSE | FALSE | 14 | 62360464 |
| rs2228213 | A | G | A | G | −0.0139 | −0.0056 | 0.3481 | 0.324329 | FALSE | FALSE | 6 | 12124855 |
| rs2235564 | T | C | T | C | 0.0131 | 0.002693 | 0.3466 | 0.332213 | FALSE | FALSE | 1 | 6713114 |
| rs2246012 | C | T | C | T | 0.0158 | 0.021509 | 0.1628 | 0.18686 | FALSE | FALSE | 6 | 131898208 |
| rs226000 | T | C | T | C | 0.0119 | 0.013932 | 0.8245 | 0.790844 | FALSE | FALSE | 14 | 30488699 |
| rs2283093 | T | C | T | C | 0.0127 | 0.004189 | 0.2066 | 0.199922 | FALSE | FALSE | 7 | 126721231 |
| rs2284746 | G | C | G | C | −0.0104 | 0.018899 | 0.5238 | 0.518277 | TRUE | TRUE | 1 | 17306675 |
| rs2285178 | C | T | C | T | 0.0112 | 0.00892 | 0.3109 | 0.313154 | FALSE | FALSE | 22 | 38205989 |
| rs2306537 | G | A | G | A | 0.0133 | 0.002936 | 0.3092 | 0.266934 | FALSE | FALSE | 12 | 133423695 |
| rs2307111 | C | T | C | T | −0.0265 | −0.00753 | 0.3962 | 0.442845 | FALSE | FALSE | 5 | 75003678 |
| rs2317299 | C | T | C | T | −0.0106 | −0.01256 | 0.5597 | 0.560217 | FALSE | FALSE | 2 | 236903093 |
| rs2325036 | C | A | C | A | −0.0181 | −0.00144 | 0.3845 | 0.412665 | FALSE | FALSE | 3 | 85819412 |
| rs2357760 | A | G | A | G | 0.0145 | 0.016612 | 0.6754 | 0.65138 | FALSE | FALSE | 6 | 120213880 |
| rs2361988 | C | T | C | T | −0.0155 | 0.009134 | 0.2539 | 0.244085 | FALSE | FALSE | 16 | 398151 |
| rs2365389 | T | C | T | C | −0.0174 | −0.01498 | 0.4143 | 0.449327 | FALSE | FALSE | 3 | 61236462 |
| rs2367112 | G | T | G | T | −0.0119 | −0.01828 | 0.4919 | 0.531024 | FALSE | FALSE | 5 | 64168193 |
| rs2411182 | A | G | A | G | 0.0123 | −0.00269 | 0.6943 | 0.684507 | FALSE | FALSE | 17 | 35059718 |
| rs2423668 | C | T | C | T | −0.0105 | −0.00624 | 0.5505 | 0.53628 | FALSE | FALSE | 20 | 12430673 |
| rs2425840 | C | A | C | A | 0.0119 | 0.003547 | 0.4059 | 0.370986 | FALSE | FALSE | 20 | 44904838 |
| rs2429150 | C | A | C | A | 0.0111 | 0.014347 | 0.4164 | 0.448942 | FALSE | FALSE | 12 | 2152655 |
| rs2479958 | G | A | G | A | −0.0154 | −0.01548 | 0.5075 | 0.485468 | FALSE | FALSE | 13 | 111984244 |
| rs2481665 | C | T | C | T | −0.0161 | −0.02466 | 0.4408 | 0.377011 | FALSE | FALSE | 1 | 62594677 |
| rs2543132 | C | G | C | G | 0.0146 | 0.005268 | 0.8134 | 0.742222 | TRUE | FALSE | 8 | 15536311 |
| rs2600226 | T | C | T | C | −0.0116 | −0.0212 | 0.6697 | 0.611419 | FALSE | FALSE | 3 | 12928762 |
| rs2605603 | A | G | A | G | −0.0103 | −0.00323 | 0.4887 | 0.521818 | FALSE | FALSE | 11 | 93221105 |
| rs2608703 | A | C | A | C | 0.0142 | 0.017367 | 0.4546 | 0.449644 | FALSE | FALSE | 12 | 41846769 |
| rs262130 | T | C | T | C | 0.0127 | −0.00221 | 0.1969 | 0.196219 | FALSE | FALSE | 6 | 142853486 |
| rs2643452 | A | T | A | T | 0.0136 | −0.01156 | 0.5448 | 0.544544 | TRUE | TRUE | 4 | 18529220 |
| rs2693826 | A | G | A | G | −0.0137 | −0.0231 | 0.4421 | 0.491526 | FALSE | FALSE | 2 | 6160943 |
| rs2694047 | G | A | G | A | 0.0188 | 0.008753 | 0.747 | 0.739223 | FALSE | FALSE | 8 | 116750548 |
| rs273504 | G | A | G | A | 0.0153 | 0.009569 | 0.4266 | 0.413118 | FALSE | FALSE | 19 | 18215247 |
| rs2744974 | T | C | T | C | 0.0249 | 0.024467 | 0.338 | 0.34597 | FALSE | FALSE | 6 | 34579431 |
| rs2791653 | G | A | G | A | −0.0141 | 0.005892 | 0.7577 | 0.745055 | FALSE | FALSE | 1 | 11129848 |
| rs2820311 | G | A | G | A | 0.0235 | 0.041506 | 0.3369 | 0.30474 | FALSE | FALSE | 1 | 201841476 |
| rs2832283 | A | G | A | G | 0.0115 | 0.017054 | 0.2208 | 0.210401 | FALSE | FALSE | 21 | 30690558 |
| rs2836964 | C | T | C | T | −0.011 | −0.00539 | 0.3576 | 0.308007 | FALSE | FALSE | 21 | 40631006 |
| rs2861683 | C | A | C | A | −0.0144 | 0.00283 | 0.407 | 0.41764 | FALSE | FALSE | 2 | 67836507 |
| rs2868975 | A | G | A | G | −0.0143 | 0.006835 | 0.178 | 0.190014 | FALSE | FALSE | 3 | 116935323 |
| rs287104 | A | G | A | G | 0.0115 | 0.01027 | 0.6604 | 0.621934 | FALSE | FALSE | 19 | 34290995 |
| rs2875762 | C | G | C | G | 0.0139 | −0.00346 | 0.2473 | 0.244374 | TRUE | FALSE | 6 | 124925032 |
| rs2907948 | A | G | A | G | −0.0141 | −0.01613 | 0.2427 | 0.211582 | FALSE | FALSE | 7 | 150638484 |
| rs2931434 | T | C | T | C | −0.0104 | −0.00965 | 0.3168 | 0.290155 | FALSE | FALSE | 5 | 73159098 |
| rs2943465 | C | T | C | T | 0.0248 | 0.017628 | 0.9444 | 0.883842 | FALSE | FALSE | 12 | 19265921 |
| rs294704 | T | G | T | G | −0.0113 | −0.01573 | 0.7239 | 0.718973 | FALSE | FALSE | 5 | 152519088 |
| rs3007105 | T | C | T | C | 0.0142 | 0.005647 | 0.4697 | 0.430729 | FALSE | FALSE | 14 | 47367616 |
| rs326896 | T | C | T | C | −0.0128 | −0.01378 | 0.3925 | 0.420065 | FALSE | FALSE | 4 | 112669571 |
| rs331966 | C | A | C | A | 0.0112 | 0.014457 | 0.3792 | 0.378037 | FALSE | FALSE | 4 | 143675717 |
| rs33500 | T | C | T | C | −0.0167 | −0.0046 | 0.8082 | 0.794673 | FALSE | FALSE | 3 | 42427191 |
| rs339991 | G | A | G | A | 0.0124 | 0.001167 | 0.5631 | 0.562092 | FALSE | FALSE | 15 | 60913637 |
| rs349088 | A | C | A | C | −0.0128 | −0.00224 | 0.4976 | 0.499349 | FALSE | FALSE | 11 | 84814393 |
| rs355777 | C | G | C | G | 0.0153 | 0.015515 | 0.4106 | 0.408963 | TRUE | FALSE | 3 | 154034950 |
| rs3731695 | C | T | C | T | 0.0116 | −0.03883 | 0.5582 | 0.526466 | FALSE | FALSE | 2 | 203820275 |
| rs3732084 | C | T | C | T | 0.0107 | −0.00079 | 0.6139 | 0.566631 | FALSE | FALSE | 2 | 207174316 |
| rs3736485 | G | A | G | A | −0.0134 | 0.001591 | 0.5443 | 0.50246 | FALSE | FALSE | 15 | 51748610 |
| rs3749897 | T | C | T | C | 0.0122 | 0.004178 | 0.4172 | 0.42251 | FALSE | FALSE | 6 | 42532102 |
| rs3754963 | T | A | T | A | −0.0123 | −0.01875 | 0.2574 | 0.258267 | TRUE | FALSE | 2 | 166185707 |
| rs3764835 | A | G | A | G | −0.0141 | −0.00768 | 0.1528 | 0.141138 | FALSE | FALSE | 2 | 159519368 |
| rs3772882 | A | C | A | C | 0.0127 | 0.03021 | 0.3661 | 0.388977 | FALSE | FALSE | 3 | 81808602 |
| rs3800229 | T | G | T | G | 0.0175 | 0.003892 | 0.7123 | 0.653147 | FALSE | FALSE | 6 | 108996963 |
| rs3800637 | C | T | C | T | 0.0115 | −0.00021 | 0.336 | 0.376245 | FALSE | FALSE | 7 | 137403432 |
| rs3806114 | A | G | A | G | −0.0113 | −0.00919 | 0.6773 | 0.690997 | FALSE | FALSE | 6 | 20482335 |
| rs3806572 | A | G | A | G | −0.0145 | 0.019996 | 0.2788 | 0.256371 | FALSE | FALSE | 2 | 55238677 |
| rs3807645 | A | G | A | G | −0.0166 | −0.00892 | 0.221 | 0.221843 | FALSE | FALSE | 7 | 77830091 |
| rs380857 | A | C | A | C | −0.0151 | −0.01647 | 0.8878 | 0.849174 | FALSE | FALSE | 9 | 101491066 |
| rs3814883 | T | C | T | C | 0.0232 | −0.00971 | 0.4764 | 0.478074 | FALSE | FALSE | 16 | 29994922 |
| rs3828783 | A | G | A | G | −0.0165 | −0.00989 | 0.1809 | 0.181709 | FALSE | FALSE | 6 | 33767727 |
| rs3829849 | T | C | T | C | 0.0098 | −0.00429 | 0.3589 | 0.339005 | FALSE | FALSE | 9 | 129390800 |
| rs38314 | A | G | A | G | −0.012 | 0.001507 | 0.4912 | 0.444611 | FALSE | FALSE | 7 | 70067315 |
| rs3844598 | G | A | G | A | 0.0095 | 0.022935 | 0.521 | 0.520817 | FALSE | FALSE | 5 | 140992235 |
| rs3902951 | G | T | G | T | 0.0134 | 0.004349 | 0.2455 | 0.298539 | FALSE | FALSE | 14 | 69789755 |
| rs3904244 | A | T | A | T | 0.0155 | −0.00512 | 0.1377 | 0.203921 | TRUE | FALSE | 10 | 27361527 |
| rs391300 | C | T | C | T | −0.0119 | −0.03367 | 0.6275 | 0.600286 | FALSE | FALSE | 17 | 2216258 |
| rs3935648 | G | C | G | C | −0.0125 | −0.03666 | 0.2328 | 0.229231 | TRUE | FALSE | 17 | 79085335 |
| rs3977755 | T | C | T | C | −0.0135 | 0.018568 | 0.2804 | 0.284965 | FALSE | FALSE | 10 | 104420210 |
| rs40067 | A | G | A | G | −0.0266 | −0.03886 | 0.1713 | 0.205156 | FALSE | FALSE | 5 | 107439012 |
| rs4012234 | G | T | G | T | 0.0141 | 0.003867 | 0.5924 | 0.600816 | FALSE | FALSE | 20 | 32553047 |
| rs4072917 | A | G | A | G | 0.0115 | 0.010825 | 0.4694 | 0.466674 | FALSE | FALSE | 8 | 143300279 |
| rs4148155 | G | A | G | A | −0.0188 | 0.023792 | 0.1127 | 0.11326 | FALSE | FALSE | 4 | 89054667 |
| rs4148866 | T | C | T | C | 0.0098 | −0.00282 | 0.4068 | 0.419861 | FALSE | FALSE | 12 | 123425575 |
| rs4237643 | G | T | G | T | −0.0223 | −0.02825 | 0.6938 | 0.683059 | FALSE | FALSE | 11 | 43648368 |
| rs427943 | C | A | C | A | 0.017 | 0.022403 | 0.5669 | 0.559153 | FALSE | FALSE | 21 | 46570896 |
| rs429343 | G | A | G | A | −0.015 | −0.01074 | 0.5813 | 0.523049 | FALSE | FALSE | 2 | 147903382 |
| rs4307239 | G | A | G | A | 0.0115 | 0.001717 | 0.4578 | 0.482379 | FALSE | FALSE | 7 | 24354300 |
| rs4310573 | T | C | T | C | 0.0116 | −0.01287 | 0.7813 | 0.725974 | FALSE | FALSE | 11 | 97855562 |
| rs4358081 | C | A | C | A | 0.0097 | −0.01045 | 0.4631 | 0.469122 | FALSE | FALSE | 2 | 29100642 |
| rs4414033 | A | G | A | G | 0.0129 | −0.01095 | 0.627 | 0.612623 | FALSE | FALSE | 1 | 156406853 |
| rs4430672 | C | T | C | T | −0.0127 | 0.002194 | 0.8004 | 0.778525 | FALSE | FALSE | 14 | 63094407 |
| rs4482463 | A | C | A | C | −0.0331 | 0.002765 | 0.9213 | 0.870696 | FALSE | FALSE | 2 | 205375909 |
| rs4495304 | C | T | C | T | −0.0194 | 0.007579 | 0.067 | 0.090083 | FALSE | FALSE | 6 | 31080718 |
| rs4516268 | A | C | A | C | −0.0217 | −0.00729 | 0.1925 | 0.17624 | FALSE | FALSE | 17 | 1846831 |
| rs4518345 | A | G | A | G | −0.0117 | 0.005034 | 0.2842 | 0.262699 | FALSE | FALSE | 5 | 27185904 |
| rs4556997 | A | C | A | C | 0.0197 | −0.00704 | 0.1349 | 0.141637 | FALSE | FALSE | 2 | 100814858 |
| rs4589691 | G | C | G | C | 0.0141 | −0.02671 | 0.1579 | 0.171766 | TRUE | FALSE | 2 | 144051398 |
| rs4639527 | G | A | G | A | 0.0172 | 0.001816 | 0.3012 | 0.310181 | FALSE | FALSE | 2 | 416815 |
| rs4653017 | T | C | T | C | 0.0122 | 0.002586 | 0.6818 | 0.670332 | FALSE | FALSE | 1 | 33776728 |
| rs4660443 | T | C | T | C | 0.0164 | 0.021668 | 0.2218 | 0.195765 | FALSE | FALSE | 1 | 39591779 |
| rs4671328 | G | T | G | T | −0.0219 | −0.00397 | 0.5533 | 0.551638 | FALSE | FALSE | 2 | 58935282 |
| rs4722398 | T | C | T | C | 0.0158 | 0.00467 | 0.1336 | 0.125981 | FALSE | FALSE | 7 | 3125220 |
| rs4740619 | C | T | C | T | −0.0186 | −0.00655 | 0.4521 | 0.467784 | FALSE | FALSE | 9 | 15634326 |
| rs4757144 | A | G | A | G | 0.0169 | 0.035584 | 0.5878 | 0.539899 | FALSE | FALSE | 11 | 13331226 |
| rs4783830 | A | G | A | G | −0.0105 | −0.00056 | 0.3074 | 0.30397 | FALSE | FALSE | 16 | 54255346 |
| rs4786903 | G | A | G | A | 0.0125 | −0.0037 | 0.7368 | 0.720083 | FALSE | FALSE | 16 | 6697104 |
| rs4800191 | C | G | C | G | 0.0103 | −0.00138 | 0.6369 | 0.63838 | TRUE | FALSE | 18 | 22461398 |
| rs4813619 | T | G | T | G | −0.0108 | 0.006251 | 0.5101 | 0.555402 | FALSE | FALSE | 20 | 2815715 |
| rs4818225 | G | A | G | A | 0.0117 | 0.016728 | 0.6606 | 0.667104 | FALSE | FALSE | 21 | 42629895 |
| rs4820408 | G | T | G | T | −0.0151 | 0.021866 | 0.592 | 0.608855 | FALSE | FALSE | 22 | 40604945 |
| rs4842491 | T | C | T | C | 0.0098 | 0.019037 | 0.7138 | 0.711835 | FALSE | FALSE | 12 | 89905537 |
| rs4851029 | G | T | G | T | 0.0121 | 0.00218 | 0.5247 | 0.481439 | FALSE | FALSE | 2 | 104159785 |
| rs4858193 | C | T | C | T | −0.0129 | −0.02254 | 0.2779 | 0.233871 | FALSE | FALSE | 3 | 20441050 |
| rs486359 | C | G | C | G | 0.0112 | −0.03376 | 0.4853 | 0.463746 | TRUE | TRUE | 6 | 160774441 |
| rs4864201 | C | T | C | T | −0.0141 | −0.02019 | 0.6469 | 0.586276 | FALSE | FALSE | 4 | 130731284 |
| rs4880341 | T | C | T | C | −0.0118 | 0.009084 | 0.5606 | 0.560472 | FALSE | FALSE | 10 | 133992689 |
| rs4906908 | G | T | G | T | 0.0103 | 0.005426 | 0.5253 | 0.518139 | FALSE | FALSE | 15 | 27040082 |
| rs491711 | C | A | C | A | −0.0115 | −0.00573 | 0.316 | 0.311323 | FALSE | FALSE | 11 | 28742220 |
| rs4929923 | C | T | C | T | 0.0181 | 0.016055 | 0.6376 | 0.597203 | FALSE | FALSE | 11 | 8639200 |
| rs4936175 | C | T | C | T | 0.0122 | 0.000541 | 0.4445 | 0.375523 | FALSE | FALSE | 11 | 132641959 |
| rs4937870 | G | A | G | A | −0.0109 | −0.01126 | 0.3172 | 0.34674 | FALSE | FALSE | 11 | 112826709 |
| rs4952843 | G | A | G | A | −0.0131 | −0.0165 | 0.3807 | 0.342253 | FALSE | FALSE | 2 | 46957845 |
| rs4954638 | C | A | C | A | −0.0118 | 0.0062 | 0.2492 | 0.328428 | FALSE | FALSE | 2 | 137435455 |
| rs4968656 | G | A | G | A | 0.0116 | 0.013302 | 0.3216 | 0.314923 | FALSE | FALSE | 17 | 61616959 |
| rs4981693 | A | G | A | G | 0.0206 | 0.030291 | 0.771 | 0.731352 | FALSE | FALSE | 14 | 29680331 |
| rs4986044 | T | C | T | C | −0.0164 | −0.01631 | 0.4687 | 0.496991 | FALSE | FALSE | 17 | 21261560 |
| rs538579 | C | G | C | G | 0.0137 | 0.013236 | 0.3228 | 0.303248 | TRUE | FALSE | 3 | 62711674 |
| rs543874 | G | A | G | A | 0.0475 | 0.0076 | 0.1952 | 0.189189 | FALSE | FALSE | 1 | 177889480 |
| rs559231 | T | G | T | G | 0.0135 | 0.011722 | 0.3956 | 0.428771 | FALSE | FALSE | 18 | 39644247 |
| rs577525 | C | T | C | T | 0.0166 | 0.017886 | 0.5676 | 0.516924 | FALSE | FALSE | 10 | 99769388 |
| rs592483 | T | C | T | C | −0.0147 | 0.023104 | 0.5716 | 0.561649 | FALSE | FALSE | 11 | 69445173 |
| rs6011457 | A | T | A | T | −0.0116 | 0.005366 | 0.4975 | 0.479706 | TRUE | TRUE | 20 | 61530915 |
| rs6050446 | G | A | G | A | 0.0343 | 0.034177 | 0.97001 | 0.954744 | FALSE | FALSE | 20 | 25195509 |
| rs6235 | G | C | G | C | 0.0175 | −0.0006 | 0.2702 | 0.27387 | TRUE | FALSE | 5 | 95728898 |
| rs6265 | T | C | T | C | −0.0412 | −0.03043 | 0.1951 | 0.199336 | FALSE | FALSE | 11 | 27679916 |
| rs6443750 | C | T | C | T | 0.0148 | 0.01903 | 0.8068 | 0.831807 | FALSE | FALSE | 3 | 181329682 |
| rs6448587 | C | A | C | A | −0.0167 | −0.0108 | 0.1891 | 0.214826 | FALSE | FALSE | 4 | 28561990 |
| rs645040 | T | G | T | G | 0.0171 | 0.038585 | 0.7762 | 0.760153 | FALSE | FALSE | 3 | 135926622 |
| rs6461115 | G | A | G | A | −0.0144 | −0.0226 | 0.2285 | 0.266321 | FALSE | FALSE | 7 | 2103668 |
| rs6471941 | A | G | A | G | 0.0156 | 0.000418 | 0.1684 | 0.221682 | FALSE | FALSE | 8 | 62117973 |
| rs6500208 | A | G | A | G | 0.014 | −0.00073 | 0.2006 | 0.223706 | FALSE | FALSE | 16 | 49011249 |
| rs6512302 | C | G | C | G | 0.0142 | −0.00829 | 0.7511 | 0.737005 | TRUE | FALSE | 20 | 62691550 |
| rs6545714 | A | G | A | G | −0.0191 | −0.01037 | 0.6139 | 0.613514 | FALSE | FALSE | 2 | 59307725 |
| rs6556301 | T | G | T | G | −0.0111 | 0.014541 | 0.3596 | 0.362576 | FALSE | FALSE | 5 | 176527577 |
| rs6561943 | T | C | T | C | 0.0119 | 0.000327 | 0.2595 | 0.224798 | FALSE | FALSE | 13 | 58356761 |
| rs657452 | G | A | G | A | −0.0188 | −0.00767 | 0.6216 | 0.566124 | FALSE | FALSE | 1 | 49589847 |
| rs6587552 | G | A | G | A | −0.0173 | −0.01278 | 0.7591 | 0.727174 | FALSE | FALSE | 1 | 151018861 |
| rs6591407 | A | C | A | C | −0.0118 | −0.00977 | 0.1861 | 0.171614 | FALSE | FALSE | 11 | 56914157 |
| rs6593688 | G | A | G | A | 0.0137 | 0.011051 | 0.3733 | 0.358931 | FALSE | FALSE | 1 | 96322205 |
| rs6595205 | G | C | G | C | −0.0114 | −0.00104 | 0.5305 | 0.551656 | TRUE | TRUE | 5 | 119372533 |
| rs663129 | A | G | A | G | 0.0545 | 0.058163 | 0.2301 | 0.256835 | FALSE | FALSE | 18 | 57838401 |
| rs6673081 | C | T | C | T | −0.01 | 0.040575 | 0.5534 | 0.582621 | FALSE | FALSE | 1 | 154989595 |
| rs6692586 | G | A | G | A | −0.0192 | −0.00875 | 0.832 | 0.770273 | FALSE | FALSE | 1 | 23299906 |
| rs6712 | C | G | C | G | 0.0138 | 0.001191 | 0.1368 | 0.138812 | TRUE | FALSE | 22 | 50637922 |
| rs6764533 | A | G | A | G | 0.0116 | 0.012193 | 0.359 | 0.358807 | FALSE | FALSE | 3 | 196088464 |
| rs6772756 | G | A | G | A | −0.0104 | −0.01455 | 0.3372 | 0.356257 | FALSE | FALSE | 3 | 182312152 |
| rs6785245 | C | T | C | T | 0.0132 | 0.003346 | 0.3969 | 0.364882 | FALSE | FALSE | 3 | 82647990 |
| rs6804842 | G | A | G | A | 0.0156 | 0.00994 | 0.572 | 0.55015 | FALSE | FALSE | 3 | 25106437 |
| rs6815910 | A | T | A | T | −0.0128 | −0.01509 | 0.5435 | 0.531143 | TRUE | TRUE | 4 | 55495793 |
| rs6841761 | T | G | T | G | −0.0131 | −0.00202 | 0.5252 | 0.47632 | FALSE | FALSE | 4 | 25423538 |
| rs685870 | C | T | C | T | 0.012 | −0.01201 | 0.7035 | 0.674132 | FALSE | FALSE | 11 | 64111928 |
| rs6985109 | A | G | A | G | −0.0177 | −0.01207 | 0.5338 | 0.485049 | FALSE | FALSE | 8 | 10761585 |
| rs7024334 | G | T | G | T | −0.0138 | −0.00445 | 0.7742 | 0.758244 | FALSE | FALSE | 9 | 109072075 |
| rs7025938 | G | C | G | C | 0.0166 | 0.008837 | 0.3187 | 0.402909 | TRUE | FALSE | 9 | 103088321 |
| rs7037266 | A | C | A | C | −0.0112 | −0.01113 | 0.3739 | 0.38683 | FALSE | FALSE | 9 | 6942940 |
| rs705217 | G | T | G | T | −0.0102 | 0.000797 | 0.3652 | 0.365399 | FALSE | FALSE | 1 | 34581472 |
| rs705704 | A | G | A | G | −0.0131 | 0.002213 | 0.3304 | 0.3024 | FALSE | FALSE | 12 | 56435412 |
| rs7084454 | A | G | A | G | 0.0193 | 0.003472 | 0.335 | 0.298698 | FALSE | FALSE | 10 | 21821274 |
| rs709400 | G | A | G | A | −0.015 | −0.01468 | 0.3818 | 0.336875 | FALSE | FALSE | 14 | 104149475 |
| rs7102454 | C | T | C | T | 0.0158 | −0.00086 | 0.3435 | 0.306318 | FALSE | FALSE | 11 | 65594820 |
| rs7117238 | A | G | A | G | −0.0131 | −0.02881 | 0.168 | 0.213073 | FALSE | FALSE | 11 | 78040259 |
| rs7124681 | A | C | A | C | 0.0263 | −0.00068 | 0.4133 | 0.388567 | FALSE | FALSE | 11 | 47529947 |
| rs7138803 | A | G | A | G | 0.03 | 0.008175 | 0.3772 | 0.371503 | FALSE | FALSE | 12 | 50247468 |
| rs7144011 | T | G | T | G | 0.0282 | 0.021424 | 0.2136 | 0.202625 | FALSE | FALSE | 14 | 79940383 |
| rs7148846 | G | T | G | T | 0.0124 | 0.008641 | 0.1896 | 0.224724 | FALSE | FALSE | 14 | 40133821 |
| rs7172627 | G | A | G | A | 0.0117 | 0.001935 | 0.4719 | 0.480772 | FALSE | FALSE | 15 | 31877690 |
| rs7181498 | C | T | C | T | −0.0163 | −0.00408 | 0.6309 | 0.632447 | FALSE | FALSE | 15 | 95271404 |
| rs7196720 | C | T | C | T | −0.0129 | 0.004959 | 0.5068 | 0.542577 | FALSE | FALSE | 16 | 24534662 |
| rs7206608 | G | C | G | C | 0.0132 | −0.00075 | 0.3146 | 0.303515 | TRUE | FALSE | 16 | 82872628 |
| rs7222349 | A | G | A | G | 0.0115 | −0.00306 | 0.3441 | 0.361168 | FALSE | FALSE | 17 | 42304644 |
| rs7239575 | C | T | C | T | −0.0202 | −0.00708 | 0.4832 | 0.488675 | FALSE | FALSE | 18 | 21120035 |
| rs7318817 | T | C | T | C | −0.0155 | −0.00732 | 0.6071 | 0.607876 | FALSE | FALSE | 13 | 28617708 |
| rs7334078 | C | T | C | T | −0.0121 | 0.002441 | 0.2882 | 0.272763 | FALSE | FALSE | 13 | 99120484 |
| rs7358465 | T | C | T | C | 0.0103 | 0.008146 | 0.6781 | 0.654225 | FALSE | FALSE | 11 | 89990280 |
| rs7488867 | T | C | T | C | −0.0204 | −0.0059 | 0.2639 | 0.290074 | FALSE | FALSE | 12 | 103699685 |
| rs7498665 | G | A | G | A | 0.0271 | 0.018987 | 0.4038 | 0.349174 | FALSE | FALSE | 16 | 28883241 |
| rs7519259 | A | G | A | G | 0.0125 | 0.019619 | 0.5356 | 0.472488 | FALSE | FALSE | 1 | 66434743 |
| rs7535528 | A | G | A | G | −0.0152 | −0.01408 | 0.3741 | 0.342233 | FALSE | FALSE | 1 | 2444414 |
| rs754635 | G | C | G | C | 0.0198 | 0.00575 | 0.8873 | 0.84189 | TRUE | FALSE | 3 | 42305131 |
| rs7550711 | T | C | T | C | 0.0649 | −0.04835 | 0.03058 | 0.02765 | FALSE | FALSE | 1 | 110082886 |
| rs7551507 | T | C | T | C | −0.0184 | 0.002007 | 0.5633 | 0.50011 | FALSE | FALSE | 1 | 74995225 |
| rs7557796 | C | T | C | T | −0.016 | −0.025 | 0.6524 | 0.632715 | FALSE | FALSE | 2 | 86766153 |
| rs756717 | A | G | A | G | −0.0148 | −0.02418 | 0.3973 | 0.365565 | FALSE | FALSE | 16 | 72996162 |
| rs7599312 | A | G | A | G | −0.0186 | −0.02665 | 0.2652 | 0.247385 | FALSE | FALSE | 2 | 213413231 |
| rs7615297 | G | C | G | C | −0.0149 | −0.01792 | 0.1465 | 0.135189 | TRUE | FALSE | 3 | 156299313 |
| rs7626079 | T | C | T | C | 0.011 | 0.002135 | 0.3434 | 0.365086 | FALSE | FALSE | 3 | 66427259 |
| rs7637852 | G | A | G | A | −0.0139 | −0.01497 | 0.6951 | 0.681568 | FALSE | FALSE | 3 | 44041777 |
| rs7640424 | T | C | T | C | −0.0136 | 0.002306 | 0.2969 | 0.28416 | FALSE | FALSE | 3 | 107820063 |
| rs765875 | T | C | T | C | −0.0121 | 0.014941 | 0.4808 | 0.492792 | FALSE | FALSE | 6 | 143185683 |
| rs7683836 | A | G | A | G | −0.0114 | −0.01475 | 0.5405 | 0.513357 | FALSE | FALSE | 4 | 180167906 |
| rs7685048 | T | C | T | C | −0.0101 | −0.00744 | 0.4654 | 0.481657 | FALSE | FALSE | 4 | 95027784 |
| rs768840 | A | G | A | G | 0.0114 | 0.009157 | 0.4183 | 0.477879 | FALSE | FALSE | 14 | 73143457 |
| rs769449 | A | G | A | G | −0.0254 | 0.089569 | 0.1161 | 0.106438 | FALSE | FALSE | 19 | 45410002 |
| rs7694732 | G | A | G | A | −0.0099 | 0.013968 | 0.4378 | 0.42103 | FALSE | FALSE | 4 | 115124089 |
| rs7703576 | C | T | C | T | 0.0103 | 0.034412 | 0.2885 | 0.252909 | FALSE | FALSE | 5 | 144543996 |
| rs7704281 | A | G | A | G | 0.0271 | −0.03095 | 0.04531 | 0.047016 | FALSE | FALSE | 5 | 50591460 |
| rs7715256 | T | G | T | G | −0.0166 | −0.00236 | 0.5781 | 0.535469 | FALSE | FALSE | 5 | 153537893 |
| rs7724675 | A | G | A | G | −0.0119 | 0.012473 | 0.2238 | 0.232785 | FALSE | FALSE | 5 | 130440010 |
| rs7730004 | T | C | T | C | 0.0148 | −0.01037 | 0.6693 | 0.659154 | FALSE | FALSE | 5 | 43191033 |
| rs7730898 | A | G | A | G | 0.0168 | 0.009197 | 0.729 | 0.734218 | FALSE | FALSE | 5 | 170459675 |
| rs774246 | G | A | G | A | 0.0153 | 0.008831 | 0.1444 | 0.124282 | FALSE | FALSE | 7 | 26990816 |
| rs7761673 | A | T | A | T | −0.0126 | −0.0137 | 0.2058 | 0.208239 | TRUE | FALSE | 6 | 70357368 |
| rs7780752 | C | T | C | T | 0.0139 | −0.00756 | 0.36 | 0.345097 | FALSE | FALSE | 7 | 93241640 |
| rs7788008 | A | G | A | G | −0.0157 | −0.00807 | 0.4445 | 0.426968 | FALSE | FALSE | 7 | 112972483 |
| rs7811342 | C | T | C | T | −0.0197 | 0.01451 | 0.1058 | 0.155023 | FALSE | FALSE | 7 | 138794618 |
| rs7819514 | A | G | A | G | −0.0107 | −0.02096 | 0.3216 | 0.350427 | FALSE | FALSE | 8 | 93204442 |
| rs7826312 | C | T | C | T | 0.0104 | −0.00182 | 0.5879 | 0.547749 | FALSE | FALSE | 8 | 32400115 |
| rs7844647 | C | T | C | T | −0.0123 | 0.003018 | 0.2681 | 0.309461 | FALSE | FALSE | 8 | 34503776 |
| rs7869771 | C | A | C | A | −0.014 | −5.80E−05 | 0.2647 | 0.286754 | FALSE | FALSE | 9 | 94180627 |
| rs7871866 | C | G | C | G | 0.0187 | 0.022587 | 0.1531 | 0.161105 | TRUE | FALSE | 9 | 131027982 |
| rs7899106 | G | A | G | A | 0.0331 | −0.00681 | 0.04777 | 0.046115 | FALSE | FALSE | 10 | 87410904 |
| rs7903146 | T | C | T | C | −0.0181 | 0.033092 | 0.2912 | 0.275894 | FALSE | FALSE | 10 | 114758349 |
| rs7925214 | T | C | T | C | 0.0147 | 0.009985 | 0.5133 | 0.52692 | FALSE | FALSE | 11 | 130794253 |
| rs7970953 | A | G | A | G | 0.0135 | 0.000922 | 0.29 | 0.328562 | FALSE | FALSE | 12 | 24075508 |
| rs7983065 | T | C | T | C | −0.0148 | −0.03397 | 0.4503 | 0.409397 | FALSE | FALSE | 13 | 33380786 |
| rs7998796 | G | A | G | A | 0.0105 | 0.000527 | 0.3373 | 0.369944 | FALSE | FALSE | 13 | 81020036 |
| rs8027205 | G | C | G | C | −0.0108 | −0.00623 | 0.3967 | 0.375518 | TRUE | FALSE | 15 | 98280959 |
| rs8036040 | A | C | A | C | 0.0109 | 0.017326 | 0.4932 | 0.488909 | FALSE | FALSE | 15 | 36402716 |
| rs8047395 | A | G | A | G | 0.0642 | 0.018195 | 0.5061 | 0.514445 | FALSE | FALSE | 16 | 53798523 |
| rs806600 | G | A | G | A | −0.0095 | 0.004794 | 0.475 | 0.471568 | FALSE | FALSE | 5 | 172914939 |
| rs8071182 | A | G | A | G | 0.0133 | −0.02538 | 0.1735 | 0.197236 | FALSE | FALSE | 17 | 55336155 |
| rs8090983 | G | A | G | A | 0.0118 | 0.004433 | 0.3314 | 0.338596 | FALSE | FALSE | 18 | 52586691 |
| rs8097672 | T | A | T | A | 0.02 | −0.00425 | 0.1528 | 0.148366 | TRUE | FALSE | 18 | 1839601 |
| rs8097783 | A | G | A | G | −0.0389 | −0.03873 | 0.07554 | 0.083335 | FALSE | FALSE | 18 | 58051294 |
| rs8123881 | G | A | G | A | 0.0196 | −0.0048 | 0.1299 | 0.124976 | FALSE | FALSE | 20 | 15819495 |
| rs8181823 | C | A | C | A | 0.0127 | 0.00582 | 0.7614 | 0.742054 | FALSE | FALSE | 13 | 65477940 |
| rs818524 | C | T | C | T | 0.0106 | −0.00112 | 0.6939 | 0.65813 | FALSE | FALSE | 1 | 85201228 |
| rs8192675 | C | T | C | T | 0.0152 | −0.01599 | 0.2888 | 0.297117 | FALSE | FALSE | 3 | 170724883 |
| rs825688 | T | C | T | C | −0.0095 | −0.01848 | 0.456 | 0.40044 | FALSE | FALSE | 16 | 73595718 |
| rs845084 | A | G | A | G | 0.014 | 0.005359 | 0.2678 | 0.285301 | FALSE | FALSE | 10 | 125220036 |
| rs852056 | C | T | C | T | −0.0128 | −0.00734 | 0.7584 | 0.680874 | FALSE | FALSE | 20 | 17102860 |
| rs865809 | G | A | G | A | −0.0127 | 0.000753 | 0.7678 | 0.732354 | FALSE | FALSE | 3 | 183997735 |
| rs872281 | T | C | T | C | −0.0151 | 0.004416 | 0.1728 | 0.178538 | FALSE | FALSE | 14 | 40834177 |
| rs876605 | G | A | G | A | −0.0108 | −0.00795 | 0.7352 | 0.718287 | FALSE | FALSE | 5 | 77801359 |
| rs879620 | T | C | T | C | 0.0231 | −0.00294 | 0.6179 | 0.545251 | FALSE | FALSE | 16 | 4015729 |
| rs889398 | T | C | T | C | −0.0196 | −0.02676 | 0.4247 | 0.380521 | FALSE | FALSE | 16 | 69556715 |
| rs895330 | G | C | G | C | −0.0201 | −0.01397 | 0.1924 | 0.170898 | TRUE | FALSE | 19 | 4060707 |
| rs901630 | T | C | T | C | −0.0146 | 0.001762 | 0.3973 | 0.361045 | FALSE | FALSE | 6 | 98539519 |
| rs902695 | A | G | A | G | −0.0103 | −0.01674 | 0.4798 | 0.479005 | FALSE | FALSE | 2 | 113955074 |
| rs9294260 | A | G | A | G | 0.0147 | −0.00191 | 0.4731 | 0.459906 | FALSE | FALSE | 6 | 83433228 |
| rs9300422 | G | A | G | A | −0.0103 | 0.010007 | 0.6903 | 0.673151 | FALSE | FALSE | 13 | 98223320 |
| rs930295 | C | A | C | A | −0.0211 | 0.001004 | 0.8417 | 0.816685 | FALSE | FALSE | 2 | 50233352 |
| rs9304665 | A | T | A | T | 0.0229 | 0.033171 | 0.7633 | 0.689503 | TRUE | FALSE | 19 | 47602577 |
| rs934224 | T | C | T | C | 0.0107 | −0.00309 | 0.7399 | 0.715649 | FALSE | FALSE | 2 | 16613889 |
| rs9362662 | G | A | G | A | −0.0112 | −0.01233 | 0.5201 | 0.482502 | FALSE | FALSE | 6 | 90296588 |
| rs9367368 | C | T | C | T | −0.0121 | 0.011478 | 0.3033 | 0.295635 | FALSE | FALSE | 6 | 13189275 |
| rs9370261 | T | C | T | C | 0.0231 | 0.009905 | 0.04601 | 0.079326 | FALSE | FALSE | 6 | 53939516 |
| rs9375702 | T | C | T | C | −0.0115 | 0.015554 | 0.705 | 0.665903 | FALSE | FALSE | 6 | 130384187 |
| rs9379827 | A | C | A | C | −0.0132 | 0.00165 | 0.2409 | 0.208028 | FALSE | FALSE | 6 | 26153335 |
| rs9408882 | A | G | A | G | −0.0093 | 0.000819 | 0.4594 | 0.458228 | FALSE | FALSE | 9 | 118664402 |
| rs946824 | C | T | C | T | −0.0206 | 0.001368 | 0.859 | 0.82585 | FALSE | FALSE | 1 | 243684019 |
| rs947612 | A | G | A | G | −0.0116 | −0.00906 | 0.7516 | 0.688003 | FALSE | FALSE | 6 | 73738661 |
| rs9478671 | G | A | G | A | 0.012 | −0.0037 | 0.2087 | 0.185819 | FALSE | FALSE | 6 | 155987825 |
| rs9522285 | A | G | A | G | 0.0127 | 0.005095 | 0.4143 | 0.397239 | FALSE | FALSE | 13 | 112230701 |
| rs9538162 | C | T | C | T | −0.0156 | 0.006955 | 0.4138 | 0.422759 | FALSE | FALSE | 13 | 59265043 |
| rs9547153 | G | A | G | A | 0.0098 | 0.009153 | 0.3839 | 0.372436 | FALSE | FALSE | 13 | 85903717 |
| rs9571687 | A | C | A | C | −0.0129 | −0.0013 | 0.329 | 0.325533 | FALSE | FALSE | 13 | 67472713 |
| rs9615905 | T | C | T | C | 0.011 | 0.036297 | 0.45 | 0.409403 | FALSE | FALSE | 22 | 48875699 |
| rs962273 | C | T | C | T | 0.0137 | 0.049154 | 0.7057 | 0.690074 | FALSE | FALSE | 17 | 46978353 |
| rs9650755 | G | A | G | A | 0.0154 | −0.00226 | 0.2664 | 0.320913 | FALSE | FALSE | 9 | 96484342 |
| rs9688431 | C | T | C | T | −0.0231 | 0.004114 | 0.06034 | 0.071955 | FALSE | FALSE | 6 | 73922654 |
| rs977747 | G | T | G | T | −0.0169 | −0.0139 | 0.5949 | 0.550761 | FALSE | FALSE | 1 | 47684677 |
| rs9783858 | T | C | T | C | 0.0091 | −0.01308 | 0.5191 | 0.494795 | FALSE | FALSE | 18 | 42534584 |
| rs9806742 | A | G | A | G | 0.0208 | 0.009074 | 0.8826 | 0.879127 | FALSE | FALSE | 15 | 73051219 |
| rs9816226 | T | A | T | A | 0.0323 | −0.00252 | 0.8199 | 0.79361 | TRUE | FALSE | 3 | 185834499 |
| rs9845966 | G | T | G | T | −0.0105 | −0.0119 | 0.5479 | 0.510761 | FALSE | FALSE | 3 | 13433158 |
| rs987237 | G | A | G | A | 0.0409 | 0.025245 | 0.1803 | 0.189188 | FALSE | FALSE | 6 | 50803050 |
| rs9926784 | C | T | C | T | −0.0258 | 0.003405 | 0.1822 | 0.21675 | FALSE | FALSE | 16 | 19941968 |
| rs9927848 | A | C | A | C | −0.0122 | 0.014777 | 0.7326 | 0.707575 | FALSE | FALSE | 16 | 23833071 |
| rs9951619 | G | T | G | T | 0.0156 | 0.002125 | 0.7643 | 0.73016 | FALSE | FALSE | 18 | 56882326 |
| rs998732 | G | A | G | A | −0.0171 | −0.0053 | 0.1578 | 0.150033 | FALSE | FALSE | 19 | 19378671 |
| rs9989141 | T | C | T | C | 0.0162 | 0.003376 | 0.6387 | 0.587188 | FALSE | FALSE | 14 | 94006257 |
| rs999889 | A | G | A | G | −0.0108 | −0.0153 | 0.2818 | 0.29367 | FALSE | FALSE | 10 | 84279949 |

EAE: allele exposure; OAE: other allele exposure, EAO: allele outcome, OAO: other allele outcome, B Ex: Beta exposure, B Out: Beta outcome, Eaf Ex: Eaf exposure, Eaf Out: Eaf outcome, chr: chromosome, pos: position.

TABLE 34
Raw data set 2. For all SNPs listed in the table below, “outcome” is “Coronary
heart disease ∥ id:ieu-a-7”, “mr_keep.outcome” is “TRUE”, “id.exposure”
is “ieu-b-40”, “exposure” is “body mass index”, “mr_keep.exposure” is “TRUE”; “action” is “2”, and “SNP_index” is “1”.

| | se. | Samplesize | Pval | Chr | Pos | Se | Pval | Samplesize | |
|---|---|---|---|---|---|---|---|---|---|
| SNP | outcome | outcome | outcome | exposure | exposure | exposure | exposure | exposure | mr_keep |
| rs10009336 | 0.0122586 | 184305 | 0.019234 | 4 | 44480783 | 0.0022 | 2.20E−10 | 794766 | TRUE |
| rs1006896 | 0.0168949 | 184305 | 0.0439117 | 3 | 88104411 | 0.0027 | 5.50E−18 | 691892 | TRUE |
| rs10132280 | 0.010664 | 184305 | 0.253816 | 14 | 25928179 | 0.0018 | 5.60E−35 | 786578 | TRUE |
| rs10169594 | 0.0100111 | 184305 | 0.243533 | 2 | 41637688 | 0.0018 | 2.00E−11 | 685712 | TRUE |
| rs10182181 | 0.009279 | 184305 | 0.0486486 | 2 | 25150296 | 0.0016 | 6.70E−90 | 792111 | TRUE |
| rs10192119 | 0.012112 | 184305 | 0.739714 | 2 | 164581241 | 0.0022 | 3.00E−14 | 795369 | TRUE |
| rs10197031 | 0.0104695 | 184305 | 0.341 | 2 | 105454590 | 0.0019 | 1.90E−18 | 691479 | TRUE |
| rs10243319 | 0.0094104 | 184305 | 0.110585 | 7 | 147674678 | 0.0018 | 1.20E−09 | 690936 | TRUE |
| rs10247983 | 0.0152503 | 184305 | 0.740488 | 7 | 114590228 | 0.0033 | 1.70E−09 | 672411 | TRUE |
| rs10248136 | 0.0093018 | 184305 | 0.0426707 | 7 | 39077397 | 0.0017 | 2.00E−08 | 686892 | TRUE |
| rs10269783 | 0.0093605 | 184305 | 0.381946 | 7 | 49616203 | 0.0017 | 1.40E−15 | 790551 | TRUE |
| rs10408324 | 0.0112788 | 184305 | 0.83004 | 19 | 51774806 | 0.0019 | 9.50E−11 | 690737 | TRUE |
| rs10478110 | 0.0094843 | 184305 | 0.385877 | 5 | 112445734 | 0.0017 | 9.60E−09 | 680441 | TRUE |
| rs1048932 | 0.0093941 | 184305 | 0.815004 | 11 | 115044850 | 0.0017 | 3.80E−22 | 795167 | TRUE |
| rs10492229 | 0.0120577 | 184305 | 0.240253 | 12 | 110602173 | 0.0019 | 7.70E−14 | 794845 | TRUE |
| rs10510419 | 0.013497 | 184305 | 0.266574 | 3 | 12426936 | 0.0023 | 2.20E−14 | 789318 | TRUE |
| rs10518694 | 0.0134177 | 184305 | 0.487724 | 15 | 53072673 | 0.0025 | 3.30E−09 | 690554 | TRUE |
| rs1064213 | 0.0095614 | 184305 | 0.588272 | 2 | 198950240 | 0.0017 | 2.40E−12 | 692576 | TRUE |
| rs10733051 | 0.0092898 | 184305 | 0.801128 | 1 | 167280354 | 0.0016 | 2.90E−09 | 781928 | TRUE |
| rs10742752 | 0.0095071 | 184305 | 0.483269 | 11 | 45438374 | 0.0017 | 1.10E−13 | 792704 | TRUE |
| rs10747488 | 0.0108541 | 184305 | 0.0902443 | 1 | 98299475 | 0.002 | 1.20E−09 | 689295 | TRUE |
| rs10750215 | 0.0095028 | 184305 | 0.055115 | 11 | 122505344 | 0.0017 | 1.30E−10 | 788895 | TRUE |
| rs1075901 | 0.0093789 | 184305 | 0.466996 | 17 | 15943910 | 0.0016 | 1.20E−13 | 794789 | TRUE |
| rs10768994 | 0.0093887 | 184305 | 0.530291 | 11 | 43936945 | 0.0017 | 6.40E−12 | 791685 | TRUE |
| rs10795422 | 0.0104228 | 184305 | 0.282785 | 10 | 16759312 | 0.0019 | 9.30E−14 | 692108 | TRUE |
| rs10811871 | 0.0096103 | 184305 | 0.0542538 | 9 | 23200766 | 0.0018 | 1.60E−09 | 686376 | TRUE |
| rs10832778 | 0.0094376 | 184305 | 0.186256 | 11 | 17394073 | 0.0017 | 1.30E−13 | 783042 | TRUE |
| rs10858334 | 0.0143226 | 184305 | 0.280995 | 9 | 137989785 | 0.0026 | 2.70E−08 | 672640 | TRUE |
| rs10867256 | 0.0093344 | 184305 | 0.812511 | 9 | 81367391 | 0.0017 | 8.70E−12 | 689493 | TRUE |
| rs10878946 | 0.0102794 | 184305 | 0.285891 | 12 | 69642315 | 0.0019 | 3.60E−13 | 685707 | TRUE |
| rs10887578 | 0.0096353 | 184305 | 0.882918 | 10 | 88096047 | 0.0017 | 1.60E−13 | 679172 | FALSE |
| rs10914462 | 0.0094723 | 184305 | 0.818636 | 1 | 32125943 | 0.0017 | 1.50E−10 | 689808 | TRUE |
| rs10915840 | 0.0108584 | 184305 | 0.485476 | 1 | 225668524 | 0.0019 | 1.30E−09 | 684857 | TRUE |
| rs10920678 | 0.0093344 | 184305 | 0.00502655 | 1 | 190239907 | 0.0016 | 1.50E−21 | 788624 | TRUE |
| rs10938397 | 0.0093485 | 184305 | 0.00106079 | 4 | 45182527 | 0.0016 | 3.40E−86 | 793518 | TRUE |
| rs10942267 | 0.010274 | 184305 | 0.968711 | 5 | 80841914 | 0.0019 | 3.90E−17 | 689084 | TRUE |
| rs10953740 | 0.0096461 | 184305 | 0.65966 | 7 | 113460282 | 0.0017 | 1.00E−18 | 684419 | TRUE |
| rs10962550 | 0.0114505 | 184305 | 0.0405303 | 9 | 16720329 | 0.0022 | 6.20E−16 | 690579 | TRUE |
| rs10968114 | 0.0093822 | 184305 | 0.477397 | 9 | 27800007 | 0.0017 | 6.10E−11 | 686025 | TRUE |
| rs10971709 | 0.0111517 | 184305 | 0.37646 | 9 | 33804813 | 0.0021 | 6.20E−10 | 688312 | TRUE |
| rs10984756 | 0.0175162 | 184305 | 0.257788 | 9 | 122651784 | 0.0029 | 1.10E−09 | 689917 | TRUE |
| rs11030618 | 0.0094267 | 184305 | 0.232167 | 11 | 29243293 | 0.0017 | 2.40E−10 | 690005 | TRUE |
| rs11066188 | 0.0108943 | 184305 | 6.72E−09 | 12 | 112610714 | 0.0017 | 8.10E−13 | 792755 | TRUE |
| rs11084553 | 0.0150276 | 184305 | 0.111652 | 19 | 31019780 | 0.0024 | 1.80E−18 | 691103 | TRUE |
| rs11105839 | 0.009367 | 184305 | 0.296491 | 12 | 91237920 | 0.0017 | 1.10E−10 | 781573 | TRUE |
| rs11115176 | 0.0111908 | 184305 | 0.290582 | 12 | 82465797 | 0.0019 | 2.00E−10 | 792384 | TRUE |
| rs11118308 | 0.0093648 | 184305 | 0.152278 | 1 | 219633869 | 0.0016 | 4.80E−10 | 794625 | TRUE |
| rs1112613 | 0.0118806 | 184305 | 0.669811 | 13 | 53651850 | 0.0023 | 3.40E−09 | 682816 | TRUE |
| rs11150911 | 0.01011 | 184305 | 0.899094 | 18 | 73498528 | 0.0018 | 4.70E−13 | 781716 | TRUE |
| rs11165643 | 0.0092844 | 184305 | 0.579105 | 1 | 96924097 | 0.0017 | 1.40E−35 | 792657 | TRUE |
| rs11170468 | 0.0122076 | 184305 | 0.661144 | 12 | 39430048 | 0.0019 | 1.90E−10 | 795265 | TRUE |
| rs11173522 | 0.010916 | 184305 | 0.549031 | 12 | 60953472 | 0.0021 | 1.10E−09 | 691593 | TRUE |
| rs11185111 | 0.0098558 | 184305 | 0.476673 | 1 | 107962328 | 0.0019 | 7.70E−12 | 686508 | TRUE |
| rs11251352 | 0.0094224 | 184305 | 0.481126 | 10 | 2585792 | 0.0018 | 7.00E−10 | 690804 | TRUE |
| rs1144387 | 0.009418 | 184305 | 0.81497 | 13 | 78365190 | 0.0017 | 1.60E−08 | 687565 | FALSE |
| rs11496125 | 0.0092551 | 184305 | 0.268873 | 7 | 103417557 | 0.0017 | 3.00E−22 | 684574 | TRUE |
| rs11505821 | 0.0176183 | 184305 | 0.0439997 | 7 | 76818677 | 0.0035 | 2.70E−19 | 758322 | TRUE |
| rs11538 | 0.0136969 | 184305 | 0.911 | 22 | 18220831 | 0.0023 | 3.30E−09 | 692349 | TRUE |
| rs1158805 | 0.009468 | 184305 | 0.773006 | 18 | 40736590 | 0.0018 | 1.20E−14 | 691776 | TRUE |
| rs11609659 | 0.0120066 | 184305 | 0.302769 | 12 | 108296260 | 0.002 | 2.20E−14 | 679177 | TRUE |
| rs11611246 | 0.0114016 | 184305 | 0.515014 | 12 | 939480 | 0.002 | 5.00E−32 | 779823 | TRUE |
| rs11615578 | 0.0117199 | 184305 | 0.563154 | 12 | 121714935 | 0.002 | 8.10E−11 | 669422 | TRUE |
| rs11656076 | 0.0105869 | 184305 | 0.0764716 | 17 | 31464270 | 0.0021 | 5.60E−12 | 691283 | TRUE |
| rs11672660 | 0.0125585 | 184305 | 0.0019162 | 19 | 46180184 | 0.0021 | 1.70E−60 | 768426 | TRUE |
| rs11713193 | 0.0096668 | 184305 | 0.00183371 | 3 | 49924424 | 0.0017 | 2.40E−44 | 692159 | TRUE |
| rs11736228 | 0.0103316 | 184305 | 0.157472 | 4 | 147376805 | 0.002 | 4.10E−12 | 691580 | TRUE |
| rs11738695 | 0.00967 | 184305 | 0.210529 | 5 | 108699161 | 0.0017 | 2.00E−08 | 691380 | TRUE |
| rs11739877 | 0.0097787 | 184305 | 0.705456 | 5 | 105876806 | 0.0018 | 6.60E−11 | 692540 | TRUE |
| rs11781699 | 0.0125639 | 184305 | 0.409517 | 8 | 118863061 | 0.0021 | 3.10E−10 | 784642 | TRUE |
| rs11855853 | 0.0115113 | 184305 | 0.00289608 | 15 | 78012618 | 0.002 | 2.40E−13 | 682564 | TRUE |
| rs1187352 | 0.0099786 | 184305 | 0.288109 | 9 | 87293457 | 0.0018 | 6.00E−11 | 688522 | TRUE |
| rs11880870 | 0.0096592 | 184305 | 0.102588 | 19 | 18830704 | 0.0017 | 1.00E−28 | 717350 | TRUE |
| rs11889536 | 0.0134633 | 184305 | 0.0484128 | 2 | 220163543 | 0.0024 | 6.40E−15 | 688977 | TRUE |
| rs11908637 | 0.0119849 | 184305 | 0.866614 | 20 | 47428485 | 0.0021 | 4.90E−09 | 691443 | TRUE |
| rs11945861 | 0.0105988 | 184305 | 0.658535 | 4 | 65700865 | 0.002 | 5.00E−13 | 682451 | TRUE |
| rs11951673 | 0.0096038 | 184305 | 0.0598412 | 5 | 95861012 | 0.0017 | 1.10E−13 | 792278 | TRUE |
| rs12033257 | 0.009947 | 184305 | 0.142659 | 1 | 112318484 | 0.0018 | 2.40E−15 | 664083 | TRUE |
| rs12041258 | 0.0110083 | 184305 | 0.0624051 | 1 | 195047936 | 0.002 | 9.50E−13 | 688602 | TRUE |
| rs12044597 | 0.009493 | 184305 | 0.156192 | 1 | 1708801 | 0.0016 | 1.70E−18 | 789125 | TRUE |
| rs12049202 | 0.0116286 | 184305 | 0.0188917 | 1 | 77967523 | 0.0022 | 1.00E−28 | 691566 | TRUE |
| rs12098284 | 0.0140912 | 184305 | 0.00458617 | 10 | 76047464 | 0.0026 | 1.80E−11 | 686167 | TRUE |
| rs12150665 | 0.009518 | 184305 | 0.59048 | 17 | 34914787 | 0.0017 | 1.60E−22 | 795501 | TRUE |
| rs1218822 | 0.0098395 | 184305 | 0.532818 | 13 | 28011963 | 0.0017 | 1.90E−22 | 794711 | TRUE |
| rs12299814 | 0.010916 | 184305 | 0.512054 | 12 | 90216146 | 0.002 | 5.20E−15 | 687962 | TRUE |
| rs12328930 | 0.0097146 | 184305 | 0.565562 | 2 | 175079125 | 0.0017 | 1.80E−08 | 690521 | TRUE |
| rs12334877 | 0.0110768 | 184305 | 0.173271 | 8 | 67194171 | 0.0022 | 7.70E−11 | 683820 | TRUE |
| rs12364470 | 0.0133656 | 184305 | 0.857963 | 11 | 134601012 | 0.0022 | 1.10E−15 | 787411 | TRUE |
| rs12369179 | 0.0193477 | 184305 | 0.601077 | 12 | 122963550 | 0.0031 | 2.50E−31 | 674260 | TRUE |
| rs12416812 | 0.0098113 | 184305 | 0.990567 | 11 | 888632 | 0.0016 | 6.10E−12 | 793338 | TRUE |
| rs1241986 | 0.0117046 | 184305 | 0.505591 | 18 | 6873954 | 0.0024 | 1.10E−08 | 687757 | TRUE |
| rs12422552 | 0.0105467 | 184305 | 0.218215 | 12 | 14413931 | 0.002 | 1.60E−11 | 689543 | TRUE |
| rs12429545 | 0.0129343 | 184305 | 0.411649 | 13 | 54102206 | 0.0025 | 9.60E−38 | 778918 | TRUE |
| rs12448257 | 0.0113483 | 184305 | 0.394595 | 16 | 3599655 | 0.002 | 8.10E−20 | 779628 | TRUE |
| rs12546578 | 0.0104511 | 184305 | 0.795106 | 8 | 85085268 | 0.002 | 1.00E−13 | 689192 | TRUE |
| rs12564992 | 0.0135557 | 184305 | 0.879214 | 1 | 174478100 | 0.0026 | 5.30E−14 | 795119 | TRUE |
| rs12593036 | 0.0100177 | 184305 | 0.306783 | 15 | 81058652 | 0.0019 | 3.80E−16 | 686055 | TRUE |
| rs12602912 | 0.0111126 | 184305 | 0.0741447 | 17 | 65870073 | 0.0021 | 9.90E−18 | 777510 | TRUE |
| rs1260326 | 0.0096201 | 184305 | 0.734939 | 2 | 27730940 | 0.0017 | 3.90E−10 | 784462 | TRUE |
| rs12629015 | 0.0109062 | 184305 | 0.36859 | 3 | 119618053 | 0.0023 | 2.10E−09 | 691059 | TRUE |
| rs1266874 | 0.0096657 | 184305 | 0.698115 | 6 | 51779638 | 0.0018 | 9.80E−15 | 691020 | TRUE |
| rs12675063 | 0.0133026 | 184305 | 0.554661 | 8 | 132879047 | 0.0026 | 1.30E−09 | 789771 | TRUE |
| rs1268065 | 0.0093691 | 184305 | 0.782867 | 6 | 126042783 | 0.0017 | 1.00E−09 | 759626 | TRUE |
| rs12680842 | 0.0096538 | 184305 | 0.304733 | 8 | 95582606 | 0.0018 | 4.40E−14 | 782549 | TRUE |
| rs12718572 | 0.0098145 | 184305 | 0.559884 | 7 | 50573325 | 0.0018 | 3.00E−11 | 686523 | TRUE |
| rs12762034 | 0.0160454 | 184305 | 0.819324 | 10 | 33969931 | 0.0032 | 7.30E−14 | 692191 | TRUE |
| rs12779328 | 0.0107943 | 184305 | 0.301238 | 10 | 12943973 | 0.0019 | 4.50E−08 | 690736 | TRUE |
| rs1285997 | 0.0105673 | 184305 | 0.0153313 | 14 | 91513029 | 0.0019 | 1.20E−13 | 684235 | TRUE |
| rs12888545 | 0.0118307 | 184305 | 0.114658 | 14 | 88308044 | 0.002 | 9.10E−12 | 688605 | TRUE |
| rs12888955 | 0.0097124 | 184305 | 0.0379796 | 14 | 103256877 | 0.0018 | 1.40E−22 | 691849 | TRUE |
| rs12905439 | 0.0112962 | 184305 | 0.115902 | 15 | 99521883 | 0.0018 | 1.40E−10 | 675205 | TRUE |
| rs12914489 | 0.016708 | 184305 | 0.0276745 | 15 | 74187937 | 0.0026 | 3.80E−10 | 795244 | TRUE |
| rs12922346 | 0.0109475 | 184305 | 0.199961 | 16 | 82438337 | 0.002 | 1.00E−11 | 679615 | TRUE |
| rs12933482 | 0.0171578 | 184305 | 0.122752 | 16 | 72189604 | 0.0028 | 4.90E−11 | 691477 | TRUE |
| rs12936083 | 0.0099264 | 184305 | 0.987059 | 17 | 4801887 | 0.0019 | 4.10E−13 | 633615 | TRUE |
| rs12939549 | 0.009455 | 184305 | 0.10125 | 17 | 78611724 | 0.0016 | 2.70E−28 | 793950 | TRUE |
| rs1296328 | 0.0098645 | 184305 | 0.811154 | 4 | 137083193 | 0.0018 | 4.90E−24 | 683488 | TRUE |
| rs12981256 | 0.010085 | 184305 | 0.16962 | 19 | 1865901 | 0.0018 | 1.10E−15 | 678327 | TRUE |
| rs13021737 | 0.0124444 | 184305 | 0.00292772 | 2 | 632348 | 0.0021 | 7.50E−157 | 789534 | TRUE |
| rs13047416 | 0.0095734 | 184305 | 0.80957 | 21 | 40309436 | 0.0018 | 2.20E−17 | 683228 | FALSE |
| rs13069244 | 0.0207447 | 184305 | 0.288298 | 3 | 180441172 | 0.0032 | 3.00E−09 | 791327 | TRUE |
| rs13107325 | 0.0223469 | 184305 | 0.765341 | 4 | 103188709 | 0.0032 | 1.10E−47 | 792045 | TRUE |
| rs13110266 | 0.0093029 | 184305 | 0.344726 | 4 | 162129844 | 0.0017 | 1.90E−12 | 791087 | TRUE |
| rs13132853 | 0.0111648 | 184305 | 0.345973 | 4 | 38680015 | 0.0018 | 4.70E−15 | 682543 | TRUE |
| rs13147390 | 0.0099329 | 184305 | 0.0272577 | 4 | 80712000 | 0.0018 | 1.00E−08 | 681032 | TRUE |
| rs13174863 | 0.0141455 | 184305 | 0.490212 | 5 | 139080745 | 0.0023 | 2.90E−16 | 773762 | TRUE |
| rs13184896 | 0.0094561 | 184305 | 0.77467 | 5 | 122734005 | 0.0016 | 3.30E−16 | 794825 | TRUE |
| rs13191362 | 0.0152568 | 184305 | 0.0162062 | 6 | 163033350 | 0.0025 | 5.90E−21 | 792699 | TRUE |
| rs1320903 | 0.0100665 | 184305 | 0.85263 | 3 | 131758077 | 0.0018 | 9.20E−32 | 691519 | TRUE |
| rs1321432 | 0.0096223 | 184305 | 0.994527 | 20 | 6614691 | 0.0018 | 3.50E−29 | 686481 | TRUE |
| rs13240600 | 0.0114591 | 184305 | 0.403246 | 7 | 99064466 | 0.0024 | 3.50E−17 | 692233 | TRUE |
| rs13250058 | 0.0099047 | 184305 | 0.324729 | 8 | 112270826 | 0.0018 | 2.90E−10 | 787870 | TRUE |
| rs13263601 | 0.0104717 | 184305 | 0.270413 | 8 | 14095900 | 0.0018 | 2.20E−17 | 686196 | TRUE |
| rs1327259 | 0.0094452 | 184305 | 0.66223 | 6 | 51177811 | 0.0018 | 1.70E−18 | 685922 | TRUE |
| rs13287131 | 0.0110909 | 184305 | 0.304478 | 9 | 92119579 | 0.002 | 6.80E−10 | 683808 | TRUE |
| rs1330052 | 0.0099601 | 184305 | 0.939177 | 13 | 86536006 | 0.0018 | 1.50E−13 | 691613 | TRUE |
| rs13329567 | 0.0106879 | 184305 | 0.18562 | 15 | 68104367 | 0.002 | 1.00E−50 | 793953 | TRUE |
| rs1365466 | 0.010223 | 184305 | 0.163342 | 18 | 36182440 | 0.0019 | 3.30E−13 | 791868 | TRUE |
| rs1371108 | 0.0107237 | 184305 | 0.277066 | 2 | 81816251 | 0.0018 | 9.00E−11 | 684620 | TRUE |
| rs138289 | 0.0096505 | 184305 | 0.291391 | 22 | 32182708 | 0.0017 | 3.30E−09 | 687258 | FALSE |
| rs1409818 | 0.0143334 | 184305 | 0.700359 | 20 | 21381121 | 0.0029 | 2.50E−12 | 690984 | TRUE |
| rs1412235 | 0.0102794 | 184305 | 0.0248519 | 9 | 28410996 | 0.0017 | 6.00E−45 | 790147 | TRUE |
| rs1421334 | 0.009594 | 184305 | 0.134155 | 8 | 30865733 | 0.0018 | 1.00E−12 | 680665 | TRUE |
| rs1430387 | 0.00943 | 184305 | 0.271932 | 18 | 58227112 | 0.0017 | 5.80E−11 | 689325 | TRUE |
| rs1431659 | 0.0100057 | 184305 | 0.918168 | 8 | 73439070 | 0.0019 | 6.00E−24 | 689739 | TRUE |
| rs1436344 | 0.0093029 | 184305 | 0.441632 | 3 | 104606144 | 0.0017 | 4.10E−16 | 692017 | FALSE |
| rs1445652 | 0.0120696 | 184305 | 0.628016 | 2 | 155668460 | 0.0022 | 4.30E−08 | 682166 | TRUE |
| rs1452075 | 0.0101665 | 184305 | 0.859854 | 3 | 62481063 | 0.0018 | 1.30E−14 | 783729 | TRUE |
| rs1454687 | 0.009267 | 184305 | 0.464002 | 3 | 94038085 | 0.0017 | 5.20E−32 | 692324 | FALSE |
| rs1465900 | 0.0112734 | 184305 | 0.442275 | 11 | 76473138 | 0.002 | 4.80E−10 | 779748 | TRUE |
| rs1472169 | 0.0096538 | 184305 | 0.286794 | 9 | 37209396 | 0.0018 | 2.80E−15 | 689945 | TRUE |
| rs1476322 | 0.0093572 | 184305 | 0.0474067 | 3 | 161446055 | 0.0017 | 5.00E−09 | 692523 | TRUE |
| rs1477199 | 0.0137545 | 184305 | 0.169614 | 16 | 53712135 | 0.0024 | 9.40E−22 | 794442 | TRUE |
| rs1492767 | 0.0093311 | 184305 | 0.552203 | 4 | 55221467 | 0.0016 | 1.00E−08 | 794161 | TRUE |
| rs1503526 | 0.0094072 | 184305 | 0.549094 | 5 | 63020706 | 0.0017 | 5.50E−17 | 747347 | TRUE |
| rs1521527 | 0.0099166 | 184305 | 0.450131 | 2 | 165427825 | 0.0017 | 3.10E−12 | 678175 | FALSE |
| rs1522569 | 0.0125411 | 184305 | 0.525876 | 4 | 171632637 | 0.0022 | 2.90E−13 | 689573 | TRUE |
| rs1528435 | 0.0097863 | 184305 | 0.0653311 | 2 | 181550962 | 0.0017 | 9.10E−23 | 794198 | TRUE |
| rs1535660 | 0.0129354 | 184305 | 0.904192 | 9 | 10371073 | 0.0025 | 5.20E−09 | 690624 | TRUE |
| rs1538247 | 0.0098189 | 184305 | 0.843454 | 6 | 153395344 | 0.0019 | 1.00E−08 | 682726 | TRUE |
| rs1552893 | 0.0100079 | 184305 | 0.247277 | 3 | 194851700 | 0.0019 | 8.10E−11 | 691313 | TRUE |
| rs156201 | 0.0102729 | 184305 | 0.997359 | 6 | 104847441 | 0.002 | 5.80E−10 | 691835 | TRUE |
| rs1624134 | 0.0093355 | 184305 | 0.732652 | 10 | 34834482 | 0.0018 | 1.10E−08 | 690572 | TRUE |
| rs1656377 | 0.009393 | 184305 | 0.841698 | 3 | 158285280 | 0.0017 | 1.60E−08 | 691955 | TRUE |
| rs1681740 | 0.0097026 | 184305 | 0.680301 | 10 | 118564313 | 0.0018 | 1.10E−10 | 673459 | TRUE |
| rs16849710 | 0.0096092 | 184305 | 0.794494 | 1 | 202106797 | 0.0018 | 6.00E−11 | 666229 | TRUE |
| rs16851483 | 0.0164441 | 184305 | 0.381063 | 3 | 141275436 | 0.0035 | 3.20E−26 | 692316 | TRUE |
| rs16871902 | 0.0094137 | 184305 | 0.558406 | 5 | 3488462 | 0.0017 | 4.60E−13 | 690830 | TRUE |
| rs16903285 | 0.0128996 | 184305 | 0.0159522 | 5 | 87978252 | 0.0026 | 7.60E−38 | 687944 | TRUE |
| rs16953563 | 0.0106238 | 184305 | 0.0715319 | 15 | 66686770 | 0.002 | 1.50E−11 | 691761 | TRUE |
| rs17001561 | 0.0136556 | 184305 | 0.244576 | 4 | 77096118 | 0.0023 | 3.80E−11 | 794327 | TRUE |
| rs17014375 | 0.0129919 | 184305 | 0.562813 | 1 | 209543560 | 0.0025 | 1.10E−11 | 690856 | TRUE |
| rs17033117 | 0.011268 | 184305 | 0.379476 | 3 | 35443653 | 0.0022 | 8.90E−10 | 691863 | TRUE |
| rs17056301 | 0.0101784 | 184305 | 0.197193 | 5 | 158271680 | 0.002 | 2.40E−09 | 688085 | TRUE |
| rs17113297 | 0.0116167 | 184305 | 0.95003 | 10 | 102395982 | 0.0021 | 2.10E−15 | 686357 | TRUE |
| rs17119937 | 0.0213193 | 184305 | 0.483122 | 8 | 14502274 | 0.0036 | 5.60E−09 | 668272 | TRUE |
| rs17203016 | 0.0122315 | 184305 | 0.0961324 | 2 | 208255518 | 0.002 | 2.10E−13 | 786272 | TRUE |
| rs17207196 | 0.01108 | 184305 | 0.177191 | 7 | 75101065 | 0.0018 | 2.10E−35 | 668894 | TRUE |
| rs17238110 | 0.019415 | 184305 | 0.661528 | 15 | 62150364 | 0.005 | 2.00E−12 | 775505 | TRUE |
| rs17311369 | 0.0098949 | 184305 | 0.997339 | 15 | 47709199 | 0.0019 | 3.10E−08 | 673102 | TRUE |
| rs17399237 | 0.0099709 | 184305 | 0.0346059 | 2 | 35471626 | 0.0017 | 6.70E−14 | 690950 | TRUE |
| rs17405819 | 0.0098786 | 184305 | 0.244494 | 8 | 76806584 | 0.0018 | 4.30E−33 | 795493 | TRUE |
| rs17424296 | 0.01006 | 184305 | 0.109643 | 5 | 60838903 | 0.0018 | 2.40E−09 | 684366 | TRUE |
| rs17425707 | 0.0163702 | 184305 | 0.583058 | 1 | 57874879 | 0.0028 | 4.40E−09 | 688867 | TRUE |
| rs17446257 | 0.0151916 | 184305 | 0.306308 | 13 | 40749213 | 0.0026 | 2.90E−09 | 690630 | TRUE |
| rs17499593 | 0.0130429 | 184305 | 0.558451 | 2 | 172649755 | 0.0022 | 1.10E−08 | 691663 | TRUE |
| rs17513613 | 0.0104359 | 184305 | 0.0311494 | 19 | 30286822 | 0.0018 | 3.60E−26 | 789575 | TRUE |
| rs175165 | 0.0096516 | 184305 | 0.520759 | 22 | 20116015 | 0.0018 | 5.20E−09 | 690545 | TRUE |
| rs17535749 | 0.0174576 | 184305 | 0.0614016 | 3 | 10027724 | 0.0027 | 2.50E−08 | 777038 | TRUE |
| rs17551974 | 0.0115069 | 184305 | 0.59941 | 2 | 142293146 | 0.0022 | 1.90E−10 | 691115 | TRUE |
| rs17636031 | 0.0122347 | 184305 | 0.83018 | 10 | 126594078 | 0.0019 | 1.20E−17 | 782807 | TRUE |
| rs17663412 | 0.014 | 184305 | 0.876877 | 5 | 167595121 | 0.0027 | 6.10E−09 | 691018 | TRUE |
| rs17710386 | 0.0101806 | 184305 | 0.576025 | 18 | 63461201 | 0.0018 | 1.00E−12 | 783810 | TRUE |
| rs17724992 | 0.0102186 | 184305 | 0.173778 | 19 | 18454825 | 0.0019 | 1.00E−22 | 785851 | TRUE |
| rs17789218 | 0.0122304 | 184305 | 0.814281 | 6 | 100600097 | 0.0019 | 7.40E−12 | 793904 | TRUE |
| rs17806379 | 0.01225 | 184305 | 0.623064 | 20 | 51107290 | 0.0022 | 1.50E−30 | 690043 | TRUE |
| rs1784460 | 0.0097211 | 184305 | 0.0656448 | 11 | 118938371 | 0.0018 | 9.00E−14 | 680042 | TRUE |
| rs1804528 | 0.0102219 | 184305 | 0.164511 | 4 | 146056320 | 0.002 | 3.00E−08 | 518856 | TRUE |
| rs1830074 | 0.0103653 | 184305 | 0.948385 | 7 | 6718674 | 0.0019 | 1.40E−09 | 689911 | TRUE |
| rs1836303 | 0.0096157 | 184305 | 0.98183 | 15 | 46539116 | 0.0018 | 5.30E−11 | 688991 | TRUE |
| rs1843328 | 0.0092757 | 184305 | 0.245866 | 12 | 17111188 | 0.0017 | 7.90E−09 | 686814 | TRUE |
| rs1863652 | 0.0096777 | 184305 | 0.00616283 | 4 | 95991417 | 0.0018 | 1.40E−10 | 692539 | TRUE |
| rs1884389 | 0.0093257 | 184305 | 0.0789587 | 20 | 1410582 | 0.0017 | 4.00E−09 | 683669 | TRUE |
| rs1885728 | 0.0101773 | 184305 | 0.127417 | 6 | 5977833 | 0.0019 | 1.00E−08 | 682316 | TRUE |
| rs1891216 | 0.0100568 | 184305 | 0.539009 | 1 | 7728391 | 0.0018 | 2.40E−09 | 685079 | TRUE |
| rs1896767 | 0.0092344 | 184305 | 0.509652 | 16 | 62838304 | 0.0017 | 2.40E−10 | 686262 | TRUE |
| rs189843 | 0.0094169 | 184305 | 0.747393 | 5 | 164600151 | 0.0017 | 1.70E−08 | 685314 | FALSE |
| rs1927790 | 0.0092247 | 184305 | 0.43918 | 13 | 96922191 | 0.0016 | 1.80E−19 | 794326 | TRUE |
| rs1928295 | 0.0092095 | 184305 | 0.468643 | 9 | 120378483 | 0.0016 | 5.40E−18 | 793649 | TRUE |
| rs1937683 | 0.0096146 | 184305 | 0.501587 | 10 | 53679060 | 0.0018 | 3.20E−09 | 692539 | TRUE |
| rs1948080 | 0.0098178 | 184305 | 0.260244 | 9 | 11852043 | 0.0018 | 1.10E−14 | 690633 | TRUE |
| rs1982441 | 0.0141944 | 184305 | 0.621557 | 8 | 28021769 | 0.0026 | 7.00E−12 | 687705 | TRUE |
| rs1982725 | 0.0094962 | 184305 | 0.485927 | 19 | 30618771 | 0.0017 | 3.30E−08 | 683155 | TRUE |
| rs1993709 | 0.0133547 | 184305 | 0.0105623 | 1 | 72838529 | 0.0021 | 1.90E−57 | 786001 | TRUE |
| rs2007231 | 0.010374 | 184305 | 0.245727 | 1 | 115266306 | 0.0018 | 5.20E−09 | 691969 | TRUE |
| rs200810 | 0.0094006 | 184305 | 0.037005 | 6 | 97922184 | 0.0017 | 5.50E−16 | 793699 | TRUE |
| rs2009416 | 0.0096874 | 184305 | 0.267001 | 5 | 92415111 | 0.0018 | 1.10E−11 | 691741 | TRUE |
| rs2033529 | 0.0104609 | 184305 | 0.547202 | 6 | 40348653 | 0.0018 | 1.90E−30 | 792112 | TRUE |
| rs2051559 | 0.0133905 | 184305 | 0.408406 | 4 | 3298800 | 0.0026 | 5.00E−12 | 689307 | TRUE |
| rs2065418 | 0.0096907 | 184305 | 0.655004 | 11 | 30422068 | 0.0018 | 3.60E−20 | 691707 | TRUE |
| rs208015 | 0.0159868 | 184305 | 0.182991 | 17 | 46252346 | 0.0034 | 1.40E−25 | 691575 | TRUE |
| rs2124499 | 0.0098688 | 184305 | 0.929591 | 3 | 123093541 | 0.0017 | 3.40E−13 | 785955 | TRUE |
| rs2143253 | 0.0124824 | 184305 | 0.342572 | 20 | 41987392 | 0.0026 | 1.10E−12 | 684760 | TRUE |
| rs215634 | 0.0094376 | 184305 | 0.040927 | 7 | 32369148 | 0.0018 | 2.60E−17 | 681296 | TRUE |
| rs2162524 | 0.0101697 | 184305 | 0.523685 | 2 | 230817437 | 0.0018 | 4.10E−17 | 691302 | TRUE |
| rs2163188 | 0.0091606 | 184305 | 0.475204 | 10 | 65314711 | 0.0017 | 2.00E−14 | 686502 | FALSE |
| rs2174307 | 0.0092431 | 184305 | 0.00759049 | 9 | 73791849 | 0.0017 | 4.90E−12 | 686559 | FALSE |
| rs217671 | 0.0101784 | 184305 | 0.419383 | 14 | 62360464 | 0.0019 | 1.30E−13 | 691456 | TRUE |
| rs2228213 | 0.0098449 | 184305 | 0.569203 | 6 | 12124855 | 0.0017 | 4.60E−16 | 795595 | TRUE |
| rs2235564 | 0.0097504 | 184305 | 0.7824 | 1 | 6713114 | 0.0018 | 3.70E−13 | 691544 | TRUE |
| rs2246012 | 0.0117839 | 184305 | 0.0679595 | 6 | 131898208 | 0.0022 | 3.10E−13 | 795598 | TRUE |
| rs226000 | 0.0119295 | 184305 | 0.242863 | 14 | 30488699 | 0.0022 | 3.60E−08 | 793480 | TRUE |
| rs2283093 | 0.0115287 | 184305 | 0.71634 | 7 | 126721231 | 0.0021 | 3.10E−09 | 691773 | TRUE |
| rs2284746 | 0.0094452 | 184305 | 0.0454015 | 1 | 17306675 | 0.0017 | 1.40E−09 | 692206 | FALSE |
| rs2285178 | 0.0099373 | 184305 | 0.369382 | 22 | 38205989 | 0.0019 | 9.40E−09 | 638268 | TRUE |
| rs2306537 | 0.0108269 | 184305 | 0.786256 | 12 | 133423695 | 0.0019 | 8.70E−13 | 691923 | TRUE |
| rs2307111 | 0.0095245 | 184305 | 0.42912 | 5 | 75003678 | 0.0016 | 1.60E−58 | 795430 | TRUE |
| rs2317299 | 0.0097113 | 184305 | 0.195753 | 2 | 236903093 | 0.0017 | 1.30E−09 | 677983 | TRUE |
| rs2325036 | 0.0094441 | 184305 | 0.879062 | 3 | 85819412 | 0.0017 | 3.60E−27 | 790870 | TRUE |
| rs2357760 | 0.0097233 | 184305 | 0.0875467 | 6 | 120213880 | 0.0017 | 6.80E−17 | 791053 | TRUE |
| rs2361988 | 0.0108563 | 184305 | 0.400147 | 16 | 398151 | 0.002 | 5.20E−15 | 690251 | TRUE |
| rs2365389 | 0.0096038 | 184305 | 0.11893 | 3 | 61236462 | 0.0017 | 1.30E−25 | 783625 | TRUE |
| rs2367112 | 0.0094973 | 184305 | 0.0543263 | 5 | 64168193 | 0.0016 | 2.30E−13 | 794305 | TRUE |
| rs2411182 | 0.0101078 | 184305 | 0.79052 | 17 | 35059718 | 0.0019 | 7.70E−11 | 691730 | TRUE |
| rs2423668 | 0.0093941 | 184305 | 0.506875 | 20 | 12430673 | 0.0019 | 2.80E−08 | 527352 | TRUE |
| rs2425840 | 0.0096125 | 184305 | 0.712128 | 20 | 44904838 | 0.0018 | 1.60E−11 | 681680 | TRUE |
| rs2429150 | 0.0094419 | 184305 | 0.128637 | 12 | 2152655 | 0.0018 | 2.70E−10 | 686276 | TRUE |
| rs2479958 | 0.0097493 | 184305 | 0.112401 | 13 | 111984244 | 0.0018 | 1.50E−17 | 664268 | TRUE |
| rs2481665 | 0.0098927 | 184305 | 0.0126689 | 1 | 62594677 | 0.0016 | 7.20E−23 | 795247 | TRUE |
| rs2543132 | 0.0110746 | 184305 | 0.6343 | 8 | 15536311 | 0.0022 | 5.00E−11 | 688326 | TRUE |
| rs2600226 | 0.009606 | 184305 | 0.0273458 | 3 | 12928762 | 0.0019 | 3.70E−10 | 685285 | TRUE |
| rs2605603 | 0.0094343 | 184305 | 0.731756 | 11 | 93221105 | 0.0016 | 2.50E−10 | 790857 | TRUE |
| rs2608703 | 0.0091856 | 184305 | 0.0586665 | 12 | 41846769 | 0.0017 | 1.90E−16 | 686700 | TRUE |
| rs262130 | 0.0115493 | 184305 | 0.848588 | 6 | 142853486 | 0.0023 | 1.80E−08 | 679542 | TRUE |
| rs2643452 | 0.0092344 | 184305 | 0.21067 | 4 | 18529220 | 0.0017 | 4.70E−15 | 690573 | FALSE |
| rs2693826 | 0.0092453 | 184305 | 0.0124888 | 2 | 6160943 | 0.0017 | 2.00E−15 | 690808 | TRUE |
| rs2694047 | 0.0107346 | 184305 | 0.414843 | 8 | 116750548 | 0.002 | 3.90E−21 | 690295 | TRUE |
| rs273504 | 0.0098308 | 184305 | 0.33037 | 19 | 18215247 | 0.0018 | 4.40E−18 | 690672 | TRUE |
| rs2744974 | 0.0099492 | 184305 | 0.0139252 | 6 | 34579431 | 0.0018 | 1.40E−45 | 789647 | TRUE |
| rs2791653 | 0.011331 | 184305 | 0.60307 | 1 | 11129848 | 0.0019 | 1.30E−13 | 795271 | TRUE |
| rs2820311 | 0.0100513 | 184305 | 3.64E−05 | 1 | 201841476 | 0.0018 | 4.10E−38 | 691876 | TRUE |
| rs2832283 | 0.0112962 | 184305 | 0.131117 | 21 | 30690558 | 0.002 | 5.80E−09 | 792324 | TRUE |
| rs2836964 | 0.0102197 | 184305 | 0.597635 | 21 | 40631006 | 0.0018 | 1.30E−09 | 692353 | TRUE |
| rs2861683 | 0.0095267 | 184305 | 0.76642 | 2 | 67836507 | 0.0017 | 1.30E−16 | 691163 | TRUE |
| rs2868975 | 0.0115808 | 184305 | 0.555056 | 3 | 116935323 | 0.0023 | 2.20E−10 | 690613 | TRUE |
| rs287104 | 0.0097189 | 184305 | 0.290648 | 19 | 34290995 | 0.0017 | 4.40E−11 | 787307 | TRUE |
| rs2875762 | 0.0108812 | 184305 | 0.750291 | 6 | 124925032 | 0.002 | 1.20E−11 | 685199 | TRUE |
| rs2907948 | 0.0114711 | 184305 | 0.159578 | 7 | 150638484 | 0.0019 | 1.30E−13 | 794299 | TRUE |
| rs2931434 | 0.0104609 | 184305 | 0.356474 | 5 | 73159098 | 0.0018 | 1.40E−08 | 690664 | TRUE |
| rs2943465 | 0.0157326 | 184305 | 0.26251 | 12 | 19265921 | 0.0039 | 2.00E−10 | 686547 | TRUE |
| rs294704 | 0.0108258 | 184305 | 0.146351 | 5 | 152519088 | 0.0019 | 4.00E−09 | 690036 | TRUE |
| rs3007105 | 0.0092996 | 184305 | 0.5437 | 14 | 47367616 | 0.0017 | 1.10E−17 | 785488 | TRUE |
| rs326896 | 0.0093072 | 184305 | 0.138865 | 4 | 112669571 | 0.0018 | 2.80E−13 | 690324 | TRUE |
| rs331966 | 0.0096005 | 184305 | 0.132104 | 4 | 143675717 | 0.0018 | 3.20E−10 | 687189 | TRUE |
| rs33500 | 0.0121848 | 184305 | 0.706092 | 3 | 42427191 | 0.0022 | 4.30E−14 | 689500 | TRUE |
| rs339991 | 0.0092573 | 184305 | 0.899682 | 15 | 60913637 | 0.0018 | 1.20E−12 | 686476 | TRUE |
| rs349088 | 0.0093333 | 184305 | 0.810247 | 11 | 84814393 | 0.0017 | 1.80E−13 | 684401 | TRUE |
| rs355777 | 0.0092909 | 184305 | 0.094938 | 3 | 154034950 | 0.0017 | 1.40E−18 | 689978 | TRUE |
| rs3731695 | 0.0096451 | 184305 | 5.67E−05 | 2 | 203820275 | 0.0016 | 7.90E−13 | 793581 | TRUE |
| rs3732084 | 0.0095158 | 184305 | 0.934086 | 2 | 207174316 | 0.0018 | 1.10E−09 | 691763 | TRUE |
| rs3736485 | 0.0095777 | 184305 | 0.868067 | 15 | 51748610 | 0.0016 | 2.50E−16 | 790404 | TRUE |
| rs3749897 | 0.0092812 | 184305 | 0.652595 | 6 | 42532102 | 0.0018 | 8.40E−12 | 638224 | TRUE |
| rs3754963 | 0.0107737 | 184305 | 0.0818634 | 2 | 166185707 | 0.002 | 3.30E−10 | 691535 | TRUE |
| rs3764835 | 0.0138251 | 184305 | 0.578693 | 2 | 159519368 | 0.0024 | 3.10E−09 | 689505 | TRUE |
| rs3772882 | 0.0096538 | 184305 | 0.0017519 | 3 | 81808602 | 0.0018 | 6.60E−13 | 691912 | TRUE |
| rs3800229 | 0.009884 | 184305 | 0.693753 | 6 | 108996963 | 0.0018 | 1.40E−22 | 792474 | TRUE |
| rs3800637 | 0.0095223 | 184305 | 0.982573 | 7 | 137403432 | 0.0018 | 5.10E−10 | 682001 | TRUE |
| rs3806114 | 0.0107889 | 184305 | 0.394428 | 6 | 20482335 | 0.0018 | 3.40E−10 | 778855 | TRUE |
| rs3806572 | 0.0110062 | 184305 | 0.0692484 | 2 | 55238677 | 0.0019 | 1.60E−14 | 687646 | TRUE |
| rs3807645 | 0.0110572 | 184305 | 0.419728 | 7 | 77830091 | 0.0021 | 2.40E−15 | 683086 | TRUE |
| rs380857 | 0.0140217 | 184305 | 0.24018 | 9 | 101491066 | 0.0027 | 3.60E−08 | 691507 | TRUE |
| rs3814883 | 0.0099297 | 184305 | 0.328334 | 16 | 29994922 | 0.0017 | 1.10E−40 | 685519 | TRUE |
| rs3828783 | 0.0119827 | 184305 | 0.408981 | 6 | 33767727 | 0.0021 | 5.60E−15 | 792749 | TRUE |
| rs3829849 | 0.0100285 | 184305 | 0.668666 | 9 | 129390800 | 0.0017 | 5.90E−09 | 793851 | TRUE |
| rs38314 | 0.0097493 | 184305 | 0.877157 | 7 | 70067315 | 0.0017 | 4.70E−12 | 689782 | TRUE |
| rs3844598 | 0.0092942 | 184305 | 0.0135997 | 5 | 140992235 | 0.0017 | 3.80E−08 | 690704 | TRUE |
| rs3902951 | 0.0105141 | 184305 | 0.679141 | 14 | 69789755 | 0.002 | 7.00E−12 | 773819 | TRUE |
| rs3904244 | 0.0114917 | 184305 | 0.656058 | 10 | 27361527 | 0.0025 | 4.30E−10 | 691180 | TRUE |
| rs391300 | 0.0097776 | 184305 | 0.0005745 | 17 | 2216258 | 0.0017 | 3.10E−12 | 791120 | TRUE |
| rs3935648 | 0.0123401 | 184305 | 0.00297112 | 17 | 79085335 | 0.0022 | 6.80E−09 | 629303 | TRUE |
| rs3977755 | 0.0101448 | 184305 | 0.0672048 | 10 | 104420210 | 0.0019 | 5.90E−13 | 731529 | TRUE |
| rs40067 | 0.0115189 | 184305 | 0.000741993 | 5 | 107439012 | 0.0023 | 7.10E−30 | 681695 | TRUE |
| rs4012234 | 0.0095875 | 184305 | 0.6867 | 20 | 32553047 | 0.0018 | 9.90E−16 | 689653 | TRUE |
| rs4072917 | 0.009833 | 184305 | 0.270946 | 8 | 143300279 | 0.0018 | 6.90E−11 | 684720 | TRUE |
| rs4148155 | 0.0148874 | 184305 | 0.110015 | 4 | 89054667 | 0.0026 | 5.00E−13 | 794889 | TRUE |
| rs4148866 | 0.0100035 | 184305 | 0.778097 | 12 | 123425575 | 0.0018 | 4.00E−08 | 676418 | TRUE |
| rs4237643 | 0.0102773 | 184305 | 0.0059888 | 11 | 43648368 | 0.0019 | 4.30E−33 | 692491 | TRUE |
| rs427943 | 0.0094148 | 184305 | 0.0173332 | 21 | 46570896 | 0.0017 | 7.30E−23 | 712095 | TRUE |
| rs429343 | 0.0097168 | 184305 | 0.268893 | 2 | 147903382 | 0.0017 | 6.80E−18 | 689345 | TRUE |
| rs4307239 | 0.009418 | 184305 | 0.855339 | 7 | 24354300 | 0.0017 | 3.90E−11 | 687289 | TRUE |
| rs4310573 | 0.0111289 | 184305 | 0.247499 | 11 | 97855562 | 0.0021 | 3.50E−08 | 682567 | TRUE |
| rs4358081 | 0.0092247 | 184305 | 0.257376 | 2 | 29100642 | 0.0017 | 1.50E−08 | 690038 | TRUE |
| rs4414033 | 0.0097559 | 184305 | 0.261561 | 1 | 156406853 | 0.0018 | 1.40E−12 | 672697 | TRUE |
| rs4430672 | 0.0112614 | 184305 | 0.845531 | 14 | 63094407 | 0.0022 | 3.90E−09 | 691400 | TRUE |
| rs4482463 | 0.0161258 | 184305 | 0.863859 | 2 | 205375909 | 0.0033 | 2.80E−23 | 635414 | TRUE |
| rs4495304 | 0.016003 | 184305 | 0.635788 | 6 | 31080718 | 0.0033 | 5.00E−09 | 774211 | TRUE |
| rs4516268 | 0.0128583 | 184305 | 0.570747 | 17 | 1846831 | 0.0021 | 5.20E−25 | 786617 | TRUE |
| rs4518345 | 0.0105966 | 184305 | 0.634748 | 5 | 27185904 | 0.0019 | 1.00E−09 | 688609 | TRUE |
| rs4556997 | 0.0136795 | 184305 | 0.606806 | 2 | 100814858 | 0.0024 | 6.90E−17 | 792972 | TRUE |
| rs4589691 | 0.0126682 | 184305 | 0.0350211 | 2 | 144051398 | 0.0024 | 4.70E−09 | 688253 | TRUE |
| rs4639527 | 0.0101882 | 184305 | 0.85853 | 2 | 416815 | 0.0019 | 3.30E−20 | 691706 | TRUE |
| rs4653017 | 0.0099872 | 184305 | 0.795689 | 1 | 33776728 | 0.0018 | 4.50E−11 | 686378 | TRUE |
| rs4660443 | 0.0118307 | 184305 | 0.0670239 | 1 | 39591779 | 0.0021 | 6.80E−15 | 687234 | TRUE |
| rs4671328 | 0.0095962 | 184305 | 0.67886 | 2 | 58935282 | 0.0017 | 2.20E−36 | 679487 | TRUE |
| rs4722398 | 0.0138859 | 184305 | 0.736634 | 7 | 3125220 | 0.0025 | 3.60E−10 | 692509 | TRUE |
| rs4740619 | 0.0092497 | 184305 | 0.479131 | 9 | 15634326 | 0.0016 | 2.30E−30 | 794491 | TRUE |
| rs4757144 | 0.0093887 | 184305 | 0.000150598 | 11 | 13331226 | 0.0018 | 5.60E−22 | 690082 | TRUE |
| rs4783830 | 0.0102892 | 184305 | 0.956286 | 16 | 54255346 | 0.0019 | 2.40E−08 | 675527 | TRUE |
| rs4786903 | 0.0108313 | 184305 | 0.73258 | 16 | 6697104 | 0.002 | 3.50E−10 | 680139 | TRUE |
| rs4800191 | 0.009909 | 184305 | 0.889638 | 18 | 22461398 | 0.0017 | 2.50E−09 | 785353 | TRUE |
| rs4813619 | 0.0097146 | 184305 | 0.519922 | 20 | 2815715 | 0.0018 | 2.30E−09 | 622760 | TRUE |
| rs4818225 | 0.0103653 | 184305 | 0.10656 | 21 | 42629895 | 0.0018 | 2.30E−10 | 688274 | TRUE |
| rs4820408 | 0.0095288 | 184305 | 0.021749 | 22 | 40604945 | 0.0017 | 2.10E−19 | 794185 | TRUE |
| rs4842491 | 0.0106314 | 184305 | 0.0733517 | 12 | 89905537 | 0.0018 | 4.00E−08 | 795312 | TRUE |
| rs4851029 | 0.0099557 | 184305 | 0.826674 | 2 | 104159785 | 0.0017 | 1.70E−12 | 689752 | TRUE |
| rs4858193 | 0.0113331 | 184305 | 0.0466874 | 3 | 20441050 | 0.0019 | 1.60E−11 | 686850 | TRUE |
| rs486359 | 0.0091986 | 184305 | 0.000242298 | 6 | 160774441 | 0.0017 | 1.60E−11 | 770999 | FALSE |
| rs4864201 | 0.0094702 | 184305 | 0.033002 | 4 | 130731284 | 0.0017 | 1.50E−16 | 795263 | TRUE |
| rs4880341 | 0.0097656 | 184305 | 0.352267 | 10 | 133992689 | 0.0017 | 1.10E−11 | 689012 | TRUE |
| rs4906908 | 0.0093159 | 184305 | 0.560268 | 15 | 27040082 | 0.0017 | 2.50E−09 | 691345 | TRUE |
| rs491711 | 0.0101849 | 184305 | 0.573443 | 11 | 28742220 | 0.0019 | 1.10E−09 | 685113 | TRUE |
| rs4929923 | 0.0095245 | 184305 | 0.0918629 | 11 | 8639200 | 0.0017 | 7.20E−27 | 794933 | TRUE |
| rs4936175 | 0.0100253 | 184305 | 0.956964 | 11 | 132641959 | 0.0017 | 1.40E−12 | 692569 | TRUE |
| rs4937870 | 0.0098601 | 184305 | 0.253466 | 11 | 112826709 | 0.0019 | 8.80E−09 | 683154 | TRUE |
| rs4952843 | 0.0098873 | 184305 | 0.0951546 | 2 | 46957845 | 0.0018 | 6.80E−14 | 692482 | TRUE |
| rs4954638 | 0.0103794 | 184305 | 0.550282 | 2 | 137435455 | 0.002 | 2.90E−09 | 689971 | TRUE |
| rs4968656 | 0.0099862 | 184305 | 0.182846 | 17 | 61616959 | 0.0019 | 8.20E−10 | 675153 | TRUE |
| rs4981693 | 0.0106184 | 184305 | 0.00433501 | 14 | 29680331 | 0.002 | 6.90E−24 | 689120 | TRUE |
| rs4986044 | 0.0097005 | 184305 | 0.0927321 | 17 | 21261560 | 0.0016 | 3.30E−23 | 787219 | TRUE |
| rs538579 | 0.010223 | 184305 | 0.195413 | 3 | 62711674 | 0.0019 | 1.30E−13 | 688452 | TRUE |
| rs543874 | 0.0117296 | 184305 | 0.517029 | 1 | 177889480 | 0.002 | 1.20E−122 | 795504 | TRUE |
| rs559231 | 0.0094202 | 184305 | 0.213372 | 18 | 39644247 | 0.0018 | 2.40E−14 | 685154 | TRUE |
| rs577525 | 0.0093474 | 184305 | 0.0556878 | 10 | 99769388 | 0.0017 | 9.70E−22 | 690616 | TRUE |
| rs592483 | 0.0102012 | 184305 | 0.0235234 | 11 | 69445173 | 0.0017 | 2.00E−18 | 781871 | TRUE |
| rs6011457 | 0.0094039 | 184305 | 0.568261 | 20 | 61530915 | 0.0017 | 2.70E−11 | 690692 | FALSE |
| rs6050446 | 0.031992 | 184305 | 0.285386 | 20 | 25195509 | 0.0047 | 4.40E−13 | 766287 | TRUE |
| rs6235 | 0.0104837 | 184305 | 0.954057 | 5 | 95728898 | 0.0019 | 1.50E−19 | 691708 | TRUE |
| rs6265 | 0.0115243 | 184305 | 0.00827199 | 11 | 27679916 | 0.0021 | 1.00E−86 | 795458 | TRUE |
| rs6443750 | 0.0149222 | 184305 | 0.20221 | 3 | 181329682 | 0.0021 | 3.20E−12 | 776837 | TRUE |
| rs6448587 | 0.0110007 | 184305 | 0.326401 | 4 | 28561990 | 0.0023 | 2.30E−13 | 691097 | TRUE |
| rs645040 | 0.0112484 | 184305 | 0.000603004 | 3 | 135926622 | 0.002 | 2.50E−18 | 795579 | TRUE |
| rs6461115 | 0.010576 | 184305 | 0.0325822 | 7 | 2103668 | 0.0019 | 1.20E−13 | 791735 | TRUE |
| rs6471941 | 0.0110974 | 184305 | 0.969954 | 8 | 62117973 | 0.0021 | 3.10E−13 | 793986 | TRUE |
| rs6500208 | 0.0110898 | 184305 | 0.947444 | 16 | 49011249 | 0.002 | 4.10E−12 | 781931 | TRUE |
| rs6512302 | 0.0117666 | 184305 | 0.480991 | 20 | 62691550 | 0.002 | 2.10E−12 | 686053 | TRUE |
| rs6545714 | 0.0097493 | 184305 | 0.287575 | 2 | 59307725 | 0.0017 | 9.10E−31 | 793368 | TRUE |
| rs6556301 | 0.0100774 | 184305 | 0.14904 | 5 | 176527577 | 0.0018 | 4.10E−10 | 734744 | TRUE |
| rs6561943 | 0.0113125 | 184305 | 0.97694 | 13 | 58356761 | 0.0019 | 4.20E−10 | 793951 | TRUE |
| rs657452 | 0.0093844 | 184305 | 0.413747 | 1 | 49589847 | 0.0017 | 7.20E−29 | 767846 | TRUE |
| rs6587552 | 0.0108324 | 184305 | 0.238007 | 1 | 151018861 | 0.002 | 1.60E−17 | 689723 | TRUE |
| rs6591407 | 0.0126171 | 184305 | 0.438961 | 11 | 56914157 | 0.0021 | 1.90E−08 | 794246 | TRUE |
| rs6593688 | 0.009544 | 184305 | 0.246907 | 1 | 96322205 | 0.0018 | 8.60E−15 | 691779 | TRUE |
| rs6595205 | 0.0095745 | 184305 | 0.913668 | 5 | 119372533 | 0.0016 | 2.00E−12 | 794525 | FALSE |
| rs663129 | 0.0105173 | 184305 | 3.20E−08 | 18 | 57838401 | 0.0019 | 1.60E−178 | 788948 | TRUE |
| rs6673081 | 0.0100361 | 184305 | 5.28E−05 | 1 | 154989595 | 0.0018 | 1.80E−08 | 677818 | TRUE |
| rs6692586 | 0.0113603 | 184305 | 0.441427 | 1 | 23299906 | 0.0023 | 1.10E−16 | 690921 | TRUE |
| rs6712 | 0.0140575 | 184305 | 0.932481 | 22 | 50637922 | 0.0025 | 4.40E−08 | 688469 | TRUE |
| rs6764533 | 0.0100926 | 184305 | 0.227004 | 3 | 196088464 | 0.0018 | 1.40E−10 | 690832 | TRUE |
| rs6772756 | 0.0106618 | 184305 | 0.172412 | 3 | 182312152 | 0.0019 | 4.00E−08 | 681709 | TRUE |
| rs6785245 | 0.0096396 | 184305 | 0.728509 | 3 | 82647990 | 0.0017 | 4.00E−14 | 692250 | TRUE |
| rs6804842 | 0.0092649 | 184305 | 0.283329 | 3 | 25106437 | 0.0017 | 3.60E−21 | 789179 | TRUE |
| rs6815910 | 0.0092029 | 184305 | 0.101024 | 4 | 55495793 | 0.0017 | 1.40E−13 | 689263 | FALSE |
| rs6841761 | 0.0094267 | 184305 | 0.830656 | 4 | 25423538 | 0.0016 | 6.40E−16 | 793477 | TRUE |
| rs685870 | 0.0102523 | 184305 | 0.24142 | 11 | 64111928 | 0.0019 | 2.40E−10 | 688423 | TRUE |
| rs6985109 | 0.009846 | 184305 | 0.220131 | 8 | 10761585 | 0.0017 | 1.50E−26 | 793993 | TRUE |
| rs7024334 | 0.0111778 | 184305 | 0.690812 | 9 | 109072075 | 0.002 | 3.10E−12 | 782431 | TRUE |
| rs7025938 | 0.0098601 | 184305 | 0.370128 | 9 | 103088321 | 0.0019 | 3.70E−19 | 691581 | TRUE |
| rs7037266 | 0.0094148 | 184305 | 0.237175 | 9 | 6942940 | 0.0018 | 3.50E−10 | 691603 | TRUE |
| rs705217 | 0.0095853 | 184305 | 0.933734 | 1 | 34581472 | 0.0018 | 9.30E−09 | 688609 | TRUE |
| rs705704 | 0.0102436 | 184305 | 0.828959 | 12 | 56435412 | 0.0018 | 1.90E−13 | 743597 | TRUE |
| rs7084454 | 0.0104652 | 184305 | 0.740065 | 10 | 21821274 | 0.0019 | 4.00E−25 | 678564 | TRUE |
| rs709400 | 0.0100003 | 184305 | 0.142034 | 14 | 104149475 | 0.0017 | 4.60E−19 | 795379 | TRUE |
| rs7102454 | 0.0103664 | 184305 | 0.93419 | 11 | 65594820 | 0.0018 | 2.40E−18 | 691134 | TRUE |
| rs7117238 | 0.0114135 | 184305 | 0.0115904 | 11 | 78040259 | 0.0022 | 2.50E−09 | 788879 | TRUE |
| rs7124681 | 0.0096146 | 184305 | 0.943285 | 11 | 47529947 | 0.0016 | 3.20E−58 | 795474 | TRUE |
| rs7138803 | 0.0094745 | 184305 | 0.388225 | 12 | 50247468 | 0.0017 | 2.30E−71 | 795588 | TRUE |
| rs7144011 | 0.0120707 | 184305 | 0.0759189 | 14 | 79940383 | 0.002 | 5.20E−47 | 794117 | TRUE |
| rs7148846 | 0.0108454 | 184305 | 0.4256 | 14 | 40133821 | 0.0022 | 2.20E−08 | 687940 | TRUE |
| rs7172627 | 0.0094463 | 184305 | 0.837695 | 15 | 31877690 | 0.0017 | 1.10E−11 | 690458 | TRUE |
| rs7181498 | 0.0096592 | 184305 | 0.673039 | 15 | 95271404 | 0.0018 | 1.00E−19 | 690980 | TRUE |
| rs7196720 | 0.0094799 | 184305 | 0.600901 | 16 | 24534662 | 0.0017 | 7.30E−14 | 689863 | TRUE |
| rs7206608 | 0.0104315 | 184305 | 0.94276 | 16 | 82872628 | 0.0019 | 1.30E−12 | 689058 | TRUE |
| rs7222349 | 0.0098504 | 184305 | 0.755837 | 17 | 42304644 | 0.0018 | 3.30E−10 | 692215 | TRUE |
| rs7239575 | 0.0091747 | 184305 | 0.440171 | 18 | 21120035 | 0.0017 | 7.40E−32 | 692313 | TRUE |
| rs7318817 | 0.0097081 | 184305 | 0.450595 | 13 | 28617708 | 0.0018 | 2.70E−18 | 691917 | TRUE |
| rs7334078 | 0.0104141 | 184305 | 0.81468 | 13 | 99120484 | 0.0019 | 2.20E−10 | 688374 | TRUE |
| rs7358465 | 0.0099351 | 184305 | 0.412261 | 11 | 89990280 | 0.0019 | 3.00E−08 | 686935 | TRUE |
| rs7488867 | 0.0100231 | 184305 | 0.555834 | 12 | 103699685 | 0.002 | 8.40E−24 | 635746 | TRUE |
| rs7498665 | 0.0100937 | 184305 | 0.0599612 | 16 | 28883241 | 0.0017 | 5.60E−60 | 790299 | TRUE |
| rs7519259 | 0.009405 | 184305 | 0.0369769 | 1 | 66434743 | 0.0017 | 3.80E−13 | 681362 | TRUE |
| rs7535528 | 0.0102371 | 184305 | 0.168919 | 1 | 2444414 | 0.0018 | 1.40E−16 | 632868 | TRUE |
| rs754635 | 0.0134405 | 184305 | 0.668789 | 3 | 42305131 | 0.0027 | 2.20E−13 | 690346 | TRUE |
| rs7550711 | 0.0305635 | 184305 | 0.113631 | 1 | 110082886 | 0.005 | 3.20E−38 | 769184 | TRUE |
| rs7551507 | 0.0094419 | 184305 | 0.831668 | 1 | 74995225 | 0.0016 | 9.30E−30 | 794579 | TRUE |
| rs7557796 | 0.0099166 | 184305 | 0.0117184 | 2 | 86766153 | 0.0018 | 2.30E−19 | 692414 | TRUE |
| rs756717 | 0.0099318 | 184305 | 0.0149169 | 16 | 72996162 | 0.0017 | 5.40E−18 | 771976 | TRUE |
| rs7599312 | 0.011193 | 184305 | 0.0172556 | 2 | 213413231 | 0.0019 | 6.90E−24 | 780823 | TRUE |
| rs7615297 | 0.0137686 | 184305 | 0.192983 | 3 | 156299313 | 0.0024 | 5.70E−10 | 689710 | TRUE |
| rs7626079 | 0.0096353 | 184305 | 0.82464 | 3 | 66427259 | 0.0018 | 1.60E−09 | 692571 | TRUE |
| rs7637852 | 0.0102545 | 184305 | 0.144438 | 3 | 44041777 | 0.0019 | 1.70E−13 | 691815 | TRUE |
| rs7640424 | 0.0107922 | 184305 | 0.830802 | 3 | 107820063 | 0.0018 | 2.30E−14 | 790612 | TRUE |
| rs765875 | 0.0092866 | 184305 | 0.107643 | 6 | 143185683 | 0.0017 | 3.00E−12 | 690961 | TRUE |
| rs7683836 | 0.0094224 | 184305 | 0.117409 | 4 | 180167906 | 0.0017 | 6.30E−11 | 686968 | TRUE |
| rs7685048 | 0.0092801 | 184305 | 0.422716 | 4 | 95027784 | 0.0017 | 4.10E−09 | 692398 | TRUE |
| rs768840 | 0.0104065 | 184305 | 0.378899 | 14 | 73143457 | 0.0018 | 2.00E−10 | 677485 | TRUE |
| rs769449 | 0.0165408 | 184305 | 6.13E−08 | 19 | 45410002 | 0.0027 | 2.30E−20 | 566857 | TRUE |
| rs7694732 | 0.0092898 | 184305 | 0.132691 | 4 | 115124089 | 0.0017 | 8.70E−09 | 690622 | TRUE |
| rs7703576 | 0.0109225 | 184305 | 0.0016296 | 5 | 144543996 | 0.0019 | 4.80E−08 | 690818 | TRUE |
| rs7704281 | 0.0228466 | 184305 | 0.175545 | 5 | 50591460 | 0.0041 | 6.50E−11 | 788585 | TRUE |
| rs7715256 | 0.0095842 | 184305 | 0.805256 | 5 | 153537893 | 0.0016 | 2.20E−24 | 795302 | TRUE |
| rs7724675 | 0.0109388 | 184305 | 0.254182 | 5 | 130440010 | 0.0021 | 9.50E−09 | 691968 | TRUE |
| rs7730004 | 0.0099677 | 184305 | 0.298125 | 5 | 43191033 | 0.0018 | 9.10E−16 | 690164 | TRUE |
| rs7730898 | 0.0113016 | 184305 | 0.415774 | 5 | 170459675 | 0.0018 | 4.50E−20 | 792975 | TRUE |
| rs774246 | 0.0143237 | 184305 | 0.537543 | 7 | 26990816 | 0.0025 | 5.40E−10 | 690818 | TRUE |
| rs7761673 | 0.0115634 | 184305 | 0.236177 | 6 | 70357368 | 0.0021 | 1.90E−09 | 691716 | TRUE |
| rs7780752 | 0.0097678 | 184305 | 0.439191 | 7 | 93241640 | 0.0018 | 1.00E−14 | 690830 | TRUE |
| rs7788008 | 0.0093485 | 184305 | 0.388065 | 7 | 112972483 | 0.0017 | 1.10E−19 | 690410 | TRUE |
| rs7811342 | 0.0127225 | 184305 | 0.254078 | 7 | 138794618 | 0.0029 | 1.10E−11 | 676265 | TRUE |
| rs7819514 | 0.0097613 | 184305 | 0.0317973 | 8 | 93204442 | 0.0018 | 5.70E−09 | 684955 | TRUE |
| rs7826312 | 0.0093583 | 184305 | 0.845633 | 8 | 32400115 | 0.0017 | 4.90E−10 | 785343 | TRUE |
| rs7844647 | 0.009959 | 184305 | 0.761858 | 8 | 34503776 | 0.0018 | 2.80E−11 | 793703 | TRUE |
| rs7869771 | 0.0101632 | 184305 | 0.995447 | 9 | 94180627 | 0.0019 | 4.90E−13 | 679436 | TRUE |
| rs7871866 | 0.012817 | 184305 | 0.0780243 | 9 | 131027982 | 0.0024 | 2.30E−14 | 683494 | TRUE |
| rs7899106 | 0.0233702 | 184305 | 0.770715 | 10 | 87410904 | 0.0037 | 1.00E−18 | 793689 | TRUE |
| rs7903146 | 0.0103902 | 184305 | 0.001448 | 10 | 114758349 | 0.0018 | 1.30E−23 | 795624 | TRUE |
| rs7925214 | 0.0094886 | 184305 | 0.292657 | 11 | 130794253 | 0.0018 | 4.40E−17 | 677603 | TRUE |
| rs7970953 | 0.0097939 | 184305 | 0.924998 | 12 | 24075508 | 0.0018 | 9.80E−14 | 788417 | TRUE |
| rs7983065 | 0.0095842 | 184305 | 0.000393097 | 13 | 33380786 | 0.0017 | 8.90E−18 | 690924 | TRUE |
| rs7998796 | 0.0095582 | 184305 | 0.95603 | 13 | 81020036 | 0.0018 | 1.10E−08 | 689430 | TRUE |
| rs8027205 | 0.0095875 | 184305 | 0.516088 | 15 | 98280959 | 0.0018 | 1.40E−09 | 685725 | TRUE |
| rs8036040 | 0.0094159 | 184305 | 0.0657552 | 15 | 36402716 | 0.0017 | 2.70E−10 | 691068 | TRUE |
| rs8047395 | 0.0092942 | 184305 | 0.0502678 | 16 | 53798523 | 0.0017 | 1.00E−200 | 788856 | TRUE |
| rs806600 | 0.0093387 | 184305 | 0.607709 | 5 | 172914939 | 0.0017 | 3.30E−08 | 691791 | TRUE |
| rs8071182 | 0.0116514 | 184305 | 0.0293731 | 17 | 55336155 | 0.0022 | 2.10E−09 | 771437 | TRUE |
| rs8090983 | 0.0097711 | 184305 | 0.650055 | 18 | 52586691 | 0.0018 | 2.00E−10 | 682470 | TRUE |
| rs8097672 | 0.013774 | 184305 | 0.757939 | 18 | 1839601 | 0.0025 | 8.40E−16 | 686063 | TRUE |
| rs8097783 | 0.016821 | 184305 | 0.0213216 | 18 | 58051294 | 0.0031 | 7.20E−36 | 795408 | TRUE |
| rs8123881 | 0.0145474 | 184305 | 0.741331 | 20 | 15819495 | 0.0024 | 4.40E−16 | 793018 | TRUE |
| rs8181823 | 0.0111278 | 184305 | 0.600965 | 13 | 65477940 | 0.002 | 4.10E−10 | 691345 | TRUE |
| rs818524 | 0.0100578 | 184305 | 0.911492 | 1 | 85201228 | 0.0019 | 3.40E−08 | 670078 | TRUE |
| rs8192675 | 0.0101024 | 184305 | 0.113581 | 3 | 170724883 | 0.0018 | 1.40E−17 | 795515 | TRUE |
| rs825688 | 0.0098862 | 184305 | 0.0616552 | 16 | 73595718 | 0.0017 | 4.70E−08 | 686144 | TRUE |
| rs845084 | 0.0102045 | 184305 | 0.599472 | 10 | 125220036 | 0.002 | 1.30E−12 | 685413 | TRUE |
| rs852056 | 0.0102947 | 184305 | 0.475851 | 20 | 17102860 | 0.002 | 1.80E−10 | 691874 | TRUE |
| rs865809 | 0.010488 | 184305 | 0.942764 | 3 | 183997735 | 0.002 | 5.40E−10 | 689186 | TRUE |
| rs872281 | 0.0122543 | 184305 | 0.718575 | 14 | 40834177 | 0.0023 | 4.70E−11 | 685310 | TRUE |
| rs876605 | 0.0106173 | 184305 | 0.453763 | 5 | 77801359 | 0.002 | 3.40E−08 | 692586 | TRUE |
| rs879620 | 0.009732 | 184305 | 0.762578 | 16 | 4015729 | 0.0018 | 5.30E−38 | 688377 | TRUE |
| rs889398 | 0.0097081 | 184305 | 0.00583553 | 16 | 69556715 | 0.0016 | 1.30E−32 | 789694 | TRUE |
| rs895330 | 0.0147745 | 184305 | 0.344344 | 19 | 4060707 | 0.0023 | 5.50E−19 | 684271 | TRUE |
| rs901630 | 0.0097895 | 184305 | 0.857162 | 6 | 98539519 | 0.0017 | 1.90E−18 | 794597 | TRUE |
| rs902695 | 0.0095017 | 184305 | 0.0781754 | 2 | 113955074 | 0.0017 | 2.20E−09 | 678635 | TRUE |
| rs9294260 | 0.009392 | 184305 | 0.838517 | 6 | 83433228 | 0.0016 | 1.80E−19 | 783533 | TRUE |
| rs9300422 | 0.0099069 | 184305 | 0.312443 | 13 | 98223320 | 0.0018 | 4.00E−09 | 795011 | TRUE |
| rs930295 | 0.0131527 | 184305 | 0.939153 | 2 | 50233352 | 0.0023 | 1.00E−19 | 690522 | TRUE |
| rs9304665 | 0.010639 | 184305 | 0.00182171 | 19 | 47602577 | 0.002 | 2.90E−29 | 689470 | TRUE |
| rs934224 | 0.0106955 | 184305 | 0.772796 | 2 | 16613889 | 0.002 | 4.70E−08 | 692568 | TRUE |
| rs9362662 | 0.0093474 | 184305 | 0.187214 | 6 | 90296588 | 0.0017 | 1.20E−10 | 683953 | TRUE |
| rs9367368 | 0.0102566 | 184305 | 0.263106 | 6 | 13189275 | 0.0018 | 1.00E−11 | 786723 | TRUE |
| rs9370261 | 0.0166396 | 184305 | 0.551666 | 6 | 53939516 | 0.0042 | 3.40E−08 | 690051 | TRUE |
| rs9375702 | 0.0099807 | 184305 | 0.119137 | 6 | 130384187 | 0.0019 | 7.90E−10 | 690564 | TRUE |
| rs9379827 | 0.0115721 | 184305 | 0.886619 | 6 | 26153335 | 0.0019 | 6.90E−12 | 795072 | TRUE |
| rs9408882 | 0.0091519 | 184305 | 0.928693 | 9 | 118664402 | 0.0016 | 1.30E−08 | 794283 | TRUE |
| rs946824 | 0.0133015 | 184305 | 0.918085 | 1 | 243684019 | 0.0026 | 1.10E−15 | 689849 | TRUE |
| rs947612 | 0.0104141 | 184305 | 0.384422 | 6 | 73738661 | 0.002 | 5.60E−09 | 692596 | TRUE |
| rs9478671 | 0.0120523 | 184305 | 0.758719 | 6 | 155987825 | 0.0021 | 1.70E−08 | 688072 | TRUE |
| rs9522285 | 0.0095082 | 184305 | 0.592059 | 13 | 112230701 | 0.0017 | 2.50E−13 | 690681 | TRUE |
| rs9538162 | 0.0093898 | 184305 | 0.458877 | 13 | 59265043 | 0.0018 | 4.80E−19 | 690345 | TRUE |
| rs9547153 | 0.0095201 | 184305 | 0.336333 | 13 | 85903717 | 0.0017 | 8.70E−09 | 775400 | TRUE |
| rs9571687 | 0.0099492 | 184305 | 0.895803 | 13 | 67472713 | 0.0018 | 2.80E−12 | 690974 | TRUE |
| rs9615905 | 0.0098189 | 184305 | 0.000218499 | 22 | 48875699 | 0.0017 | 2.70E−10 | 690274 | TRUE |
| rs962273 | 0.0102186 | 184305 | 1.51E−06 | 17 | 46978353 | 0.0019 | 2.60E−13 | 692594 | TRUE |
| rs9650755 | 0.0100546 | 184305 | 0.822388 | 9 | 96484342 | 0.002 | 2.80E−15 | 691183 | TRUE |
| rs9688431 | 0.0182386 | 184305 | 0.821539 | 6 | 73922654 | 0.0035 | 2.40E−11 | 789356 | TRUE |
| rs977747 | 0.0096146 | 184305 | 0.148375 | 1 | 47684677 | 0.0017 | 1.30E−24 | 793546 | TRUE |
| rs9783858 | 0.0092583 | 184305 | 0.157594 | 18 | 42534584 | 0.0017 | 3.30E−08 | 770874 | TRUE |
| rs9806742 | 0.0155522 | 184305 | 0.559589 | 15 | 73051219 | 0.0026 | 1.40E−15 | 692509 | TRUE |
| rs9816226 | 0.0122543 | 184305 | 0.837198 | 3 | 185834499 | 0.0021 | 1.60E−52 | 778333 | TRUE |
| rs9845966 | 0.009254 | 184305 | 0.198618 | 3 | 13433158 | 0.0017 | 2.50E−10 | 778076 | TRUE |
| rs987237 | 0.0115895 | 184305 | 0.029386 | 6 | 50803050 | 0.0021 | 9.30E−84 | 795612 | TRUE |
| rs9926784 | 0.011294 | 184305 | 0.763043 | 16 | 19941968 | 0.0021 | 9.90E−35 | 789617 | TRUE |
| rs9927848 | 0.0103403 | 184305 | 0.152984 | 16 | 23833071 | 0.002 | 6.40E−10 | 687060 | TRUE |
| rs9951619 | 0.010664 | 184305 | 0.842053 | 18 | 56882326 | 0.002 | 1.40E−15 | 772643 | TRUE |
| rs998732 | 0.0136067 | 184305 | 0.697114 | 19 | 19378671 | 0.0022 | 2.00E−14 | 793852 | TRUE |
| rs9989141 | 0.0096842 | 184305 | 0.727381 | 14 | 94006257 | 0.0017 | 3.60E−21 | 752768 | TRUE |
| rs999889 | 0.0103033 | 184305 | 0.137684 | 10 | 84279949 | 0.0019 | 1.40E−08 | 690572 | TRUE |

1. A method of identifying a causal relationship between a drug and an unrelated disease, the method comprising:
a) Obtaining genetic instruments for therapeutic targets of the drug;
b) Identifying genes (or alleles, or variants thereof) associated with the genetic instruments obtained from step a);
c) Obtaining effect sizes of genetic instruments for targets associated with the unrelated disease;
d) Identifying genes (or alleles, or variants thereof) associated with the genetic instruments obtained from step c);
e) Harmonising the genes obtained from step b) and the genes identified from step d);
f) Identifying overlapping genes in the harmonised genes obtained in step e); and
g) Performing two-sample Mendelian randomization using the overlapping genes identified in step e), wherein the results obtained from the two-sample Mendelian randomization identify the causal relationship, if present;
wherein the drug is a specific drug of a group of structurally or functionally related drugs.

2. The method of claim 1, wherein the genetic instruments from step a) are obtained using at least two different methods.

3. The method of claim 2, wherein genetic instruments shown to be statistically significant in both of the at least two different methods are selected.

4. The method of claim 3, wherein the method further comprises a step of validating the genetic instruments.

5. The method of claim 2, wherein the at least two different methods are GTEx instruments and GWAS instruments.

6. The method of claim 1, wherein the genetic instruments of step a) are selected from the group consisting of single nucleotide polymorphisms (SNPs), gene expression, and protein expression.

7. The method of claim 1, wherein the genetic instruments have a weak linkage disequilibrium with each other.

8. The method of claim 1, wherein the therapeutic target of the drug of step a) is a drug target gene or a protein affected by the drug.

9. The method of claim 8, wherein the drug target gene is identified using protein-protein-interaction (PPI)-based gene identification.

10. The method of claim 1, wherein the target associated with the unrelated disease is selected from the group consisting of a gene, a protein, a mutant gene, a mutant protein, a dysregulated gene, or a dysregulated a protein.

11. The method of claim 1, wherein the genetic instruments of step c) are obtained from a database selected from the group consisting of the GWAS Catalogue, Integrative Epidemiology Unit (IEU) Open GWAS, FinnGen Consortium, the Breast Cancer Association Consortium, and combinations thereof.

12. The method of claim 1, wherein the harmonisation of step e) comprises aligning the direction of alleles identified in step b) with an exposure dataset and aligning the direction of alleles identified in step d) with an outcome dataset.

13. The method of claim 1, wherein the genetic instruments of step a) are obtained from DrugBank.

14. The method of claim 1, wherein the method further comprises a step of validating the results obtained in step g).

15. The method of claim 14, wherein the step of validation is selected from the group consisting of in vitro assays, in vivo assays, and in silico assays.
